(19) United States
(12) Patent Application Publication
(10) Pub. No.: US 2025/0230441 A1
(43) Pub. Date: Jul. 17, 2025

(54) Nucleic acids for inhibiting expression of MASP-2 in a cell

(71) Applicant: SILENCE THERAPEUTICS GMBH, Berlin (DE)

(72) Inventors: Eliot Morrison, Berlin (DE); Sibylle Dames, Berlin (DE); Maciej Czajkowski, Berlin (DE); Verena Aumiller, Berlin (DE); Timo Johannssen, Berlin (DE)

(21) Appl. No.: 18/837,538

(22) PCT Filed: Feb. 10, 2023
(86) PCT No.: PCT/EP2023/053298

§ 371 Date: Aug. 9, 2024

(30) Foreign Application Priority Data
Feb. 10, 2022 (EP) 22156214.3

(51) Int. Cl. C12N 15/113

(52) U.S. Cl. CPC C12N 15/1137; C12N 2310/14; C12N 2310/321; C12N 2310/335; C12N 2310/351

(57) ABSTRACT

The invention relates to double-stranded nucleic acid molecules that interfere with or inhibit mannan-binding lectin serine protease 2 (MASP-2) gene expression. It further relates to therapeutic uses of such inhibition such as for the treatment of diseases and disorders associated with complement pathway deregulation, particularly of the Lectin pathway, and/or with over-activation or with ectopic expression or localisation or accumulation, of MASP-2 in the body.

### FIELD OF THE INVENTION

The invention relates to double-stranded nucleic acid molecules that interfere with or inhibit mannan-binding lectin serine protease 2 (MASP-2) gene expression. It further relates to therapeutic uses of such inhibition such as for the treatment of diseases and disorders associated with complement pathway deregulation, particularly of the lectin pathway, and/or with over-activation or with ectopic expression or localisation or accumulation, of MASP-2 in the body.

### BACKGROUND

Double-stranded RNAs (dsRNA) able to bind through complementary base pairing to expressed mRNAs have been shown to block gene expression (Fire et al., 1998, Nature. 1998 Feb. 19; 391(6669):806-11 and Elbashir et al., 2001, Nature. 2001 May 24; 411(6836):494-8) by a mechanism that has been termed “RNA interference (RNAi)”. Short dsRNAs direct gene specific, post transcriptional silencing in many organisms, including vertebrates, and have become a useful tool for studying gene function. RNAi is mediated by the RNA induced silencing complex (RISC), a sequence specific, multi component nuclease that degrades messenger RNAs having sufficient complementary or homology to the silencing trigger loaded into the RISC complex. Interfering RNAs such as siRNAs, antisense RNAs, and micro RNAs, are oligonucleotides that prevent the formation of proteins by gene silencing, i.e., inhibiting gene translation of the protein through degradation of mRNA molecules. Gene silencing agents are becoming increasingly important for therapeutic applications in medicine.

According to Watts and Corey in the Journal of Pathology (2012; Vol 226, p 365-379), there are algorithms that can be used to design nucleic acid silencing triggers, but all of these have severe limitations. It may take various experimental methods to identify potent siRNAs, as algorithms do not take into account factors such as tertiary structure of the target mRNA or the involvement of RNA binding proteins. Therefore, the discovery of a potent nucleic acid silencing trigger with minimal off-target effects is a complex process. For the pharmaceutical development of these highly charged molecules, it is necessary that they can be synthesised economically, distributed to target tissues, enter cells and function within acceptable limits of toxicity.

The complement system or pathway is part of the innate immune system of host defence against invading pathogens. It mainly consists of a number of proteins that circulate in the bloodstream in the form of precursors. Most of the proteins that form the complement system, including the complement component protein C3 (also referred to herein simply as C3), are largely synthesised and secreted into the bloodstream by hepatocytes in the liver. Activation of the system leads to inflammatory responses resulting in phagocyte attraction and opsonization and consequently clearance of pathogens, immune complexes and cellular debris (Janeway's Immunobiology 9th Edition). The complement system consists of 3 pathways (Classical, Lectin and Alternative pathways), which all converge at the formation of so-called complement component 3 convertase enzyme complexes. These enzyme complexes cleave the complement component C3 protein into C3a and C3b. Once cleaved, C3b forms part of a complex that in turn cleaves C5 into C5a and C5b. After cleavage, C5b is one of the key components of the main complement pathway effectors, the membrane attack complex. C3 is therefore a key component of the complement system activation pathway.

Complement Factor B (CFB or “factor B”) is involved in activation of the Alternative pathway. Binding of CFB to C3b (e.g., on a cell surface) renders CFB susceptible to cleavage by Factor D, forming the serine protease C3Bb, which is itself a C3 convertase, leading to an amplification loop for C3 activation. CFB is primarily synthesised in the liver, as well as in low levels at several extrahepatic sites.

The mannan-binding lectin serine protease 2 (also known as mannose-binding protein-associated serine protease 2, or as mannan-binding lectin serine peptidase 2, or MBL associated serine protease 2) (MASP-2) gene, encoding the protein MASP-2, is the main enzyme involved in the activation of lectin pathway of the complement system. MASP-2 can also activate the Alternative pathway of complement system through a C4/C2 bypass route that leads to MASP-2-dependent activation of C3 by an unknown mechanism. The MASP-2 gene encodes a member of the peptidase S1 family of serine proteases. The encoded preproprotein is proteolytically processed to generate A and B chains that heterodimerize to form the mature protease. This protease cleaves complement components C2 and C4 to generate C3 convertase in the lectin pathway of the complement system. The encoded protease also plays a role in the coagulation cascade through cleavage of prothrombin to form thrombin. Alternative splicing results in multiple transcript variants, at least one of which encodes an isoform that is proteolytically processed.

The MASP-2 gene encodes a 76 kDa serine protease, MASP-2 (long isoform), which is highly expressed in the liver, and a 19 kDa alternative splice product, Map19 (short isoform), which is present in plasma.

Several diseases are associated with aberrant acquired or genetic activation of the complement pathway as well as with aberrant or over-expression of MASP-2.

There are currently only few treatments for complement system mediated diseases, disorders and syndromes. The monoclonal humanized antibody Eculizumab is one of them. It is known to bind complement protein C5, thereby blocking the membrane attack complex at the end of the complement cascade (Hillmen et al., 2006 NEJM). However, only a subset of patients suffering from complement system mediated diseases respond to Eculizumab therapy. There is thus a high unmet need for medical treatments of complement mediated or associated diseases. Inhibiting expression of MASP-2 presents a promising therapeutic strategy for many complement-mediated diseases. Targeting of MASP-2 expression or activity, e.g., via antisense oligonucleotides or inhibitors, has been proposed as a potential therapeutic strategy for various complement-mediated conditions including rheumatoid arthritis (Holers et al., Front Immunol. 2020 Feb. 21; 11:201), IgA nephropathy, lupus nephritis (Lafayette et al., Kidney Int Rep. 2020 Aug. 13; 5(11):2032-2041), thrombotic microangiopathy and atypical hemolytic uremic syndrome (Elhadad et al., Clin Exp Immunol. 2021 January; 203(1):96-104; Young et al., Bone Marrow Transplant. 2021 August; 56(8):1805-1817). It has been suggested to investigate MASP-2 inhibition (via antibody) to treat pneumococcal meningitis (Kasanmoentalib et al., J Neuroinflammation. 2017 Apr. 6; 14(1):77) and coronavirus infection (Flude et al., Viruses, 2021 Feb. 17; 13(2):312).

WO2012139081A2, Lafayette et al. (Kidney Int Rep. 2020 Aug. 13; 5(11):2032-2041), Young et al. (Bone Marrow Transplant. 2021 August; 56(8):1805-1817 and Elhadad et al. (Clin Exp Immunol. 2021 January; 203(1):96-104) describe MASP-2 antibodies (e.g., narsoplimab) for treating conditions with MASP-2 dependent complement activation. US20200032270A1 and WO2021168148A1 describe double-stranded RNA agents targeting MASP-2 gene.

### SUMMARY OF THE INVENTION

One aspect of the invention is a double-stranded nucleic acid for inhibiting expression of mannan-binding lectin serine protease 2 (MASP-2), wherein the nucleic acid comprises a first strand and a second strand, wherein the unmodified equivalent of the first strand sequence comprises a sequence of at least 15 nucleotides differing by no more than 3 nucleotides from any one of the first strand sequences shown in Table 5a.

The unmodified equivalent of the first strand sequence may, for example, comprise a sequence of at least 15 nucleotides differing by no more than 3 nucleotides from any one of the first strand sequences listed in Table 1.

The nucleic acids described herein are thus double-stranded nucleic acids capable of inhibiting expression of MASP-2, preferably in a cell, and may find use as a therapeutic agent or diagnostic agent, e.g., in associated diagnostic or therapeutic methods. The nucleic acid comprises or consists of a first strand and a second strand, and the first strand typically comprises sequences sufficiently complementary to MASP-2 mRNA, so as to mediate RNA interference.

One aspect relates to a composition comprising a nucleic acid as disclosed herein and a solvent (preferably water) and/or a delivery vehicle and/or a physiologically acceptable excipient and/or a carrier and/or a salt and/or a diluent and/or a buffer and/or a preservative.

One aspect relates to a composition comprising a nucleic acid as disclosed herein and a further therapeutic agent selected from e.g., an oligonucleotide, a small molecule, a monoclonal antibody, a polyclonal antibody and a peptide.

One aspect relates to a nucleic acid or a composition comprising it as disclosed herein for use as a therapeutic agent or diagnostic agent, e.g., in associated methods.

One aspect relates to a nucleic acid or a composition comprising it as disclosed herein for use in the prophylaxis or treatment of a disease, disorder or syndrome.

One aspect relates to the use of a nucleic acid or a composition comprising it as disclosed herein in the prophylaxis or treatment of a disease, disorder or syndrome.

One aspect relates to the use of a nucleic acid or a composition comprising it as disclosed herein in the preparation of a medicament for the prophylaxis or treatment of a disease, disorder or syndrome.

One aspect relates to a method of prophylaxis or treatment of a disease, disorder or syndrome comprising administering a pharmaceutically effective dose or amount of a nucleic acid or composition comprising it as disclosed herein to an individual in need of treatment, preferably wherein the nucleic acid or composition is administered to the subject subcutaneously, intravenously or by oral, rectal, pulmonary, intramuscular or intraperitoneal administration.

### DETAILED DESCRIPTION OF THE INVENTION

The present invention relates to a nucleic acid which is double-stranded, and which comprises a sequence substantially homologous to an expressed RNA transcript of MASP-2, and compositions thereof. These nucleic acids, conjugates thereof, and compositions comprising them, may be used in the prophylaxis and treatment of a variety of diseases, disorders and syndromes in which reduced expression of the MASP-2 gene product is desirable.

A first aspect of the invention is a double-stranded nucleic acid for inhibiting expression of MASP-2, preferably in a cell, wherein the nucleic acid comprises a first strand and a second strand, wherein the unmodified equivalent of the first strand sequence comprises a sequence of at least 15 nucleotides differing by no more than 3 nucleotides from any one of the first strand sequences shown in Table 5a. These nucleic acids among others have the advantage of being active in various species that are relevant for pre-clinical and clinical development and/or of having few relevant off-target effects. Having few relevant off-target effects means that a nucleic acid specifically inhibits the intended target and does not significantly inhibit other genes or inhibits only one or few other genes at a therapeutically acceptable level.

For example, the unmodified equivalent of the first strand sequence may comprise a sequence of at least 15 nucleotides differing by no more than 3 nucleotides from any one of the first strand sequences listed in Table 1:

TABLE 1

| First strand | Second strand |
|---|---|
| sequence (SEQ ID No.) | sequence (SEQ ID No.) |
| 147 | 148 |
| 385 | 178 |
| 199 | 200 |
| 235 | 236 |
| 237 | 238 |
| 245 | 246 |
| 386 | 310 |
| 387 | 346 |
| 388 | 382 |
| 389 | 384 |
| 289 | 290 |
| 309 | 310 |
| 381 | 382 |

Preferably, the unmodified equivalent of the first strand sequence comprises a sequence of at least 16, more preferably at least 17, yet more preferably at least 18 and most preferably all 19 nucleotides differing by no more than 3 nucleotides, preferably by no more than 2 nucleotides, more preferably by no more than 1 nucleotide, and most preferably not differing by any nucleotide from any one of the first strand sequences shown in Table 1 or in Table 5a.

For example, the unmodified equivalent of the first strand sequence may comprise a sequence of at least 16, more preferably at least 17, yet more preferably at least 18 and most preferably all 19 nucleotides differing by no more than 3 nucleotides, preferably by no more than 2 nucleotides, more preferably by no more than 1 nucleotide, and most preferably not differing by any nucleotide from any one of the first strand sequences listed in Table 1 or in Table 5a.

Preferably, the unmodified equivalent of the first strand sequence of the nucleic acid consists of one of the first strand sequences shown in Table 1 or in Table 5a. The sequence may however be modified by a number of nucleic acid modifications that do not change the identity of the nucleotide. For example, modifications of the backbone or sugar residues of the nucleic acid do not change the identity of the nucleotide because the base itself remains the same as in the reference sequence.

For example, the unmodified equivalent of the first strand sequence of the nucleic acid may consist of one of the first strand sequences shown in Table 1 or in Table 5a, optionally modified by one or more of said nucleic acid modifications.

A nucleic acid that comprises a sequence according to a reference sequence herein means that the nucleic acid comprises a sequence of contiguous nucleotides in the order as defined in the reference sequence.

When reference is made herein to a reference sequence comprising, consisting essentially of, or consisting of nucleotides, this reference is not limited to the sequence with unmodified nucleotides. The same reference also encompasses the same nucleotide sequence in which one, several, such as two, three, four, five, six, seven or more, including all, nucleotides are modified by modifications such as 2′-OMe, 2′-F, a ligand, a linker, a 3′ end or 5′ end modification or any other modification. It also refers to sequences in which two or more nucleotides are linked to each other by the natural phosphodiester linkage or by any other linkage such as a phosphorothioate or a phosphorodithioate linkage.

A double-stranded nucleic acid is a nucleic acid in which the first strand and the second strand hybridise to each other over at least part of their lengths and are therefore capable of forming a duplex region under physiological conditions, such as in PBS at 37° C. at a concentration of 1 μM of each strand. The first and second strand are preferably able to hybridise to each other and therefore to form a duplex region over a region of at least 15 nucleotides, preferably 16, 17, 18 or 19 nucleotides. This duplex region comprises nucleotide base parings between the two strands, preferably based on Watson-Crick base pairing and/or wobble base pairing (such as GU base pairing). All the nucleotides of the two strands within a duplex region do not have to base pair to each other to form a duplex region. A certain number of mismatches, deletions or insertions between the nucleotide sequences of the two strands are acceptable. Overhangs on either end of the first or second strand or unpaired nucleotides at either end of the double-stranded nucleic acid are also possible. The double-stranded nucleic acid is preferably a double-stranded nucleic acid under physiological conditions, and preferably has a melting temperature (Tm) of 45° C. or more, preferably 50° C. or more, and more preferably 55° C. or more for example in PBS at a concentration of 1 μM of each strand.

A stable double-stranded nucleic acid under physiological conditions is a double-stranded nucleic acid that has a Tm of 45° C. or more, preferably 50° C. or more, and more preferably 55° C. or more, for example in PBS at a concentration of 1 μM of each strand.

The first strand and the second strand are preferably capable of forming a duplex region (i.e., are complementary to each other) over i) at least a portion of their lengths, preferably over at least 15 nucleotides of both of their lengths, ii) over the entire length of the first strand, iii) over the entire length of the second strand or iv) over the entire length of both the first and the second strand. Strands being complementary to each other over a certain length means that the strands are able to base pair to each other, either via Watson-Crick or wobble base pairing, over that length. Each nucleotide of the length does not necessarily have to be able to base pair with its counterpart in the other strand over the entire given length as long as a stable double-stranded nucleotide under physiological conditions can be formed. It is however, preferred, in certain embodiments, if each nucleotide of the length can base pair with its counterpart in the other strand over the entire given length.

A certain number of mismatches, deletions or insertions between the first strand and the target sequence, or between the first strand and the second strand can be tolerated in the context of the nucleic acids according to the present invention and even have the potential in certain cases to increase RNA interference (e.g., inhibition) activity.

The inhibition activity of the nucleic acids according to the present invention relies on the formation of a duplex region between all or a portion of the first strand and a portion of a target nucleic acid. The portion of the target nucleic acid that forms a duplex region with the first strand, defined as beginning with the first base pair formed between the first strand and the target sequence and ending with the last base pair formed between the first strand and the target sequence, inclusive, is the target nucleic acid sequence or simply, target sequence. The duplex region formed between the first strand and the second strand need not be the same as the duplex region formed between the first strand and the target sequence. That is, the second strand may have a sequence different from the target sequence; however, the first strand must be able to form a duplex structure with both the second strand and the target sequence, at least under physiological conditions.

The complementarity between the first strand and the target sequence may be perfect (i.e., 100% identity with no nucleotide mismatches or insertions or deletions in the first strand as compared to the target sequence).

The complementarity between the first strand and the target sequence may not be perfect. The complementarity may be from about 70% to about 100%. More specifically, the complementarity may be at least 70%, 80%, 85%, 90% or 95% and intermediate values.

The identity between the first strand and the complementary sequence of the target sequence may range from about 75% to about 100%. More specifically, the complementarity may be at least 75%, 80%, 85%, 90% or 95% and intermediate values, provided a nucleic acid is capable of reducing or inhibiting the expression of MASP-2.

A nucleic acid having less than 100% complementarity between the first strand and the target sequence may be able to reduce the expression of MASP-2 to the same level as a nucleic acid having perfect complementarity between the first strand and target sequence. Alternatively, it may be able to reduce expression of MASP-2 to a level that is 15%, 20%, 25%, 30%, 35%, 40%, 45%, 50%, 55%, 60%, 65%, 70%, 75%, 80%, 85%, 90%, 95%, 98%, 99% or 100% of the level of reduction achieved by the nucleic acid with perfect complementarity.

A nucleic acid of the present disclosure may be an isolated nucleic acid.

A nucleic acid of the present disclosure may be a nucleic acid wherein:
(a) the unmodified equivalent of the first strand sequence comprises a sequence differing by no more than 3 nucleotides from any one of the first strand sequences of Table 5a, and optionally wherein the unmodified equivalent of the second strand sequence comprises a sequence differing by no more than 3 nucleotides from the corresponding second strand sequence of Table 5a;
(b) the unmodified equivalent of the first strand sequence comprises a sequence differing by no more than 2 nucleotides from any one of the first strand sequences of Table 5a, and optionally wherein the unmodified equivalent of the second strand sequence comprises a sequence differing by no more than 2 nucleotides from the corresponding second strand sequence of Table 5a;
(c) the unmodified equivalent of the first strand sequence comprises a sequence differing by no more than 1 nucleotide from any one of the first strand sequences of Table 5a, and optionally wherein the unmodified equivalent of the second strand sequence comprises a sequence differing by no more than 1 nucleotide from the corresponding second strand sequence of Table 5a;
(d) the unmodified equivalent of the first strand sequence comprises a sequence corresponding to nucleotides 2 to 17 from the 5′ end of any one of the first strand sequences with a given SEQ ID No. shown in Table 5a, and optionally wherein the unmodified equivalent of the second strand sequence comprises a sequence corresponding to nucleotides 2 to 17 from the 5′ end of the corresponding second strand sequence with a given SEQ ID No. shown in Table 5a;
(e) the unmodified equivalent of the first strand sequence comprises a sequence corresponding to nucleotides 2 to 18 from the 5′ end of any one of the first strand sequences with a given SEQ ID No. shown in Table 5a, and optionally wherein the unmodified equivalent of the second strand sequence comprises a sequence corresponding to nucleotides 2 to 18 from the 5′ end of the corresponding second strand sequence with a given SEQ ID No. shown in Table 5a;
(f) the unmodified equivalent of the first strand sequence comprises a sequence corresponding to nucleotides 2 to 19 from the 5′ end of any one of the first strand sequences with a given SEQ ID No. shown in Table 5a, and optionally wherein the unmodified equivalent of the second strand sequence comprises a sequence corresponding to nucleotides 2 to 19 from the 5′ end of the corresponding second strand sequence with a given SEQ ID No. shown in Table 5a;
(g) the unmodified equivalent of the first strand sequence comprises a sequence corresponding to nucleotides 2 to 19 from the 5′ end of any one of the first strand sequences with a given SEQ ID No. shown in Table 5a, and optionally wherein the unmodified equivalent of the second strand sequence comprises a sequence corresponding to nucleotides 1 to 18 from the 5′ end of the corresponding second strand sequence with a given SEQ ID No. shown in Table 5a;
(h) the unmodified equivalent of the first strand sequence comprises a sequence of any one of the first strand sequences with a given SEQ ID No. shown in Table 5a, and optionally wherein the unmodified equivalent of the second strand sequence comprises a sequence of the corresponding second strand sequence with a given SEQ ID No. shown in Table 5a;
(i) the unmodified equivalent of the first strand sequence consists essentially of any one of the first strand sequences with a given SEQ ID No. shown in Table 5a, and optionally wherein the unmodified equivalent of the second strand sequence consists essentially of the sequence of the corresponding second strand sequence with a given SEQ ID No. shown in Table 5a;
(j) the unmodified equivalent of the first strand sequence consists of any one of the first strand sequences with a given SEQ ID No. shown in Table 5a, and optionally wherein the unmodified equivalent of the second strand sequence consists of the sequence of the corresponding second strand sequence with a given SEQ ID No. shown in Table 5a;
(k) the unmodified equivalent of the first strand sequence consists of a sequence corresponding to nucleotides 1 to 19 from the 5′ end of any one of the first strand sequences with a given SEQ ID No. shown in Table 5a, wherein said unmodified equivalent of the first strand sequence further consists of 1 (nucleotide 20 counted from the 5′end), 2 (nucleotides 20 and 21), 3 (nucleotides 20, 21 and 22), 4 (nucleotides 20, 21, 22 and 23), 5 (nucleotides 20, 21, 22, 23 and 24) or 6 (nucleotides 20, 21, 22, 23, 24 and 25) additional nucleotide(s) at the 3′end of any one of the first strand sequences with a given SEQ ID No. shown in Table 5a, and optionally wherein the unmodified equivalent of the second strand sequence comprises or consists essentially of or consists of a sequence of the corresponding second strand sequence with a given SEQ ID No. shown Table 5a;
(l) the unmodified equivalent of the first strand sequence consists of a sequence corresponding to nucleotides 1 to 19 from the 5′ end of any one of the first strand sequences with a given SEQ ID No. shown in Table 5a, wherein said unmodified equivalent of the first strand sequence further consists of 1 (nucleotide 20 counted from the 5′end), 2 (nucleotides 20 and 21), 3 (nucleotides 20, 21 and 22), 4 (nucleotides 20, 21, 22 and 23), 5 (nucleotides 20, 21, 22, 23 and 24) or 6 (nucleotides 20, 21, 22, 23, 24 and 25) additional nucleotide(s) at the 3′end of any one of the first strand sequences with a given SEQ ID No. shown in Table 5a, and wherein said unmodified equivalent of the first strand sequence consists of a contiguous region of from 17-25 nucleotides in length, preferably of from 18-24 nucleotides in length, complementary to the MASP-2 transcript of SEQ ID NO. 821; and optionally wherein the unmodified equivalent of the second strand sequence comprises or consists essentially of or consists of a sequence of the corresponding second strand sequence with a given SEQ ID No. shown in Table 5a;
(m) unmodified equivalent of the first strand and the unmodified equivalent of the second strand of any one of the nucleic acid molecules of subsections (a) to (l) above are present on a single strand wherein the unmodified equivalent of the first strand and the unmodified equivalent of the second strand are able to hybridise to each other and to thereby form a double-stranded nucleic acid with a duplex region of 17, 18, 19, 20, 21, 22, 23, 24 or 25 nucleotides in length; or
(n) the unmodified equivalent of the first strand and the unmodified equivalent of the second strand of any one of the nucleic acid molecules of subsections (a) to (l) above are on two separate strands that are able to hybridise to each other and to thereby form a double-stranded nucleic acid with a duplex region of 17, 18, 19, 20, 21, 22, 23, 24 or 25 nucleotides in length.

For example, a nucleic acid of the present disclosure may be a nucleic acid wherein:
(a) the unmodified equivalent of the first strand sequence comprises a sequence differing by no more than 3 nucleotides from any one of the first strand sequences of Table 1, and optionally wherein the unmodified equivalent of the second strand sequence comprises a sequence differing by no more than 3 nucleotides from the corresponding second strand sequence of Table 1;
(b) the unmodified equivalent of the first strand sequence comprises a sequence differing by no more than 2 nucleotides from any one of the first strand sequences of Table 1, and optionally wherein the unmodified equivalent of the second strand sequence comprises a sequence differing by no more than 2 nucleotides from the corresponding second strand sequence of Table 1;
(c) the unmodified equivalent of the first strand sequence comprises a sequence differing by no more than 1 nucleotide from any one of the first strand sequences of Table 1, and optionally wherein the unmodified equivalent of the second strand sequence comprises a sequence differing by no more than 1 nucleotide from the corresponding second strand sequence of Table 1;
(d) the unmodified equivalent of the first strand sequence comprises a sequence corresponding to nucleotides 2 to 17 from the 5′ end of any one of the first strand sequences with a given SEQ ID No. shown in Table 1, and optionally wherein the unmodified equivalent of the second strand sequence comprises a sequence corresponding to nucleotides 2 to 17 from the 5′ end of the corresponding second strand sequence with a given SEQ ID No. shown in Table 1;
(e) the unmodified equivalent of the first strand sequence comprises a sequence corresponding to nucleotides 2 to 18 from the 5′ end of any one of the first strand sequences with a given SEQ ID No. shown in Table 1, and optionally wherein the unmodified equivalent of the second strand sequence comprises a sequence corresponding to nucleotides 2 to 18 from the 5′ end of the corresponding second strand sequence with a given SEQ ID No. shown in Table 1;
(f) the unmodified equivalent of the first strand sequence comprises a sequence corresponding to nucleotides 2 to 19 from the 5′ end of any one of the first strand sequences with a given SEQ ID No. shown in Table 1, and optionally wherein the unmodified equivalent of the second strand sequence comprises a sequence corresponding to nucleotides 2 to 19 from the 5′ end of the corresponding second strand sequence with a given SEQ ID No. shown in Table 1;
(g) the unmodified equivalent of the first strand sequence comprises a sequence corresponding to nucleotides 2 to 19 from the 5′ end of any one of the first strand sequences with a given SEQ ID No. shown in Table 1, and optionally wherein the unmodified equivalent of the second strand sequence comprises a sequence corresponding to nucleotides 1 to 18 from the 5′ end of the corresponding second strand sequence with a given SEQ ID No. shown in Table 1;
(h) the unmodified equivalent of the first strand sequence comprises a sequence of any one of the first strand sequences with a given SEQ ID No. shown in Table 1, and optionally wherein the unmodified equivalent of the second strand sequence comprises a sequence of the corresponding second strand sequence with a given SEQ ID No. shown in Table 1;
(i) the unmodified equivalent of the first strand sequence consists of any one of the first strand sequences with a given SEQ ID No. shown in Table 1, and optionally wherein the unmodified equivalent of the second strand sequence consists of the sequence of the corresponding second strand sequence with a given SEQ ID No. shown in Table 1;
(j) the unmodified equivalent of the first strand sequence consists essentially of any one of the first strand sequences with a given SEQ ID No. shown in Table 1, and optionally wherein the unmodified equivalent of the second strand sequence consists essentially of the sequence of the corresponding second strand sequence with a given SEQ ID No. shown in Table 1;
(k) the unmodified equivalent of the first strand sequence consists of a sequence corresponding to nucleotides 1 to 19 from the 5′ end of any one of the first strand sequences with a given SEQ ID No. shown in Table 1, wherein said unmodified equivalent of the first strand sequence further consists of 1 (nucleotide 20 counted from the 5′end), 2 (nucleotides 20 and 21), 3 (nucleotides 20, 21 and 22), 4 (nucleotides 20, 21, 22 and 23), 5 (nucleotides 20, 21, 22, 23 and 24) or 6 (nucleotides 20, 21, 22, 23, 24 and 25) additional nucleotide(s) at the 3′end of any one of the first strand sequences with a given SEQ ID No. shown in Table 1, and optionally wherein the unmodified equivalent of the second strand sequence comprises or consists essentially of or consists of a sequence of the corresponding second strand sequence with a given SEQ ID No. shown Table 1;
(l) the unmodified equivalent of the first strand sequence consists of a sequence corresponding to nucleotides 1 to 19 from the 5′ end of any one of the first strand sequences with a given SEQ ID No. shown in Table 1, wherein said unmodified equivalent of the first strand sequence further consists of 1 (nucleotide 20 counted from the 5′end), 2 (nucleotides 20 and 21), 3 (nucleotides 20, 21 and 22), 4 (nucleotides 20, 21, 22 and 23), 5 (nucleotides 20, 21, 22, 23 and 24) or 6 (nucleotides 20, 21, 22, 23, 24 and 25) additional nucleotide(s) at the 3′end of any one of the first strand sequences with a given SEQ ID No. shown in Table 1, and wherein said unmodified equivalent of the first strand sequence consists of a contiguous region of from 17-25 nucleotides in length, preferably of from 18-24 nucleotides in length, complementary to the MASP-2 transcript of SEQ ID NO. 821; and optionally wherein the unmodified equivalent of the second strand sequence comprises or consists essentially of or consists of a sequence of the corresponding second strand sequence with a given SEQ ID No. shown in Table 1;
(m) unmodified equivalent of the first strand and the unmodified equivalent of the second strand of any one of the nucleic acid molecules of subsections (a) to (l) above are present on a single strand wherein the unmodified equivalent of the first strand and the unmodified equivalent of the second strand are able to hybridise to each other and to thereby form a double-stranded nucleic acid with a duplex region of 17, 18, 19, 20, 21, 22, 23, 24 or 25 nucleotides in length; or
(n) the unmodified equivalent of the first strand and the unmodified equivalent of the second strand of any one of the nucleic acid molecules of subsections (a) to (l) above are on two separate strands that are able to hybridise to each other and to thereby form a double-stranded nucleic acid with a duplex region of 17, 18, 19, 20, 21, 22, 23, 24 or 25 nucleotides in length.

By a “corresponding” second strand is meant a second strand present in the same duplex as a given first strand in Table 5a, 5b or 5c, or listed as a corresponding second strand sequence in Table 1 or Table 2, as the case may be. That is to say, a first strand and its corresponding second strand are designated as the “A” and “B” strands respectively of a duplex having a given Duplex ID in Table 5a, 5b or 5c, or are described as such in Tables 1 and 2.

In one aspect, if the 5′-most nucleotide of the first strand is a nucleotide other than A or U, this nucleotide is replaced by an A or U. Preferably, if the 5′-most nucleotide of the first strand is a nucleotide other than U, this nucleotide is replaced by U, and more preferably by U with a 5′ vinylphosphonate.

When a nucleic acid of the invention does not comprise the entire sequence of a reference first strand and/or second strand sequence (as for example given in Tables 1, 2, 5a, 5b or 5c), or one or both strands differ from the corresponding reference sequence by one, two or three nucleotides, this nucleic acid preferably retains at least 30%, more preferably at least 50%, more preferably at least 70%, more preferably at least 80%, even more preferably at least 90%, yet more preferably at least 95% and most preferably at least 100% of the MASP-2 inhibition activity compared to the inhibition activity of the corresponding nucleic acid that comprises the entire first strand and second strand reference sequences in a comparable experiment.

Nucleic acids that are capable of hybridising under physiological conditions are nucleic acids that are capable of forming base pairs, preferably Watson-Crick or wobble base-pairs, between at least a portion of the opposed nucleotides in the strands so as to form at least a duplex region. Such a double-stranded nucleic acid is preferably a stable double-stranded nucleic acid under physiological conditions (for example in PBS at 37° C. at a concentration of 1 μM of each strand), meaning that under such conditions, the two strands stay hybridised to each other. The Tm of the double-stranded nucleotide is preferably 45° C. or more, preferably 50° C. or more and more preferably 55° C. or more.

One aspect of the present invention relates to a nucleic acid for inhibiting expression of MASP-2, wherein the nucleic acid comprises a first sequence of at least 15, preferably at least 16, more preferably at least 17, yet more preferably at least 18 and most preferably all nucleotides differing by no more than 3 nucleotides, preferably no more than 2 nucleotides, more preferably no more than 1 nucleotide and most preferably not differing by any nucleotide from any of the first strand unmodified equivalent sequences of Table 5a, or of Table 1, the first sequence being able to hybridise to a target gene transcript (such as an mRNA) under physiological conditions. Preferably, the nucleic acid further comprises a second sequence of at least 15, preferably at least 16, more preferably at least 17, yet more preferably at least 18 and most preferably all nucleotides differing by no more than 3 nucleotides, preferably no more than 2 nucleotides, more preferably no more than 1 nucleotide and most preferably not differing by any nucleotide from any of the corresponding second strand unmodified equivalent sequences of Table 5a, or of Table 1, the second sequence being able to hybridise to the first sequence under physiological conditions and preferably the nucleic acid being an siRNA that is capable of inhibiting MASP-2 expression via the RNAi pathway.

One aspect relates to any double-stranded nucleic acid as disclosed in Tables 1, 2, 5a, 5b or 5c, each of which may be referred to by a given Duplex ID, preferably for inhibiting expression of MASP-2, provided that the double-stranded nucleic acid is able to inhibit expression of MASP-2. These nucleic acids are all siRNAs.

One aspect relates to a double-stranded nucleic acid that is capable of inhibiting expression of MASP-2, preferably in a cell, for use as a therapeutic or diagnostic agent, e.g., in associated therapeutic or diagnostic methods, wherein the nucleic acid preferably comprises or consists of a first strand and a second strand and preferably wherein the first strand comprises sequences sufficiently complementary to a MASP-2 mRNA so as to mediate RNA interference.

The nucleic acids described herein may be capable of inhibiting the expression of MASP-2. Inhibition may be complete, i.e., 0% remaining expression. Inhibition of MASP-2 expression may be partial, i.e., it may be 15%, 20%, 30%, 40%, 50%, 60%, 70%, 75%, 80%, 85%, 90%, 95% or more, or intermediate values of inhibition of the level of MASP-2 expression in the absence of a nucleic acid of the invention. The level of inhibition may be measured by comparing a treated sample with an untreated sample or with a sample treated with a control such as for example a siRNA that does not target MASP-2. Inhibition may be measured by measuring MASP-2 mRNA and/or protein levels or levels of a biomarker or indicator that correlates with MASP-2 presence or activity. It may be measured in cells that may have been treated in vitro with a nucleic acid described herein. Alternatively, or in addition, inhibition may be measured in cells, such as hepatocytes, or tissue, such as liver tissue, or an organ, such as the liver, or in a body fluid such as blood, serum, lymph or any other body part or fluid that has been taken from a subject previously treated with a nucleic acid disclosed herein. Preferably, inhibition of MASP-2 expression is determined by comparing the MASP-2 mRNA level measured in MASP-2-expressing cells after 24- or 48-hours in vitro treatment with a double-stranded RNA disclosed herein under ideal conditions (see the examples for appropriate concentrations and conditions) to the MASP-2 mRNA level measured in control cells that were untreated or mock treated or treated with a control double-stranded RNA under the same conditions.

One aspect of the present invention relates to a nucleic acid, wherein the first strand and the second strand are present on a single strand of a nucleic acid that loops around so that the first strand and the second strand are able to hybridise to each other and to thereby form a double-stranded nucleic acid with a duplex region.

Preferably, the first strand and the second strand of the nucleic acid are separate strands. The two separate strands are preferably each 17-25 nucleotides in length, more preferably 18-25 nucleotides in length. The two strands may be of the same or different lengths. The first strand may be 17-25 nucleotides in length, preferably it may be 18-24 nucleotides in length, it may be 18, 19, 20, 21, 22, 23 or 24 nucleotides in length. Most preferably, the first strand is 19 nucleotides in length. The second strand may independently be 17-25 nucleotides in length, preferably it may be 18-24 nucleotides in length, it may be 18, 19, 20, 21, 22, 23 or 24 nucleotides in length. More preferably, the second strand is 18 or 19 or 20 nucleotides in length, and most preferably it is 19 nucleotides in length.

Preferably, the first strand and the second strand of the nucleic acid form a duplex region of 17-25 nucleotides in length. More preferably, the duplex region is 18-24 nucleotides in length. The duplex region may be 17, 18, 19, 20, 21, 22, 23, 24 or 25 nucleotides in length. In the most preferred embodiment, the duplex region is 18 or 19 nucleotides in length. The duplex region is defined here as the region between and including the 5′-most nucleotide of the first strand that is base paired to a nucleotide of the second strand to the 3′-most nucleotide of the first strand that is base paired to a nucleotide of the second strand. The duplex region may comprise nucleotides in either or both strands that are not base-paired to a nucleotide in the other strand. It may comprise one, two, three or four such nucleotides on the first strand and/or on the second strand. However, preferably, the duplex region consists of 17-25 consecutive nucleotide base pairs. That is to say that it preferably comprises 17-25 consecutive nucleotides on both of the strands that all base pair to a nucleotide in the other strand. More preferably, the duplex region consists of 18 or 19 consecutive nucleotide base pairs, most preferably 18.

In each of the embodiments disclosed herein, the nucleic acid may be blunt ended at both ends; have an overhang at one end and a blunt end at the other end; or have an overhang at both ends.

The nucleic acid may have an overhang at one end and a blunt end at the other end. The nucleic acid may have an overhang at both ends. The nucleic acid may be blunt ended at both ends. The nucleic acid may be blunt ended at the end with the 5′ end of the first strand and the 3′ end of the second strand or at the 3′ end of the first strand and the 5′ end of the second strand.

The nucleic acid may comprise an overhang at a 3′ or 5′ end. The nucleic acid may have a 3′ overhang on the first strand. The nucleic acid may have a 3′ overhang on the second strand. The nucleic acid may have a 5′ overhang on the first strand. The nucleic acid may have a 5′ overhang on the second strand. The nucleic acid may have an overhang at both the 5′ end and 3′ end of the first strand. The nucleic acid may have an overhang at both the 5′ end and 3′ end of the second strand. The nucleic acid may have a 5′ overhang on the first strand and a 3′ overhang on the second strand. The nucleic acid may have a 3′ overhang on the first strand and a 5′ overhang on the second strand. The nucleic acid may have a 3′ overhang on the first strand and a 3′ overhang on the second strand. The nucleic acid may have a 5′ overhang on the first strand and a 5′ overhang on the second strand.

An overhang at the 3′ end or 5′ end of the second strand or the first strand may consist of 1, 2, 3, 4 and 5 nucleotides in length. Optionally, an overhang may consist of 1 or 2 nucleotides, which may or may not be modified.

In one embodiment, the 5′ end of the first strand is a single-stranded overhang of one, two or three nucleotides, preferably of one nucleotide.

Preferably, the nucleic acid is an siRNA. siRNAs are short interfering or short silencing RNAs that are able to inhibit the expression of a target gene through the RNA interference (RNAi) pathway. Inhibition occurs through targeted degradation of mRNA transcripts of the target gene after transcription. The siRNA forms part of the RISC complex. The RISC complex specifically targets the target RNA by sequence complementarity of the first (antisense) strand with the target sequence.

Preferably, the nucleic acid mediates RNA interference (RNAi). Preferably, the nucleic acid mediates RNA interference with an efficacy of at least 50% inhibition, more preferably at least 70%, more preferably at least 80%, even more preferably at least 90%, yet more preferably at least 95% and most preferably 100% inhibition. The inhibition efficacy is preferably measured by comparing the MASP-2 mRNA level in cells, such as hepatocytes, treated with a MASP-2 specific siRNA to the MASP-2 mRNA level in cells treated with a control in a comparable experiment. The control can be a treatment with a non-MASP-2 targeting siRNA or without a siRNA. The nucleic acid, or at least the first strand of the nucleic acid, is therefore preferably able to be incorporated into the RISC complex. As a result, the nucleic acid, or at least the first strand of the nucleic acid, is therefore able to guide the RISC complex to a specific target RNA with which the nucleic acid, or at least the first strand of the nucleic acid, is at least partially complementary. The RISC complex then specifically cleaves this target RNA and, as a result, leads to inhibition of the expression of the gene from which the RNA stems.

### Nucleic Acid Modifications

Nucleic acids discussed herein include unmodified RNA as well as RNA which has been modified, e.g., to improve efficacy or stability. Unmodified RNA refers to a molecule in which the components of the nucleic acid, namely sugars, bases, and phosphate moieties, are the same or essentially the same as those which occur in nature, for example as occur naturally in the human body. The term “modified nucleotide” as used herein refers to a nucleotide in which one or more of the components of the nucleotide, namely the sugar, base, and phosphate moiety, is/are different from those which occur in nature. The term “modified nucleotide” also refers in certain cases to molecules that are not nucleotides in the strict sense of the term because they lack, or have a substitute of, an essential component of a nucleotide, such as the sugar, base or phosphate moiety. A nucleic acid comprising such modified nucleotides is still to be understood as being a nucleic acid, even if one or more of the nucleotides of the nucleic acid has been replaced by a modified nucleotide that lacks, or has a substitution of, an essential component of a nucleotide.

Modifications of the nucleic acid of the present invention generally provide a powerful tool in overcoming potential limitations including, but not limited to, in vitro and in vivo stability and bioavailability inherent to native RNA molecules. The nucleic acids according to the invention may be modified by chemical modifications. Modified nucleic acids can also minimise the possibility of inducing interferon activity in humans. Modifications can further enhance the functional delivery of a nucleic acid to a target cell. The modified nucleic acids of the present invention may comprise one or more chemically modified ribonucleotides of either or both of the first strand or the second strand. A ribonucleotide may comprise a chemical modification of the base, sugar or phosphate moieties. The ribonucleic acid may be modified by substitution with or insertion of analogues of nucleic acids or bases.

Throughout the description of the invention, “same or common modification” means the same modification to any nucleotide, be that A, G, C or U modified with a group such as a methyl group (2′-OMe) or a fluoro group (2′-F). For example, 2′-F-dU, 2′-F-dA, 2′-F-dC, 2′-F-dG are all considered to be the same or common modification, as are 2′-OMe-rU, 2′-OMe-rA; 2′-OMe-rC; 2′-OMe-rG. In contrast, a 2′-F modification is a different modification compared to a 2′-OMe modification.

Preferably, at least one nucleotide of the first and/or second strand of the nucleic acid is a modified nucleotide, preferably a non-naturally occurring nucleotide such as preferably a 2′-F modified nucleotide.

A modified nucleotide can be a nucleotide with a modification of the sugar group. The 2′ hydroxyl group (OH) can be modified or replaced with a number of different “oxy” or “deoxy” substituents.

Examples of “oxy”-2′ hydroxyl group modifications include alkoxy or aryloxy (OR, e.g., R═H, alkyl (such as methyl), cycloalkyl, aryl, aralkyl, heteroaryl or sugar); polyethyleneglycols (PEG), O(CH₂CH₂O)ₙCH₂CH₂OR; “locked” nucleic acids (LNA) in which the 2′ hydroxyl is connected, e.g., by a methylene bridge, to the 4′ carbon of the same ribose sugar; O-AMINE (AMINE=NH₂, alkylamino, dialkylamino, heterocyclyl, arylamino, diaryl amino, heteroaryl amino, or diheteroaryl amino, ethylene diamine, or polyamino) and aminoalkoxy, O(CH₂)nAMINE, (e.g., AMINE=NH₂, alkylamino, dialkylamino, heterocyclyl, arylamino, diaryl amino, heteroaryl amino, or diheteroaryl amino, ethylene diamine, or polyamino).

“Deoxy” modifications include hydrogen, halogen, amino (e.g., NH₂, alkylamino, dialkylamino, heterocyclyl, arylamino, diaryl amino, heteroaryl amino, diheteroaryl amino, or amino acid); NH(CH₂CH₂NH), CH₂CH₂-AMINE (AMINE=NH₂, alkylamino, dialkylamino, heterocyclyl, arylamino, diaryl amino, heteroaryl amino, or diheteroaryl amino), —NHC(O)R (R=alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino functionality. Other substituents of certain embodiments include 2′-methoxyethyl, 2′-OCH₃, 2′-O-allyl, 2′-C-allyl, and 2′-fluoro.

The sugar group can also contain one or more carbons that possess the opposite stereochemical configuration than that of the corresponding carbon in ribose. Thus, a modified nucleotide may contain a sugar such as arabinose.

Modified nucleotides can also include “abasic” sugars, which lack a nucleobase at C-1′. These abasic sugars can further contain modifications at one or more of the constituent sugar atoms.

The 2′ modifications may be used in combination with one or more phosphate internucleoside linker modifications (e.g., phosphorothioate or phosphorodithioate).

One or more nucleotides of a nucleic acid of the present invention may be modified. The nucleic acid may comprise at least one modified nucleotide. The modified nucleotide may be in the first strand. The modified nucleotide may be in the second strand. The modified nucleotide may be in the duplex region. The modified nucleotide may be outside the duplex region, i.e., in a single-stranded region. The modified nucleotide may be on the first strand and may be outside the duplex region. The modified nucleotide may be on the second strand and may be outside the duplex region. The 3′-terminal nucleotide of the first strand may be a modified nucleotide. The 3′-terminal nucleotide of the second strand may be a modified nucleotide. The 5′-terminal nucleotide of the first strand may be a modified nucleotide. The 5′-terminal nucleotide of the second strand may be a modified nucleotide.

A nucleic acid of the invention may have 1 modified nucleotide or a nucleic acid of the invention may have about 2-4 modified nucleotides, or a nucleic acid may have about 4-6 modified nucleotides, about 6-8 modified nucleotides, about 8-10 modified nucleotides, about 10-12 modified nucleotides, about 12-14 modified nucleotides, about 14-16 modified nucleotides about 16-18 modified nucleotides, about 18-20 modified nucleotides, about 20-22 modified nucleotides, about 22-24 modified nucleotides, about 24-26 modified nucleotides or about 26-28 modified nucleotides. In each case the nucleic acid comprising said modified nucleotides retains at least 50% of its activity as compared to the same nucleic acid but without said modified nucleotides or vice versa. The nucleic acid may retain 55%, 60%, 65%, 70%, 75%, 80%, 85%, 90%, 95% or 100% and intermediate values of its activity as compared to the same nucleic acid but without said modified nucleotides, or may have more than 100% of the activity of the same nucleic acid without said modified nucleotides.

The modified nucleotide may be a purine or a pyrimidine. At least half of the purines may be modified. At least half of the pyrimidines may be modified. All of the purines may be modified. All of the pyrimidines may be modified. The modified nucleotides may be selected from the group consisting of a 3′ terminal deoxy thymine (dT) nucleotide, a 2′-O-methyl (2′-OMe) modified nucleotide, a 2′ modified nucleotide, a 2′ deoxy modified nucleotide, a locked nucleotide, an abasic nucleotide, a 2′ amino modified nucleotide, a 2′ alkyl modified nucleotide, a 2′-deoxy-2′-fluoro (2′-F) modified nucleotide, a morpholino nucleotide, a phosphoramidate, a non-natural base comprising nucleotide, a nucleotide comprising a 5′-phosphorothioate group, a nucleotide comprising a 5′ phosphate or 5′ phosphate mimic and a terminal nucleotide linked to a cholesteryl derivative or a dodecanoic acid bisdecylamide group.

The nucleic acid may comprise a nucleotide comprising a modified base, wherein the base is selected from 2-aminoadenosine, 2,6-diaminopurine, inosine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidine (e.g., 5-methylcytidine), 5-alkyluridine (e.g., ribothymidine), 5-halouridine (e.g., 5-bromouridine), 6-azapyrimidine, 6-alkylpyrimidine (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid and 2-thiocytidine.

Many of the modifications described herein and that occur within a nucleic acid will be repeated within a polynucleotide molecule, such as a modification of a base, or a phosphate moiety, or a non-linking O of a phosphate moiety. In some cases, the modification will occur at all of the possible positions/nucleotides in the polynucleotide but in many cases it will not. A modification may only occur at a 3′ or 5′ terminal position, may only occur in a terminal region, such as at a position on a terminal nucleotide or in the last 2, 3, 4, 5, or 10 nucleotides of a strand. A modification may occur in a double-strand region, a single-strand region, or in both. A modification may occur only in the double-strand region of a nucleic acid of the invention or may only occur in a single-strand region of a nucleic acid of the invention. A phosphorothioate or phosphorodithioate modification at a non-linking O position may only occur at one or both termini, may only occur in a terminal region, e.g., at a position on a terminal nucleotide or in the last 2, 3, 4 or 5 nucleotides of a strand, or may occur in duplex and/or in single-strand regions, particularly at termini. The 5′ end and/or 3′ end may be phosphorylated.

Stability of a nucleic acid of the invention may be increased by including particular bases in overhangs, or by including modified nucleotides, in single-strand overhangs, e.g., in a 5′ or 3′ overhang, or in both. Purine nucleotides may be included in overhangs. All or some of the bases in a 3′ or 5′ overhang may be modified. Modifications can include the use of modifications at the 2′ OH group of the ribose sugar, the use of deoxyribonucleotides, instead of ribonucleotides, and modifications in the phosphate group, such as phosphorothioate or phosphorodithioate modifications. Overhangs need not be homologous with the target sequence.

Nucleases can hydrolyse nucleic acid phosphodiester bonds. However, chemical modifications to nucleic acids can confer improved properties, and can render oligoribonucleotides more stable to nucleases.

Modified nucleic acids, as used herein, can include one or more of:
(i) alteration, e.g., replacement, of one or both of the non-linking phosphate oxygens and/or of one or more of the linking phosphate oxygens (referred to as linking even if at the 5′ and 3′ terminus of the nucleic acid of the invention);
(ii) alteration, e.g., replacement, of a constituent of the ribose sugar, e.g., of the 2′ hydroxyl on the ribose sugar;
(iii) replacement of the phosphate moiety with “dephospho” linkers;
(iv) modification or replacement of a naturally occurring base;
(v) replacement or modification of the ribose-phosphate backbone; and
(vi) modification of the 3′ end or 5′ end of the first strand and/or the second strand, e.g., removal, modification or replacement of a terminal phosphate group or conjugation of a moiety, e.g., a fluorescently labelled moiety, to either the 3′ or 5′ end of one or both strands.

The terms “replacement”, “modification” and “alteration” indicate a difference from a naturally occurring molecule.

Specific modifications are discussed in more detail below.

The nucleic acid may comprise one or more nucleotides on the second and/or first strands that are modified. Alternating nucleotides may be modified, to form modified nucleotides.

“Alternating” as described herein means to occur one after another in a regular way. In other words, alternating means to occur in turn repeatedly. For example, if one nucleotide is modified, the next contiguous nucleotide is not modified and the following contiguous nucleotide is modified and so on. One nucleotide may be modified with a first modification, the next contiguous nucleotide may be modified with a second modification and the following contiguous nucleotide is modified with the first modification and so on, where the first and second modifications are different.

Some representative modified nucleic acid sequences of the present invention are shown in the examples. These examples are meant to be representative and not limiting.

In one aspect of the nucleic acid, at least nucleotides 2 and 14 of the first strand are modified, preferably by a first common modification, the nucleotides being numbered consecutively starting with nucleotide number 1 at the 5′ end of the first strand. The first modification is preferably 2′-F.

In one aspect, at least one, several or preferably all the even-numbered nucleotides of the first strand are modified, preferably by a first common modification, the nucleotides being numbered consecutively starting with nucleotide number 1 at the 5′ end of the first strand. The first modification is preferably 2′-F.

In one aspect, at least one, several or preferably all the odd-numbered nucleotides of the first strand are modified, the nucleotides being numbered consecutively starting with nucleotide number 1 at the 5′ end of the first strand. Preferably, they are modified by a second modification. This second modification is preferably different from the first modification if the nucleic acid also comprises a first modification, for example of nucleotides 2 and 14 or of all the even-numbered nucleotides of the first strand. The first modification is preferably any 2′ ribose modification that is of the same size or smaller in volume than a 2′-OH group, or a locked nucleic acid (LNA), or an unlocked nucleic acid (UNA), or a 2′-Fluoroarabino Nucleic Acid (FANA) modification. A 2′ ribose modification that is of the same size or smaller in volume than a 2′-OH group can for example be a 2′-F, 2′-H, 2′-halo, or 2′-NH₂. The second modification is preferably any 2′ ribose modification that is larger in volume than a 2′-OH group. A 2′ ribose modification that is larger in volume than a 2′-OH group can for example be a 2′-OMe, 2′-O-MOE (2′-O-methoxyethyl), 2′-O-allyl or 2′-O-alkyl, with the proviso that the nucleic is capable of reducing the expression of the target gene to at least the same extent as the same nucleic acid without the modification(s) under comparable conditions. The first modification is preferably 2′-F and/or the second modification is preferably 2′-OMe.

In the context of this disclosure, the size or volume of a substituent, such as a 2′ ribose modification, is preferably measured as the van der Waals volume.

In one aspect, at least one, several or preferably all the nucleotides of the second strand in a position corresponding to an even-numbered nucleotide of the first strand are modified, preferably by a third modification. Preferably in the same nucleic acid nucleotides 2 and 14 or all the even numbered nucleotides of the first strand are modified with a first modification. In addition, or alternatively, the odd-numbered nucleotides of the first strand are modified with a second modification. Preferably, the third modification is different from the first modification and/or the third modification is the same as the second modification. The first modification is preferably any 2′ ribose modification that is of the same size or smaller in volume than a 2′-OH group, or a locked nucleic acid (LNA), or an unlocked nucleic acid (UNA), or a 2′-Fluoroarabino Nucleic Acid (FANA) modification. A 2′ ribose modification that is of the same size or smaller in volume than a 2′-OH group can for example be a 2′-F, 2′-H, 2′-halo, or 2′-NH₂. The second and/or third modification is preferably any 2′ ribose modification that is larger in volume than a 2′-OH group. A 2′ ribose modification that is larger in volume than a 2′-OH group can for example be a 2′-OMe, 2′-O-MOE (2′-O-methoxyethyl), 2′-O-allyl or 2′-O-alkyl, with the proviso that the nucleic is capable of reducing the expression of the target gene to at least the same extent as the same nucleic acid without the modification(s) under comparable conditions. The first modification is preferably 2′-F and/or the second and/or third modification is/are preferably 2′-OMe. The nucleotides on the first strand are numbered consecutively starting with nucleotide number 1 at the 5′ end of the first strand.

A nucleotide of the second strand that is in a position corresponding, for example, to an even-numbered nucleotide of the first strand is a nucleotide of the second strand that is base-paired to an even-numbered nucleotide of the first strand.

In one aspect, at least one, several or preferably all the nucleotides of the second strand in a position corresponding to an odd-numbered nucleotide of the first strand are modified, preferably by a fourth modification. Preferably in the same nucleic acid nucleotides 2 and 14 or all the even numbered nucleotides of the first strand are modified with a first modification. In addition, or alternatively, the odd-numbered nucleotides of the first strand are modified with a second modification. In addition, or alternatively, all the nucleotides of the second strand in a position corresponding to an even-numbered nucleotide of the first strand are modified with a third modification. The fourth modification is preferably different from the second modification and preferably different from the third modification and the fourth modification is preferably the same as the first modification. The first and/or fourth modification is preferably any 2′ ribose modification that is of the same size or smaller in volume than a 2′-OH group, or a locked nucleic acid (LNA), or an unlocked nucleic acid (UNA), or a 2′-Fluoroarabino Nucleic Acid (FANA) modification. A 2′ ribose modification that is of the same size or smaller in volume than a 2′-OH group can for example be a 2′-F, 2′-H, 2′-halo, or 2′-NH₂. The second and/or third modification is preferably any 2′ ribose modification that is larger in volume than a 2′-OH group. A 2′ ribose modification that is larger in volume than a 2′-OH group can for example be a 2′-OMe, 2′-O-MOE (2′-O-methoxyethyl), 2′-O-allyl or 2′-O-alkyl, with the proviso that the nucleic is capable of reducing the expression of the target gene to at least the same extent as the same nucleic acid without the modification(s) under comparable conditions. The first and/or the fourth modification is/are preferably a 2′-OMe modification and/or the second and/or third modification is/are preferably a 2′-F modification. The nucleotides on the first strand are numbered consecutively starting with nucleotide number 1 at the 5′ end of the first strand.

In one aspect of the nucleic acid, the nucleotide/nucleotides of the second strand in a position corresponding to nucleotide 11 or nucleotide 13 or nucleotides 11 and 13 or nucleotides 11-13 of the first strand is/are modified by a fourth modification. Preferably, all the nucleotides of the second strand other than the nucleotide/nucleotides in a position corresponding to nucleotide 11 or nucleotide 13 or nucleotides 11 and 13 or nucleotides 11-13 of the first strand is/are modified by a third modification. Preferably in the same nucleic acid nucleotides 2 and 14 or all the even numbered nucleotides of the first strand are modified with a first modification. In addition, or alternatively, the odd-numbered nucleotides of the first strand are modified with a second modification. The fourth modification is preferably different from the second modification and preferably different from the third modification and the fourth modification is preferably the same as the first modification. The first and/or fourth modification is preferably any 2′ ribose modification that is of the same size or smaller in volume than a 2′-OH group, or a locked nucleic acid (LNA), or an unlocked nucleic acid (UNA), or a 2′-Fluoroarabino Nucleic Acid (FANA) modification. A 2′ ribose modification that is of the same size or smaller in volume than a 2′-OH group can for example be a 2′-F, 2′-H, 2′-halo, or 2′-NH₂. The second and/or third modification is preferably any 2′ ribose modification that is larger in volume than a 2′-OH group. A 2′ ribose modification that is larger in volume than a 2′-OH group can for example be a 2′-OMe, 2′-O-MOE (2′-O-methoxyethyl), 2′-O-allyl or 2′-O-alkyl, with the proviso that the nucleic is capable of reducing the expression of the target gene to at least the same extent as the same nucleic acid without the modification(s) under comparable conditions. The first and/or the fourth modification is/are preferably a 2′-OMe modification and/or the second and/or third modification is/are preferably a 2′-F modification. The nucleotides on the first strand are numbered consecutively starting with nucleotide number 1 at the 5′ end of the first strand.

In one aspect of the nucleic acid, all the even-numbered nucleotides of the first strand are modified by a first modification, all the odd-numbered nucleotides of the first strand are modified by a second modification, all the nucleotides of the second strand in a position corresponding to an even-numbered nucleotide of the first strand are modified by a third modification, all the nucleotides of the second strand in a position corresponding to an odd-numbered nucleotide of the first strand are modified by a fourth modification, wherein the first and/or fourth modification is/are 2′-F and/or the second and/or third modification is/are 2′-OMe.

In one aspect of the nucleic acid, all the even-numbered nucleotides of the first strand are modified by a first modification, all the odd-numbered nucleotides of the first strand are modified by a second modification, all the nucleotides of the second strand in positions corresponding to nucleotides 11-13 of the first strand are modified by a fourth modification, all the nucleotides of the second strand other than the nucleotides corresponding to nucleotides 11-13 of the first strand are modified by a third modification, wherein the first and fourth modification are 2′-F and the second and third modification are 2′-OMe. In one embodiment in this aspect, the 3′ terminal nucleotide of the second strand is an inverted RNA nucleotide (i.e., the nucleotide is linked to the 3′ end of the strand through its 3′ carbon, rather than through its 5′ carbon as would normally be the case). When the 3′ terminal nucleotide of the second strand is an inverted RNA nucleotide, the inverted RNA nucleotide is preferably an unmodified nucleotide in the sense that it does not comprise any modifications compared to the natural nucleotide counterpart. Specifically, the inverted RNA nucleotide is preferably a 2′-OH nucleotide. Preferably, in this aspect when the 3′ terminal nucleotide of the second strand is an inverted RNA nucleotide, the nucleic acid is blunt-ended at least at the end that comprises the 5′ end of the first strand.

One aspect of the present invention is a nucleic acid as disclosed herein for inhibiting expression of the MASP-2 gene, preferably in a cell, wherein said first strand includes modified nucleotides or unmodified nucleotides at a plurality of positions in order to facilitate processing of the nucleic acid by RISC.

In one aspect, “facilitate processing by RISC” means that the nucleic acid can be processed by RISC, for example any modification present will permit the nucleic acid to be processed by RISC and preferably, will be beneficial to processing by RISC, suitably such that siRNA activity can take place.

A nucleic acid as disclosed herein, wherein the nucleotides at positions 2 and 14 from the 5′ end of the first strand are not modified with a 2′-OMe modification, and the nucleotide/nucleotides on the second strand which corresponds to position 11 or position 13 or positions 11 and 13 or positions 11, 12 and 13 of the first strand is/are not modified with a 2′-OMe modification (in other words, they are not modified or are modified with a modification other than 2′-OMe).

In one aspect, the nucleotide on the second strand which corresponds to position 13 of the first strand is the nucleotide that forms a base pair with position 13 (from the 5′ end) of the first strand.

In one aspect, the nucleotide on the second strand which corresponds to position 11 of the first strand is the nucleotide that forms a base pair with position 11 (from the 5′ end) of the first strand.

In one aspect, the nucleotide on the second strand which corresponds to position 12 of the first strand is the nucleotide that forms a base pair with position 12 (from the 5′ end) of the first strand.

For example, in a 19-mer nucleic acid which is double-stranded and blunt ended, position 13 (from the 5′ end) of the first strand would pair with position 7 (from the 5′ end) of the second strand. Position 11 (from the 5′ end) of the first strand would pair with position 9 (from the 5′ end) of the second strand. This nomenclature may be applied to other positions of the second strand.

In one aspect, in the case of a partially complementary first and second strand, the nucleotide on the second strand that “corresponds to” a position on the first strand may not necessarily form a base pair if that position is the position in which there is a mismatch, but the principle of the nomenclature still applies.

One aspect is a nucleic acid as disclosed herein, wherein the nucleotides at positions 2 and 14 from the 5′ end of the first strand are not modified with a 2′-OMe modification, and the nucleotides on the second strand which correspond to position 11, or 13, or 11 and 13, or 11-13 of the first strand are modified with a 2′-F modification.

One aspect is a nucleic acid as disclosed herein, wherein the nucleotides at positions 2 and 14 from the 5′ end of the first strand are modified with a 2′-F modification, and the nucleotides on the second strand which correspond to position 11, or 13, or 11 and 13, or 11-13 of the first strand are not modified with a 2′-OMe modification.

One aspect is a nucleic acid as disclosed herein, wherein the nucleotides at positions 2 and 14 from the 5′ end of the first strand are modified with a 2′-F modification, and the nucleotides on the second strand which correspond to position 11, or 13, or 11 and 13, or 11-13 of the first strand are modified with a 2′-F modification.

One aspect is a nucleic acid as disclosed herein wherein greater than 50% of the nucleotides of the first and/or second strand comprise a 2′-OMe modification, such as greater than 55%, 60%, 65%, 70%, 75%, 80%, or 85%, or more, of the first and/or second strand comprise a 2′-OMe modification, preferably measured as a percentage of the total nucleotides of both the first and second strands.

One aspect is a nucleic acid as disclosed herein wherein greater than 50% of the nucleotides of the first and/or second strand comprise a naturally occurring RNA modification, such as wherein greater than 55%, 60%, 65%, 70%, 75%, 80%, or 85% or more of the first and/or second strands comprise such a modification, preferably measured as a percentage of the total nucleotides of both the first and second strands. Suitable naturally occurring modifications include, as well as 2′-OMe, other 2′ sugar modifications, in particular a 2′-H modification resulting in a DNA nucleotide.

One aspect is a nucleic acid as disclosed herein comprising no more than 20%, such as no more than 15% such as no more than 10%, of nucleotides which have 2′ modifications that are not 2′-OMe modifications on the first and/or second strand, preferably as a percentage of the total nucleotides of both the first and second strands.

One aspect is a nucleic acid as disclosed herein, wherein the number of nucleotides in the first and/or second strand with a 2′-modification that is not a 2′-OMe modification is no more than 7, more preferably no more than 5, and most preferably no more than 3.

One aspect is a nucleic acid as disclosed herein comprising no more than 20%, (such as no more than 15% or no more than 10%) of 2′-F modifications on the first and/or second strand, preferably as a percentage of the total nucleotides of both strands.

One aspect is a nucleic acid as disclosed herein, wherein the number of nucleotides in the first 35 and/or second strand with a 2′-F modification is no more than 7, more preferably no more than 5, and most preferably no more than 3.

One aspect is a nucleic acid as disclosed herein, wherein all nucleotides are modified with a 2′-OMe modification except positions 2 and 14 from the 5′ end of the first strand and the nucleotides on the second strand which correspond to position 11, or 13, or 11 and 13, or 11-13 of the first strand. Preferably the nucleotides that are not modified with 2′-OMe are modified with fluoro at the 2′ position (2′-F modification).

In certain embodiments, a preferred aspect is a nucleic acid as disclosed herein wherein all nucleotides of the nucleic acid are modified at the 2′ position of the sugar. Preferably these nucleotides are modified with a 2′-F modification where the modification is not a 2′-OMe modification.

In one aspect the nucleic acid is modified on the first strand with alternating 2′-OMe modifications and 2-F modifications, and positions 2 and 14 (starting from the 5′ end) are modified with 2′-F. Preferably the second strand is modified with 2′-F modifications at nucleotides on the second strand which correspond to position 11, or 13, or 11 and 13, or 11-13 of the first strand. Preferably the second strand is modified with 2′-F modifications at positions 11-13 counting from the 3′ end starting at the first position of the complementary (double-stranded) region, and the remaining modifications are naturally occurring modifications, preferably 2′-OMe. The complementary region at least in this case starts at the first position of the second strand that has a corresponding nucleotide in the first strand, regardless of whether the two nucleotides are able to base pair to each other.

In one aspect of the nucleic acid, each of the nucleotides of the first strand and of the second strand is a modified nucleotide.

The term “odd numbered” as described herein means a number not divisible by two. Examples of odd numbers are 1, 3, 5, 7, 9, 11 and so on. The term “even numbered” as described herein means a number which is evenly divisible by two. Examples of even numbers are 2, 4, 6, 8, 10, 12, 14 and so on.

Unless specifically stated otherwise, herein the nucleotides of the first strand are numbered contiguously starting with nucleotide number 1 at the 5′ end of the first strand. Nucleotides of the second strand are numbered contiguously starting with nucleotide number 1 at the 3′ end of the second strand.

One or more nucleotides on the first and/or second strand may be modified, to form modified nucleotides. One or more of the odd-numbered nucleotides of the first strand may be modified.

One or more of the even-numbered nucleotides of the first strand may be modified by at least a second modification, wherein the at least second modification is different from the modification on the one or more odd nucleotides. At least one of the one or more modified even numbered-nucleotides may be adjacent to at least one of the one or more modified odd-numbered nucleotides.

A plurality of odd-numbered nucleotides in the first strand may be modified in the nucleic acid of the invention. A plurality of even-numbered nucleotides in the first strand may be modified by a second modification. The first strand may comprise adjacent nucleotides that are modified by a common modification. The first strand may also comprise adjacent nucleotides that are modified by a second different modification (i.e., the first strand may comprise nucleotides that are adjacent to each other and modified by a first modification as well as other nucleotides that are adjacent to each other and modified by a second modification that is different to the first modification).

One or more of the odd-numbered nucleotides of the second strand (wherein the nucleotides are numbered contiguously starting with nucleotide number 1 at the 3′ end of the second strand) may be modified by a modification that is different to the modification of the odd-numbered nucleotides on the first strand (wherein the nucleotides are numbered contiguously starting with nucleotide number 1 at the 5′ end of the first strand) and/or one or more of the even-numbered nucleotides of the second strand may be modified by the same modification of the odd-numbered nucleotides of the first strand. At least one of the one or more modified even-numbered nucleotides of the second strand may be adjacent to the one or more modified odd-numbered nucleotides. A plurality of odd-numbered nucleotides of the second strand may be modified by a common modification and/or a plurality of even-numbered nucleotides may be modified by the same modification that is present on the first stand odd-numbered nucleotides. A plurality of odd-numbered nucleotides on the second strand may be modified by a modification that is different from the modification of the first strand odd-numbered nucleotides.

The second strand may comprise adjacent nucleotides that are modified by a common modification, which may be a modification that is different from the modification of the odd-numbered nucleotides of the first strand.

In some aspects of the nucleic acid of the invention, each of the odd-numbered nucleotides in the first strand and each of the even-numbered nucleotides in the second strand may be modified with a common modification and, each of the even-numbered nucleotides may be modified in the first strand with a different modification and each of the odd-numbered nucleotides may be modified in the second strand with the different modification.

The nucleic acid of the invention may have the modified nucleotides of the first strand shifted by at least one nucleotide relative to the unmodified or differently modified nucleotides of the second strand.

In certain aspects, one ne or more or each of the odd numbered-nucleotides may be modified in the first strand and one or more or each of the even-numbered nucleotides may be modified in the second strand. One or more or each of the alternating nucleotides on either or both strands may be modified by a second modification. One or more or each of the even-numbered nucleotides may be modified in the first strand and one or more or each of the even-numbered nucleotides may be modified in the second strand. One or more or each of the alternating nucleotides on either or both strands may be modified by a second modification. One or more or each of the odd-numbered nucleotides may be modified in the first strand and one or more of the odd-numbered nucleotides may be modified in the second strand by a common modification. One or more or each of the alternating nucleotides on either or both strands may be modified by a second modification. One or more or each of the even-numbered nucleotides may be modified in the first strand and one or more or each of the odd-numbered nucleotides may be modified in the second strand by a common modification. One or more or each of the alternating nucleotides on either or both strands may be modified by a second modification.

The nucleic acid of the invention may comprise single- or double-stranded constructs that comprise at least two regions of alternating modifications in one or both of the strands. These alternating regions can comprise up to about 12 nucleotides but preferably comprise from about 3 to about 10 nucleotides. The regions of alternating nucleotides may be located at the termini of one or both strands of the nucleic acid of the invention. The nucleic acid may comprise from 4 to about 10 nucleotides of alternating nucleotides at each of the termini (3′ and 5′) and these regions may be separated by from about 5 to about 12 contiguous unmodified or differently or commonly modified nucleotides.

The odd numbered nucleotides of the first strand may be modified and the even numbered nucleotides may be modified with a second modification. The second strand may comprise adjacent nucleotides that are modified with a common modification, which may be the same as the modification of the odd-numbered nucleotides of the first strand. One or more nucleotides of the second strand may also be modified with the second modification. One or more nucleotides with the second modification may be adjacent to each other and to nucleotides having a modification that is the same as the modification of the odd-numbered nucleotides of the first strand. The first strand may also comprise phosphorothioate linkages between the two nucleotides at the 3′ end and at the 5′ end or a phosphorodithioate linkage between the two nucleotides at the 3′ end. The second strand may comprise a phosphorothioate or phosphorodithioate linkage between the two nucleotides at the 5′ end. The second strand may also be conjugated to a ligand at the 5′ end.

The nucleic acid of the invention may comprise a first strand comprising adjacent nucleotides that are modified with a common modification. One or more such nucleotides may be adjacent to one or more nucleotides which may be modified with a second modification. One or more nucleotides with the second modification may be adjacent. The second strand may comprise adjacent nucleotides that are modified with a common modification, which may be the same as one of the modifications of one or more nucleotides of the first strand. One or more nucleotides of the second strand may also be modified with the second modification. One or more nucleotides with the second modification may be adjacent. The first strand may also comprise phosphorothioate linkages between the two nucleotides at the 3′ end and at the 5′ end or a phosphorodithioate linkage between the two nucleotides at the 3′ end. The second strand may comprise a phosphorothioate or phosphorodithioate linkage between the two nucleotides at the 3′ end. The second strand may also be conjugated to a ligand at the 5′ end. The nucleotides numbered from 5′ to 3′ on the first strand and 3′ to 5′ on the second strand, 1, 3, 5, 7, 9, 11, 13, 15, 17, 19, 21, 23 and 25 may be modified by a modification on the first strand. The nucleotides numbered 2, 4, 6, 8, 10, 12, 14, 16, 18, 20, 22 and 24 may be modified by a second modification on the first strand. The nucleotides numbered 1, 3, 5, 7, 9, 11, 13, 15, 17, 19, 21, 23 may be modified by a modification on the second strand. The nucleotides numbered 2, 4, 6, 8, 10, 12, 14, 16, 18, 20, 22 and 24 may be modified by a second modification on the second strand. Nucleotides are numbered for the sake of the nucleic acid of the present invention from 5′ to 3′ on the first strand and 3′ to 5′ on the second strand.

The nucleotides numbered 2, 4, 6, 8, 10, 12, 14, 16, 18, 20, 22 and 24 may be modified by a modification on the first strand. The nucleotides numbered 1, 3, 5, 7, 9, 11, 13, 15, 17, 19, 21, 23 may be modified by a second modification on the first strand. The nucleotides numbered 1, 3, 5, 7, 9, 11, 13, 15, 17, 19, 21, 23 may be modified by a modification on the second strand. The nucleotides numbered 2, 4, 6, 8, 10, 12, 14, 16, 18, 20, 22 and 24 may be modified by a second modification on the second strand.

Clearly, if the first and/or the second strand are shorter than 25 nucleotides in length, such as 19 nucleotides in length, there are no nucleotides numbered 20, 21, 22, 23, 24 and 25 to be modified. The skilled person understands the description above to apply to shorter strands, accordingly.

One or more modified nucleotides on the first strand may be paired with modified nucleotides on the second strand having a common modification. One or more modified nucleotides on the first strand may be paired with modified nucleotides on the second strand having a different modification. One or more modified nucleotides on the first strand may be paired with unmodified nucleotides on the second strand. One or more modified nucleotides on the second strand may be paired with unmodified nucleotides on the first strand. In other words, the alternating nucleotides can be aligned on the two strands such as, for example, all the modifications in the alternating regions of the second strand are paired with identical modifications in the first strand or alternatively the modifications can be offset by one nucleotide with the common modifications in the alternating regions of one strand pairing with dissimilar modifications (i.e., a second or further modification) in the other strand. Another option is to have dissimilar modifications in each of the strands.

The modifications on the first strand may be shifted by one nucleotide relative to the modified nucleotides on the second strand, such that common modified nucleotides are not paired with each other.

The modification and/or modifications may each and individually be selected from the group consisting of 3′ terminal deoxy thymine, 2′-OMe, a 2′ deoxy modification, a 2′ amino modification, a 2′ alkyl modification, a morpholino modification, a phosphoramidate modification, 5′-phosphorothioate group modification, a 5′ phosphate or 5′ phosphate mimic modification and a cholesteryl derivative or a dodecanoic acid bisdecylamide group modification and/or the modified nucleotide may be any one of a locked nucleotide, an abasic nucleotide or a non-natural base comprising nucleotide. At least one modification may be 2′-OMe and/or at least one modification may be 2′-F. Further modifications as described herein may be present on the first and/or second strand.

The nucleic acid of the invention may comprise an inverted RNA nucleotide at one or several of the strand ends. Such inverted nucleotides provide stability to the nucleic acid. Preferably, the nucleic acid comprises at least an inverted nucleotide at the 3′ end of the first and/or the second strand and/or at the 5′ end of the second strand. More preferably, the nucleic acid comprises an inverted nucleotide at the 3′ end of the second strand. Most preferably, the nucleic acid comprises an inverted RNA nucleotide at the 3′ end of the second strand and this nucleotide is preferably an inverted A. An inverted nucleotide is a nucleotide that is linked to the 3′ end of a nucleic acid through its 3′ carbon, rather than its 5′ carbon as would normally be the case or is linked to the 5′ end of a nucleic acid through its 5′ carbon, rather than its 3′ carbon as would normally be the case. The inverted nucleotide is preferably present at an end of a strand not as an overhang but opposite a corresponding nucleotide in the other strand. Accordingly, the nucleic acid is preferably blunt-ended at the end that comprises the inverted RNA nucleotide. An inverted RNA nucleotide being present at the end of a strand preferably means that the last nucleotide at this end of the strand is the inverted RNA nucleotide. A nucleic acid with such a nucleotide is stable and easy to synthesise. The inverted RNA nucleotide is preferably an unmodified nucleotide in the sense that it does not comprise any modifications compared to the natural nucleotide counterpart. Specifically, the inverted RNA nucleotide is preferably a 2′-OH nucleotide.

Nucleic acids of the invention may comprise one or more nucleotides modified at the 2′ position with a 2′-H, and therefore having a DNA nucleotide within the nucleic acid. Nucleic acids of the invention may comprise DNA nucleotides at positions 2 and/or 14 of the first strand counting from the 5′ end of the first strand. Nucleic acids may comprise DNA nucleotides on the second strand which correspond to position 11, or 13, or 11 and 13, or 11-13 of the first strand.

In one aspect there is no more than one DNA nucleotide per nucleic acid of the invention. Nucleic acids of the invention may comprise one or more LNA nucleotides. Nucleic acids of the invention may comprise LNA nucleotides at positions 2 and/or 14 of the first strand counting from the 5′ end of the first strand. Nucleic acids may comprise LNA on the second strand which correspond to position 11, or 13, or 11 and 13, or 11-13 of the first strand.

Some representative modified nucleic acid sequences of the present invention are shown in the examples. These examples are meant to be representative and not limiting.

In certain preferred embodiments, the nucleic acid may comprise a first modification and a second or further modification which are each and individually selected from the group comprising 2′-OMe modification and 2′-F modification. The nucleic acid may comprise a modification that is 2′-OMe that may be a first modification, and a second modification that is 2′-F. The nucleic acid of the invention may also include a phosphorothioate or phosphorodithioate modification and/or a deoxy modification which may be present in or between the terminal 2 or 3 nucleotides of each or any end of each or both strands.

In one aspect of the nucleic acid, at least one nucleotide of the first and/or second strand is a modified nucleotide, wherein if the first strand comprises at least one modified nucleotide:
(i) at least one or both of the nucleotides 2 and 14 of the first strand is/are modified by a first modification; and/or
(ii) at least one, several, or all the even-numbered nucleotides of the first strand is/are modified by a first modification; and/or
(iii) at least one, several, or all the odd-numbered nucleotides of the first strand is/are modified by a second modification; and/or
wherein if the second strand comprises at least one modified nucleotide:
(iv) at least one, several, or all the nucleotides of the second strand in a position corresponding to an even-numbered nucleotide of the first strand is/are modified by a third modification; and/or
(v) at least one, several, or all the nucleotides of the second strand in a position corresponding to an odd-numbered nucleotide of the first strand is/are modified by a fourth modification; and/or
(vi) at least one, several, or all the nucleotides of the second strand in a position corresponding to nucleotide 11 or nucleotide 13 or nucleotides 11 and 13 or nucleotides 11-13 of the first strand is/are modified by a fourth modification; and/or
(vii) at least one, several, or all the nucleotides of the second strand in a position other than the position corresponding to nucleotide 11 or nucleotide 13 or nucleotides 11 and 13 or nucleotides 11-13 of the first strand is/are modified by a third modification;
wherein the nucleotides on the first strand are numbered consecutively starting with nucleotide number 1 at the 5′ end of the first strand;
wherein the modifications are preferably at least one of the following:
(a) the first modification is preferably different from the second and from the third modification;
(b) the first modification is preferably the same as the fourth modification;
(c) the second and the third modification are preferably the same modification;
(d) the first modification is preferably a 2′-F modification;
(e) the second modification is preferably a 2′-OMe modification;
(f) the third modification is preferably a 2′-OMe modification; and/or
(g) the fourth modification is preferably a 2′-F modification; and
wherein optionally the nucleic acid is conjugated to a ligand.

One aspect is a double-stranded nucleic acid for inhibiting expression of MASP-2, preferably in a cell, wherein the nucleic acid comprises a first strand and a second strand, wherein the unmodified equivalent of the first strand sequence comprises a sequence of at least 15 nucleotides differing by no more than 3 nucleotides from any one of the first strand sequences shown in Table 5a, or in Table 1, wherein all the even-numbered nucleotides of the first strand are modified by a first modification, all the odd-numbered nucleotides of the first strand are modified by a second modification, all the nucleotides of the second strand in a position corresponding to an even-numbered nucleotide of the first strand are modified by a third modification, all the nucleotides of the second strand in a position corresponding to an odd-numbered nucleotide of the first strand are modified by a fourth modification, wherein the first and fourth modification are 2′-F and the second and third modification are 2′-OMe.

One aspect is a double-stranded nucleic acid for inhibiting expression of MASP-2, preferably in a cell, wherein the nucleic acid comprises a first strand and a second strand, wherein the unmodified equivalent of the first strand sequence comprises a sequence of at least 15 nucleotides differing by no more than 3 nucleotides from any one of the first strand sequences shown in Table 5a, or in Table 1, wherein all the even-numbered nucleotides of the first strand are modified by a first modification, all the odd-numbered nucleotides of the first strand are modified by a second modification, all the nucleotides of the second strand in positions corresponding to nucleotides 11-13 of the first strand are modified by a fourth modification, all the nucleotides of the second strand other than the nucleotides corresponding to nucleotides 11-13 of the first strand are modified by a third modification, wherein the first and fourth modification are 2′-F and the second and third modification are 2′-OMe.

The 3′ and 5′ ends of an oligonucleotide can be modified. Such modifications can be at the 3′ end or the 5′ end or both ends of the molecule. They can include modification or replacement of an entire terminal phosphate or of one or more of the atoms of the phosphate group. For example, the 3′ and 5′ ends of an oligonucleotide can be conjugated to other functional molecular entities such as labelling moieties, e.g., fluorophores (e.g., pyrene, TAMRA, fluorescein, Cy3 or Cy5 dyes) or protecting groups (based, e.g., on sulfur, silicon, boron or ester). The functional molecular entities can be attached to the sugar through a phosphate group and/or a linker. The terminal atom of the linker can connect to or replace the linking atom of the phosphate group or the C-3′ or C-5′ O, N, S or C group of the sugar. Alternatively, the linker can connect to or replace the terminal atom of a nucleotide surrogate (e.g., PNAs). These spacers or linkers can include e.g., —(CH₂)ₙ—, —(CH₂)ₙN—, —(CH₂)ₙO—, —(CH₂)ₙS—, —(CH₂CH₂O)ₙCH₂CH₂O— (e.g., n=3 or 6), abasic sugars, amide, carboxy, amine, oxyamine, oxyimine, thioether, disulfide, thiourea, sulfonamide, or morpholino, or biotin and fluorescein reagents. The 3′ end can be an —OH group.

Other examples of terminal modifications include dyes, intercalating agents (e.g., acridines), cross-linkers (e.g., psoralene, mitomycin C), porphyrins (TPPC4, texaphyrin, Sapphyrin), polycyclic aromatic hydrocarbons (e.g., phenazine, dihydrophenazine), artificial endonucleases, EDTA, lipophilic carriers (e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]2, polyamino, alkyl, substituted alkyl, radiolabeled markers, enzymes, haptens (e.g., biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles).

Terminal modifications can also be useful for monitoring distribution, and in such cases the groups to be added may include fluorophores, e.g., fluorescein or an Alexa dye. Terminal modifications can also be useful for enhancing uptake, useful modifications for this include cholesterol. Terminal modifications can also be useful for cross-linking an RNA agent to another moiety.

Terminal modifications can be added for a number of reasons, including to modulate activity or to modulate resistance to degradation. Terminal modifications useful for modulating activity include modification of the 5′ end with phosphate or phosphate analogues. Nucleic acids of the invention, on the first or second strand, may be 5′ phosphorylated or include a phosphoryl analogue at the 5′ prime terminus. 5′-phosphate modifications include those which are compatible with RISC mediated gene silencing. Suitable modifications include: 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)₂(O)P—S-5′); any additional combination of oxygen/sulfur replaced monophosphate, diphosphate and triphosphates (e.g., 5′-alpha-thiotriphosphate, 5′-gamma-thiotriphosphate, etc.), 5′-phosphoramidates ((HO)₂(O)P—NH-5′, (HO)(NH₂)(O)P—O-5′), 5′-alkylphosphonates (alkyl=methyl, ethyl, isopropyl, propyl, etc., e.g. RP(OH)(O)—O-5′- (wherein R is an alkyl), (OH)₂(O)P-5′-CH₂—), 5′ vinylphosphonate, 5′-alkyletherphosphonates (alkylether=methoxymethyl (MeOCH₂—), ethoxymethyl, etc., e.g. RP(OH)(O)—O-5′- (wherein R is an alkylether)).

Certain moieties may be linked to the 5′ terminus of the first strand or the second strand. These include abasic ribose moiety, abasic deoxyribose moiety, modifications abasic ribose and abasic deoxyribose moieties including 2′-O alkyl modifications; inverted abasic ribose and abasic deoxyribose moieties and modifications thereof, C6-imino-Pi; a mirror nucleotide including L-DNA and L-RNA; 5′OMe nucleotide; and nucleotide analogues including 4′,5′-methylene nucleotide; 1-(β-D-erythrofuranosyl)nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 12-aminododecyl phosphate; hydroxypropyl phosphate; 1,5-anhydrohexitol nucleotide; alpha-nucleotide; threo-pentofuranosyl nucleotide; acyclic 3′,4′-seco nucleotide; 3,4-dihydroxybutyl nucleotide; 3,5-dihydroxypentyl nucleotide, 5′-5′-inverted abasic moiety; 1,4-butanediol phosphate; 5′-amino; and bridging or non-bridging methylphosphonate and 5′-mercapto moieties.

In each sequence described herein, a C-terminal “—OH” moiety may be substituted for a C-terminal “—NH₂” moiety, and vice-versa.

The invention also provides a nucleic acid according to any aspect of the invention described herein, wherein the first strand has a terminal 5′ (E)-vinylphosphonate nucleotide at its 5′ end. This terminal 5′ (E)-vinylphosphonate nucleotide is preferably linked to the second nucleotide in the first strand by a phosphodiester linkage. Preferably, the terminal 5′ (E)-vinylphosphonate (“vp”) nucleotide is a uridine (“vp-U”).

The first strand of the nucleic acid may comprise formula (I):

(vp)-N₍ₚₒ₎[N₍ₚₒ₎]ₙ- (1)

where ‘(vp)-’ is the 5′ (E)-vinylphosphonate, ‘N’ is a nucleotide, ‘po’ is a phosphodiester linkage, and n is from 1 to (the total number of nucleotides in the first strand—2), preferably wherein n is from 1 to (the total number of nucleotides in the first strand—3), more preferably wherein n is from 1 to (the total number of nucleotides in the first strand—4).

Preferably, the terminal 5′ (E)-vinylphosphonate nucleotide is an RNA nucleotide, preferably a (vp)-U.

A terminal 5′ (E)-vinylphosphonate nucleotide is a nucleotide wherein the phosphate group at the 5′-end of the ribose has been replaced with a E-vinylphosphonate group:

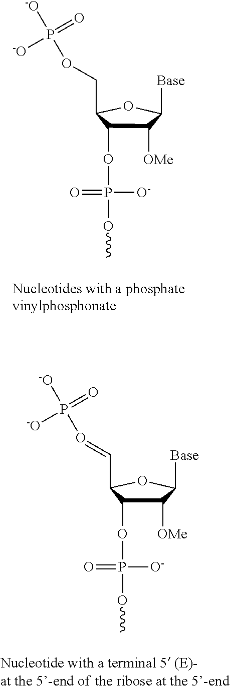

In one aspect, the first strand has a terminal 5′ (E)-vinylphosphonate nucleotide at its 5′ end, the terminal 5′ (E)-vinylphosphonate nucleotide is linked to the second nucleotide in the first strand by a phosphodiester linkage and the first strand comprises a) more than 1 phosphodiester linkage; b) phosphodiester linkages between at least the terminal three 5′ nucleotides and/or c) phosphodiester linkages between at least the terminal four 5′ nucleotides.

In one aspect, the first strand and/or the second strand of the nucleic acid comprises at least one phosphorothioate (ps) and/or at least one phosphorodithioate (ps2) linkage between two nucleotides.

In one aspect, the first strand and/or the second strand of the nucleic acid comprises more than one phosphorothioate and/or more than one phosphorodithioate linkage.

In one aspect, the first strand and/or the second strand of the nucleic acid comprises a phosphorothioate or phosphorodithioate linkage between the terminal two 3′ nucleotides or phosphorothioate or phosphorodithioate linkages between the terminal three 3′ nucleotides.

Preferably, the linkages between the other nucleotides in the first strand and/or the second strand are phosphodiester linkages.

In one aspect, the first strand and/or the second strand of the nucleic acid comprises a phosphorothioate linkage between the terminal two 5′ nucleotides or a phosphorothioate linkages between the terminal three 5′ nucleotides.

In one aspect, the nucleic acid of the present invention comprises one or more phosphorothioate or phosphorodithioate modifications on one or more of the terminal ends of the first and/or the second strand. Optionally, each or either end of the first strand may comprise one or two or three phosphorothioate or phosphorodithioate modified nucleotides (internucleoside linkage). Optionally, each or either end of the second strand may comprise one or two or three phosphorothioate or phosphorodithioate modified nucleotides (internucleoside linkage).

In one aspect, the nucleic acid comprises a phosphorothioate linkage between the terminal two or three 3′ nucleotides and/or 5′ nucleotides of the first and/or the second strand. Preferably, the nucleic acid comprises a phosphorothioate linkage between each of the terminal three 3′ nucleotides and the terminal three 5′ nucleotides of the first strand and of the second strand. Preferably, all remaining linkages between nucleotides of the first and/or of the second strand are phosphodiester linkages.

In one aspect, the nucleic acid comprises a phosphorodithioate linkage between each of the two, three or four terminal nucleotides at the 3′ end of the first strand and/or comprises a phosphorodithioate linkage between each of the two, three or four terminal nucleotides at the 3′ end of the second strand and/or a phosphorodithioate linkage between each of the two, three or four terminal nucleotides at the 5′ end of the second strand and comprises a linkage other than a phosphorodithioate linkage between the two, three or four terminal nucleotides at the 5′ end of the first strand.

In one aspect, the nucleic acid comprises a phosphorothioate linkage between the terminal three 3′ nucleotides and the terminal three 5′ nucleotides of the first strand and of the second strand. Preferably, all remaining linkages between nucleotides of the first and/or of the second strand are phosphodiester linkages.

In one aspect, the nucleic acid:
(i) has a phosphorothioate linkage between the terminal three 3′ nucleotides and the terminal three 5′ nucleotides of the first strand;
(ii) is conjugated to a triantennary ligand either on the 3′ end nucleotide or on the 5′ end nucleotide of the second strand;
(iii) has a phosphorothioate linkage between the terminal three nucleotides of the second strand at the end opposite to the one conjugated to the triantennary ligand; and
(iv) optionally all remaining linkages between nucleotides of the first and/or of the second strand are phosphodiester linkages.

In one aspect, the nucleic acid:
(i) has a terminal 5′ (E)-vinylphosphonate nucleotide at the 5′ end of the first strand;
(ii) has a phosphorothioate linkage between the terminal three 3′ nucleotides on the first and second strand and between the terminal three 5′ nucleotides on the second strand or it has a phosphorodithioate linkage between the terminal two 3′ nucleotides on the first and second strand and between the terminal two 5′ nucleotides on the second strand; and
(iii) optionally all remaining linkages between nucleotides of the first and/or of the second strand are phosphodiester linkages.

In one aspect, the nucleic acid has a terminal 5′ (E)-vinylphosphonate nucleotide at the 5′ end of the first strand and has a phosphorothioate linkage between the terminal three 3′ nucleotides on the first and between the terminal three 3′nucleotides on the second strand; and optionally all remaining linkages between nucleotides of the first and/or of the second strand are phosphodiester linkages.

The use of a phosphorodithioate linkage in the nucleic acid of the invention reduces the variation in the stereochemistry of a population of nucleic acid molecules compared to molecules comprising a phosphorothioate in that same position. Phosphorothioate linkages introduce chiral centres and it is difficult to control which non-linking oxygen is substituted for sulphur. The use of a phosphorodithioate ensures that no chiral centre exists in that linkage and thus reduces or eliminates any variation in the population of nucleic acid molecules, depending on the number of phosphorodithioate and phosphorothioate linkages used in the nucleic acid molecule.

In one aspect, the nucleic acid comprises a phosphorodithioate linkage between the two terminal nucleotides at the 3′ end of the first strand and a phosphorodithioate linkage between the two terminal nucleotides at the 3′ end of the second strand and a phosphorodithioate linkage between the two terminal nucleotides at the 5′ end of the second strand and comprises a linkage other than a phosphorodithioate linkage between the two, three or four terminal nucleotides at the 5′ end of the first strand. Preferably, the first strand has a terminal 5′ (E)-vinylphosphonate nucleotide at its 5′ end. This terminal 5′ (E)-vinylphosphonate nucleotide is preferably linked to the second nucleotide in the first strand by a phosphodiester linkage. Preferably, all the linkages between the nucleotides of both strands other than the linkage between the two terminal nucleotides at the 3′ end of the first strand and the linkages between the two terminal nucleotides at the 3′ end and at the 5′ end of the second strand are phosphodiester linkages.

In one aspect, the nucleic acid comprises a phosphorothioate linkage between each of the three terminal 3′ nucleotides and/or between each of the three terminal 5′ nucleotides on the first strand, and/or between each of the three terminal 3′ nucleotides and/or between each of the three terminal 5′ nucleotides of the second strand when there is no phosphorodithioate linkage present at that end. No phosphorodithioate linkage being present at an end means that the linkage between the two terminal nucleotides, or preferably between the three terminal nucleotides of the nucleic acid end in question are linkages other than phosphorodithioate linkages.

In one aspect, all the linkages of the nucleic acid between the nucleotides of both strands other than the linkage between the two terminal nucleotides at the 3′ end of the first strand and the linkages between the two terminal nucleotides at the 3′ end and at the 5′ end of the second strand are phosphodiester linkages.

Other phosphate linkage modifications are possible.

The phosphate linker can also be modified by replacement of a linking oxygen with nitrogen (bridged phosphoroamidates), sulfur (bridged phosphorothioates) and carbon (bridged methylenephosphonates). The replacement can occur at a terminal oxygen. Replacement of the non-linking oxygens with nitrogen is possible.

The phosphate groups can also individually be replaced by non-phosphorus containing connectors.

Examples of moieties which can replace the phosphate group include siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino. In certain embodiments, replacements may include the methylenecarbonylamino and methylenemethylimino groups.

The phosphate linker and ribose sugar may be replaced by nuclease resistant nucleotides. Examples include the morpholino, cyclobutyl, pyrrolidine and peptide nucleic acid (PNA) nucleoside surrogates. In certain embodiments, PNA surrogates may be used.

In one aspect, the nucleic acid, which is preferably an siRNA that inhibits expression of MASP-2, preferably via RNAi, and preferably in a cell, comprises one or more or all of:
(i) a modified nucleotide;
(ii) a modified nucleotide other than a 2′-OMe modified nucleotide at positions 2 and 14 from the 5′ end of the first strand with a given SEQ ID No., preferably a 2′-F modified nucleotide;
(iii) each of the odd-numbered nucleotides of the first strand as numbered starting from one at the 5′ end of the first strand with a given SEQ ID No. are 2′-OMe modified nucleotides;
(iv) each of the even-numbered nucleotides of the first strand as numbered starting from one at the 5′ end of the first strand with a given SEQ ID No. are 2′-F modified nucleotides;
(v) the second strand nucleotide corresponding to position 11 and/or 13 or 11-13 of the first strand with a given SEQ ID No. is modified by a modification other than a 2′-OMe modification, preferably wherein one or both or all of these positions comprise a 2′-F modification;
(vi) an inverted nucleotide, preferably a 3′-3′ linkage at the 3′ end of the second strand with a given SEQ ID No.;
(vii) one or more phosphorothioate linkages;
(viii) one or more phosphorodithioate linkages; and/or
(ix) the first strand with a given SEQ ID No. has a terminal 5′ (E)-vinylphosphonate nucleotide at its 5′ end, in which case the terminal 5′ (E)-vinylphosphonate nucleotide is preferably a uridine and is preferably linked to the second nucleotide in the first strand by a phosphodiester linkage.

A nucleic acid of the present disclosure may comprise a first strand and a second strand, wherein the first strand sequence comprises a sequence of at least 15 nucleotides differing by no more than 3 nucleotides from any one of the first strand sequences with a given SEQ ID No. shown in Table 5b.

A nucleic acid of the present disclosure may be a nucleic acid wherein:
(a) the first strand sequence comprises a sequence differing by no more than 3 nucleotides from any one of the first strand sequences of Table 5b, and optionally wherein the second strand sequence comprises a sequence differing by no more than 3 nucleotides from the corresponding second strand sequence of Table 5b;
(b) the first strand sequence comprises a sequence differing by no more than 2 nucleotides from any one of the first strand sequences of Table 5b, and optionally wherein the second strand sequence comprises a sequence differing by no more than 2 nucleotides from the corresponding second strand sequence of Table 5b;
(c) the first strand sequence comprises a sequence differing by no more than 1 nucleotide from any one of the first strand sequences of Table 5b, and optionally wherein the second strand sequence comprises a sequence differing by no more than 1 nucleotide from the corresponding second strand sequence of Table 5b;
(d) the first strand sequence comprises a sequence corresponding to nucleotides 2 to 17 from the 5′ end of any one of the first strand sequences with a given SEQ ID No. shown in Table 5b, and optionally wherein the second strand sequence comprises a sequence corresponding to nucleotides 2 to 17 from the 5′ end of the corresponding second strand sequence with a given SEQ ID No. shown in Table 5b;
(e) the first strand sequence comprises a sequence corresponding to nucleotides 2 to 18 from the 5′ end of any one of the first strand sequences with a given SEQ ID No. shown in Table 5b, and optionally wherein the second strand sequence comprises a sequence corresponding to nucleotides 2 to 18 from the 5′ end of the corresponding second strand sequence with a given SEQ ID No. shown in Table 5b;
(f) the first strand sequence comprises a sequence corresponding to nucleotides 2 to 19 from the 5′ end of any one of the first strand sequences with a given SEQ ID No. shown in Table 5b, and optionally wherein the second strand sequence comprises a sequence corresponding to nucleotides 2 to 19 from the 5′ end of the corresponding second strand sequence with a given SEQ ID No. shown in Table 5b;
(g) the first strand sequence comprises a sequence corresponding to nucleotides 2 to 19 from the 5′ end of any one of the first strand sequences with a given SEQ ID No. shown in Table 5b, and optionally wherein the second strand sequence comprises a sequence corresponding to nucleotides 1 to 18 from the 5′ end of the corresponding second strand sequence with a given SEQ ID No. shown in Table 5b;
(h) the first strand sequence comprises a sequence of any one of the first strand sequences with a given SEQ ID No. shown in Table 5b, and optionally wherein the second strand sequence comprises a sequence of the corresponding second strand sequence with a given SEQ ID No. shown in Table 5b;
(i) the first strand sequence consists essentially of any one of the first strand sequences with a given SEQ ID No. shown in Table 5b, and optionally wherein the second strand sequence consists of the sequence of the corresponding second strand sequence with a given SEQ ID No. shown in Table 5b;
(j) the first strand sequence consists of any one of the first strand sequences with a given SEQ ID No. shown in Table 5b, and optionally wherein the second strand sequence consists of the sequence of the corresponding second strand sequence with a given SEQ ID No. shown in Table 5b;
(k) the first strand sequence consists of a sequence corresponding to nucleotides 1 to 19 from the 5′ end of any one of the first strand sequences with a given SEQ ID No. shown in Table 5b,
   wherein said first strand sequence further consists of 1 (nucleotide 20 counted from the 5′end), 2 (nucleotides 20 and 21), 3 (nucleotides 20, 21 and 22), 4 (nucleotides 20, 21, 22 and 23), 5 (nucleotides 20, 21, 22, 23 and 24) or 6 (nucleotides 20, 21, 22, 23, 24 and 25) additional nucleotide(s) at the 3′end of any one of the first strand sequences with a given SEQ ID No. shown in Table 5b, and
   optionally wherein the second strand sequence comprises or consists essentially of or consists of a sequence of the corresponding second strand sequence with a given SEQ ID No. shown in Table 5b;
(l) the first strand sequence consists of a sequence corresponding to nucleotides 1 to 19 from the 5′ end of any one of the first strand sequences with a given SEQ ID No. shown in Table 5b,
   wherein said first strand sequence further consists of 1 (nucleotide 20 counted from the 5′end), 2 (nucleotides 20 and 21), 3 (nucleotides 20, 21 and 22), 4 (nucleotides 20, 21, 22 and 23), 5 (nucleotides 20, 21, 22, 23 and 24) or 6 (nucleotides 20, 21, 22, 23, 24 and 25) additional nucleotide(s) at the 3′end of any one of the first strand sequences with a given SEQ ID No. shown in Table 5b, and
   wherein said first strand sequence consists of a contiguous region of from 17-25 nucleotides in length, preferably of from 18-24 nucleotides in length, complementary to the MASP-2 transcript of SEQ ID NO. 821, and
   optionally wherein the second strand sequence comprises or consists essentially of or consists of a sequence of the corresponding second strand sequence with a given SEQ ID No. shown in Table 5b;
(m) the first strand and the second strand of any one of the nucleic acid molecules of subsections (a) to (l) above are present on a single strand wherein the first strand and the second strand are able to hybridise to each other and to thereby form a double-stranded nucleic acid with a duplex region of 17, 18, 19, 20, 21, 22, 23, 24 or 25 nucleotides in length; or
(n) the first strand and the second strand of any one of the nucleic acid molecules of subsections (a) to (l) above are on two separate strands that are able to hybridise to each other and to thereby form a double-stranded nucleic acid with a duplex region of 17, 18, 19, 20, 21, 22, 23, 24 or 25 nucleotides in length.

A nucleic acid of the present disclosure may comprise a first strand and a second strand, wherein the first strand sequence comprises a sequence of at least 15 nucleotides differing by no more than 3 nucleotides from any one of the first strand sequences with a given SEQ ID No. shown in Table 2.

TABLE 2

| First strand | Second strand |
|---|---|
| sequence (SEQ ID No.) | sequence (SEQ ID No.) |
| 794 | 816 |
| 774 | 775 |
| 798 | 817 |
| 800 | 818 |
| 802 | 788 |
| 804 | 789 |
| 776 | 777 |
| 778 | 779 |
| 780 | 781 |
| 782 | 783 |
| 804 | 820 |
| 806 | 807 |
| 810 | 811 |

A nucleic acid of the present disclosure may comprise a first strand and a second strand, wherein the first strand sequence comprises a sequence of at least 17 nucleotides differing by no more than 3 nucleotides from any one of the first strand sequences with a given SEQ ID No. shown in Table 2.

A nucleic acid of the present disclosure may comprise a first strand and a second strand, wherein the first strand sequence comprises a sequence of at least 17 nucleotides differing by no more than 2 nucleotides from any one of the first strand sequences with a given SEQ ID No. shown in Table 2.

A nucleic acid of the present disclosure may comprise a first strand and a second strand, wherein the first strand sequence comprises a sequence of at least 17 nucleotides differing by no more than 1 nucleotide from any one of the first strand sequences with a given SEQ ID No. shown in Table 2.

A nucleic acid of the present disclosure may comprise a first strand and a second strand, wherein the first strand sequence comprises a sequence of at least 18 nucleotides differing by no more than 3 nucleotides from any one of the first strand sequences with a given SEQ ID No. shown in Table 2.

A nucleic acid of the present disclosure may comprise a first strand and a second strand, wherein the first strand sequence comprises a sequence of at least 18 nucleotides differing by no more than 2 nucleotides from any one of the first strand sequences with a given SEQ ID No. shown in Table 2.

A nucleic acid of the present disclosure may comprise a first strand and a second strand, wherein the first strand sequence comprises a sequence of at least 18 nucleotides differing by no more than 1 nucleotide from any one of the first strand sequences with a given SEQ ID No. shown in Table 2.

A nucleic acid of the present disclosure may comprise a first strand and a second strand, wherein the first strand sequence comprises a sequence of at least 19 nucleotides differing by no more than 3 nucleotides from any one of the first strand sequences with a given SEQ ID No. shown in Table 2.

A nucleic acid of the present disclosure may comprise a first strand and a second strand, wherein the first strand sequence comprises a sequence of at least 19 nucleotides differing by no more than 2 nucleotides from any one of the first strand sequences with a given SEQ ID No. shown in Table 2.

A nucleic acid of the present disclosure may comprise a first strand and a second strand, wherein the first strand sequence comprises a sequence of at least 19 nucleotides differing by no more than 1 nucleotide from any one of the first strand sequences with a given SEQ ID No. shown in Table 2.

A nucleic acid of the present disclosure may comprise a first strand and a second strand, wherein the first strand sequence consists essentially of, or consists of a sequence from any one of the first strand sequences with a given SEQ ID No. shown in Table 2.

For example, a nucleic acid of the present disclosure may be a nucleic acid wherein:
(a) the first strand sequence comprises a sequence differing by no more than 3 nucleotides from any one of the first strand sequences of Table 2, and optionally wherein the second strand sequence comprises a sequence differing by no more than 3 nucleotides from the corresponding second strand sequence of Table 2;
(b) the first strand sequence comprises a sequence differing by no more than 2 nucleotides from any one of the first strand sequences of Table 2, and optionally wherein the second strand sequence comprises a sequence differing by no more than 2 nucleotides from the corresponding second strand sequence of Table 2;
(c) the first strand sequence comprises a sequence differing by no more than 1 nucleotide from any one of the first strand sequences of Table 2, and optionally wherein the second strand sequence comprises a sequence differing by no more than 1 nucleotide from the corresponding second strand sequence of Table 2;
(d) the first strand sequence comprises a sequence corresponding to nucleotides 2 to 17 from the 5′ end of any one of the first strand sequences with a given SEQ ID No. shown in Table 2, and optionally wherein the second strand sequence comprises a sequence corresponding to nucleotides 2 to 17 from the 5′ end of the corresponding second strand sequence with a given SEQ ID No. shown in Table 2;
(e) the first strand sequence comprises a sequence corresponding to nucleotides 2 to 18 from the 5′ end of any one of the first strand sequences with a given SEQ ID No. shown in Table 2, and optionally wherein the second strand sequence comprises a sequence corresponding to nucleotides 2 to 18 from the 5′ end of the corresponding second strand sequence with a given SEQ ID No. shown in Table 2;
(f) the first strand sequence comprises a sequence corresponding to nucleotides 2 to 19 from the 5′ end of any one of the first strand sequences with a given SEQ ID No. shown in Table 2, and optionally wherein the second strand sequence comprises a sequence corresponding to nucleotides 2 to 19 from the 5′ end of the corresponding second strand sequence with a given SEQ ID No. shown in Table 2;
(g) the first strand sequence comprises a sequence corresponding to nucleotides 2 to 19 from the 5′ end of any one of the first strand sequences with a given SEQ ID No. shown in Table 2, and optionally wherein the second strand sequence comprises a sequence corresponding to nucleotides 1 to 18 from the 5′ end of the corresponding second strand sequence with a given SEQ ID No. shown in Table 2;
(h) the first strand sequence comprises a sequence of any one of the first strand sequences of Table 2, and optionally wherein the second strand sequence comprises a sequence of the corresponding second strand sequence of Table 2;
(i) the first strand sequence consists essentially of any one of the first strand sequences of Table 2, and optionally wherein the second strand sequence consists essentially of the sequence of the corresponding second strand sequence of Table 2;
(j) the first strand sequence consists of any one of the first strand sequences of Table 2, and optionally wherein the second strand sequence consists of the sequence of the corresponding second strand sequence of Table 2;
(k) the first strand sequence consists of a sequence corresponding to nucleotides 1 to 19 from the 5′ end of any one of the first strand sequences with a given SEQ ID No. shown in Table 2,
   wherein said first strand sequence further consists of 1 (nucleotide 20 counted from the 5′end), 2 (nucleotides 20 and 21), 3 (nucleotides 20, 21 and 22), 4 (nucleotides 20, 21, 22 and 23), 5 (nucleotides 20, 21, 22, 23 and 24) or 6 (nucleotides 20, 21, 22, 23, 24 and 25) additional nucleotide(s) at the 3′end of any one of the first strand sequences with a given SEQ ID No. shown in Table 2, and
   optionally wherein the second strand sequence comprises or consists essentially of or consists of a sequence of the corresponding second strand sequence with a given SEQ ID No. shown in Table 2;
(l) the first strand sequence consists of a sequence corresponding to nucleotides 1 to 19 from the 5′ end of any one of the first strand sequences with a given SEQ ID No. shown in Table 2,
   wherein said first strand sequence further consists of 1 (nucleotide 20 counted from the 5′end), 2 (nucleotides 20 and 21), 3 (nucleotides 20, 21 and 22), 4 (nucleotides 20, 21, 22 and 23), 5 (nucleotides 20, 21, 22, 23 and 24) or 6 (nucleotides 20, 21, 22, 23, 24 and 25) additional nucleotide(s) at the 3′end of any one of the first strand sequences with a given SEQ ID No. shown in Table 2, and
   wherein said first strand sequence consists of a contiguous region of from 17-25 nucleotides in length, preferably of from 18-24 nucleotides in length, complementary to the MASP-2 transcript of SEQ ID NO. 821, and
   optionally wherein the second strand sequence comprises or consists essentially of or consists of a sequence of the corresponding second strand sequence with a given SEQ ID No. shown in Table 2;
(m) the first strand and the second strand of any one of the nucleic acid molecules of subsections (a) to (l) above are present on a single strand wherein the first strand and the second strand are able to hybridise to each other and to thereby form a double-stranded nucleic acid with a duplex region of 17, 18, 19, 20, 21, 22, 23, 24 or 25 nucleotides in length; or
(n) the first strand and the second strand of any one of the nucleic acid molecules of subsections (a) to (l) above are on two separate strands that are able to hybridise to each other and to thereby form a double-stranded nucleic acid with a duplex region of 17, 18, 19, 20, 21, 22, 23, 24 or 25 nucleotides in length.

All the features of the nucleic acids can be combined with all other aspects of the invention disclosed herein.

### Heterologous Moieties

The nucleic acids of the invention may be conjugated to a heterologous moiety. A heterologous moiety is any moiety which is not a nucleic acid molecule capable of inhibiting expression of MASP-2. A heterologous moiety may be, or may comprise, a peptide (or polypeptide), a saccharide (or polysaccharide), a lipid, a different nucleic acid, or any other suitable molecule.

Any given nucleic acid may be conjugated to a plurality of heterologous moieties, which may be the same or different.

An individual heterologous moiety may itself comprise one or more functional moieties (such as targeting agents as described in more detail below), each optionally covalently associated to the nucleic acid via a linker.

A heterologous moiety, or the functional component thereof, may serve for example to modulate bioavailability or pharmacokinetics. For example, it may increase half-life in vivo. Alternatively, a heterologous moiety (or the functional component thereof) may comprise a targeting agent. Efficient delivery of oligonucleotides, in particular double-stranded nucleic acids of the invention, to cells in vivo is important and requires specific targeting and substantial protection from the extracellular environment, particularly serum proteins. One method of achieving specific targeting is to conjugate a targeting agent to the nucleic acid, wherein the targeting agent helps in targeting the nucleic acid to a target cell which has a cell surface receptor that binds to the targeting agent.

In this context, the term “receptor” is used to include any molecule on the surface of a target cell capable of binding to the targeting agent, and should not be taken to imply any particular function for the cell surface receptor. The targeting agent may be regarded as a “ligand” for the cell surface receptor. The terms “targeting agent” and “ligand” may be used interchangeably. Again, this terminology should not be taken to imply any particular function for the targeting agent or the cell surface receptor, or any particular relationship between the two molecules other than the ability of one to bind to the other.

Thus, the targeting agent may be any moiety having affinity for the chosen receptor. It may, for example, be an affinity protein (such as an antibody or a fragment thereof having affinity for the chosen receptor), an aptamer, or any other suitable moiety. In some embodiments, the targeting agent may be a physiological ligand for the receptor.

Binding between the targeting agent and the receptor may promote uptake of the conjugated nucleic acid by the target cell, e.g., via internalisation of the receptor, or any other suitable mechanism. Thus, appropriate ligands for the desired receptor molecules may be used as targeting agents in order for the conjugated nucleic acids to be taken up by the target cells by mechanisms such as different receptor-mediated endocytosis pathways or functionally analogous processes. In other embodiments, a ligand which can mediate internalization of the nucleic acid into a target cell by mechanisms other than receptor mediated endocytosis may alternatively be conjugated to a nucleic acid of the invention for cell or tissue specific targeting.

One example of a ligand that mediates receptor mediated endocytosis is the GalNAc moiety described herein, which has high affinity to the asialoglycoprotein receptor complex (ASGP-R). The ASGP-R complex is composed of varying ratios of multimers of membrane ASGR1 and ASGR2 receptors, which are highly abundant on hepatocytes. One of the first disclosures of the use of triantennary cluster glycosides as conjugated ligands was in U.S. Pat. No. 5,885,968. Conjugates having three GalNAc ligands and comprising phosphate groups are known and are described in Dubber et al. (Bioconjug. Chem. 2003 January-February; 14(1):239-46.). The ASGP-R complex shows a 50-fold higher affinity for N-Acetyl-D-Galactosamine (GalNAc) than D-Gal.

The ASGP-R complex recognizes specifically terminal β-galactosyl subunits of glycosylated proteins or other oligosaccharides (Weigel, P. H. et. al., Biochim. Biophys. Acta. 2002 Sep. 19; 1572(2-3):341-63) and can be used for delivering a drug to the liver's hepatocytes expressing the receptor complex by covalent coupling of galactose or galactosamine to the drug substance (Ishibashi, S.; et. al., J Biol. Chem. 1994 Nov. 11; 269(45):27803-6). Furthermore, the binding affinity can be significantly increased by the multi-valency effect, which is achieved by the repetition of the targeting moiety (Biessen E A, et al., J Med Chem. 1995 Apr. 28; 38(9):1538-46).

The ASGP-R complex is a mediator for an active uptake of terminal β-galactosyl containing glycoproteins to the cell's endosomes. Thus, the ASGPR is highly suitable for targeted delivery of drug candidates conjugated to such ligands like, e.g., nucleic acids into receptor-expressing cells (Akinc et al., Mol Ther. 2010 July; 18(7):1357-64).

More generally the ligand can comprise a saccharide that is selected to have an affinity for at least one type of receptor on a target cell. In particular, the receptor is on the surface of a mammalian liver cell, for example, the hepatic asialoglycoprotein receptor complex described before (ASGP-R).

The saccharide may be selected from N-acetyl galactosamine, mannose, galactose, glucose, glucosamine and fucose. The saccharide may be N-acetyl galactosamine (GalNAc). The heterologous moiety may comprise a plurality of such saccharides, e.g., two or especially three such saccharides, e.g., three GalNAc groups.

A heterologous moiety may therefore comprise (i) one or more functional components, and (ii) a linker, wherein the linker conjugates the functional components to a nucleic acid as defined in any preceding aspects. The linker may be a monovalent structure or bivalent or trivalent or tetravalent branched structure. The nucleotides may be modified as defined herein.

The functional components may therefore be ligands (or targeting agents). Where multiple functional components are present, they may be the same or different. Where the functional components are ligands, they may be saccharides, and may therefore be (or comprise) GalNAc.

In one aspect, the nucleic acid is conjugated to a heterologous moiety comprising a compound of formula (II):

[S—X¹—P—X²]₃-A-X³— (II)

wherein:
S represents a functional component, e.g., a ligand, such as a saccharide, preferably wherein the saccharide is N-acetyl galactosamine;
X¹ represents C₃-C₆ alkylene or (—CH₂—CH₂—O)m(—CH₂)₂— wherein m is 1, 2, or 3;
P is a phosphate or modified phosphate, preferably a thiophosphate;
X² is alkylene or an alkylene ether of the formula (—CH₂)ₙ—O—CH₂— where n=1-6;
A is a branching unit;
X³ represents a bridging unit;
wherein a nucleic acid according to the present invention is conjugated to X³ via a phosphate or modified phosphate, preferably a thiophosphate.

In formula (II), the branching unit “A” preferably branches into three in order to accommodate three saccharide ligands. The branching unit is preferably covalently attached to the remaining tethered portions of the ligand and the nucleic acid. The branching unit may comprise a branched aliphatic group comprising groups selected from alkyl, amide, disulphide, polyethylene glycol, ether, thioether and hydroxyamino groups. The branching unit may comprise groups selected from alkyl and ether groups.

The branching unit A may have a structure selected from:

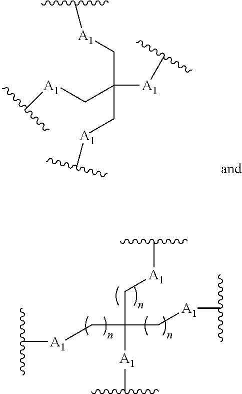

wherein each A₁ independently represents O, S, C═O or NH; and each n independently represents an integer from 1 to 20.

The branching unit may have a structure selected from:

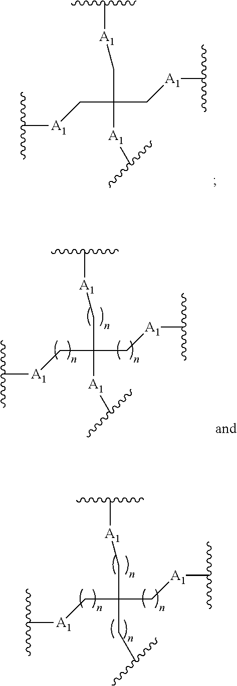

wherein each A₁ independently represents O, S, C═O or NH; and each n independently represents an integer from 1 to 20.

The branching unit may have a structure selected from:

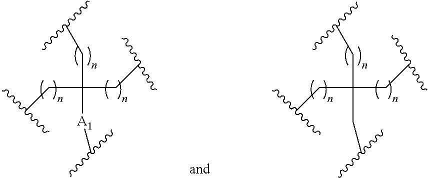

wherein A₁ is O, S, C═O or NH; and each n independently represents an integer from 1 to 20. The branching unit may have the structure:

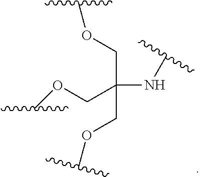 [SMILES: *NC(CO*)(CO*)CO*]

The branching unit may have the structure:

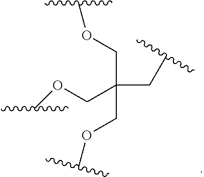 [SMILES: *CC(CO*)(CO*)CO*]

The branching unit may have the structure:

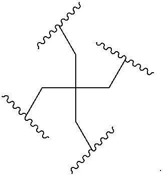 [SMILES: *CC(C*)(C*)C*]

Alternatively, the branching unit A may have a structure selected from:

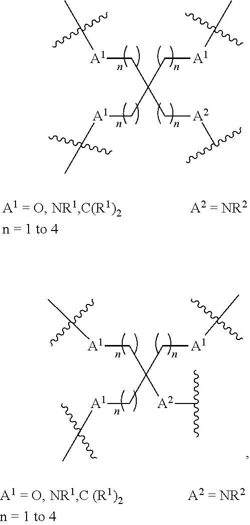

wherein:
R1 is hydrogen or C1-C10 alkylene;
and R2 is C1-C10 alkylene.

Optionally, the branching unit consists of only a carbon atom.

The “X³” portion is a bridging unit. The bridging unit is linear and is covalently bound to the branching unit and the nucleic acid.

X³ may be selected from —C₁-C₂₀ alkylene-, —C₂-C₂₀ alkenylene-, an alkylene ether of formula —(C₁-C₂₀ alkylene)-O—(C₁-C₂₀ alkylene)-, —C(O)—C₁-C₂₀ alkylene-, —C₀-C₄ alkylene(Cy)C₀-C₄ alkylene- wherein Cy represents a substituted or unsubstituted 5 or 6 membered cycloalkylene, arylene, heterocyclylene or heteroarylene ring, —C₁-C₄ alkylene-NHC(O)—C₁-C₄ alkylene-, —C₁-C₄ alkylene-C(O)NH—C₁-C₄ alkylene-, —C₁-C₄ alkylene-SC(O)—C₁-C₄ alkylene-, —C₁-C₄ alkylene-C(O)S—C₁-C₄ alkylene-, —C₁-C₄ alkylene-OC(O)—C₁-C₄ alkylene-, —C₁-C₄ alkylene-C(O)O—C₁-C₄ alkylene-, and —C₁-C₆ alkylene-S—S—C₁-C₆ alkylene-.

X³ may be an alkylene ether of formula —(C₁-C₂₀ alkylene)-O—(C₁-C₂₀ alkylene)-. X³ may be an alkylene ether of formula —(C₁-C₂₀ alkylene)-O—(C₄-C₂₀ alkylene)-, wherein said (C₄-C₂₀ alkylene) is linked to Z. X³ may be selected from the group consisting of —CH₂—O—C₃H₆—, —CH₂—O—C₄H₈—, —CH₂—O—C₆H₁₂— and —CH₂—O—C₈H₁₆—, especially —CH₂—O—C₄H₈—, —CH₂—O—C₆H₁₂— and —CH₂—O—C₈H₁₆—, wherein in each case the —CH₂— group is linked to A.

In one aspect, the nucleic acid is conjugated to a heterologous moiety of formula (III):

[S—X¹—P—X²]₃-A-X³— (III)

wherein:
S represents a functional component, e.g., a ligand, such as a saccharide, preferably GalNAc;
X¹ represents C₃-C₆ alkylene or (—CH₂—CH₂—O)ₘ(—CH₂)₂— wherein m is 1, 2, or 3;
P is a phosphate or modified phosphate, preferably a thiophosphate;
X² is C₁-C₈ alkylene;
A is a branching unit selected from:

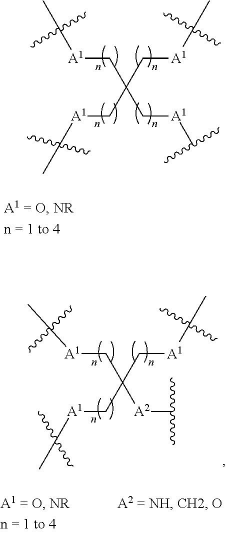

X³ is a bridging unit;
wherein a nucleic acid according to the present invention is conjugated to X³ via a phosphate or a modified phosphate, preferably a thiophosphate.

The branching unit A may have the structure:

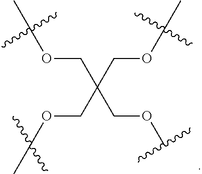 [SMILES: *OCC(CO*)(CO*)CO*]

The branching unit A may have the structure:

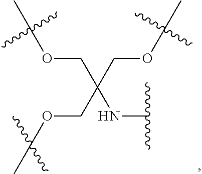

wherein X³ is attached to the nitrogen atom.

X³ may be C₁-C₂₀ alkylene. Preferably, X³ is selected from the group consisting of —C₃H₆—, —C₄H₈—, —C₆H₁₂— and —C₈H₁₆—, especially —C₄H₈—, —C₆H₁₂— and —C₈H₁₆—.

In one aspect, the nucleic acid is conjugated to a ligand comprising a compound of formula (IV):

[S—X¹—P—X²]₃-A-X³— (IV)

wherein:
S represents a functional component, e.g., a ligand, such as a saccharide, preferably GalNAc;
X¹ represents C₃-C₆ alkylene or (—CH₂—CH₂—O)ₘ(—CH₂)₂— wherein m is 1, 2, or 3;
P is a phosphate or modified phosphate, preferably a thiophosphate;
X² is an alkylene ether of formula —C₃H₆—O—CH₂—;
A is a branching unit;
X³ is an alkylene ether of formula selected from the group consisting of —CH₂—O—CH₂—, —CH₂—O—C₂H₄—, —CH₂—O—C₃H₆—, —CH₂—O—C₄H₈—, —CH₂—O—C₅H₁₀—, —CH₂—O—C₆H₁₂—, —CH₂—O—C₇H₁₄—, and —CH₂—O—C₈H₁₆—, wherein in each case the —CH₂— group is linked to A,
and wherein X³ is conjugated to a nucleic acid according to the present invention by a phosphate or modified phosphate, preferably a thiophosphate.

The branching unit may comprise carbon. Preferably, the branching unit is a carbon.

X³ may be selected from the group consisting of —CH₂—O—C₄H₈—, —CH₂—O—C₅H₁₀—, —CH₂—O—C₆H₁₂—, —CH₂—O—C₇H₁₄—, and —CH₂—O—C₈H₁₆—. Preferably, X³ is selected from the group consisting of —CH₂—O—C₄H₈—, —CH₂—O—C₆H₁₂— and —CH₂—O—C₈H₁₆.

X¹ may be (—CH₂—CH₂—O)(—CH₂)₂—. X¹ may be (—CH₂—CH₂—O)₂(—CH₂)₂—. X¹ may be (—CH₂—CH₂—O)₃(—CH₂)₂—. Preferably, X¹ is (—CH₂—CH₂—O)₂(—CH₂)₂—. Alternatively, X¹ represents C₃-C₆ alkylene. X¹ may be propylene. X¹ may be butylene. X¹ may be pentylene. X¹ may be hexylene. Preferably the alkyl is a linear alkylene. In particular, X¹ may be butylene.

X² represents an alkylene ether of formula —C₃H₆—O—CH₂— i.e., C₃ alkoxy methylene, or —CH₂CH₂CH₂OCH₂—.

For any of the above aspects, when P represents a modified phosphate group, P can be represented by:

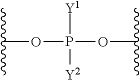

wherein Y¹ and Y² each independently represent ═O, ═S, —O—, —OH, —SH, —BH₃, —OCH₂CO₂, —OCH₂CO₂R^{x}, —OCH₂C(S)OR^{x}, and —OR^{x}, wherein R^{x} represents C₁-C₆ alkyl and wherein [symbol] indicates attachment to the remainder of the compound.

By modified phosphate it is meant a phosphate group wherein one or more of the non-linking oxygens is replaced. Examples of modified phosphate groups include phosphorothioate, phosphorodithioates, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulphur. One, each or both non-linking oxygens in the phosphate group can be independently any one of S, Se, B, C, H, N, or OR (R is alkyl or aryl).

The phosphate can also be modified by replacement of a linking oxygen with nitrogen (bridged phosphoroamidates), sulfur (bridged phosphorothioates) and carbon (bridged methylenephosphonates). The replacement can occur at a terminal oxygen. Replacement of the non-linking oxygens with nitrogen is possible.

For example, Y¹ may represent —OH and Y² may represent ═O or ═S; or
Y¹ may represent —O— and Y² may represent ═O or ═S;
Y¹ may represent ═O and Y² may represent —CH₃, —SH, —OR^{x}, or —BH₃
Y¹ may represent ═S and Y² may represent —CH₃, OR^{x} or —SH.

It will be understood by the skilled person that in certain instances there will be delocalisation between Y¹ and Y².

Preferably, the modified phosphate group is a thiophosphate group. Thiophosphate groups include bithiophosphate (i.e., where Y¹ represents ═S and Y² represents —S—) and monothiophosphate (i.e., where Y¹ represents —O— and Y² represents ═S, or where Y¹ represents ═O and Y² represents —S—). Preferably, P is a monothiophosphate. The inventors have found that conjugates having thiophosphate groups in replacement of phosphate groups have improved potency and duration of action in vivo.

P may also be an ethylphosphate (i.e., where Y¹ represents ═O and Y² represents OCH₂CH₃).

The ligand, e.g., saccharide, may be selected to have an affinity for at least one type of receptor on a target cell. In particular, the receptor is on the surface of a mammalian liver cell, for example, the hepatic asialoglycoprotein receptor complex (ASGP-R).

For any of the above or below aspects, the saccharide may be selected from N-acetyl with one or more of galactosamine, mannose, galactose, glucose, glucosamine and fructose. Typically, a ligand to be used in the present invention may include N-acetyl galactosamine (GalNAc). Preferably the compounds of the invention may have 3 ligands, which will each preferably include N-acetyl galactosamine.

“GalNAc” refers to 2-(Acetylamino)-2-deoxy-D-galactopyranose, commonly referred to in the literature as N-acetyl galactosamine. Reference to “GalNAc” or “N-acetyl galactosamine” includes both the β-form: 2-(Acetylamino)-2-deoxy-β-D-galactopyranose and the α-form: 2-(Acetylamino)-2-deoxy-α-D-galactopyranose. In certain embodiments, both the β-form: 2-(Acetylamino)-2-deoxy-β-D-galactopyranose and α-form: 2-(Acetylamino)-2-deoxy-α-D-galactopyranose may be used interchangeably. Preferably, the compounds of the invention comprise the β-form, 2-(Acetylamino)-2-deoxy-β-D-galactopyranose.

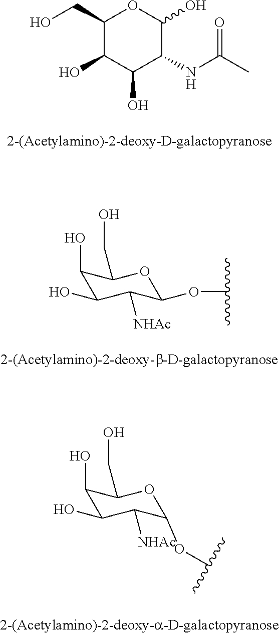 [SMILES: CC(=O)N[C@H]1C(O)O[C@H](CO)[C@H](O)[C@@H]1O ; *OC1OC(CO)C(O)C(O)C1NC(C)=O ; *OC1OC(CO)C(O)C(O)C1NC(C)=O]

In one aspect, the nucleic acid is a conjugated nucleic acid, wherein the nucleic acid is conjugated to a heterologous moiety with one of the following structures, which may be referred to as “triantennary ligands” for ease of reference:

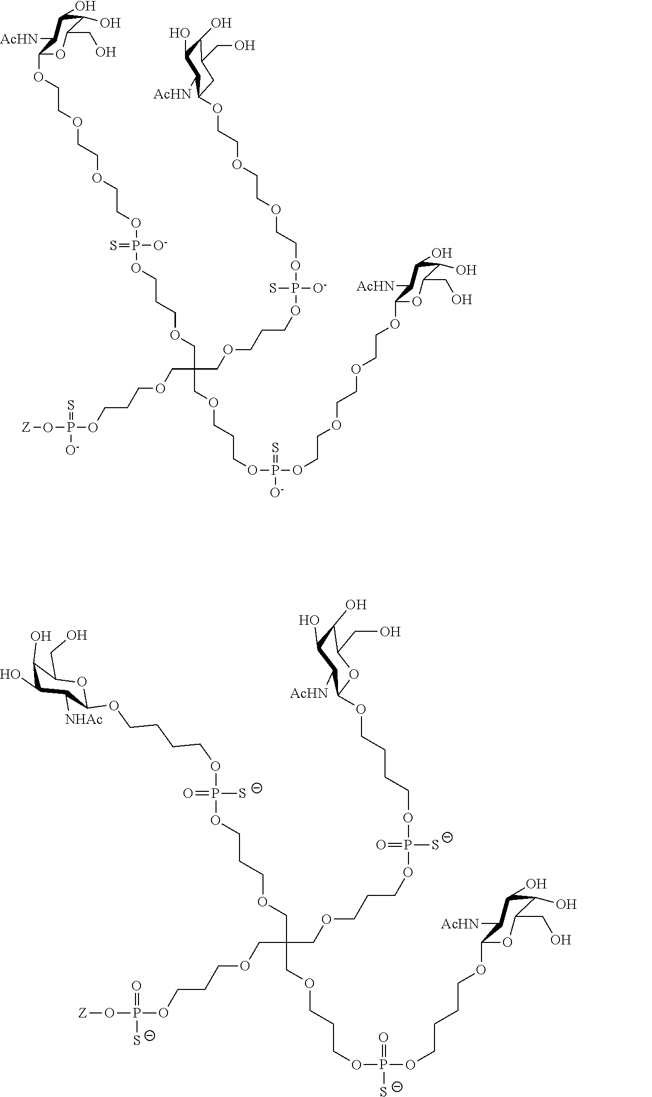

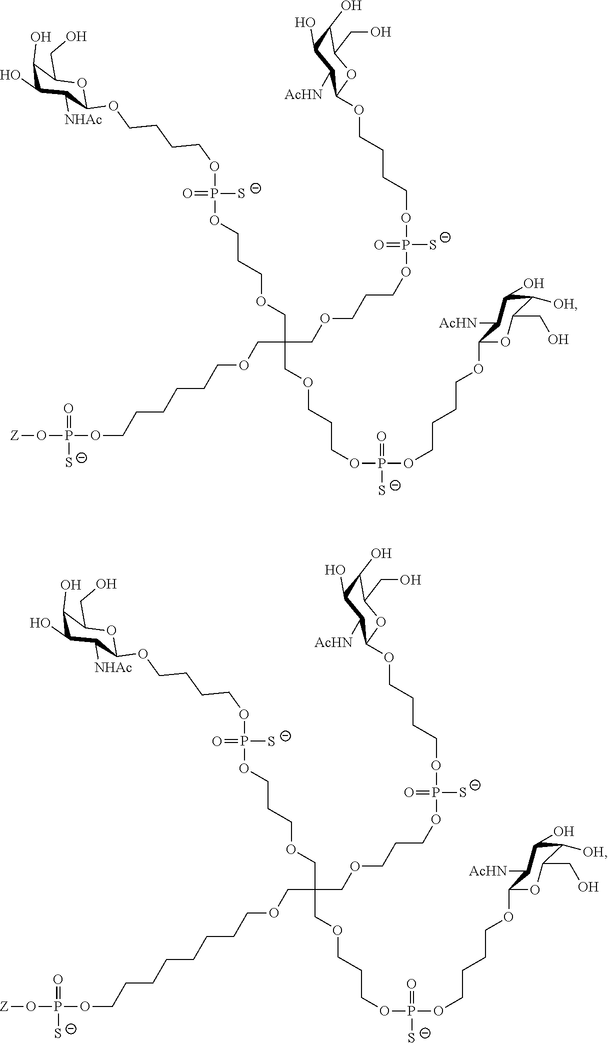

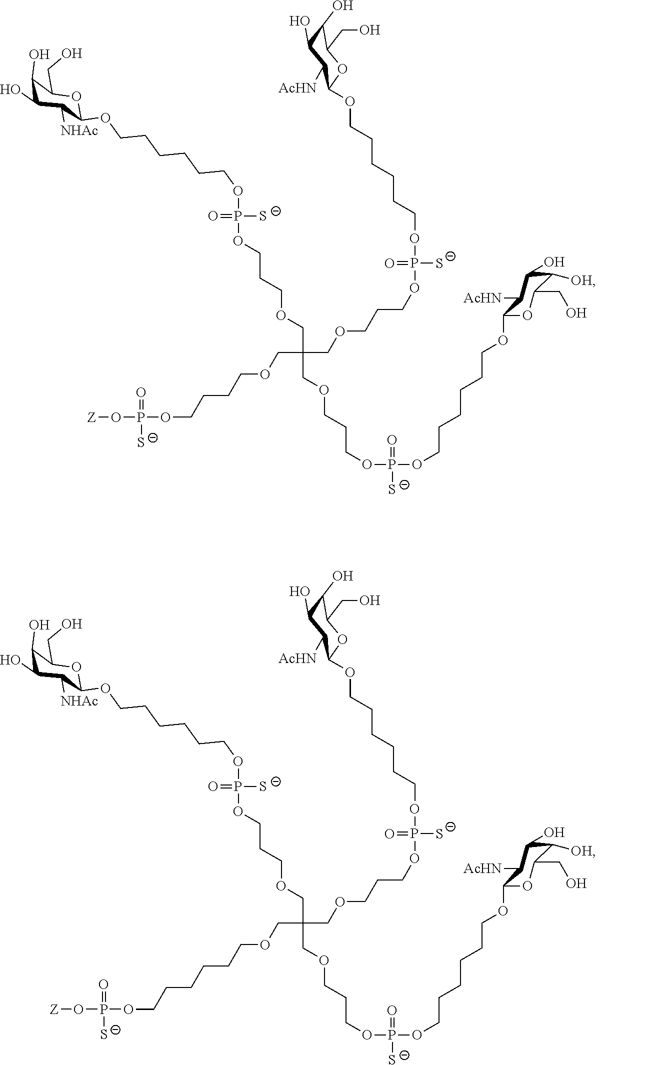

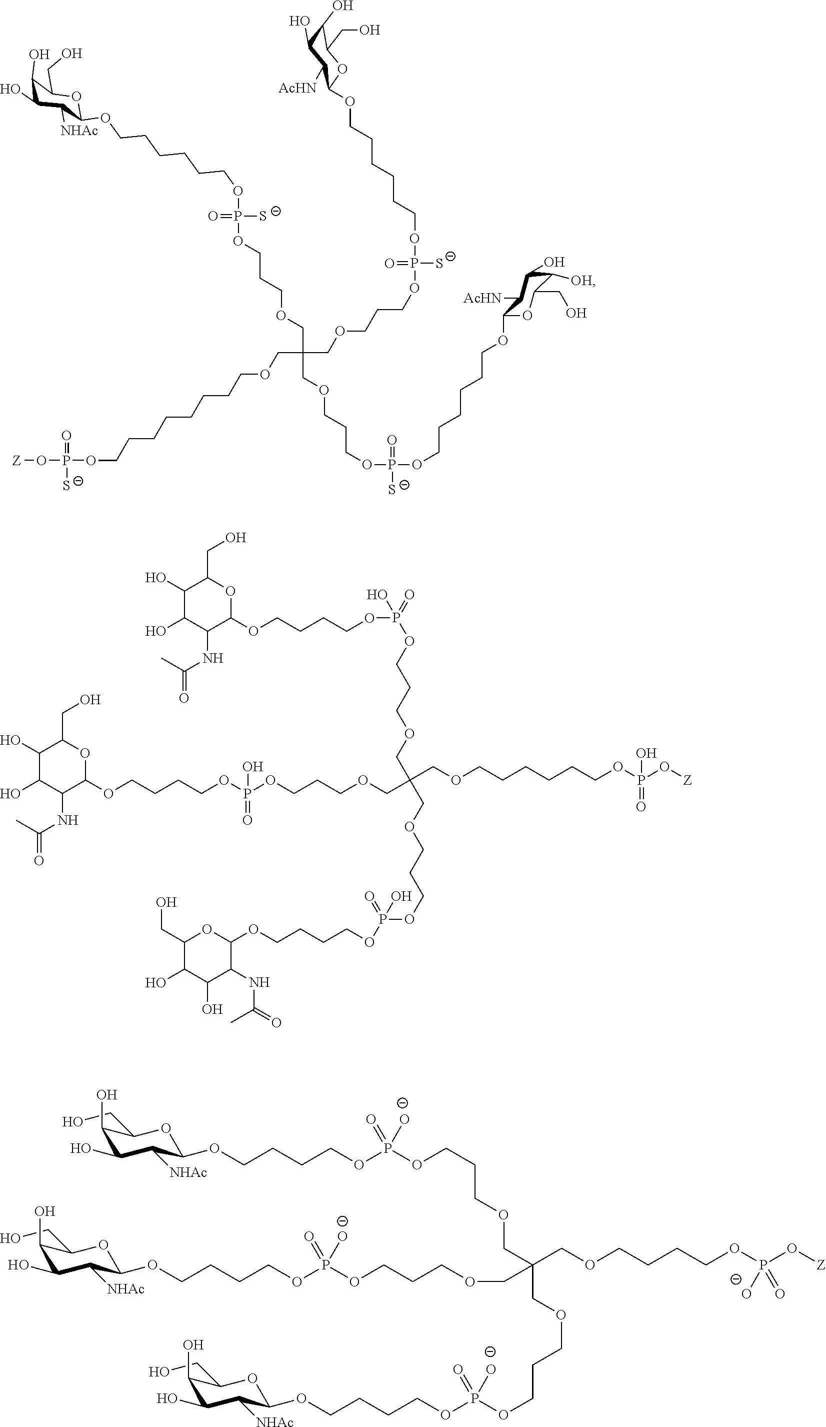

wherein Z is any nucleic acid as defined herein.

In certain embodiments the nucleic acid Z is conjugated to the triantennary ligand via the phosphate or thiophosphate group which links the triantennary ligand to the 3′ or 5′ position of the sugar, particularly to the 3′ or 5′ position of the ribose, of the terminal nucleotide of said nucleic acid Z.

In certain embodiments the heterologous moiety (“triantennary ligand”) is conjugated to the 3′ position of the ribose of the terminal nucleotide of the second (sense) strand of Z (which is also referred to as strand “B” in Tables 5a, 5b, 5c).

In other embodiments, the heterologous moiety (“triantennary ligand”) is conjugated to the 5′ position of the ribose of the terminal nucleotide of the second (sense) strand of Z (which is also referred to as strand “B” in Tables 5a, 5b, 5c).

In other embodiments, the heterologous moiety (“triantennary ligand”) is conjugated to the 3′ position of the ribose of the terminal nucleotide of the first (antisense) strand of Z (which is also referred to as strand “A” in Tables 5a, 5b, 5c).

Preferably, the nucleic acid is a conjugated nucleic acid, wherein the nucleic acid is conjugated to a triantennary ligand with one of the following structures:

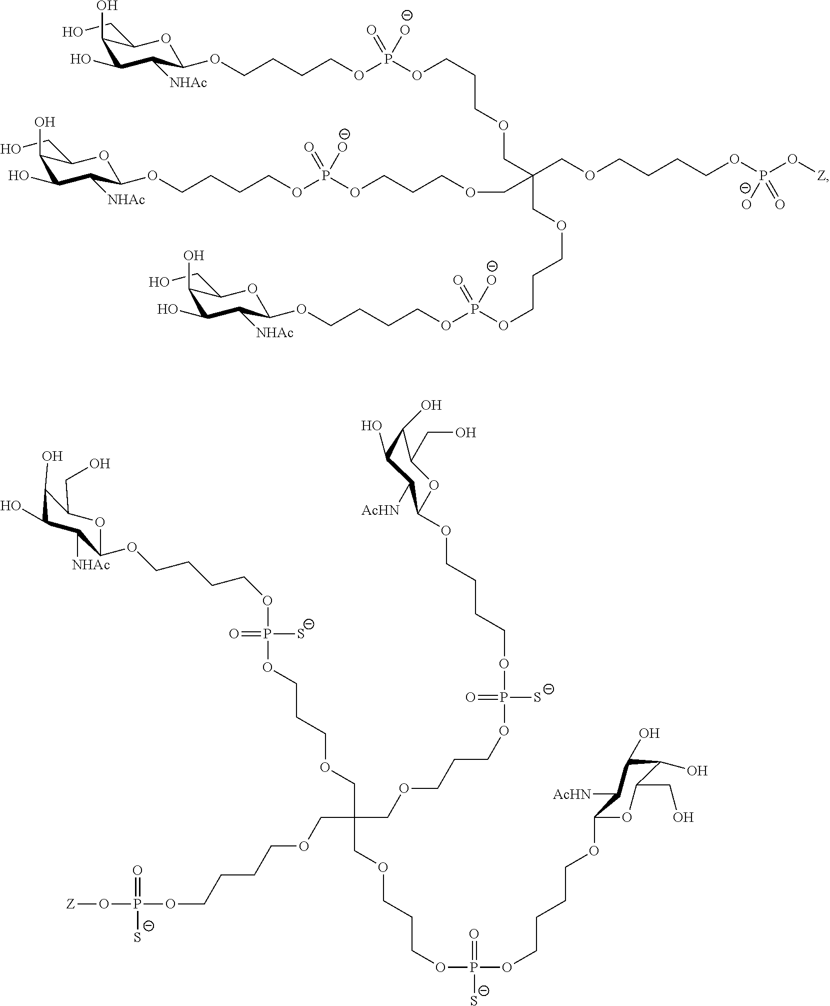

wherein Z is any nucleic acid as defined herein.

In a preferred embodiment the nucleic acid Z is conjugated to the triantennary ligand via the phosphate or thiophosphate group which links the triantennary ligand to the 3′ or 5′ position of the ribose of the terminal nucleotide of said nucleic acid Z.

Preferably, the triantennary ligand” is conjugated to the 5′ position of the ribose of the terminal nucleotide of the second (sense) strand of Z (which is also referred to as strand “B” in Tables 5a. 5b. 5c).

A heterologous moiety of formula (II), (III) or (IV) or any one of the triantennary ligands disclosed herein can be attached at the 3′-end of the first (antisense) strand and/or at any of the 3′ and/or 5′ end of the second (sense) strand. The nucleic acid can comprise more than one heterologous moiety of formula (II), (III) or (IV) or any one of the triantennary ligands disclosed herein. However, a single heterologous moiety of formula (II), (III) or (IV) or any one of the triantennary ligands disclosed herein is preferred because a single such moiety is sufficient for efficient targeting of the nucleic acid to the target cells. Preferably in that case, at least the last two, preferably at least the last three and more preferably at least the last four nucleotides at the end of the nucleic acid to which the ligand is attached are linked by a phosphodiester linkage.

Preferably, the 5′-end of the first (antisense) strand is not attached to a heterologous moiety, since attachment at this position can potentially interfere with the biological activity of the nucleic acid.

A nucleic acid with a single heterologous moiety (e.g., of formula (II), (III) or (IV) or any one of the triantennary ligands disclosed herein) at the 5′ end of a strand is easier and therefore cheaper to synthesise than the same nucleic acid with the same group at the 3′ end. Preferably therefore, a single heterologous moiety (e.g., of any of formulae (II), (III) or (IV) or any one of the triantennary ligands disclosed herein) is covalently attached to (conjugated with) the 5′ end of the second strand of the nucleic acid.

In one aspect, the first strand of the nucleic acid is a compound of formula (V):

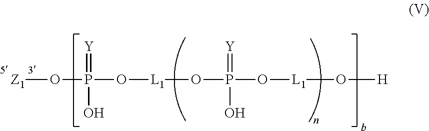

wherein b is preferably 0 or 1; and
   the second strand is a compound of formula (VI):

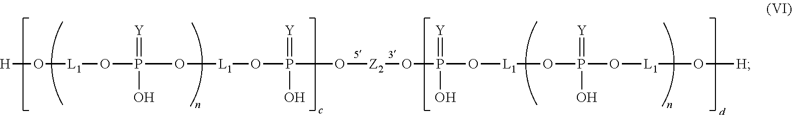

wherein:
c and d are independently preferably 0 or 1;
Z₁ and Z₂ are respectively the first and second strand of the nucleic acid;
Y is independently O or S;
n is independently 0, 1, 2 or 3; and
L₁ is a linker to which a ligand is attached, wherein L₁ is the same or different in formulae (V) and (VI), and is the same or different within formulae (V) and (VI) when L₁ is present more than once within the same formula, wherein L₁ is preferably of formula (VII);
   and wherein b+c+d is preferably 2 or 3.

Preferably, L₁ in formulae (V) and (VI) is of formula (VII):

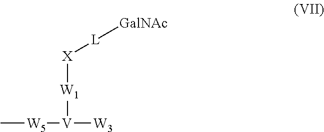

wherein:
L is selected from the group comprising, or preferably consisting of:
   —(CH₂)ᵣC(O)—, wherein r=2-12;
   —(CH₂—CH₂—O)ₛ—CH₂—C(O)—, wherein s=1-5;
   —(CH₂)ₜ—CO—NH—(CH₂)ᵣ—NH—C(O)—, wherein t is independently 1-5;
   —(CH₂)ᵤ—CO—NH—(CH₂)ᵤ—C(O)—, wherein u is independently 1-5; and
   —(CH₂)ᵥ—NH—C(O)—, wherein v is 2-12; and
wherein the terminal C(O), if present, is attached to X of formula (VII), or if X is absent, to W₁ of formula (VII), or if W₁ is absent, to V of formula (VII);
W₁, W₃ and W₅ are individually absent or selected from the group comprising, or preferably consisting of:
   —(CH₂)ᵣ, wherein r=1-7;
   —(CH₂)ₛ—O—(CH₂)ₛ—, wherein s is independently 0-5;
   —(CH₂)ₜ—S—(CH₂)ₜ—, wherein t is independently 0-5;
X is absent or is selected from the group comprising, or preferably consisting of: NH, NCH₃ or NC₂H₅;
V is selected from the group comprising, or preferably consisting of:
CH, N,

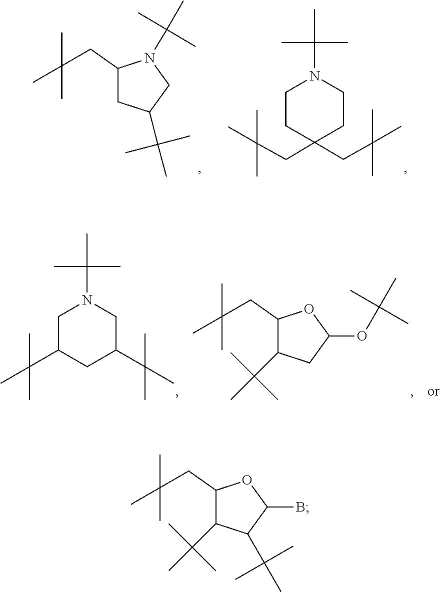

wherein B, if present, is a modified or natural nucleobase.

In one aspect, the first strand is a compound of formula (VIII)

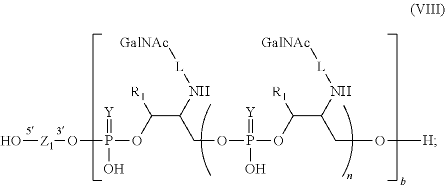

wherein b is preferably 0 or 1; and
   the second strand is a compound of formula (IX):

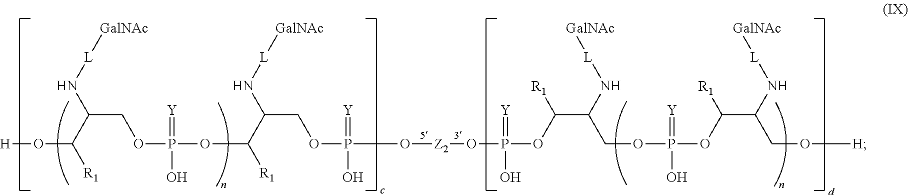

wherein c and d are independently preferably 0 or 1;
   wherein:
Z₁ and Z₂ are respectively the first and second strand of the nucleic acid;
Y is independently O or S;
R₁ is H or methyl;
n is independently preferably 0, 1, 2 or 3; and
L is the same or different in formulae (VIII) and (IX), and is the same or different within formulae (VIII) and (IX) when L is present more than once within the same formula, and is selected from the group comprising, or preferably consisting of:
   —(CH₂)ᵣ—C(O)—, wherein r=2-12;
   —(CH₂—CH₂—O)S—CH₂—C(O)—, wherein s=1-5;
   —(CH₂)ₜ—CO—NH—(CH₂)ₜ—NH—C(O)—, wherein t is independently 1-5;
   —(CH₂)ᵤ—CO—NH—(CH₂)ᵤ—C(O)—, wherein u is independently 1-5; and
   —(CH₂)ᵥ—NH—C(O)—, wherein v is 2-12; and
wherein the terminal C(O), if present, is attached to the NH group (of the linker, not of the targeting ligand);
   and wherein b+c+d is preferably 2 or 3.

In one aspect, the first strand of the nucleic acid is a compound of formula (X):

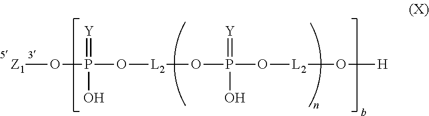

wherein b is preferably 0 or 1; and
   the second strand is a compound of formula (XI):

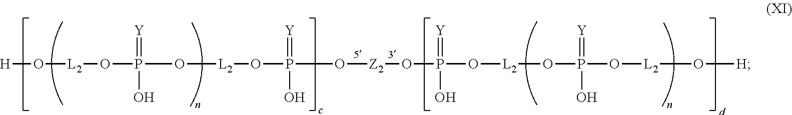

wherein:
c and d are independently preferably 0 or 1;
Z₁ and Z₂ are respectively the first and second RNA strand of the nucleic;
Y is independently O or S;
n is independently preferably 0, 1, 2 or 3; and
L₂ is the same or different in formulae (X) and (XI) and is the same or different in moieties bracketed by b, c and d, and is selected from the group comprising, or preferably consisting of:

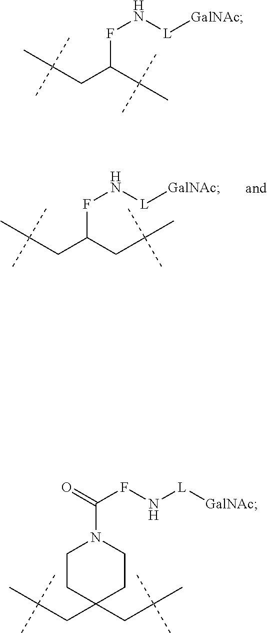

or
n is 0 and L₂ is:

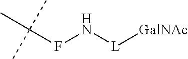

and the terminal OH group is absent such that the following moiety is formed:

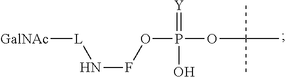

wherein:
F is a saturated branched or unbranched (such as unbranched) C₁₋₆alkyl (e.g., C₁₋₆alkyl) chain wherein one of the carbon atoms is optionally replaced with an oxygen atom provided that said oxygen atom is separated from another heteroatom (e.g. an O or N atom) by at least 2 carbon atoms;
L is the same or different in formulae (X) and (XI) and is selected from the group comprising, or preferably consisting of:
   —(CH₂)ᵣC(O)—, wherein r=2-12;
   —(CH₂—CH₂—O)ₛ—CH₂—C(O)—, wherein s=1-5;
   —(CH₂)ₜ—CO—NH—(CH₂)ₜ—NH—C(O)—, wherein t is independently 1-5;
   —(CH₂)ᵤ—CO—NH—(CH₂)ᵤ—C(O)—, wherein u is independently 1-5; and
   —(CH₂)ᵥ—NH—C(O)—, wherein v is 2-12; and
wherein the terminal C(O), if present, is attached to the NH group (of the linker, not of the targeting ligand);
   and wherein b+c+d is preferably 2 or 3.

In one aspect, b is 0, c is 1 and d is 1; b is 1, c is 0 and d is 1; b is 1, c is 1 and d is 0; or b is 1, c is 1 and d is 1 in any of the nucleic acids of formulae (V) and (VI) or (VIII) and (IX) or (X) and (XI). Preferably, b is 0, c is 1 and d is 1; b is 1, c is 0 and d is 1; or b is 1, c is 1 and d is 1. Most preferably, b is 0, c is 1 and d is 1.

In one aspect, Y is O in any of the nucleic acids of formulae (V) and (VI) or (VIII) and (IX) or (X) and (XI). In another aspect, Y is S. In a preferred aspect, Y is independently selected from O or S in the different positions in the formulae.

In one aspect, R₁ is H or methyl in any of the nucleic acids of formulae (VIII) and (IX). In one aspect, R₁ is H. In another aspect, R₁ is methyl.

In one aspect, n is 0, 1, 2 or 3 in any of the nucleic acids of formulae (V) and (VI) or (VIII) and (IX) or (X) and (XI). Preferably, n is 0.

Examples of F moieties in any of the nucleic acids of formulae (X) and (XI) include (CH₂)₁₋₆ e.g. (CH₂)₁₋₄ e.g., CH₂, (CH₂)₄, (CH₂)₅ or (CH₂)₆, or CH₂O(CH₂)₂₋₃, e.g. CH₂O(CH₂)CH₃.

In one aspect, L₂ in formulae (X) and (XI) is:

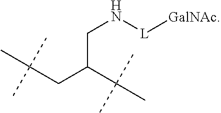

In one aspect, L₂ is:

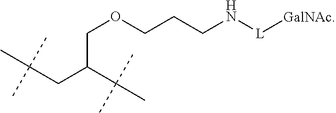

In one aspect, L₂ is:

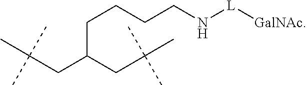

In one aspect, L₂ is:

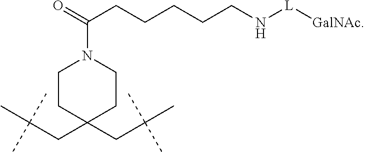

In one aspect, n is 0 and L₂ is:

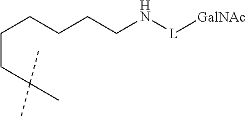

and the terminal OH group is absent such that the following moiety is formed:

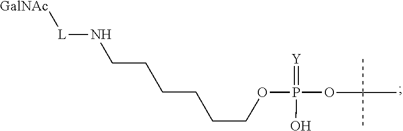

wherein Y is O or S.

In one aspect, L in the nucleic acids of formulae (V) and (VI) or (VIII) and (IX) or (X) and (XI), is selected from the group comprising, or preferably consisting of:
—(CH₂)ᵣC(O)—, wherein r=2-12;
—(CH₂—CH₂—O)ₛ—CH₂—C(O)—, wherein s=1-5;
—(CH₂)ᵣ—CO—NH—(CH₂)ₜ—NH—C(O)—, wherein t is independently 1-5;
—(CH₂)ᵤ—CO—NH—(CH₂)ᵤ—C(O)—, wherein u is independently 1-5; and
—(CH₂)ᵥ—NH—C(O)—, wherein v is 2-12;
wherein the terminal C(O) is attached to the NH group.

Preferably, L is —(CH₂)ᵣC(O)—, wherein r=2-12, more preferably r=2-6 even more preferably, r=4 or 6 e.g., 4.

Preferably, L is:

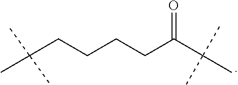

Within the moiety bracketed by b, c and d, L₂ in the nucleic acids of formulae (X) and (XI) is typically the same. Between moieties bracketed by b, c and d, L₂ may be the same or different. In an embodiment, L₂ in the moiety bracketed by c is the same as the L₂ in the moiety bracketed by d. In an embodiment, L₂ in the moiety bracketed by c is not the same as L₂ in the moiety bracketed by d. In an embodiment, the L₂ in the moieties bracketed by b, c and d is the same, for example when the linker moiety is a serinol-derived linker moiety.

Serinol derived linker moieties may be based on serinol in any stereochemistry i.e., derived from L-serine isomer, D-serine isomer, a racemic serine or other combination of isomers. In a preferred aspect of the invention, the serinol-GalNAc moiety (SerGN) has the following stereochemistry:

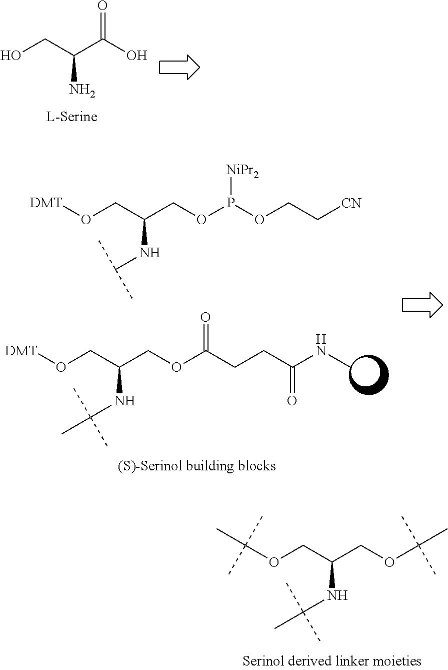

i.e., is based on an (S)-serinol-amidite or (S)-serinol succinate solid supported building block derived from L-serine isomer.

In a preferred aspect, the first strand of the nucleic acid is a compound of formula (VIII) and the second strand of the nucleic acid is a compound of formula (IX), wherein:
b is 0;
c and d are 1,
n is 0,
Z₁ and Z₂ are respectively the first and second strand of the nucleic acid,
Y is S,
R₁ is H, and
L is —(CH₂)₄—C(O)—, wherein the terminal C(O) of L is attached to the N atom of the linker (ie not a possible N atom of a targeting ligand).

In another preferred aspect, the first strand of the nucleic acid is a compound of formula (V) and the second strand of the nucleic acid is a compound of formula (VI), wherein:
b is 0,
c and d are 1,
n is 0,
Z₁ and Z₂ are respectively the first and second strand of the nucleic acid,
Y is S,
L₁ is of formula (VII), wherein:
   W₁ is —CH₂—O—(CH₂)₃—,
   W₃ is —CH₂—,
   W₅ is absent,
   V is CH,
   X is NH, and
   L is —(CH₂)₄—C(O)— wherein the terminal C(O) of L is attached to the N atom of X in formula (VII).

In another preferred aspect, the first strand of the nucleic acid is a compound of formula (V) and the second strand of the nucleic acid is a compound of formula (VI), wherein:
b is 0,
c and d are 1,
n is 0,
Z₁ and Z₂ are respectively the first and second strand of the nucleic acid,
Y is S,
L₁ is of formula (VII), wherein:
   W₁, W₃ and W₅ are absent,
   V is

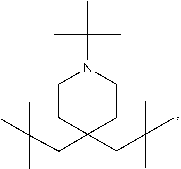 [SMILES: CC(C)(C)CC1(CC(C)(C)C)CCN(C(C)(C)C)CC1]

X is absent, and
L is —(CH₂)₄—C(O)—NH—(CH₂)₅—C(O)—, wherein the terminal C(O) of L is attached to the N atom of V in formula (VII).

In one aspect, the nucleic acid is conjugated to a triantennary ligand with the following structure:

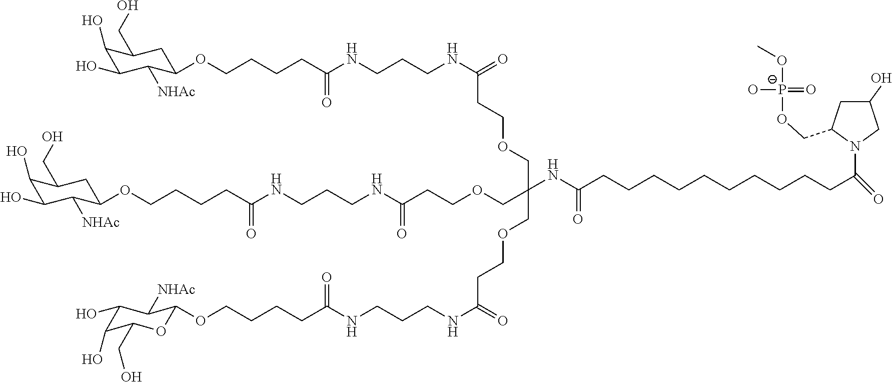

wherein the nucleic acid is conjugated to the triantennary ligand via the phosphate group of the ligand to the
a) 3′ position of the ribose of the terminal nucleotide of the second (sense) strand of Z (which is also referred to as strand “B” in Tables 5a, 5b, 5c), or
b) 5′ position of the ribose of the terminal nucleotide of the second (sense) strand of Z (which is also referred to as strand “B” in Tables 5a, 5b, 5c), or
c) 3′ position of the ribose of the terminal nucleotide of the first (antisense) strand of Z (which is also referred to as strand “A” in Tables 5a, 5b.

In one aspect of the nucleic acid, the cells that are targeted by the nucleic acid with a ligand are hepatocytes.

In any one of the above ligands where GalNAc is present, the GalNAc may be substituted for any other selected targeting ligand, such as, e.g., those mentioned herein, and in particular, mannose, galactose, glucose, glucosamine and fucose.

In one aspect, the nucleic acid is conjugated to a heterologous moiety that comprises a lipid, and more preferably, a cholesterol.

In one aspect, the double-stranded nucleic acid for inhibiting expression of MASP-2 is one of the duplexes shown in Table 5c, which may be referred to by their Duplex ID number.

In one preferred aspect, the double-stranded nucleic acid for inhibiting expression of MASP-2 is the duplex with Duplex ID number EM1203, EM1204, EM1205, EM1206, EM1207, EM1208, EM1209, EM1210, EM1211, EM1212 or EM1213.

In one preferred aspect the double-stranded nucleic acid for inhibiting expression of MASP-2 is a nucleic acid, wherein the first strand sequence comprises
(vp)-mU fC mG fG mA fG mC fG mG fA mA fG mG fU mA fA mU (ps) fG (ps) mU (SEQ ID No. 804) and optionally wherein the second strand sequence comprises
[ST23 (ps)]₃ ST41 (ps) mA mC mA mU mU mA fC fC fU mU mC mC mG mC mU mC mC (ps) mG (ps) mA (SEQ ID No: 805).

In one preferred aspect the double-stranded nucleic acid for inhibiting expression of MASP-2 is a nucleic acid, wherein the first strand sequence consists of
(vp)-mU fC mG fG mA fG mC fG mG fA mA fG mG fU mA fA mU (ps) fG (ps) mU (SEQ ID No. 804) and optionally wherein the second strand sequence consists of
[ST23 (ps)]₃ ST41 (ps) mA mC mA mU mU mA fC fC fU mU mC mC mG mC mU mC mC (ps) mG (ps) mA (SEQ ID No: 805).

In one preferred aspect the double-stranded nucleic acid for inhibiting expression of MASP-2 is a nucleic acid, wherein the first strand sequence comprises
(vp)-mU fA mC fG mA fA mG fU mC fG mU fA mC fU mC fG mC (ps) fA (ps) mG (SEQ ID No. 806) and optionally wherein the second strand sequence comprises
[ST23 (ps)]₃ ST41 (ps) mC mU mG mC mG mA fG fU fA mC mG mA mC mU mU mC mG (ps) mU (ps) mC (SEQ ID No: 807).

In one preferred aspect the double-stranded nucleic acid for inhibiting expression of MASP-2 is a nucleic acid, wherein the first strand sequence consists of
(vp)-mU fA mC fG mA fA mG fU mC fG mU fA mC fU mC fG mC (ps) fA (ps) mG (SEQ ID No. 806) and optionally wherein the second strand sequence consists of
[ST23 (ps)]₃ ST41 (ps) mC mU mG mC mG mA fG fU fA mC mG mA mC mU mU mC mG (ps) mU (ps) mC (SEQ ID No: 807).

In one preferred aspect the double-stranded nucleic acid for inhibiting expression of MASP-2 is a nucleic acid, wherein the first strand sequence comprises
(vp)-mU fA mA fG mG fU mA fA mU fG mU fC mC fA mG fG mC (ps) fU (ps) mG (SEQ ID No. 810) and optionally wherein the second strand sequence comprises
[ST23 (ps)]₃ ST41 (ps) mC mA mG mC mC mU fG fG fA mC mA mU mU mA mC mC mU (ps) mU (ps) mC (SEQ ID No: 811).

In one preferred aspect the double-stranded nucleic acid for inhibiting expression of MASP-2 is a nucleic acid, wherein the first strand sequence consists of
(vp)-mU fA mA fG mG fU mA fA mU fG mU fC mC fA mG fG mC (ps) fU (ps) mG (SEQ ID No. 810) and optionally wherein the second strand sequence consists of
[ST23 (ps)]₃ ST41 (ps) mC mA mG mC mC mU fG fG fA mC mA mU mU mA mC mC mU (ps) mU (ps) mC (SEQ ID No: 811).

### Compositions, Uses and Methods

The present invention also provides compositions comprising a nucleic acid of the invention. The nucleic acids and compositions may be used as therapeutic or diagnostic agents, alone or in combination with other agents. For example, one or more nucleic acid(s) of the invention can be combined with a delivery vehicle (e.g., liposomes) and/or excipients, such as carriers, diluents. Other agents such as preservatives and stabilizers can also be added. Pharmaceutically acceptable salts or solvates of any of the nucleic acids of the invention are likewise within the scope of the present invention. Methods for the delivery of nucleic acids are known in the art and within the knowledge of the person skilled in the art.

Compositions disclosed herein are particularly pharmaceutical compositions. Such compositions are suitable for administration to a subject.

In one aspect, the composition comprises a nucleic acid disclosed herein, or a pharmaceutically acceptable salt or solvate thereof, and a solvent (preferably water) and/or a delivery vehicle and/or a physiologically acceptable excipient and/or a carrier and/or a salt and/or a diluent and/or a buffer and/or a preservative.

Pharmaceutically acceptable carriers or diluents include those used in formulations suitable for oral, rectal, nasal or parenteral (including subcutaneous, intramuscular, intravenous, intradermal, and transdermal) administration. The formulations may conveniently be presented in unit dosage form and may be prepared by any of the methods well known in the art of pharmacy. Subcutaneous or transdermal modes of administration may be particularly suitable for the compounds described herein.

The prophylactically or therapeutically effective amount of a nucleic acid of the present invention will depend on the route of administration, the type of mammal being treated, and the physical characteristics of the specific mammal under consideration. These factors and their relationship to determining this amount are well known to skilled practitioners in the medical arts. This amount and the method of administration can be tailored to achieve optimal efficacy, and may depend on such factors as weight, diet, concurrent medication and other factors, well known to those skilled in the medical arts. The dosage sizes and dosing regimen most appropriate for human use may be guided by the results obtained by the present invention and may be confirmed in properly designed clinical trials.

An effective dosage and treatment protocol may be determined by conventional means, starting with a low dose in laboratory animals and then increasing the dosage while monitoring the effects, and systematically varying the dosage regimen as well. Numerous factors may be taken into consideration by a clinician when determining an optimal dosage for a given subject. Such considerations are known to the skilled person.

Nucleic acids of the present invention, or salts thereof, may be formulated as pharmaceutical compositions prepared for storage or administration, which typically comprise a prophylactically or therapeutically effective amount of a nucleic acid of the invention, or a salt thereof, in a pharmaceutically acceptable carrier.

The nucleic acid or conjugated nucleic acid of the present invention can also be administered in combination with other therapeutic compounds, either administrated separately or simultaneously, e.g., as a combined unit dose. The invention also includes a composition comprising one or more nucleic acids according to the present invention in a physiologically/pharmaceutically acceptable excipient, such as a stabilizer, preservative, diluent, buffer, and the like.

In one aspect, the composition comprises a nucleic acid disclosed herein and a further therapeutic agent selected from the group comprising an oligonucleotide, a small molecule, a monoclonal antibody, a polyclonal antibody and a peptide. Preferably, the further therapeutic agent is an agent that targets, preferably inhibits the expression or the activity, of MASP-2 or of another element, such as a protein, of the immune system or more specifically of the complement pathway. Preferably, the further therapeutic agent is one of the following: a) a peptide that inhibits the expression or activity of one of the components of the complement pathway, preferably MASP-2, C4, C2, C3, C5 or one of their subunits or proteolytic cleavage products; b) an antibody that specifically binds under physiological conditions to one of the components of the complement pathway, preferably MASP-2, C4, C2, C3, C5 or one of their subunits or proteolytic cleavage products; c) Eculizumab or an antigen-binding derivative thereof; d) Narsoplimab (OMS721) or an antigen-binding derivative.

Eculizumab is a humanised monoclonal antibody that specifically binds to the complement component C5 and is commercialised under the trade name SOLIRIS®. It specifically binds the complement component C5 with high affinity and inhibits cleavage of C5 to C5a and C5b. The antibody is for example described in the patent EP 0 758 904 B1 and its family members.

In certain embodiments, two or more nucleic acids of the invention with different sequences may be administered simultaneously or sequentially.

In another aspect, the present invention provides a composition, e.g., a pharmaceutical composition, comprising one or a combination of different nucleic acids of the invention and at least one pharmaceutically acceptable carrier.

Dosage levels for the therapeutic agents and compositions of the invention can be determined by those skilled in the art by experimentation. In one aspect, a unit dose may contain between about 0.01 mg/kg and about 100 mg/kg body weight of nucleic acid or conjugated nucleic acid. Alternatively, the dose can be from 10 mg/kg to 25 mg/kg body weight, or 1 mg/kg to 10 mg/kg body weight, or 0.05 mg/kg to 5 mg/kg body weight, or 0.1 mg/kg to 5 mg/kg body weight, or 0.1 mg/kg to 1 mg/kg body weight, or 0.1 mg/kg to 0.5 mg/kg body weight, or 0.5 mg/kg to 1 mg/kg body weight. Alternatively, the dose can be from about 0.5 mg/kg to about 10 mg/kg body weight, or about 0.6 mg/kg to about 8 mg/kg body weight, or about 0.7 mg/kg to about 7 mg/kg body weight, or about 0.8 mg/kg to about 6 mg/kg body weight, or about 0.9 mg/kg to about 5.5 mg/kg body weight, or about 1 mg/kg to about 5 mg/kg body weight, or about 1 mg/kg body weight, or about 3 mg/kg body weight, or about 5 mg/kg body weight, wherein “about” is a deviation of up to 30%, preferably up to 20%, more preferably up to 10%, yet more preferably up to 5% and most preferably 0% from the indicated value. Dosage levels may also be calculated via other parameters such as, e.g., body surface area.

A dose unit of these nucleic acids preferably comprises about 1 mg/kg to about 5 mg/kg body weight, or about 1 mg/kg to about 3 mg/kg body weight, or about 1 mg/kg body weight, or about 3 mg/kg body weight, or about 5 mg/kg body weight. The MASP-2 mRNA level in the liver and/or the MASP-2 protein level in the plasma or blood of a subject treated by a dose unit of the nucleic acid is preferably decreased at the time point of maximum effect by at least 30%, at least 40%, at least 50%, at least 60% or at least 70% as compared to a control that was not treatment with the nucleic acid or treated with a control nucleic acid under comparable conditions.

The dosage and frequency of administration may vary depending on whether the treatment is therapeutic or prophylactic (e.g., preventative), and may be adjusted during the course of treatment. In certain prophylactic applications, a relatively low dosage is administered at relatively infrequent intervals over a relatively long period of time. Some subjects may continue to receive treatment over their lifetime. In certain therapeutic applications, a relatively high dosage at relatively short intervals is sometimes required until progression of the disease is reduced or until the patient shows partial or complete amelioration of symptoms of disease. Thereafter, the patient may be switched to a suitable prophylactic dosing regimen.

Actual dosage levels of a nucleic acid of the invention alone or in combination with one or more other active ingredients in the pharmaceutical compositions of the present invention may be varied so as to obtain an amount of the active ingredient which is effective to achieve the desired therapeutic response for a particular patient, composition, and mode of administration, without causing deleterious side effects to the subject or patient. A selected dosage level will depend upon a variety of factors, such as pharmacokinetic factors, including the activity of the particular nucleic acid or composition employed, the route of administration, the time of administration, the rate of excretion of the particular nucleic acid being employed, the duration of the treatment, other drugs, compounds and/or materials used in combination with the particular compositions employed, the age, sex, weight, condition, general health and prior medical history of the subject or patient being treated, and similar factors well known in the medical arts.

The pharmaceutical composition may be a sterile injectable aqueous suspension or solution, or in a lyophilized form.

The pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms. It may be provided in single dose injectable form, for example in the form of a pen. Compositions may be formulated for any suitable route and means of administration.

The therapeutic agents and pharmaceutical compositions of the present invention may be administered to a mammalian subject in a pharmaceutically effective dose. The mammal may be selected from a human, a non-human primate, a simian or prosimian, a dog, a cat, a horse, cattle, a pig, a goat, a sheep, a mouse, a rat, a hamster, a hedgehog and a guinea pig, or other species of relevance. On this basis, “MASP-2” as used herein denotes nucleic acid or protein in any of the above-mentioned species, if expressed therein naturally or artificially, but preferably this wording denotes human nucleic acids or proteins.

Pharmaceutical compositions of the invention may be administered alone or in combination with one or more other therapeutic or diagnostic agents. A combination therapy may include a nucleic acid of the present invention combined with at least one other therapeutic agent selected based on the particular patient, disease or condition to be treated. Examples of other such agents include, inter alia, a therapeutically active small molecule or polypeptide, a single chain antibody, a classical antibody or fragment thereof, or a nucleic acid molecule which modulates gene expression of one or more additional genes, and similar modulating therapeutics which may complement or otherwise be beneficial in a therapeutic or prophylactic treatment regimen.

Pharmaceutical compositions are typically sterile and stable under the conditions of manufacture and storage. The composition may be formulated as a solution, microemulsion, liposome, or other ordered structure suitable to high drug concentration. The carrier may be a solvent or dispersion medium containing, for example, water, alcohol such as ethanol, polyol (e.g., glycerol, propylene glycol, and liquid polyethylene glycol), or any suitable mixtures. The proper fluidity may be maintained, for example, by the use of a coating such as lecithin, by the maintenance of the required particle size in the case of dispersion and by use of surfactants according to formulation chemistry well known in the art. In certain embodiments, isotonic agents, e.g., sugars, polyalcohols such as mannitol, sorbitol, or sodium chloride may be desirable in the composition. Prolonged absorption of injectable compositions may be brought about by including in the composition an agent that delays absorption for example, monostearate salts and gelatine.

One aspect of the invention is a nucleic acid, or a composition disclosed herein for use as a therapeutic agent. The nucleic acid or composition is preferably for use in the prophylaxis or treatment of a disease, disorder or syndrome.

The present invention provides a nucleic acid for use, alone or in combination with one or more additional therapeutic agents in a pharmaceutical composition, for treatment or prophylaxis of conditions, diseases and disorders responsive to inhibition of MASP-2 expression.

One aspect of the invention is the use of a nucleic acid, or a composition as disclosed herein in the prophylaxis or treatment of a disease, disorder or syndrome.

Nucleic acids and pharmaceutical compositions of the invention may be used in the treatment of a variety of conditions, disorders or diseases. Treatment with a nucleic acid of the invention preferably leads to in vivo MASP-2 depletion, preferably in the liver and/or in blood. As such, nucleic acids of the invention, and compositions comprising them, will be useful in methods for treating a variety of pathological disorders in which inhibiting the expression of MASP-2 may be beneficial. The present invention provides methods for treating a disease, disorder or syndrome comprising the step of administering to a subject in need thereof a prophylactically or therapeutically effective amount of a nucleic acid of the invention.

The invention thus provides methods of prophylaxis or treatment of a disease, disorder or syndrome, the method comprising the step of administering to a subject (e.g., a patient) in need thereof a therapeutically effective amount of a nucleic acid or pharmaceutical composition comprising a nucleic acid of the invention.

The most desirable therapeutically effective amount is an amount that will produce a desired efficacy of a particular treatment selected by one of skill in the art for a given subject in need thereof. This amount will vary depending upon a variety of factors understood by the skilled worker, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through experimentation, namely by monitoring a subject's response to administration of a compound and adjusting the dosage accordingly. See, e.g., Remington: The Science and Practice of Pharmacy 21st Ed., Univ. of Sciences in Philadelphia (USIP), Lippincott Williams & Wilkins, Philadelphia, PA, 2005.

In certain embodiments, nucleic acids and pharmaceutical compositions of the invention may be used to treat or prevent a disease, disorder or syndrome.

In certain embodiments, the present invention provides methods for prophylaxis or treatment of a disease, disorder or syndrome in a mammalian subject, such as a human, the method comprising the step of administering to a subject in need thereof a prophylactically or therapeutically effective amount of a nucleic acid as disclosed herein.

Administration of a “therapeutically effective dosage” of a nucleic acid of the invention may result in a decrease in severity of disease symptoms, an increase in frequency and duration of disease symptom-free periods, or a prevention of impairment or disability due to the disease affliction.

Nucleic acids of the invention may be beneficial in treating or diagnosing a disease, disorder or syndrome that may be diagnosed or treated using the methods described herein. Treatment and diagnosis of other diseases, disorders or syndromes are also considered to fall within the scope of the present invention.

One aspect of the invention is a method of prophylaxis or treatment of a disease, disorder or syndrome comprising administering a pharmaceutically effective dose or amount a nucleic acid or a composition disclosed herein to an individual in need of treatment, preferably wherein the nucleic acid or composition is administered to the subject subcutaneously, intravenously or by oral, rectal, pulmonary, intramuscular or intraperitoneal administration. Preferably, it is administered subcutaneously.

The relevant disease, disorder or syndrome is preferably a complement-mediated disease, disorder or syndrome or a disease disorder or syndrome associated with the complement pathway, and particularly the Alternative complement pathway.

The disease, disorder or syndrome is typically associated with aberrant activation and/or over-activation (hyper-activation) of the complement pathway (particularly the Lectin pathway) and/or with over-expression or ectopic expression or localisation or accumulation of MASP-2. One example of a disease that involves accumulation of MASP-2 is IgA nephropathy where Lectin pathway contributes to activation and deposition of C3 and C4 and induction of renal damage. The aberrant or over activation of the complement pathway may have genetic causes or may be acquired.

The disease, disorder or syndrome may be a) selected from the group comprising, and preferably consisting of C3 glomerulopathy (C3G), paroxysmal nocturnal hemoglobinuria (PNH), atypical hemolytic uremic syndrome (aHUS), lupus nephritis, IgA nephropathy (IgA N), cold agglutinin disease (CAD), myasthenia gravis (MG), primary membranous nephropathy, immune complex-mediated glomerulonephritis (IC-mediated GN), post-infectious glomerulonephritis (PIGN), systemic lupus erythematosus (SLE), ischemia/reperfusion injury, age-related macular degeneration (AMD), rheumatoid arthritis (RA), antineutrophil cytoplasmic autoantibodies-associated vasculitis (ANCA-AV), dysbiotic periodontal disease, malarial anaemia, neuromyelitis optica, post-HCT/solid organ transplant (TMAs), Guillain-Barré syndrome, membranous glomerulonephritis, thrombotic thrombocytopenic purpura and sepsis; or b) selected from the group comprising, or preferably consisting of C3 glomerulopathy (C3G), paroxysmal nocturnal hemoglobinuria (PNH), atypical hemolytic uremic syndrome (aHUS), lupus nephritis, IgA nephropathy (IgA N) and primary membranous nephropathy; or c) selected from the group comprising, or preferably consisting of C3 glomerulopathy (C3G), antineutrophil cytoplasmic autoantibodies-associated vasculitis (ANCA-AV), atypical hemolytic uremic syndrome (aHUS), cold agglutinin disease (CAD), myasthenia gravis (MG), IgA nephropathy (IgA N), paroxysmal nocturnal hemoglobinuria (PNH); d) selected from the group comprising, or preferably consisting of C3 glomerulopathy (C3G), cold agglutinin disease (CAD), myasthenia gravis (MG), neuromyelitis optica, atypical hemolytic uremic syndrome (aHUS), antineutrophil cytoplasmic autoantibodies-associated vasculitis (ANCA-AV), IgA nephropathy (IgA N), post-HCT/solid organ transplant (TMAs), Guillain-Barré syndrome, paroxysmal nocturnal hemoglobinuria (PNH), membranous glomerulonephritis, lupus nephritis and thrombotic thrombocytopenic purpura; e) C3 glomerulopathy (C3G), cold agglutinin disease (CAD) and IgA nephropathy (IgA N) or f) it is C3 glomerulopathy (C3G). The subjects to be treated with a nucleic acid or composition according to the invention are preferably subjects that are affected by or are at risk of being affected by one of these diseases, disorders or syndromes.

A nucleic acid or compositions disclosed herein may be for use in a regimen comprising treatments once or twice weekly, every week, every two weeks, every three weeks, every four weeks, every five weeks, every six weeks, every seven weeks, every eight weeks, every nine weeks, every ten weeks, every eleven weeks, every twelve weeks, every three months, every four months, every five months, every six months or in regimens with varying dosing frequency such as combinations of the before-mentioned intervals. The nucleic acid or composition may be for use subcutaneously, intravenously or using any other application routes such as oral, rectal, pulmonary, or intraperitoneal. Preferably, it is for use subcutaneously.

In cells and/or subjects treated with or receiving a nucleic acid or composition as disclosed herein, the MASP-2 expression may be inhibited compared to untreated cells and/or subjects by a range from 15% up to 100% but at least about 30%, 35%, 40%, 45%, 50%, 55%, 60%, 65%, 70%, 75%, 80%, 85%, 90%, 95%, 98%, or 100% or intermediate values. The level of inhibition may allow treatment of a disease associated with MASP-2 expression or overexpression or complement over-activation, or may serve to further investigate the functions and physiological roles of the MASP-2 gene products. The level of inhibition is preferably measured in the liver or in the blood or in the kidneys, preferably in the blood, of the subject treated with the nucleic acid or composition.

One aspect is the use of a nucleic acid or composition as disclosed herein in the manufacture of a medicament for treating a disease, disorder or syndromes, such as those as listed above or additional pathologies associated with elevated levels of MASP-2, preferably in the blood or in the kidneys, or over activation of the complement pathway, or additional therapeutic approaches where inhibition of MASP-2 expression is desired. A medicament is a pharmaceutical composition.

Each of the nucleic acids of the invention and pharmaceutically acceptable salts and solvates thereof constitutes an individual embodiment of the invention.

Also included in the invention is a method of prophylaxis or treatment of a disease, disorder or syndrome, such as those listed above, comprising administration of a composition comprising a nucleic acid or composition as described herein, to an individual in need thereof. The nucleic acid or composition may, for example, be administered in a regimen comprising treatments twice every week, once every week, every two weeks, every three weeks, every four weeks, every five weeks, every six weeks, every seven weeks, or every eight to twelve or more weeks or in regimens with varying dosing frequency such as combinations of the before-mentioned intervals. The nucleic acid or conjugated nucleic acid may be for use subcutaneously or intravenously or other application routes such as oral, rectal or intraperitoneal.

A nucleic acid of the invention may be administered by any appropriate administration pathway known in the art, including but not limited to aerosol, enteral, nasal, ophthalmic, oral, parenteral, rectal, vaginal, or transdermal (e.g., topical administration of a cream, gel or ointment, or by means of a transdermal patch). “Parenteral administration” is typically associated with injection at or in communication with the intended site of action, including infraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal administration.

The use of a chemical modification pattern of the nucleic acids confers nuclease stability in serum and makes for example subcutaneous application route feasible.

Solutions or suspensions used for intradermal or subcutaneous application typically include one or more of: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; and/or tonicity adjusting agents such as, e.g., sodium chloride or dextrose. The pH can be adjusted with acids or bases, such as hydrochloric acid or sodium hydroxide, or buffers with citrate, phosphate, acetate and the like. Such preparations may be enclosed in ampoules, disposable syringes or multiple dose vials made of glass or plastic.

Sterile injectable solutions may be prepared by incorporating a nucleic acid in the required amount in an appropriate solvent with one or a combination of ingredients described above, as required, followed by sterilization microfiltration. Dispersions may be prepared by incorporating the active compound into a sterile vehicle that contains a dispersion medium and optionally other ingredients, such as those described above. In the case of sterile powders for the preparation of sterile injectable solutions, the methods of preparation are vacuum drying and freeze-drying (lyophilization) that yield a powder of the active ingredient in addition to any additional desired ingredient from a sterile-filtered solution thereof.

When a prophylactically or therapeutically effective amount of a nucleic acid of the invention is administered by, e.g., intravenous, cutaneous or subcutaneous injection, the nucleic acid will be in the form of a pyrogen-free, parenterally acceptable aqueous solution. Methods for preparing parenterally acceptable solutions, taking into consideration appropriate pH, isotonicity, stability, and the like, are within the skill in the art. A preferred pharmaceutical composition for intravenous, cutaneous, or subcutaneous injection will contain, in addition to a nucleic acid, an isotonic vehicle such as sodium chloride injection, Ringer's injection, dextrose injection, dextrose and sodium chloride injection, lactated Ringer's injection, or other vehicle as known in the art. A pharmaceutical composition of the present invention may also contain stabilizers, preservatives, buffers, antioxidants, or other additives well known to those of skill in the art.

The amount of nucleic acid which can be combined with a carrier material to produce a single dosage form will vary depending on a variety of factors, including the subject being treated, and the particular mode of administration. In general, it will be an amount of the composition that produces an appropriate therapeutic effect under the particular circumstances. Generally, out of one hundred percent, this amount will range from about 0.01% to about 99% of nucleic acid, from about 0.1% to about 70%, or from about 1% to about 30% of nucleic acid in combination with a pharmaceutically acceptable carrier.

The nucleic acid may be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Dosage regimens may be adjusted to provide the optimum desired response (e.g., a therapeutic response). For example, a dose may be administered, several divided doses may be administered overtime, or the dose may be proportionally reduced or increased as indicated by the particular circumstances of the therapeutic situation, on a case-by-case basis. It is especially advantageous to formulate parenteral compositions in dosage unit forms for ease of administration and uniformity of dosage when administered to the subject or patient. As used herein, a dosage unit form refers to physically discrete units suitable as unitary dosages for the subjects to be treated; each unit containing a predetermined quantity of active compound calculated to produce a desired therapeutic effect. The specification for the dosage unit forms of the invention depends on the specific characteristics of the active compound and the particular therapeutic or prophylactic effect(s) to be achieved and the treatment and sensitivity of any individual patient.

The nucleic acid or composition of the present invention can be produced using routine methods in the art including chemical synthesis, such as solid phase chemical synthesis.

Nucleic acids or compositions of the invention may be administered with one or more of a variety of medical devices known in the art. For example, in one embodiment, a nucleic acid of the invention may be administered with a needleless hypodermic injection device. Examples of well-known implants and modules useful in the present invention are in the art, including e.g., implantable micro-infusion pumps for controlled rate delivery; devices for administering through the skin; infusion pumps for delivery at a precise infusion rate; variable flow implantable infusion devices for continuous drug delivery; and osmotic drug delivery systems. These and other such implants, delivery systems, and modules are known to those skilled in the art.

In certain embodiments, the nucleic acid or composition of the invention may be formulated to ensure a desired distribution in vivo. To target a therapeutic compound or composition of the invention to a particular in vivo location, they can be formulated, for example, in liposomes which may comprise one or more moieties that are selectively transported into specific cells or organs, thus enhancing targeted drug delivery.

The invention is characterized by high specificity at the molecular and tissue-directed delivery level. The sequences of the nucleic acids of the invention are highly specific for their target, meaning that they do not inhibit the expression of genes that they are not designed to target or only minimally inhibit the expression of genes that they are not designed to target and/or only inhibit the expression of a low number of genes that they are not designed to target. A further level of specificity is achieved when nucleic acids are linked to a ligand that is specifically recognised and internalised by a particular cell type. This is for example the case when a nucleic acid is linked to a ligand comprising GalNAc moieties, which are specifically recognised and internalised by hepatocytes. This leads to the nucleic acid inhibiting the expression of their target only in the cells that are targeted by the ligand to which they are linked. These two levels of specificity potentially confer a better safety profile than the currently available treatments. In certain embodiments, the present invention thus provides nucleic acids of the invention linked to a ligand comprising one or more GalNAc moieties, or comprising one or more other moieties that confer cell-type or tissue-specific internalisation of the nucleic acid thereby conferring additional specificity of target gene knockdown by RNA interference.

The nucleic acid as described herein may be formulated with a lipid in the form of a liposome. Such a formulation may be described in the art as a lipoplex. The composition with a lipid/liposome may be used to assist with delivery of the nucleic acid of the invention to the target cells. The lipid delivery system herein described may be used as an alternative to a conjugated ligand. The modifications herein described may be present when using the nucleic acid of the invention with a lipid delivery system or with a ligand conjugate delivery system.

Such a lipoplex may comprise a lipid composition comprising:
i) a cationic lipid, or a pharmaceutically acceptable salt thereof;
ii) a steroid;
iii) a phosphatidylethanolamine phospholipid; and/or
iv) a PEGylated lipid.

The cationic lipid may be an amino cationic lipid.

The cationic lipid may have the formula (XII):

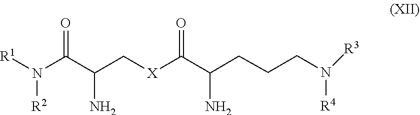

or a pharmaceutically acceptable salt thereof, wherein:
X represents O, S or NH;
R¹ and R² each independently represents a C₄-C₂₂ linear or branched alkyl chain or a C₄-C₂₂ linear or branched alkenyl chain with one or more double bonds, wherein the alkyl or alkenyl chain optionally contains an intervening ester, amide or disulfide;
when X represents S or NH, R³ and R⁴ each independently represent hydrogen, methyl, ethyl, a mono- or polyamine moiety, or R³ and R⁴ together form a heterocyclyl ring;
when X represents O, R3 and R4 each independently represent hydrogen, methyl, ethyl, a mono- or polyamine moiety, or R3 and R4 together form a heterocyclyl ring, or R³ represents hydrogen and R⁴ represents C(NH)(NH₂).

The cationic lipid may have the formula (XIII):

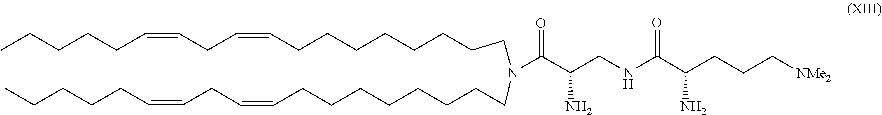 [SMILES: CCCCC/C=C\C/C=C\CCCCCCCCN(CCCCCCCC/C=C\C/C=C\CCCCC)C(=O)[C@@H](N)CNC(=O)[C@@H](N)CCCN(C)C]

or a pharmaceutically acceptable salt thereof.

The cationic lipid may have the formula (XIV):

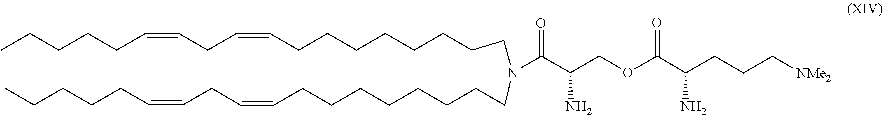 [SMILES: CCCCC/C=C\C/C=C\CCCCCCCCN(CCCCCCCC/C=C\C/C=C\CCCCC)C(=O)[C@@H](N)COC(=O)[C@@H](N)CCCN(C)C]

or a pharmaceutically acceptable salt thereof.

The content of the cationic lipid component may be from about 55 mol % to about 65 mol % of the overall lipid content of the composition. In particular, the cationic lipid component is about 59 mol % of the overall lipid content of the composition.

The compositions can further comprise a steroid. The steroid may be cholesterol. The content of the steroid may be from about 26 mol % to about 35 mol % of the overall lipid content of the lipid composition. More particularly, the content of steroid may be about 30 mol % of the overall lipid content of the lipid composition.

The phosphatidylethanolamine phospholipid may be selected from the group consisting of 1,2-diphytanoyl-sn-glycero-3-phosphoethanolamine (DPhyPE), 1,2-dioleoyl-sn-glycero-3-phosphoethanolamine (DOPE), 1,2-distearoyl-sn-glycero-3-phosphoethanolamine (DSPE), 1,2-Dilauroyl-sn-glycero-3-phosphoethanolamine (DLPE), 1,2-Dimyristoyl-sn-glycero-3-phosphoethanolamine (DMPE), 1,2-Dipalmitoyl-sn-glycero-3-phosphoethanolamine (DPPE), 1,2-Dilinoleoyl-sn-glycero-3-phosphoethanolamine (DLoPE), 1-Palmitoyl-2-oleoyl-sn-glycero-3-phosphoethanolamine (POPE), 1,2-Dierucoyl-sn-glycero-3-phosphoethanolamine (DEPE), 1,2-Disqualeoyl-sn-glycero-3-phosphoethanolamine (DSQPE) and 1-Stearoyl-2-linoleoyl-sn-glycero-3-phosphoethanolamine (SLPE). The content of the phospholipid may be about 10 mol % of the overall lipid content of the composition.

The PEGylated lipid may be selected from the group consisting of 1,2-dimyristoyl-sn-glycerol, methoxypolyethylene glycol (DMG-PEG) and C16-Ceramide-PEG. The content of the PEGylated lipid may be about 1 to 5 mol % of the overall lipid content of the composition.

The content of the cationic lipid component in the composition may be from about 55 mol % to about 65 mol % of the overall lipid content of the lipid composition, preferably about 59 mol % of the overall lipid content of the lipid composition.

The composition may have a molar ratio of the components of i):ii):iii):iv) selected from 55:34:10:1; 56:33:10:1; 57:32:10:1; 58:31:10:1; 59:30:10:1; 60:29:10:1; 61:28:10:1; 62:27:10:1; 63:26:10:1; 64:25:10:1; and 65:24:10:1.

The composition may comprise a cationic lipid having the structure

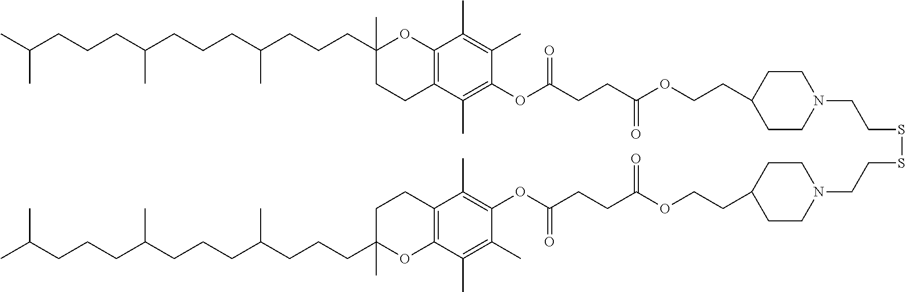 [SMILES: Cc1c(C)c2c(c(C)c1OC(=O)CCC(=O)OCCC1CCN(CCSSCCN3CCC(CCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2]

a steroid having the structure

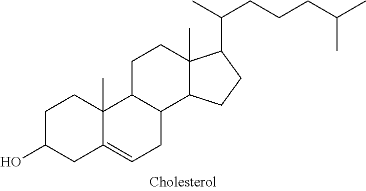 [SMILES: CC(C)CCCC(C)C1CCC2C3CC=C4CC(O)CCC4(C)C3CCC12C]

a phosphatidylethanolamine phospholipid having the structure

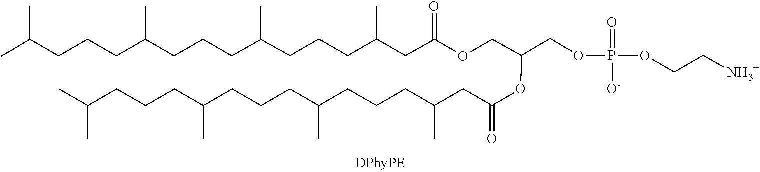 [SMILES: CC(C)CCCC(C)CCCC(C)CCCC(C)CC(=O)OCC(COP(=O)([O-])OCC[NH3+])OC(=O)CC(C)CCCC(C)CCCC(C)CCCC(C)C]

and a PEGylated lipid having the structure

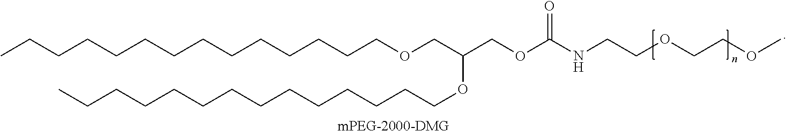

Neutral liposome compositions may be formed from, for example, dimyristoyl phosphatidylcholine (DMPC) or dipalmitoyl phosphatidylcholine (DPPC). Anionic liposome compositions may be formed from dimyristoyl phosphatidylglycerol, while anionic fusogenic liposomes may be formed primarily from dioleoyl phosphatidylethanolamine (DOPE). Another type of liposomal composition may be formed from phosphatidylcholine (PC) such as, for example, soybean PC, and egg PC. Another type is formed from mixtures of phospholipid and/or phosphatidylcholine and/or cholesterol.

A positively charged synthetic cationic lipid, N-[1-(2,3-dioleyloxy)propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells. DOTMA analogues can also be used to form liposomes.

Derivatives and analogues of lipids described herein may also be used to form liposomes.

A liposome containing a nucleic acid can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The nucleic acid preparation is then added to the micelles that include the lipid component. The cationic groups on the lipid interact with the nucleic acid and condense around the nucleic acid to form a liposome. After condensation, the detergent is removed, e.g., by dialysis, to yield a liposomal preparation of nucleic acid.

If necessary, a carrier compound that assists in condensation can be added during the condensation reaction, e.g., by controlled addition. For example, the carrier compound can be a polymer other than a nucleic acid (e.g., spermine or spermidine). pH can also be adjusted to favour condensation.

Nucleic acid formulations of the present invention may include a surfactant. In one embodiment, the nucleic acid is formulated as an emulsion that includes a surfactant.

A surfactant that is not ionized is a non-ionic surfactant. Examples include non-ionic esters, such as ethylene glycol esters, propylene glycol esters, glyceryl esters etc., nonionic alkanolamides, and ethers such as fatty alcohol ethoxylates, propoxylated alcohols, and ethoxylated/propoxylated block polymers.

A surfactant that carries a negative charge when dissolved or dispersed in water is an anionic surfactant. Examples include carboxylates, such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates.

A surfactant that carries a positive charge when dissolved or dispersed in water is a cationic surfactant. Examples include quaternary ammonium salts and ethoxylated amines.

A surfactant that has the ability to carry either a positive or negative charge is an amphoteric surfactant. Examples include acrylic acid derivatives, substituted alkylamides, N-alkylbetaines and phosphatides.

“Micelles” are defined herein as a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic. A micelle may be formed by mixing an aqueous solution of the nucleic acid, an alkali metal alkyl sulphate, and at least one micelle forming compound.

Exemplary micelle forming compounds include lecithin, hyaluronic acid, pharmaceutically acceptable salts of hyaluronic acid, glycolic acid, lactic acid, chamomile extract, cucumber extract, oleic acid, linoleic acid, linolenic acid, monoolein, monooleates, monolaurates, borage oil, evening of primrose oil, menthol, trihydroxy oxo cholanyl glycine and pharmaceutically acceptable salts thereof, glycerol, polyglycerol, lysine, polylysine, triolein, polyoxyethylene ethers and analogues thereof, polidocanol alkyl ethers and analogues thereof, chenodeoxycholate, deoxycholate, and mixtures thereof.

Phenol and/or m-cresol may be added to the mixed micellar composition to act as a stabiliser and preservative. An isotonic agent such as glycerine may as be added.

A nucleic acid preparation may be incorporated into a particle such as a microparticle. Microparticles can be produced by spray-drying, lyophilisation, evaporation, fluid bed drying, vacuum drying, or a combination of these methods.

### Definitions

As used herein, the terms “inhibit”, “down-regulate”, or “reduce” with respect to gene expression mean that the expression of the gene, or the level of RNA molecules or equivalent RNA molecules encoding one or more proteins or protein subunits (e.g., mRNA), or the activity of one or more proteins or protein subunits, is reduced below that observed either in the absence of the nucleic acid or conjugated nucleic acid of the invention or as compared to that obtained with an siRNA molecule with no known homology to the human transcript (herein termed non-silencing control). Such control may be conjugated and modified in an analogous manner to the molecule of the invention and delivered into the target cell by the same route. The expression after treatment with the nucleic acid of the invention may be reduced to 95%, 90%, 80%, 70%, 60%, 50%, 40%, 30%, 20%, 15%, 10%, 5% or 0% or to intermediate values, or less than that observed in the absence of the nucleic acid or conjugated nucleic acid. The expression may be measured in the cells to which the nucleic acid is applied. Alternatively, especially if the nucleic acid is administered to a subject, the level can be measured in a different group of cells or in a tissue or an organ or in a body fluid such as blood or plasma. The level of inhibition is preferably measured in conditions that have been selected because they show the greatest effect of the nucleic acid on the target mRNA level in cells treated with the nucleic acid in vitro. The level of inhibition may for example be measured after 24 hours or 48 hours of treatment with a nucleic acid at a concentration of between 0.038 nM-10 μM, preferably 0.5 nM, 1 nM, 10 nM or 100 nM. These conditions may be different for different nucleic acid sequences or for different types of nucleic acids, such as for nucleic acids that are unmodified or modified or conjugated to a ligand or not. Examples of suitable conditions for determining levels of inhibition are described in the examples.

By nucleic acid it is meant a nucleic acid comprising two strands comprising nucleotides, that is able to interfere with gene expression. Inhibition may be complete or partial and results in down regulation of gene expression in a targeted manner. The nucleic acid comprises two separate polynucleotide strands; the first strand, which may also be a guide strand; and a second strand, which may also be a passenger strand. The first strand and the second strand may be part of the same polynucleotide molecule that is self-complementary which ‘folds’ back to form a double-stranded molecule. The nucleic acid may be an siRNA molecule.

The nucleic acid may comprise ribonucleotides, modified ribonucleotides, deoxynucleotides, deoxyribonucleotides, or nucleotide analogues non-nucleotides that are able to mimic nucleotides such that they may ‘pair’ with the corresponding base on the target sequence or complementary strand. The nucleic acid may further comprise a double-stranded nucleic acid portion or duplex region formed by all or a portion of the first strand (also known in the art as a guide strand) and all or a portion of the second strand (also known in the art as a passenger strand). The duplex region is defined as beginning with the first base pair formed between the first strand and the second strand and ending with the last base pair formed between the first strand and the second strand, inclusive.

By duplex region it is meant the region in two complementary or substantially complementary oligonucleotides that form base pairs with one another, either by Watson-Crick base pairing or any other manner that allows for a duplex between oligonucleotide strands that are complementary or substantially complementary. For example, an oligonucleotide strand having 21 nucleotide units can base pair with another oligonucleotide of 21 nucleotide units, yet only 19 nucleotides on each strand are complementary or substantially complementary, such that the “duplex region” consists of 19 base pairs. The remaining base pairs may exist as 5′ and 3′ overhangs, or as single-stranded regions. Further, within the duplex region, 100% complementarity is not required; substantial complementarity is allowable within a duplex region. Substantial complementarity refers to complementarity between the strands such that they are capable of annealing under biological conditions. Techniques to empirically determine if two strands are capable of annealing under biological conditions are well known in the art.

Alternatively, two strands can be synthesised and added together under biological conditions to determine if they anneal to one another. The portion of the first strand and second strand that forms at least one duplex region may be fully complementary and is at least partially complementary to each other. Depending on the length of a nucleic acid, a perfect match in terms of base complementarity between the first strand and the second strand is not necessarily required. However, the first and second strands must be able to hybridise under physiological conditions.

As used herein, the terms “non-pairing nucleotide analogue” means a nucleotide analogue which includes a non-base pairing moiety including but not limited to: 6 des amino adenosine (Nebularine), 4-Me-indole, 3-nitropyrrole, 5-nitroindole, Ds, Pa, N3-Me ribo U, N3-Me riboT, N3-Me dC, N3-Me-dT, N1-Me-dG, N1-Me-dA, N3-ethyl-dC, and N3-Me dC. In some embodiments the non-base pairing nucleotide analogue is a ribonucleotide. In other embodiments it is a deoxyribonucleotide.

As used herein, the term, “terminal functional group” includes without limitation a halogen, alcohol, amine, carboxylic, ester, amide, aldehyde, ketone, and ether groups.

An “overhang” as used herein has its normal and customary meaning in the art, i.e., a single-stranded portion of a nucleic acid that extends beyond the terminal nucleotide of a complementary strand in a double-strand nucleic acid. The term “blunt end” includes double-stranded nucleic acid whereby both strands terminate at the same position, regardless of whether the terminal nucleotide(s) are base-paired. The terminal nucleotide of a first strand and a second strand at a blunt end may be base paired. The terminal nucleotide of a first strand and a second strand at a blunt end may not be paired. The terminal two nucleotides of a first strand and a second strand at a blunt end may be base-paired. The terminal two nucleotides of a first strand and a second strand at a blunt end may not be paired.

The term “serinol-derived linker moiety” means the linker moiety comprises the following structure:

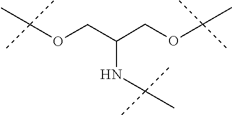

An O atom of said structure typically links to an RNA strand and the N atom typically links to the targeting ligand.

The terms “patient,” “subject,” and “individual” may be used interchangeably and refer to either a human or a non-human animal. These terms include mammals such as humans, primates, livestock animals (e.g., bovines, porcines), companion animals (e.g., canines, felines) and rodents (e.g., mice and rats).

As used herein, “treating” or “treatment” and grammatical variants thereof refer to an approach for obtaining beneficial or desired clinical results. The term may refer to slowing the onset or rate of development of a condition, disorder or disease, reducing or alleviating symptoms associated with it, generating a complete or partial regression of the condition, or some combination of any of the above. For the purposes of this invention, beneficial or desired clinical results include, but are not limited to, reduction or alleviation of symptoms, diminishment of extent of disease, stabilization (i.e., not worsening) of state of disease, delay or slowing of disease progression, amelioration or palliation of the disease state, and remission (whether partial or total), whether detectable or undetectable. “Treatment” can also mean prolonging survival relative to expected survival time if not receiving treatment. A subject (e.g., a human) in need of treatment may thus be a subject already afflicted with the disease or disorder in question. The term “treatment” includes inhibition or reduction of an increase in severity of a pathological state or symptoms relative to the absence of treatment, and is not necessarily meant to imply complete cessation of the relevant disease, disorder or condition.

As used herein, the terms “prophylaxis” and grammatical variants thereof refer to an approach for inhibiting or preventing the development, progression, or time or rate of onset of a condition, disease or disorder, and may relate to pathology and/or symptoms. For the purposes of this invention, beneficial or desired clinical results include, but are not limited to, prevention, inhibition or slowing of symptoms, progression or development of a disease, whether detectable or undetectable. A subject (e.g., a human) in need of prophylaxis may thus be a subject not yet afflicted with the disease or disorder in question. The term “prophylaxis” includes slowing the onset of disease relative to the absence of treatment, and is not necessarily meant to imply permanent prevention of the relevant disease, disorder or condition. Thus “prophylaxis” of a condition may in certain contexts refer to reducing the risk of developing the condition, or preventing, inhibiting or delaying the development of symptoms associated with the condition. It will be understood that prophylaxis may be considered as treatment or therapy.

As used herein, an “effective amount,” “prophylactically effective amount”, “therapeutically effective amount” or “effective dose” is an amount of a composition (e.g., a therapeutic composition or agent) that produces at least one desired therapeutic effect in a subject, such as preventing or treating a target condition or beneficially alleviating a symptom associated with the condition.

As used herein, the term “pharmaceutically acceptable salt” refers to a salt that is not harmful to a patient or subject to which the salt in question is administered. It may be a salt chosen, e.g., among acid addition salts and basic salts. Examples of acid addition salts include chloride salts, citrate salts and acetate salts. Examples of basic salts include salts wherein the cation is selected from alkali metal cations, such as sodium or potassium ions, alkaline earth metal cations, such as calcium or magnesium ions, as well as substituted ammonium ions, such as ions of the type N(R¹)(R²)(R³)(R⁴)+, wherein R¹, R², R³ and R⁴ independently will typically designate hydrogen, optionally substituted C1-6-alkyl groups or optionally substituted C2-6-alkenyl groups. Examples of relevant C1-6-alkyl groups include methyl, ethyl, 1-propyl and 2-propyl groups. Examples of C2-6-alkenyl groups of possible relevance include ethenyl, 1-propenyl and 2-propenyl. Other examples of pharmaceutically acceptable salts are described in “Remington's Pharmaceutical Sciences”, 17th edition, Alfonso R. Gennaro (Ed.), Mark Publishing Company, Easton, PA, USA, 1985 (and more recent editions thereof), in the “Encyclopaedia of Pharmaceutical Technology”, 3rd edition, James Swarbrick (Ed.), Informa Healthcare USA (Inc.), NY, USA, 2007, and in J. Pharm. Sci. 66: 2 (1977). A “pharmaceutically acceptable salt” retains qualitatively a desired biological activity of the parent compound without imparting any undesired effects relative to the compound. Examples of pharmaceutically acceptable salts include acid addition salts and base addition salts. Acid addition salts include salts derived from nontoxic inorganic acids, such as hydrochloric, nitric, phosphorous, phosphoric, sulfuric, hydrobromic, hydroiodic and the like, or from nontoxic organic acids such as aliphatic mono- and di-carboxylic acids, phenyl-substituted alkanoic acids, hydroxy alkanoic acids, aromatic acids, aliphatic and aromatic sulfonic acids and the like. Base addition salts include salts derived from alkaline earth metals, such as sodium, potassium, magnesium, calcium and the like, as well as from nontoxic organic amines, such as N, N′-dibenzylethylenediamine, N-methylglucamine, chloroprocaine, choline, diethanolamine, ethylenediamine, procaine and the like.

The term “pharmaceutically acceptable carrier” includes any of the standard pharmaceutical carriers. Pharmaceutically acceptable carriers for therapeutic use are well known in the pharmaceutical art, and are described, for example, in Remington's Pharmaceutical Sciences, Mack Publishing Co. (A. R. Gennaro edit. 1985). For example, sterile saline and phosphate-buffered saline at slightly acidic or physiological pH may be used. Exemplary pH buffering agents include phosphate, citrate, acetate, tris/hydroxymethyl)aminomethane (TRIS), N-Tris(hydroxymethyl)methyl-3-aminopropanesulphonic acid (TAPS), ammonium bicarbonate, diethanolamine, histidine, which is a preferred buffer, arginine, lysine, or acetate or mixtures thereof. The term further encompasses any agents listed in the US Pharmacopeia for use in animals, including humans. A “pharmaceutically acceptable carrier” includes any and all physiologically acceptable, i.e., compatible, solvents, dispersion media, coatings, antimicrobial agents, isotonic and absorption delaying agents, and the like. In certain embodiments, the carrier is suitable for intravenous, intramuscular, subcutaneous, parenteral, spinal or epidermal administration (e.g., by injection or infusion). Depending on selected route of administration, the nucleic acid may be coated in a material or materials intended to protect the compound from the action of acids and other natural inactivating conditions to which the nucleic acid may be exposed when administered to a subject by a particular route of administration.

The term “solvate” in the context of the present invention refers to a complex of defined stoichiometry formed between a solute (in casu, a nucleic acid compound or pharmaceutically acceptable salt thereof according to the invention) and a solvent. The solvent in this connection may, for example, be water or another pharmaceutically acceptable, typically small-molecular organic species, such as, but not limited to, acetic acid or lactic acid. When the solvent in question is water, such a solvate is normally referred to as a hydrate.

The invention will now be described with reference to the following non-limiting Figures and Examples.

### BRIEF DESCRIPTION OF THE FIGURES

FIG. 1. Relative MASP-2 mRNA expression normalized to PP/B mRNA in primary cynomolgus monkey hepatocytes after treatment with GalNAc-siRNA conjugates targeting MASP-2 at five different concentrations of 100 nM, 20 nM, 4 nM, 0.8 nM, and 0.16 nM. Mean values relative to untreated (ut). Cells treated with MASP-2-unspecific siRNA were used as controls (Ctr).

FIG. 2. Relative MASP-2 mRNA expression normalized to PPIB mRNA in primary cynomolgus monkey hepatocytes after treatment with GalNAc-siRNA conjugates targeting MASP-2 at five different concentrations of 100 nM, 20 nM, 4 nM, 0.8 nM, and 0.16 nM. Mean values relative to untreated (ut). Cells treated with MASP-2-unspecific siRNA were used as controls (Ctr).

FIG. 3. Relative MASP-2 mRNA expression normalized to PPIB mRNA in primary human hepatocytes after treatment with GalNAc-siRNA conjugates targeting MASP-2 at five different concentrations of 100 nM, 20 nM, 4 nM, 0.8 nM, and 0.16 nM. Mean values relative to untreated (ut). Cells treated with MASP-2-unspecific siRNA were used as controls (Ctr).

FIG. 4. Relative MASP-2 mRNA expression normalized to PPIB mRNA in primary human hepatocytes after treatment with GalNAc-siRNA conjugates targeting MASP-2 at five different concentrations of 100 nM, 20 nM, 4 nM, 0.8 nM, and 0.16 nM. Mean values relative to untreated (ut). Cells treated with MASP-2-unspecific siRNA were used as controls (Ctr).

FIG. 5. Relative MASP-2 mRNA expression normalized to PPIB mRNA in primary mouse hepatocytes after treatment with GalNAc-siRNA conjugates targeting MASP-2 at five different concentrations of 100 nM, 10 nM, 1 nM, 0.1 nM, and 0.01 nM. Mean values relative to untreated (ut). Cells treated with MASP-2-unspecific siRNA were used as controls (Ctr).

FIG. 6. Relative MASP-2 mRNA expression normalized to APOB mRNA in liver samples isolated on day 15 following subcutaneous injection of GalNAc-siRNA conjugates targeting MASP-2 at two concentrations of 1 or 5 mg/kg. Mean values relative to PBS-treated controls.

FIG. 7. Relative MASP-2 mRNA expression normalized to PPIB mRNA in primary Cynomolgus monkey hepatocytes after treatment with GalNAc-siRNA conjugates targeting MASP-2 at five different concentrations of 100 nM, 20 nM, 4 nM, 0.8 nM, and 0.16 nM. Mean values relative to untreated (ut). Cells treated with MASP-2-unspecific siRNA were used as controls (Ctr).

FIG. 8. Relative MASP-2 mRNA expression normalized to PPIB mRNA in primary human hepatocytes after treatment with GalNAc-siRNA conjugates targeting MASP-2 at five different concentrations of 100 nM, 20 nM, 4 nM, 0.8 nM, and 0.16 nM. Mean values relative to untreated (ut). Cells treated with MASP-2-unspecific siRNA used as controls (Ctr).

FIG. 9. Relative MASP-2 mRNA expression normalized to PPIB mRNA in primary mouse hepatocytes after treatment with GalNAc-siRNA conjugates targeting Masp-2 at five different concentrations of 100 nM, 10 nM, 1 nM, 0.1 nM, and 0.01 nM. Mean values relative to untreated (ut). Cells treated with MASP-2-unspecific siRNA were used as controls (Ctr).

FIG. 10. MASP-2 protein levels change in cynomolgus monkey serum isolated at day 2, 3, 8, 15, 22, 29, and 57, normalized to pre-dose values, after a single subcutaneous injection of vehicle or GalNAc conjugated siRNAs EM1208, EM1209, or EM1211 at 3 mg/kg. Mean and SEM is shown.

FIG. 11. MASP-2 protein levels change in cynomolgus monkey serum isolated at day 3, 8, 15, 22, 29, 43, 57, 71, and 85, normalized to pre-dose values, after a single subcutaneous injection of vehicle or GalNAc conjugated siRNA EM1208 at 0.3, 1, 3, or 10 mg/kg. Mean and SEM is shown.

### EXAMPLES

### Example 1

In vitro study in HepG2 cells showing MASP-2 knockdown efficacy of tested siRNAs after transfection of 0.5 nM or 10 nM siRNA.

MASP-2 knockdown efficacy of siRNAs EM1001-EM1192 (Table A and Table 5b) was determined after transfection of 0.5 or 10 nM siRNA in HepG2 cells. The results are depicted in Table A below. At 10 nM remaining MASP-2 levels after knockdown reached a minimum of 42% and at 0.5 nM remaining MASP-2 levels reached a minimum of 63%. At 10 nM the most potent siRNAs were EM1172, EM1128, EM1137, EM1189, and EM1192.

For transfection of HepG2 cells with siRNAs, cells were seeded at a density of 15,000 cells/well in 96-well tissue culture plates (TPP, Cat. 92096, Switzerland). Transfection of siRNA was carried out with Lipofectamine RNAiMax (Invitrogen/Life Technologies, Cat. 13778-500, Germany) according to manufacturer's instructions directly before seeding. The dual dose screen was performed with MASP-2-siRNAs in triplicates at 10 nM and 0.5 nM, respectively, with scrambled siRNA and luciferase-targeting siRNA as unspecific controls. After 24 h of incubation with siRNAs, medium was removed, and cells were lysed in 250 μL Lysis Buffer (InviTrap RNA Cell HTS96 Kit/C (Stratec, Cat. 7061300400, Germany)) and then frozen at −80° C. RNA was isolated using the InviTrap RNA Cell HTS96 Kit/C (Stratec, Cat. 7061300400, Germany). RT-qPCR was performed using MASP-2 and HPRT specific primer probe sets and Takyon™ One-Step Low Rox Probe 5× MasterMix dTTP on the QuantStudio6 device from Applied Biosystems in single-plex 384 well format. Expression differences were calculated using the delta delta Ct method and relative expression of MASP-2 normalized to the house keeping gene HPRT was determined. Results are expressed as % remaining MASP-2 mRNA after siRNA transfection in Table A.

TABLE A
Results of dual dose screening (10 nM
and 0.5 nM) of siRNAs targeting MASP-2.
The identity of the single strands forming each of
the siRNA duplexes as well as their sequences and
modifications are to be found in Tables 5a and 5b.

| | |
|---|---|
| % remaining mRNA | % remaining mRNA |
| at 10 nM | at 0.5 nM |

| Duplex | Mean | SD | Mean | SD |
|---|---|---|---|---|
| EM1001 | 109 | 3 | 114 | 6 |
| EM1002 | 106 | 22 | 109 | 24 |
| EM1003 | 113 | 8 | 106 | 2 |
| EM1004 | 115 | 9 | 110 | 7 |
| EM1005 | 96 | 10 | 89 | 15 |
| EM1006 | 100 | 9 | 98 | 6 |
| EM1007 | 82 | 6 | 104 | 9 |
| EM1008 | 99 | 6 | 109 | 12 |
| EM1009 | 128 | 23 | 132 | 6 |
| EM1010 | 125 | 14 | 131 | 2 |
| EM1011 | 122 | 10 | 124 | 11 |
| EM1012 | 123 | 16 | 125 | 2 |
| EM1013 | 98 | 14 | 107 | 24 |
| EM1014 | 87 | 6 | 95 | 14 |
| EM1015 | 95 | 5 | 113 | 4 |
| EM1016 | 98 | 13 | 119 | 20 |
| EM1017 | 79 | 2 | 114 | 14 |
| EM1018 | 106 | 8 | 121 | 3 |
| EM1019 | 70 | 11 | 105 | 18 |
| EM1020 | 99 | 10 | 109 | 12 |
| EM1021 | 95 | 9 | 129 | 57 |
| EM1022 | 80 | 11 | 82 | 10 |
| EM1023 | 81 | 5 | 100 | 12 |
| EM1024 | 85 | 9 | 123 | 7 |
| EM1025 | 95 | 2 | 109 | 8 |
| EM1026 | 99 | 13 | 111 | 9 |
| EM1027 | 98 | 6 | 100 | 10 |
| EM1028 | 81 | 5 | 74 | 3 |
| EM1029 | 82 | 12 | 73 | 6 |
| EM1030 | 86 | 7 | 83 | 1 |
| EM1031 | 102 | 24 | 92 | 4 |
| EM1032 | 91 | 9 | 90 | 1 |
| EM1033 | 96 | 7 | 98 | 11 |
| EM1034 | 94 | 6 | 86 | 12 |
| EM1035 | 120 | 5 | 103 | 1 |
| EM1036 | 86 | 11 | 82 | 1 |
| EM1037 | 80 | 6 | 79 | 2 |
| EM1038 | 89 | 13 | 85 | 0 |
| EM1039 | 104 | 7 | 98 | 2 |
| EM1040 | 66 | 4 | 95 | 2 |
| EM1041 | 92 | 5 | 103 | 17 |
| EM1042 | 110 | 13 | 106 | 6 |
| EM1043 | 120 | 12 | 101 | 1 |
| EM1044 | 113 | 8 | 97 | 1 |
| EM1045 | 63 | 6 | 82 | 3 |
| EM1046 | 81 | 4 | 86 | 12 |
| EM1047 | 91 | 4 | 93 | 3 |
| EM1048 | 105 | 14 | 105 | 1 |
| EM1049 | 99 | 13 | 107 | 2 |
| EM1050 | 90 | 5 | 113 | 3 |
| EM1051 | 129 | 10 | 91 | 18 |
| EM1052 | 11 | 23 | 95 | 9 |
| EM1053 | 10 | 13 | 109 | 7 |
| EM1054 | 96 | 10 | 97 | 22 |
| EM1055 | 80 | 10 | 80 | 12 |
| EM1056 | 84 | 16 | 79 | 20 |
| EM1057 | 82 | 3 | 90 | 12 |
| EM1058 | 94 | 18 | 101 | 18 |
| EM1059 | 117 | 13 | 105 | 28 |
| EM1060 | 113 | 25 | 102 | 17 |
| EM1061 | 104 | 19 | 99 | 18 |
| EM1062 | 118 | 18 | 90 | 16 |
| EM1063 | 75 | 6 | 76 | 20 |
| EM1064 | 70 | 5 | 92 | 25 |
| EM1065 | 79 | 7 | 92 | 22 |
| EM1066 | 107 | 4 | 102 | 22 |
| EM1067 | 123 | 15 | 115 | 20 |
| EM1068 | 109 | 3 | 97 | 12 |
| EM1069 | 98 | 6 | 91 | 24 |
| EM1070 | 81 | 10 | 90 | 12 |
| EM1071 | 60 | 9 | 83 | 13 |
| EM1072 | 56 | 10 | 94 | 22 |
| EM1073 | 77 | 8 | 91 | 20 |
| EM1074 | 62 | 9 | 86 | 17 |
| EM1075 | 99 | 3 | 97 | 14 |
| EM1076 | 118 | 14 | 106 | 18 |
| EM1077 | 96 | 7 | 94 | 21 |
| EM1078 | 67 | 13 | 81 | 14 |
| EM1079 | 63 | 10 | 79 | 13 |
| EM1080 | 75 | 4 | 85 | 8 |
| EM1081 | 77 | 6 | 86 | 15 |
| EM1082 | 82 | 8 | 85 | 15 |
| EM1083 | 73 | 7 | 90 | 16 |
| EM1084 | 97 | 8 | 97 | 9 |
| EM1085 | 96 | 5 | 95 | 13 |
| EM1086 | 64 | 5 | 67 | 14 |
| EM1087 | 79 | 6 | 91 | 20 |
| EM1088 | 72 | 8 | 83 | 14 |
| EM1089 | 53 | 3 | 83 | 13 |
| EM1090 | 80 | 2 | 87 | 16 |
| EM1091 | 72 | 3 | 93 | 22 |
| EM1092 | 96 | 7 | 109 | 8 |
| EM1093 | 101 | 22 | 104 | 16 |
| EM1094 | 88 | 5 | 106 | 20 |
| EM1095 | 57 | 3 | 91 | 17 |
| EM1096 | 75 | 3 | 94 | 17 |
| EM1097 | 74 | 5 | 93 | 19 |
| EM1098 | 62 | 5 | 98 | 15 |
| EM1099 | 83 | 4 | 102 | 11 |
| EM1100 | 73 | 9 | 101 | 13 |
| EM1101 | 111 | 38 | 111 | 14 |
| EM1102 | 102 | 21 | 97 | 15 |
| EM1103 | 115 | 26 | 106 | 17 |
| EM1104 | 85 | 9 | 116 | 16 |
| EM1105 | 95 | 7 | 151 | 42 |
| EM1106 | 79 | 10 | 122 | 27 |
| EM1107 | 94 | 17 | 108 | 18 |
| EM1108 | 82 | 9 | 105 | 8 |
| EM1109 | 103 | 39 | 121 | 9 |
| EM1110 | 75 | 18 | 106 | 12 |
| EM1111 | 108 | 12 | 116 | 19 |
| EM1112 | 84 | 8 | 105 | 17 |
| EM1113 | 93 | 15 | 106 | 15 |
| EM1114 | 90 | 3 | 84 | 24 |
| EM1115 | 95 | 7 | 96 | 21 |
| EM1116 | 98 | 4 | 89 | 14 |
| EM1117 | 109 | 21 | 117 | 4 |
| EM1118 | 56 | 19 | 86 | 11 |
| EM1119 | 62 | 10 | 86 | 13 |
| EM1120 | 124 | 8 | 114 | 12 |
| EM1121 | 73 | 5 | 94 | 13 |
| EM1122 | 69 | 3 | 87 | 16 |
| EM1123 | 58 | 2 | 74 | 2 |
| EM1124 | 85 | 17 | 102 | 15 |
| EM1125 | 98 | 18 | 103 | 28 |
| EM1126 | 120 | 37 | 119 | 5 |
| EM1127 | 80 | 7 | 95 | 10 |
| EM1128 | 50 | 6 | 82 | 10 |
| EM1129 | 69 | 7 | 79 | 2 |
| EM1130 | 84 | 3 | 99 | 4 |
| EM1131 | 86 | 6 | 95 | 16 |
| EM1132 | 104 | 18 | 103 | 15 |
| EM1133 | 109 | 25 | 98 | 34 |
| EM1134 | 112 | 26 | 116 | 8 |
| EM1135 | 117 | 6 | 98 | 14 |
| EM1136 | 82 | 15 | 63 | 8 |
| EM1137 | 51 | 19 | 64 | 10 |
| EM1138 | 88 | 6 | 85 | 17 |
| EM1139 | 88 | 18 | 89 | 8 |
| EM1140 | 113 | 30 | 92 | 2 |
| EM1141 | 96 | 12 | 94 | 22 |
| EM1142 | 133 | 31 | 107 | 6 |
| EM1143 | 106 | 11 | 96 | 4 |
| EM1144 | 118 | 9 | 88 | 5 |
| EM1145 | 82 | 13 | 72 | 13 |
| EM1146 | 91 | 11 | 88 | 12 |
| EM1147 | 90 | 13 | 80 | 17 |
| EM1148 | 141 | 46 | 101 | 27 |
| EM1149 | 161 | 26 | 81 | 11 |
| EM1150 | 144 | 32 | 86 | 12 |
| EM1151 | 105 | 21 | 121 | 12 |
| EM1152 | 119 | 14 | 125 | 16 |
| EM1153 | 80 | 15 | 130 | 22 |
| EM1154 | 76 | 14 | 138 | 16 |
| EM1155 | 66 | 14 | 124 | 10 |
| EM1156 | 75 | 12 | 117 | 16 |
| EM1157 | 76 | 8 | 128 | 9 |
| EM1158 | 110 | 48 | 125 | 22 |
| EM1159 | 97 | 30 | 126 | 9 |
| EM1160 | 108 | 10 | 146 | 1 |
| EM1161 | 90 | 5 | 165 | 23 |
| EM1162 | 102 | 10 | 144 | 17 |
| EM1163 | 96 | 5 | 119 | 36 |
| EM1164 | 93 | 11 | 133 | 10 |
| EM1165 | 106 | 15 | 123 | 20 |
| EM1166 | 125 | 11 | 108 | 17 |
| EM1167 | 118 | 30 | 119 | 8 |
| EM1168 | 105 | 15 | 122 | 15 |
| EM1169 | 87 | 7 | 115 | 17 |
| EM1170 | 58 | 8 | 107 | 21 |
| EM1171 | 68 | 16 | 112 | 24 |
| EM1172 | 42 | 7 | 111 | 18 |
| EM1173 | 64 | 4 | 95 | 5 |
| EM1174 | 109 | 17 | 101 | 6 |
| EM1175 | 119 | 19 | 88 | 15 |
| EM1176 | 105 | 18 | 103 | 27 |
| EM1177 | 126 | 27 | 111 | 17 |
| EM1178 | 70 | 6 | 122 | 18 |
| EM1179 | 70 | 6 | 110 | 6 |
| EM1180 | 89 | 7 | 113 | 5 |
| EM1181 | 85 | 7 | 102 | 6 |
| EM1182 | 93 | 11 | 97 | 4 |
| EM1183 | 112 | 10 | 98 | 11 |
| EM1184 | 102 | 10 | 109 | 19 |
| EM1185 | 70 | 10 | 127 | 19 |
| EM1186 | 74 | 11 | 106 | 9 |
| EM1187 | 84 | 7 | 90 | 7 |
| EM1188 | 87 | 5 | 116 | 21 |
| EM1189 | 91 | 17 | 126 | 10 |
| EM1190 | 74 | 12 | 104 | 24 |
| EM1191 | 78 | 7 | 68 | 6 |
| EM1192 | 54 | 6 | 107 | 9 |

### Example 2

In vitro study in HepG2 cells showing MASP-2 knockdown efficacy of tested siRNAs after transfection of 5 nM or 10 nM siRNA.

MASP-2 knockdown efficacy of siRNAs EM1001-EM1192 (Table B and Table 5b) was determined after transfection of 5 or 10 nM siRNA in HepG2 cells. The results are depicted in Table B below. At 10 nM, remaining MASP-2 levels after knockdown reached a minimum of 42% and at 5 nM remaining MASP-2 levels reached a minimum of 45%, 10 nM the most potent siRNAs were EM1172, EM1128, EM1137, EM1089, and EM1192.

For transfection of HepG2 cells with siRNAs, cells were seeded at a density of 40,000 cells/well in 96-well tissue culture plates (TPP, Cat. 92096, Switzerland). Transfection of siRNA was carried out with Lipofectamine RNAiMax (Invitrogen/Life Technologies, Cat. 13778-500, Germany) according to manufacturer's instructions directly before seeding. The dual dose screen was performed with MASP-2-siRNAs in triplicates at 10 nM and 5 nM, respectively, with scrambled siRNA and luciferase-targeting siRNA as unspecific controls. After 24 h of incubation with siRNAs, medium was removed, and cells were lysed in 250 μL Lysis Buffer (InviTrap RNA Cell HTS96 Kit/C (Stratec, Cat. 7061300400, Germany)) and then frozen at −80° C. RNA was isolated using the InviTrap RNA Cell HTS96 Kit/C (Stratec, Cat. 7061300400, Germany). RT-qPCR was performed using MASP-2 and HPRT specific primer probe sets and Takyon™ One-Step Low Rox Probe 5× MasterMix dTTP on the QuantStudio6 device from Applied Biosystems in single-plex 384 well format. Expression differences were calculated using the delta delta Ct method and relative expression of MASP-2 normalized to the house keeping gene HPRT was determined. Results are expressed as % remaining MASP-2 mRNA after siRNA transfection in Table B.

TABLE B
Results of dual dose screening (10 nM
and 5 nM) of siRNAs targeting MASP-2.
The identity of the single strands forming each of
the siRNA duplexes as well as their sequences and
modifications are to be found in Tables 5a and 5b.

| | |
|---|---|
| % remaining mRNA | % remaining mRNA |
| at 10 nM | at 5 nM |

| Duplex | Mean | SD | Mean | SD |
|---|---|---|---|---|
| EM1001 | 109 | 3 | 123 | 20 |
| EM1002 | 106 | 22 | 140 | 32 |
| EM1003 | 113 | 8 | 154 | 35 |
| EM1004 | 115 | 9 | 159 | 16 |
| EM1005 | 96 | 10 | 151 | 18 |
| EM1006 | 100 | 9 | 184 | 37 |
| EM1007 | 82 | 6 | 123 | 17 |
| EM1008 | 99 | 6 | 146 | 19 |
| EM1009 | 128 | 23 | 161 | 39 |
| EM1010 | 125 | 14 | 144 | 37 |
| EM1011 | 122 | 10 | 139 | 45 |
| EM1012 | 123 | 16 | 134 | 17 |
| EM1013 | 98 | 14 | 183 | 2 |
| EM1014 | 87 | 6 | 176 | 15 |
| EM1015 | 95 | 5 | 134 | 9 |
| EM1016 | 98 | 13 | 111 | 22 |
| EM1017 | 79 | 2 | 83 | 5 |
| EM1018 | 106 | 8 | 100 | 37 |
| EM1019 | 70 | 11 | 50 | 6 |
| EM1020 | 99 | 10 | 134 | 17 |
| EM1021 | 95 | 9 | 154 | 37 |
| EM1022 | 80 | 11 | 125 | 35 |
| EM1023 | 81 | 5 | 113 | 27 |
| EM1024 | 85 | 9 | 133 | 41 |
| EM1025 | 95 | 2 | 105 | 16 |
| EM1026 | 99 | 13 | 134 | 18 |
| EM1027 | 98 | 6 | 124 | 9 |
| EM1028 | 81 | 5 | 140 | 30 |
| EM1029 | 82 | 12 | 149 | 26 |
| EM1030 | 86 | 7 | 103 | 6 |
| EM1031 | 102 | 24 | 121 | 38 |
| EM1032 | 91 | 9 | 92 | 7 |
| EM1033 | 96 | 7 | 109 | 25 |
| EM1034 | 94 | 6 | 110 | 27 |
| EM1035 | 120 | 5 | 121 | 34 |
| EM1036 | 86 | 11 | 132 | 33 |
| EM1037 | 80 | 6 | 115 | 11 |
| EM1038 | 89 | 13 | 108 | 26 |
| EM1039 | 104 | 7 | 105 | 10 |
| EM1040 | 66 | 4 | 74 | 18 |
| EM1041 | 92 | 5 | 102 | 22 |
| EM1042 | 110 | 13 | 148 | 36 |
| EM1043 | 120 | 12 | 136 | 29 |
| EM1044 | 113 | 8 | 164 | 48 |
| EM1045 | 63 | 6 | 84 | 10 |
| EM1046 | 81 | 4 | 149 | 66 |
| EM1047 | 91 | 4 | 129 | 51 |
| EM1048 | 105 | 14 | 113 | 11 |
| EM1049 | 99 | 13 | 104 | 14 |
| EM1050 | 90 | 5 | 105 | 6 |
| EM1051 | 129 | 10 | 111 | 2 |
| EM1052 | 111 | 23 | 138 | 17 |
| EM1053 | 101 | 13 | 132 | 13 |
| EM1054 | 96 | 10 | 126 | 30 |
| EM1055 | 80 | 10 | 180 | 17 |
| EM1056 | 84 | 16 | 216 | 51 |
| EM1057 | 82 | 3 | 126 | 12 |
| EM1058 | 94 | 18 | 103 | 4 |
| EM1059 | 117 | 13 | 123 | 6 |
| EM1060 | 113 | 25 | 126 | 23 |
| EM1061 | 104 | 19 | 124 | 29 |
| EM1062 | 118 | 18 | 151 | 10 |
| EM1063 | 75 | 6 | 144 | 10 |
| EM1064 | 70 | 5 | 152 | 26 |
| EM1065 | 79 | 7 | 110 | 5 |
| EM1066 | 107 | 4 | 104 | 8 |
| EM1067 | 123 | 15 | 117 | 21 |
| EM1068 | 109 | 3 | 102 | 5 |
| EM1069 | 98 | 6 | 112 | 20 |
| EM1070 | 81 | 10 | 109 | 18 |
| EM1071 | 60 | 9 | 104 | 2 |
| EM1072 | 56 | 10 | 119 | 13 |
| EM1073 | 77 | 8 | 98 | 13 |
| EM1074 | 62 | 9 | 69 | 5 |
| EM1075 | 99 | 3 | 88 | 4 |
| EM1076 | 118 | 14 | 112 | 8 |
| EM1077 | 96 | 7 | 119 | 17 |
| EM1078 | 67 | 13 | 123 | 13 |
| EM1079 | 63 | 10 | 140 | 14 |
| EM1080 | 75 | 4 | 100 | 23 |
| EM1081 | 77 | 6 | 103 | 16 |
| EM1082 | 82 | 8 | 90 | 16 |
| EM1083 | 73 | 7 | 71 | 4 |
| EM1084 | 97 | 8 | 117 | 19 |
| EM1085 | 96 | 5 | 118 | 27 |
| EM1086 | 64 | 5 | 104 | 23 |
| EM1087 | 79 | 6 | 124 | 26 |
| EM1088 | 72 | 8 | 103 | 17 |
| EM1089 | 53 | 3 | 74 | 17 |
| EM1090 | 80 | 2 | 115 | 19 |
| EM1091 | 72 | 3 | 86 | 20 |
| EM1092 | 96 | 7 | 130 | 2 |
| EM1093 | 101 | 22 | 108 | 14 |
| EM1094 | 88 | 5 | 110 | 15 |
| EM1095 | 57 | 3 | 93 | 24 |
| EM1096 | 75 | 3 | 98 | 39 |
| EM1097 | 74 | 5 | 99 | 6 |
| EM1098 | 62 | 5 | 83 | 6 |
| EM1099 | 83 | 4 | 83 | 12 |
| EM1100 | 73 | 9 | 61 | 13 |
| EM1101 | 111 | 38 | 103 | 6 |
| EM1102 | 102 | 21 | 141 | 18 |
| EM1103 | 115 | 26 | 145 | 41 |
| EM1104 | 85 | 9 | 165 | 20 |
| EM1105 | 95 | 7 | 218 | 42 |
| EM1106 | 79 | 10 | 227 | 79 |
| EM1107 | 94 | 17 | 108 | 13 |
| EM1108 | 82 | 9 | 101 | 11 |
| EM1109 | 103 | 39 | 101 | 5 |
| EM1110 | 75 | 18 | 73 | 3 |
| EM1111 | 108 | 12 | 124 | 38 |
| EM1112 | 84 | 8 | 106 | 16 |
| EM1113 | 93 | 15 | 185 | 39 |
| EM1114 | 90 | 3 | 187 | 50 |
| EM1115 | 95 | 7 | 134 | 27 |
| EM1116 | 98 | 4 | 105 | 29 |
| EM1117 | 109 | 21 | 103 | 10 |
| EM1118 | 56 | 19 | 49 | 16 |
| EM1119 | 62 | 10 | 77 | 17 |
| EM1120 | 124 | 8 | 179 | 20 |
| EM1121 | 73 | 5 | 104 | 22 |
| EM1122 | 69 | 3 | 83 | 19 |
| EM1123 | 58 | 2 | 45 | 11 |
| EM1124 | 85 | 17 | 93 | 11 |
| EM1125 | 98 | 18 | 100 | 13 |
| EM1126 | 120 | 37 | 126 | 9 |
| EM1127 | 80 | 7 | 77 | 6 |
| EM1128 | 50 | 6 | 72 | 23 |
| EM1129 | 69 | 7 | 104 | 21 |
| EM1130 | 84 | 3 | 117 | 30 |
| EM1131 | 86 | 6 | 92 | 12 |
| EM1132 | 104 | 18 | 100 | 8 |
| EM1133 | 109 | 25 | 86 | 9 |
| EM1134 | 112 | 26 | 116 | 12 |
| EM1135 | 117 | 6 | 99 | 6 |
| EM1136 | 82 | 15 | 117 | 30 |
| EM1137 | 51 | 19 | 52 | 18 |
| EM1138 | 88 | 6 | 117 | 31 |
| EM1139 | 88 | 18 | 103 | 20 |
| EM1140 | 113 | 30 | 105 | 29 |
| EM1141 | 96 | 12 | 88 | 10 |
| EM1142 | 133 | 31 | 138 | 10 |
| EM1143 | 106 | 11 | 78 | 21 |
| EM1144 | 118 | 9 | 113 | 6 |
| EM1145 | 82 | 13 | 92 | 8 |
| EM1146 | 91 | 11 | 66 | 31 |
| EM1147 | 90 | 13 | 93 | 6 |
| EM1148 | 141 | 46 | 87 | 9 |
| EM1149 | 161 | 26 | 95 | 5 |
| EM1150 | 144 | 32 | 97 | 14 |
| EM1151 | 105 | 21 | 126 | 9 |
| EM1152 | 119 | 14 | 124 | 15 |
| EM1153 | 80 | 15 | 100 | 8 |
| EM1154 | 76 | 14 | 97 | 8 |
| EM1155 | 66 | 14 | 57 | 19 |
| EM1156 | 75 | 12 | 112 | 27 |
| EM1157 | 76 | 8 | 94 | 8 |
| EM1158 | 110 | 48 | 112 | 8 |
| EM1159 | 97 | 30 | 110 | 27 |
| EM1160 | 108 | 10 | 135 | 12 |
| EM1161 | 90 | 5 | 118 | 6 |
| EM1162 | 102 | 10 | 126 | 15 |
| EM1163 | 96 | 5 | 126 | 22 |
| EM1164 | 93 | 11 | 113 | 20 |
| EM1165 | 106 | 15 | 100 | 5 |
| EM1166 | 125 | 11 | 109 | 8 |
| EM1167 | 118 | 30 | 116 | 18 |
| EM1168 | 105 | 15 | 118 | 23 |
| EM1169 | 87 | 7 | 94 | 15 |
| EM1170 | 58 | 8 | 70 | 7 |
| EM1171 | 68 | 16 | 105 | 23 |
| EM1172 | 42 | 7 | 52 | 8 |
| EM1173 | 64 | 4 | 54 | 8 |
| EM1174 | 109 | 17 | 94 | 14 |
| EM1175 | 119 | 19 | 89 | 5 |
| EM1176 | 105 | 18 | 121 | 6 |
| EM1177 | 126 | 27 | 106 | 15 |
| EM1178 | 70 | 6 | 90 | 9 |
| EM1179 | 70 | 6 | 74 | 16 |
| EM1180 | 89 | 7 | 84 | 11 |
| EM1181 | 85 | 7 | 89 | 9 |
| EM1182 | 93 | 11 | 107 | 13 |
| EM1183 | 112 | 10 | 108 | 10 |
| EM1184 | 102 | 10 | 113 | 12 |
| EM1185 | 70 | 10 | 92 | 6 |
| EM1186 | 74 | 11 | 81 | 26 |
| EM1187 | 84 | 7 | 90 | 4 |
| EM1188 | 87 | 5 | 101 | 17 |
| EM1189 | 91 | 17 | 104 | 0 |
| EM1190 | 74 | 12 | 76 | 8 |
| EM1191 | 78 | 7 | 51 | 4 |
| EM1192 | 54 | 6 | 69 | 6 |

### Example 3

In vitro study in HepG2 cells showing MASP-2 knockdown efficacy of tested siRNAs after transfection of 20, 4, 0.8, or 0.16 nM siRNA.

MASP-2 knockdown efficacy of selected siRNAs (Table 3 and Table 5b) was determined after transfection of 20, 4, 0.8, or 0.16 nM siRNA in HepG2 cells. The results are depicted in Table 3 below. At 20 nM, remaining MASP-2 levels after knockdown reached a minimum of 22% and at 4 nM reached a minimum of 32%. At 20 nM the most potent siRNAs were EM1192, EM1074, EM1119, EM1089, and EM1155.

For transfection of HepG2 cells with siRNAs, cells were seeded at a density of 40,000 cells/well in 96-well tissue culture plates (TPP, Cat. 92096, Switzerland). Transfection of siRNA was carried out with Lipofectamine RNAiMax (Invitrogen/Life Technologies, Cat. 13778-500, Germany) according to manufacturer's instructions directly before seeding. The dose-response screen was performed with MASP-2 siRNAs in triplicates at 20, 4, 0.8, or 0.16 nM, respectively, with scrambled siRNA and luciferase-targeting siRNA as unspecific controls.

After 24 h of incubation with siRNAs, medium was removed, and cells were lysed in 250 μL Lysis Buffer (InviTrap RNA Cell HTS96 Kit/C (Stratec, Cat. 7061300400, Germany)) and then frozen at −80° C. RNA was isolated using the InviTrap RNA Cell HTS96 Kit/C (Stratec, Cat. 7061300400, Germany). RT-qPCR was performed using MASP-2 and HPRT specific primer probe sets and Takyon™ One-Step Low Rox Probe 5× MasterMix dTTP on the QuantStudio6 device from Applied Biosystems in single-plex 384 well format. Expression differences were calculated using the delta delta Ct method and relative expression of MASP-2 normalized to the house keeping gene HPRT was determined. Results are expressed as % remaining MASP-2 mRNA after siRNA transfection in Table 3.

TABLE 3
Results of dose-response screening (20, 4,
0.8, or 0.16 nM) of siRNAs targeting MASP-2.
The identity of the single strands forming each of
the siRNA duplexes as well as their sequences and
modifications are to be found in Tables 5a and 5b.

| | |
|---|---|
| Concentration | % remaining mRNA |

| Duplex | (nM) | Mean | SD |
|---|---|---|---|
| EM1019 | 20 | 32 | 11 |
| | 4 | 39 | 8 |
| | 0.8 | 46 | 5 |
| | 0.16 | 75 | 27 |
| EM1040 | 20 | 46 | 6 |
| | 4 | 50 | 9 |
| | 0.8 | 60 | 12 |
| | 0.16 | 78 | 22 |
| EM1045 | 20 | 34 | 10 |
| | 4 | 45 | 8 |
| | 0.8 | 58 | 8 |
| | 0.16 | 72 | 8 |
| EM1074 | 20 | 24 | 2 |
| | 4 | 33 | 4 |
| | 0.8 | 63 | 14 |
| | 0.16 | 100 | 20 |
| EM1089 | 20 | 25 | 3 |
| | 4 | 32 | 6 |
| | 0.8 | 62 | 14 |
| | 0.16 | 76 | 7 |
| EM1095 | 20 | 45 | 11 |
| | 4 | 62 | 11 |
| | 0.8 | 72 | 15 |
| | 0.16 | 80 | 19 |
| EM1098 | 20 | 44 | 4 |
| | 4 | 49 | 2 |
| | 0.8 | 67 | 5 |
| | 0.16 | 77 | 18 |
| EM1100 | 20 | 31 | 1 |
| | 4 | 41 | 5 |
| | 0.8 | 68 | 19 |
| | 0.16 | 82 | 9 |
| EM1118 | 20 | 31 | 6 |
| | 4 | 37 | 1 |
| | 0.8 | 64 | 5 |
| | 0.16 | 109 | 43 |
| EM1119 | 20 | 24 | 3 |
| | 4 | 37 | 1 |
| | 0.8 | 54 | 7 |
| | 0.16 | 81 | 5 |
| EM1123 | 20 | 27 | 9 |
| | 4 | 43 | 9 |
| | 0.8 | 64 | 9 |
| | 0.16 | 113 | 29 |
| EM1128 | 20 | 42 | 6 |
| | 4 | 48 | 4 |
| | 0.8 | 76 | 2 |
| | 0.16 | 83 | 4 |
| EM1137 | 20 | 60 | 9 |
| | 4 | 69 | 13 |
| | 0.8 | 89 | 4 |
| | 0.16 | 120 | 13 |
| EM1146 | 20 | 100 | 20 |
| | 4 | 106 | 9 |
| | 0.8 | 114 | 10 |
| | 0.16 | 75 | 4 |
| EM1155 | 20 | 27 | 5 |
| | 4 | 33 | 5 |
| | 0.8 | 44 | 2 |
| | 0.16 | 78 | 4 |
| EM1170 | 20 | 38 | 8 |
| | 4 | 52 | 14 |
| | 0.8 | 94 | 16 |
| | 0.16 | 116 | 13 |
| EM1172 | 20 | 41 | 0 |
| | 4 | 62 | 8 |
| | 0.8 | 105 | 29 |
| | 0.16 | 131 | 26 |
| EM1173 | 20 | 33 | 4 |
| | 4 | 46 | 3 |
| | 0.8 | 58 | 8 |
| | 0.16 | 88 | 10 |
| EM1191 | 20 | 28 | 5 |
| | 4 | 38 | 7 |
| | 0.8 | 62 | 8 |
| | 0.16 | 99 | 18 |
| EM1192 | 20 | 22 | 5 |
| | 4 | 33 | 7 |
| | 0.8 | 48 | 3 |
| | 0.16 | 63 | 5 |

### Example 4

In vitro study in primary cynomolgus monkey hepatocytes showing MASP-2 knockdown efficacy of tested GalNAc-siRNA conjugates.

Expression of MASP-2 mRNA was assessed after incubation with the GalNAc-siRNA conjugates EM1193, EM1194, EM1195, EM1196, EM1197, EM1198, EM1199, EM1200, EM1201, EM1202 (further described in Table 5c) at 100 nM, 20 nM, 4 nM, 0.8 nM and 0.16 nM. mRNA level of the house keeping gene PPIB served as control.

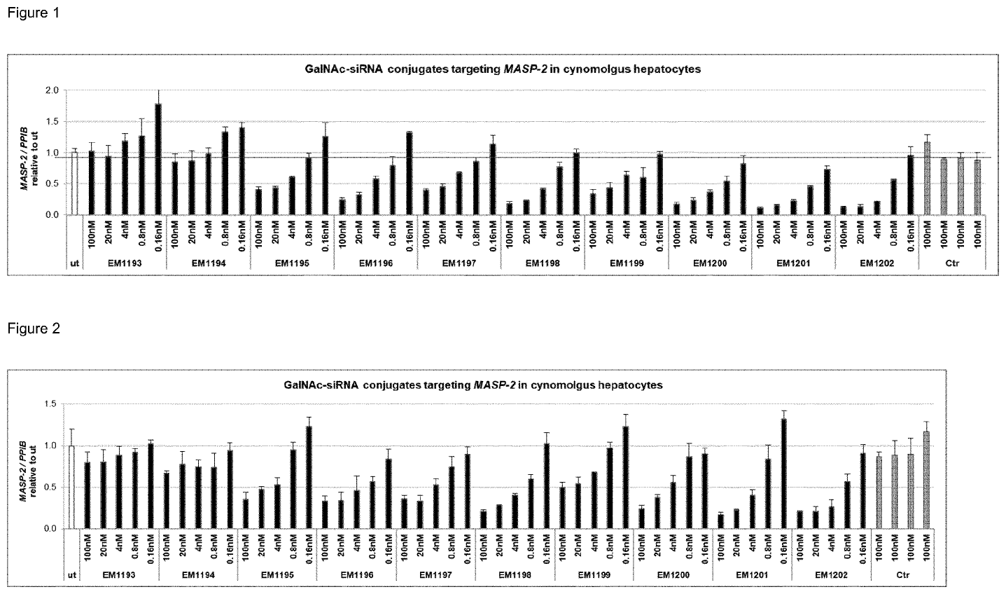

To test the knockdown efficacy of the GalNAc-conjugated siRNAs targeting MASP-2 in primary cynomolgus monkey hepatocytes, 45,000 cells per well (Supplier: Life Technologies) were added to siRNAs in plating medium (Life Technologies) for final concentrations between 100 nM and 0.16 nM in collagen-coated 96-well plates (Life Technologies). 24 hours post treatment, cells were lysed using InviTrap RNA Cell HTS96 Kit/C (Stratec). RT-qPCR was performed using mRNA-specific primers and probes against MASP-2 and PPIB. Expression differences were calculated using the delta delta Ct method and relative expression of MASP-2 normalized to the house keeping gene PPIB were determined. Results are expressed as ratio of MASP-2 to PPIB mRNA relative to untreated levels and can be found in FIG. 1.

Dose-dependent knockdown of MASP-2 mRNA was observed for eight tested GalNAc conjugates. At 100 nM remaining MASP-2 levels after knockdown were in the range of 11% to 86%. The strongest knockdown was observed at 100 nM with EM1201, EM1202, EM1200, and EM1198, with the remaining MASP-2 levels of 11%, 12%, 17%, and 18%, respectively.

### Example 5

In vitro study in primary cynomolgus monkey hepatocytes showing MASP-2 knockdown efficacy of tested GalNAc-siRNA conjugates.

Expression of MASP-2 mRNA was assessed after incubation with the GalNAc-siRNA conjugates EM1193, EM1194, EM1195, EM1196, EM1197, EM1198, EM1199, EM1200, EM1201, EM1202 (further described in Table 5c) at 100 nM, 20 nM, 4 nM, 0.8 nM and 0.16 nM. mRNA level of the house keeping gene PPIB served as control.

To test the knockdown efficacy of the GalNAc-conjugated siRNAs targeting MASP-2 in primary cynomolgus monkey hepatocytes, 45,000 cells per well (Supplier: Life Technologies) were added to siRNAs in plating medium (Life Technologies) for final concentrations between 100 nM and 0.16 nM in collagen-coated 96-well plates (Life Technologies). 24 hours post treatment, cells were lysed using InviTrap RNA Cell HTS96 Kit/C (Stratec). RT-qPCR was performed using mRNA-specific primers and probes against MASP-2 and PPIB. Expression differences were calculated using the delta delta Ct method and relative expression of MASP-2 normalized to the house keeping gene PPIB were determined. Results are expressed as ratio of MASP-2 to PPIB mRNA relative to untreated levels and can be found in FIG. 2.

Dose-dependent knockdown of MASP-2 mRNA was observed for eight tested GalNAc conjugates. At 100 nM remaining MASP-2 levels after knockdown were in the range of 17% to 80%. The strongest knockdown was observed at 100 nM with EM1201, EM1202, EM1200, and EM1198, with the remaining MASP-2 levels of 17%, 21%, 21%, and 24%, respectively.

### Example 6

In vitro study in primary human hepatocytes showing MASP-2 knockdown efficacy of tested GalNAc-siRNA conjugates.

Expression of MASP-2 mRNA was assessed after incubation with the GalNAc-siRNA conjugates EM1193, EM1194, EM1195, EM1196, EM1197, EM1198, EM1199, EM1200, EM1201, EM1202 (further described in Table 5c) at 100 nM, 20 nM, 4 nM, 0.8 nM and 0.16 nM. mRNA levels of the house keeping gene PPIB served as control.

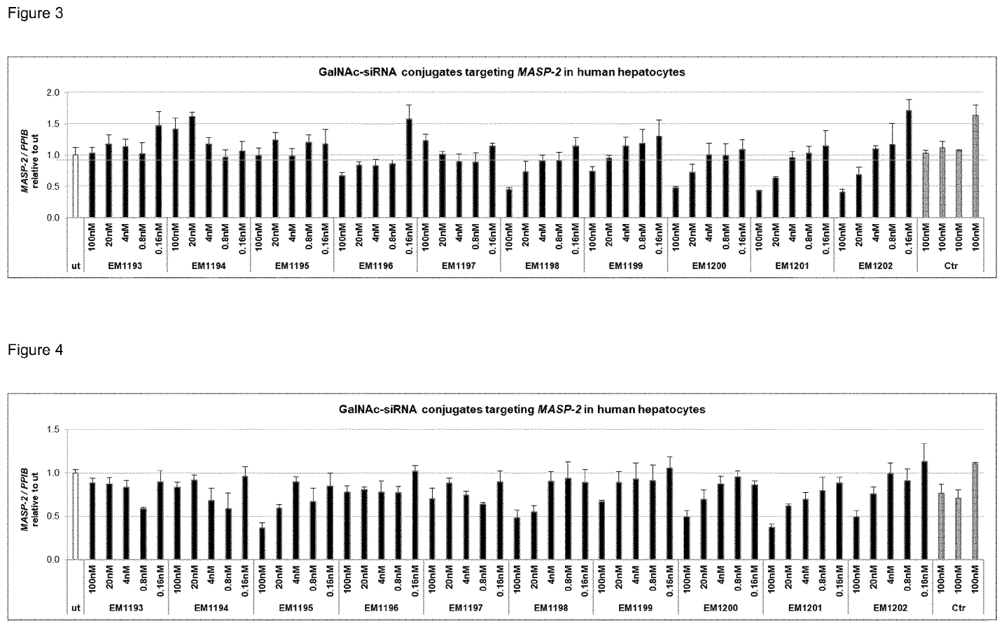

To test the knockdown efficacy of the GalNAc-conjugated siRNAs targeting MASP-2 in primary human hepatocytes, 35,000 cells per well (Supplier: Life technologies) were added to siRNAs in plating medium (Life Technologies) for final concentrations between 100 nM and 0.16 nM in collagen-coated 96-well plates (Life technologies). 24 hours post treatment, cells were lysed using InviTrap RNA Cell HTS96 Kit/C (Stratec). RT-qPCR was performed using mRNA-specific primers and probes against MASP-2 and PPIB. Expression differences were calculated using the delta delta Ct method and relative expression of MASP-2 was normalized to expression of the house keeping gene PPIB. Results are expressed as ratio of MASP-2 to PPIB mRNA relative to untreated levels and can be found in FIG. 3.

Dose-dependent knockdown of MASP-2 mRNA was observed for five tested GalNAc conjugates. At 100 nM remaining MASP-2 levels after knockdown were in the range of 34% to 99%. The strongest knockdown was observed at 100 nM with EM1202, EM1201, EM1198, and EM1200, with the remaining MASP-2 levels of 40%, 42%, 45%, and 47%, respectively.

### Example 7

In vitro study in primary human hepatocytes showing MASP-2 knockdown efficacy of tested GalNAc-siRNA conjugates.

Expression of MASP-2 mRNA was assessed after incubation with the GalNAc-siRNA conjugates EM1193, EM1194, EM1195, EM1196, EM1197, EM1198, EM1199, EM1200, EM1201, EM1202 (further described in Table 5c) at 100 nM, 20 nM, 4 nM, 0.8 nM and 0.16 nM. mRNA levels of the house keeping gene PPIB served as control.

To test the knockdown efficacy of the GalNAc-conjugated siRNAs targeting MASP-2 in primary human hepatocytes, 35,000 cells per well (Supplier: Life technologies) were added to siRNAs in plating medium (Life Technologies) for final concentrations between 100 nM and 0.16 nM in collagen-coated 96-well plates (Life technologies). 24 hours post treatment, cells were lysed using InviTrap RNA Cell HTS96 Kit/C (Stratec). RT-qPCR was performed using mRNA-specific primers and probes against MASP-2 and PPIB. Expression differences were calculated using the delta delta Ct method and relative expression of MASP-2 was normalized to expression of the house keeping gene PPIB. Results are expressed as ratio of MASP-2 to PPIB mRNA relative to untreated levels and can be found in FIG. 4.

Dose-dependent knockdown of MASP-2 mRNA was observed for five tested GalNAc conjugates. At 100 nM remaining MASP-2 levels after knockdown were in the range of 37% to 88%. The strongest knockdown was observed at 100 nM with EM1201, EM1195, and EM1198, with the remaining MASP-2 levels of 37%, 37%, and 48%, respectively.

### Example 8

In vitro study in primary mouse hepatocytes showing MASP-2 knockdown efficacy of tested GalNAc-siRNA conjugates.

Expression of MASP-2 mRNA was assessed after incubation with the GalNAc-siRNA conjugates EM1193, EM1194, EM1195 (further described in Table 5c) at 100 nM, 10 nM, 1 nM, 0.1 nM and 0.01 nM. mRNA level of the house keeping gene PPIB served as control.

To test the knockdown efficacy of the GalNAc conjugated siRNAs for MASP-2 in primary mouse hepatocytes, 25,000 cells per well (Supplier: Life Technologies) were added to siRNAs in plating medium (Life Technologies) for final concentrations between 100 nM and 0.01 nM in collagen-coated 96-well plates (Life technologies). 24 hours post treatment, cells were lysed using InviTrap RNA Cell HTS96 Kit/C (Stratec). qPCR was performed using mRNA-specific primers and probes against MASP-2 and PPIB. Expression differences were calculated using the delta delta Ct method and relative expression of MASP-2 was normalized to expression of the house keeping gene PPIB. Results are expressed as ratio of MASP-2 to PPIB mRNA relative to untreated levels and can be found in FIG. 5.

Dose-dependent knockdown of MASP-2 mRNA was observed for all tested GalNAc conjugates. At 100 nM remaining MASP-2 levels after knockdown were in the range of 23% to 39%. The strongest knockdown was observed at 100 nM with EM1195 and EM1194, with the remaining MASP-2 levels of 23% and 25%, respectively.

### Example 9

In vivo study showing knockdown of MASP-2 mRNA in murine liver tissue after single subcutaneous dosing of 1 or 5 mg/kg GalNAc conjugated siRNA at two weeks post-dosing.

GalNAc-siRNA conjugates EM1193, EM1194, EM1195 (further described in Table 5c) were tested. mRNA level of the house keeping gene APOB served as housekeeping control.

Male C57BL/6 mice with an age of 8 weeks were obtained from Janvier, France. Animal experiments were performed according to ethical guidelines of the German Protection of Animals Act in its version of July 2013. Mice were randomized according to weight into groups of 4-5 mice. On day 1 of the study animals received a single subcutaneous dose of 1 or 5 mg/kg siRNA dissolved in phosphate buffered saline (PBS) or PBS only as control. The viability, body weight and behaviour of the mice was monitored during the study without pathological findings.

At day 15, the study was terminated, animals were euthanized, and liver samples were snap frozen and stored at −80° C. until further analysis. For analysis, in summary, total RNA was prepared with RNeasy Fibrous Tissue Mini Kit (QIAGEN, Venlo, Netherlands) according to the manufacturer's instruction. To assess the integrity of isolated RNA, automated electrophoresis was performed using a 2100 Bioanalyzer (Agilent Technologies, Inc., Santa Clara, USA). Per reaction, 100 ng total RNA was used for RT-qPCR with the amplicon sets specific for MASP-2 and APOB.

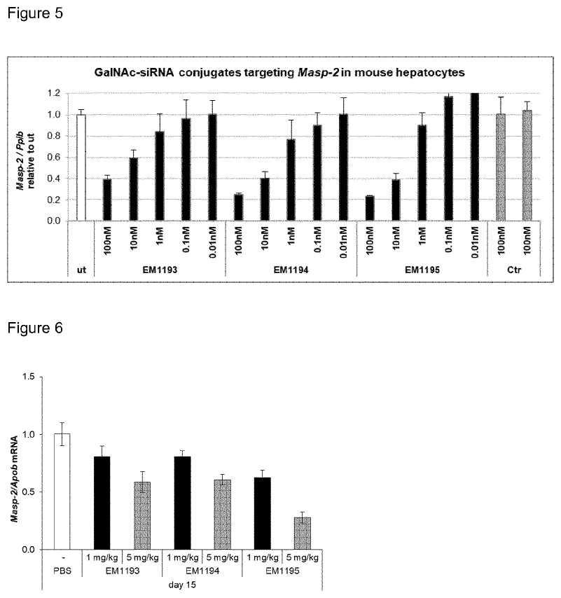

Expression differences were calculated using the delta delta Ct method and relative expression of MASP-2 versus the house keeping gene APOB normalized to the PBS was used for comparison of the different siRNAs. All tested GalNAc conjugates induced a dose-dependent knockdown of MASP-2 mRNA in liver. At 5 mg/kg remaining MASP-2 levels after knockdown were in the range of 28% to 61%. The maximum achieved knockdown of 72% was observed using EM1195 at 5 mg/kg siRNA (FIG. 6).

### Example 10

In vitro study in primary cynomolgus monkey hepatocytes showing MASP-2 knockdown efficacy of tested GalNAc-siRNA conjugates.

Expression of MASP-2 mRNA was assessed after incubation with siRNA conjugates EM1203, EM1204, EM1205, EM1206, EM1207, EM1208, EM1209, EM1210, EM1211, EM1212, EM1213 (further described in Table 5c) at 100 nM, 20 nM, 4 nM, 0.8 nM and 0.16 nM. mRNA level of the house keeping gene PPIB served as control.

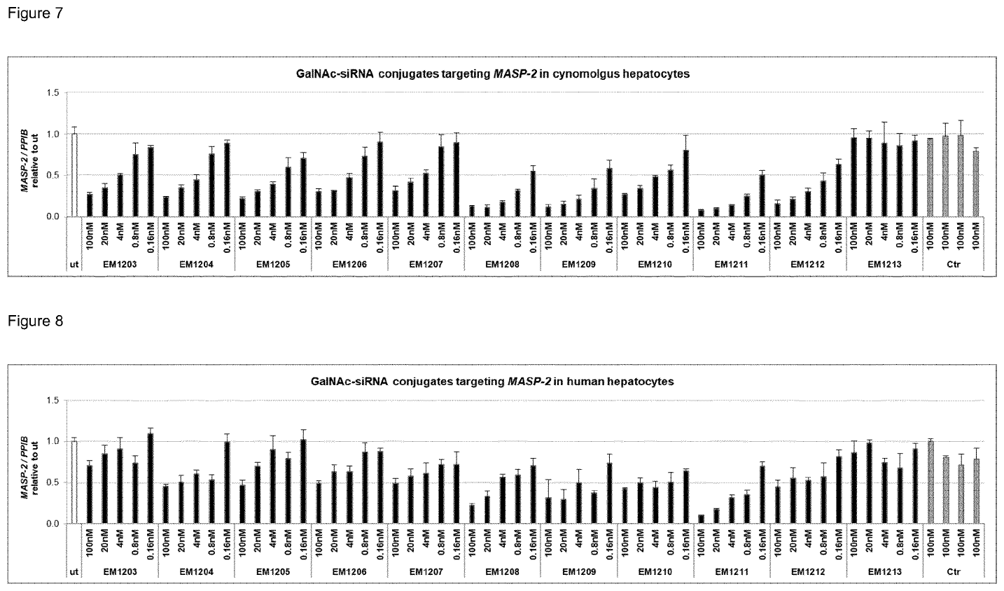

To test the knockdown efficacy of the GalNAc conjugated siRNAs targeting MASP-2 in primary cynomolgus monkey hepatocytes, 45,000 cells per well (Supplier: Life Technologies and Primacyt) were seeded on collagen-coated 96-well plates (Life Technologies). siRNAs in concentrations between 100 nM and 0.16 nM were added immediately after seeding. 24 hours post treatment, cells were lysed using InviTrap RNA Cell HTS96 Kit/C (Stratec). RT-qPCR was performed using mRNA-specific primers and probes against MASP-2 and PPIB. Expression differences were calculated using the delta delta Ct method and relative expression of MASP-2 normalized to the house keeping gene PPIB were determined. Results are expressed as ratio of MASP-2 to PPIB mRNA relative to untreated levels and can be found in FIG. 7.

Dose-dependent knockdown of MASP-2 mRNA was observed for eight tested GalNAc conjugates. At 100 nM remaining MASP-2 levels after knockdown were in the range of 8% to 96%. The strongest knockdown was observed at 100 nM with EM1211, EM1208, EM1209, and EM1212, with the remaining MASP-2 levels of 8%, 11%, 12%, and 16%, respectively.

### Example 11

In vitro study in primary human hepatocytes showing MASP-2 knockdown efficacy of tested GalNAc-siRNA conjugates.

Expression of MASP-2 mRNA was assessed after incubation with GalNAc siRNA conjugates EM1203, EM1204, EM1205, EM1206, EM1207, EM1208, EM1209, EM1210, EM1211, EM1212, EM1213 (further described in Table 5c) at 100 nM, 20 nM, 4 nM, 0.8 nM and 0.16 nM. mRNA levels of the house keeping gene PPIB served as control.

To test the knockdown efficacy of the GalNAc conjugated siRNAs targeting MASP-2 in primary human hepatocytes 35,000 cells per well (Supplier: Life technologies) were seeded on collagen-coated 96-well plates (Life technologies). siRNAs in concentrations between 100 nM and 0.16 nM were added immediately after seeding. 24 hours post treatment, cells were lysed using InviTrap RNA Cell HTS96 Kit/C (Stratec). RT-qPCR was performed using mRNA-specific primers and probes against MASP-2 and PPIB. Expression differences were calculated using the delta delta Ct method and relative expression of MASP-2 was normalized to expression of the house keeping gene PPIB. Results are expressed as ratio of MASP-2 to PPIB mRNA relative to untreated levels and can be found in FIG. 8.

Dose-dependent knockdown of MASP-2 mRNA was observed for all tested GalNAc conjugates. At 100 nM remaining MASP-2 levels after knockdown were in the range of 10% to 86%. The strongest knockdown was observed at 100 nM with EM1211, EM1208, and EM1209, with the remaining MASP-2 levels of 10%, 22%, and 31%, respectively.

### Example 12

In vitro study in primary mouse hepatocytes showing MASP-2 knockdown efficacy of tested GalNAc-siRNA conjugates.

Expression of MASP-2 mRNA was assessed after incubation with GalNAc siRNA conjugates EM1203, EM1204 and EM1205 (further described in Table 5c) at 100 nM, 10 nM, 1 nM, 0.1 nM and 0.01 nM. mRNA level of the house keeping gene PPIB served as control.

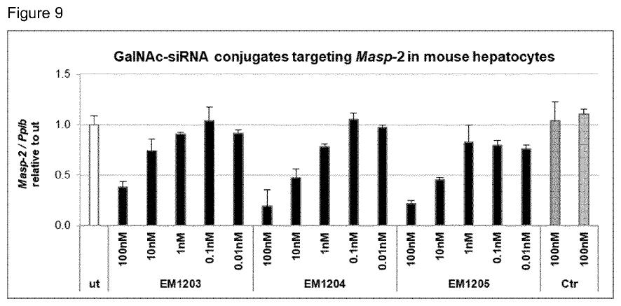

To test the knockdown efficacy of the GalNAc conjugated siRNAs targeting MASP-2 in primary mouse hepatocytes, 25,000 cells per well (Supplier: Life Technologies and Primacyt) were seeded on collagen-coated 96-well plates (Life echnologies). siRNAs in concentrations between 100 nM and 0.01 nM were added immediately after seeding. 24 hours post treatment, cells were lysed using InviTrap RNA Cell HTS96 Kit/C (Stratec). qPCR was performed using mRNA-specific primers and probes against MASP-2 and PPIB. Expression differences were calculated using the delta delta Ct method and relative expression of MASP-2 was normalized to expression of the house keeping gene Ppib. Results are expressed as ratio of MASP-2 to PPIB mRNA relative to untreated levels and can be found in FIG. 9.

Dose-dependent knockdown of MASP-2 mRNA was observed for all tested GalNAc conjugates. At 100 nM remaining MASP-2 levels after knockdown were in the range of 19% to 38%. The strongest knockdown was observed at 100 nM with EM1204 and EM1205, with the remaining MASP-2 levels of 19% and 22%, respectively.

### Example 13

Synthesis of (vp)-mU-phos (Table 4) was performed as described in Prakash, Nucleic Acids Res. 2015, 43(6), 2993-3011 and Haraszti, Nucleic Acids Res. 2017, 45(13), 7581-7592. Synthesis of the phosphoramidite derivatives of ST41 (ST41-phos) (Table 4) as well as ST23 (ST23-phos) (Table 4) can be performed as described in WO2017/174657. Synthesis of phosphorthioamidites was performed as described in Caruthers, J. Org. Chem. 1996, 61, 4272-4281.

### Example 14

Example compounds were synthesized according to methods described below and known to persons of skill in the art. Assembly of the oligonucleotide chain and linker building blocks was performed by solid phase synthesis applying phosphoramidite methodology.

Downstream cleavage, deprotection and purification were performed following standard procedures that are well known in the art.

Oligonucleotide syntheses was performed on an AKTA oligopilot 10 using commercially available 2′O-Methyl RNA and 2′Fluoro-2′Deoxy RNA base loaded CPG (=controlled pore glass) solid support and phosphoramidites (all standard protection, ChemGenes, LinkTech) were used.

Ancillary reagents were purchased from EMP Biotech and Biosolve. Synthesis was performed using a 0.1 M solution of the phosphoramidite in dry acetonitrile (<20 ppm H₂O) and benzylthiotetrazole (BTT) was used as activator (0.3M in acetonitrile). Coupling time was 10 min. If phosphorthioamidites were used to introduce a phosphordithioate linkage (PS2) a repeated coupling wash cycle over 60 min was performed. A Cap/OX/Cap or Cap/Thio/Cap cycle was applied (Cap: Ac₂O (=acetic anhydride)/NMI (═N-methylimidazole)/Lutidine/Acetonitrile; OX (=Oxidizer): 0.05M I₂ in pyridine/H₂O; Thio=thiolation reagent, 50 mM EDITH (=3-ethoxy-1,2,4-dithiazoline-5-one)). Phosphorothioates and phosphordithioates were introduced using commercially available thiolation reagent 50 mM EDITH in acetonitrile (Link technologies). DMT (=dimethoxytrityl) cleavage was achieved by treatment with 3% dichloroacetic acid in toluene. Upon completion of the programmed synthesis cycles a diethylamine (DEA) wash was performed. All oligonucleotides were synthesized in DMT-off mode.

To synthesize tri-antennary GalNAc clusters ([ST23 (ps)]₃ ST41 (ps) and [ST23]₃ ST41, Table 4) ST23 (Table 4) and ST41 (Table 4) were introduced by successive coupling of the branching trebler amidite derivative (C4XLT-phos) followed by the GalNAc amidite (ST23-phos, Table 4). Attachment of (vp)-mU moiety (Table 4) was achieved by use of (vp)-mU-phos (Table 4) in the last synthesis cycle. The (vp)-mU-phos does not provide a hydroxy group suitable for further synthesis elongation and therefore, does not possess an DMT-group. Hence coupling of (vp)-mU-phos results in synthesis termination.

For the removal of the methyl esters masking the vinylphosphonate (vp), the CPG carrying the fully assembled oligonucleotide was dried under reduced pressure and transferred into a 20 mL PP (=polypropylene) syringe reactor for solid phase peptide synthesis equipped with a disc frit (Carl Roth GmbH). The CPG was then brought into contact with a solution of 250 μL TMSBr (=bromotrimethylsilane) and 177 μL pyridine in CH₂Cl₂ (0.5 mL/μmol solid support bound oligonucleotide) at room temperature and the reactor was sealed with a Luer cap. The reaction vessels were slightly agitated over a period of 2×15 min, the excess reagent discarded, and the residual CPG washed 2× with 10 mL acetonitrile. Further downstream processing did not alter from any other example compound.

The single strands were cleaved off the CPG by 40% aq. methylamine treatment (in presence of 20 mM DTT if phosphorodithioate linkages were present) in 90 min at RT (=room temperature). The resulting crude oligonucleotide was purified by ion exchange chromatography (Resource Q, 6 mL, GE Healthcare) on an AKTA Pure HPLC System using a sodium chloride gradient. Product containing fractions were pooled, desalted on a size exclusion column (Zetadex, EMP Biotech) and lyophilized until further use.

All final single-stranded products were analysed by AEX-HPLC to prove their purity. Identity of the respective single-stranded products was proved by LC-MS analysis.

### Example 15

Individual single strands were dissolved in a concentration of 60 OD/mL in H₂O. Both individual oligonucleotide solutions were added together in a reaction vessel. For easier reaction monitoring a titration was performed. The first strand was added in 25% excess over the second strand as determined by UV-absorption at 260 nm. The reaction mixture was heated to 80° C. for 5 min and then slowly cooled to RT. Double-strand formation was monitored by ion pairing reverse phase HPLC. From the UV-area of the residual single strand the needed amount of the second strand was calculated and added to the reaction mixture. The reaction was heated to 80° C. again and slowly cooled to RT. This procedure was repeated until less than 10% of residual single strand was detected.

### Example 16

In vitro study in HepG2 cells showing MASP-2 knockdown efficacy of tested siRNAs after transfection of 20, 4, 0.8, 0.16, or 0.032 nM siRNA.

MASP-2 knockdown efficacy of selected siRNAs (Table 5b) was determined after transfection of 20, 4, 0.8, or 0.16 nM siRNA in HepG2 cells. The results are depicted in Table 6 below. At 20 nM, remaining MASP-2 levels after knockdown reached a minimum of 22% and at 4 nM reached a minimum of 20%. At 20 nM the most potent siRNAs were EM1223 and EM1217.

For transfection of HepG2 cells with siRNAs, cells were seeded at a density of 40,000 cells/well in 96-well tissue culture plates (TPP, Cat. 92096, Switzerland). Transfection of siRNA was carried out with Lipofectamine RNAiMax (Invitrogen/Life Technologies, Cat. 13778-500, Germany) according to manufacturer's instructions directly before seeding. The dose-response screen was performed with MASP-2 siRNAs in triplicates at 20, 4, 0.8, 0.16, or 0.032 nM, respectively, with scrambled siRNA and luciferase-targeting siRNA as unspecific controls. After 24 h of incubation with siRNAs, medium was removed, and cells were lysed in 250 μL Lysis Buffer (InviTrap RNA Cell HTS96 Kit/C (Stratec, Cat. 7061300400, Germany)) and then frozen at −80° C. RNA was isolated using the InviTrap RNA Cell HTS96 Kit/C (Stratec, Cat. 7061300400, Germany). RT-qPCR was performed using MASP-2 and PPIB specific primer probe sets and Takyon™ One-Step Low Rox Probe 5× MasterMix dTTP on the QuantStudio6 device from Applied Biosystems in single-plex 384 well format. Expression differences were calculated using the delta delta Ct method and relative expression of MASP-2 normalized to the house keeping gene PPIB was determined. Results are expressed as % remaining MASP-2 mRNA after siRNA transfection in Table 6.

TABLE 6
Results of dose-response screening (20, 4,
0.8, 0.16, 0.032 nM) of siRNAs targeting MASP-2.
The identity of the single strands forming each of
the siRNA duplexes as well as their sequences and
modifications are to be found in Tables 5b.

| | |
|---|---|
| Concentration | % remaining mRNA |

| Duplex | (nM) | Mean | SD |
|---|---|---|---|
| EM1223 | 20 | 22 | 4 |
| | 4 | 20 | 4 |
| | 0.8 | 29 | 10 |
| | 0.16 | 40 | 8 |
| | 0.032 | 64 | 4 |
| EM1215 | 20 | 33 | 6 |
| | 4 | 28 | 3 |
| | 0.8 | 32 | 6 |
| | 0.16 | 47 | 4 |
| | 0.032 | 75 | 15 |
| EM1217 | 20 | 28 | 5 |
| | 4 | 32 | 4 |
| | 0.8 | 37 | 6 |
| | 0.16 | 50 | 7 |
| | 0.032 | 58 | 13 |

### Example 17

In vivo study showing knockdown of MASP-2 mRNA in non-human primates (NHP).

The objective of this experiment was to determine protein knockdown efficacy of siRNA GalNAc conjugates targeting MASP-2 in vivo in non-human primates (NHPs).

Purpose bred cynomolgus monkeys (8- to 15-year-old females) were allocated to different treatment groups (4 animals per group). On day 1, each group was treated with a single dose of 3 mg GalNAc siRNA per kg body weight by subcutaneous injection, while control animals received the vehicle, 0.9% saline, by subcutaneous injection. Series bleeding was conducted at a pre-dose (day −7 and day 1), day 2, 3, 8, 15, 22, 29, and 57. The expression of MASP-2 protein was measured in serum using specific ELISA assay (Abcam ab278121).

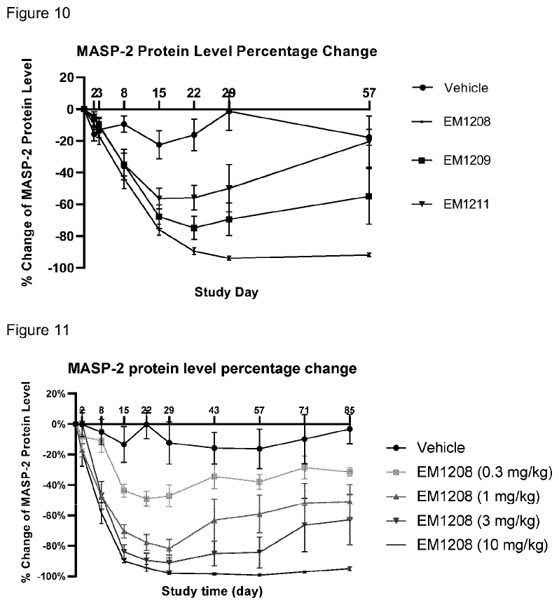

Results of MASP-2 protein level reduction over 8 weeks post single subcutaneous dose of GalNAc conjugated siRNAs EM1208, EM1209, and EM1211 are shown in FIG. 10. MASP-2 protein levels in cynomolgus monkey serum collected 3 weeks after single treatment with EM1208 were reduced on average by 89% and after 8 weeks by 91%. MASP-2 protein levels in cynomolgus monkey serum collected 3 weeks after single treatment with EM1209 were reduced on average by 74% and after 8 weeks by 55%. MASP-2 protein levels in cynomolgus monkey serum collected 3 weeks after single treatment with EM1211 were reduced on average by 55% and after 8 weeks by 20%.

### Example 18

The objective of the second experiment in NHPs was to determine the duration-of-action of a single siRNA GalNAc conjugate targeting MASP-2 by analyzing the protein knockdown efficacy.

Purpose bred cynomolgus monkeys (8- to 15-year-old naïve females) were allocated to different treatment groups (4 animals per group). On day 1, each group was treated with a single dose of either 0.3, 1, 3, or 10 mg GalNAc siRNA per kg body weight by subcutaneous injection, while control animals received the vehicle, 0.9% saline, by subcutaneous injection. Series bleeding was conducted at a pre-dose (day −7 and day 1), day 3, 8, 15, 22, 29, 43, 57, 71, and 85. The expression of MASP-2 protein was measured in serum using specific ELISA assay (Abcam ab278121).

Results of MASP-2 protein level reduction over 12 weeks post single subcutaneous dose of GalNAc conjugated siRNA EM1208 are shown in FIG. 11. Dose-dependent MASP-2 protein knockdown in cynomolgus monkey serum was observed after a single dose of EM1208. MASP-2 protein levels at 4 weeks after treatment with EM1208 at 10 mg/kg were reduced on average by 98%, after 8 weeks by 99%, and after 12 weeks by 95%. When EM1208 was dosed at 3 mg/kg, MASP-2 protein levels in serum collected 4 weeks were reduced on average by 91%, 8 weeks by 84%, and after 12 weeks by 62%.

TABLE 1
Summary of abbreviations

| Abbreviation | Meaning |
|---|---|
| mA, mU, | 2′-O-Methyl RNA nucleotides |
| mC, mG | |
| 2′-OMe | 2′-O-Methyl modification |
| fA, fU, | 2′ deoxy-2′-F RNA nucleotides |
| fC, fG | |
| 2′-F | 2′-fluoro modification |
| (ps) | phosphorothioate |
| (vp) | Vinyl-(E)-phosphonate |
| (vp)-mU | 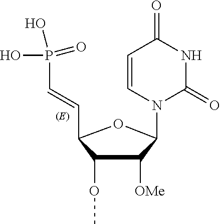 |
| (vp)-mU- phos | 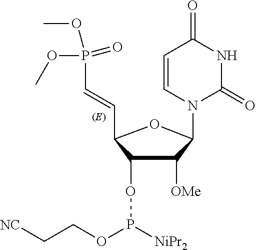 |
| ivA, ivC, ivU, ivG | inverted RNA (3′-3′) nucleotides |
| ST23 | 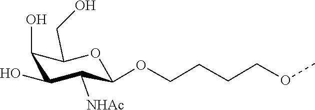 |
| ST23-phos | 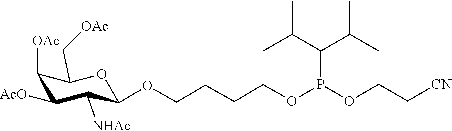 |
| ST41 (or C4XLT) | 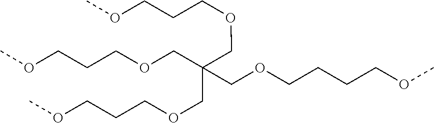 |
| ST41-phos (or C4XLT- phos) | 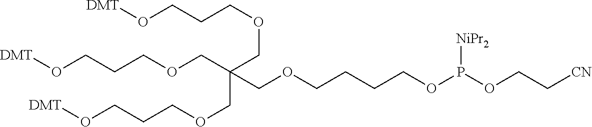 |
| Ser(GN) (when at the end of a chain, one of the O— is OH) | 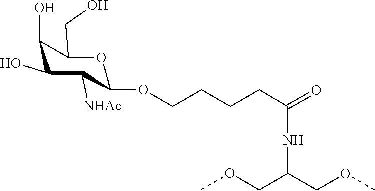 |
| [ST23 (ps)]3 ST41 (ps) | 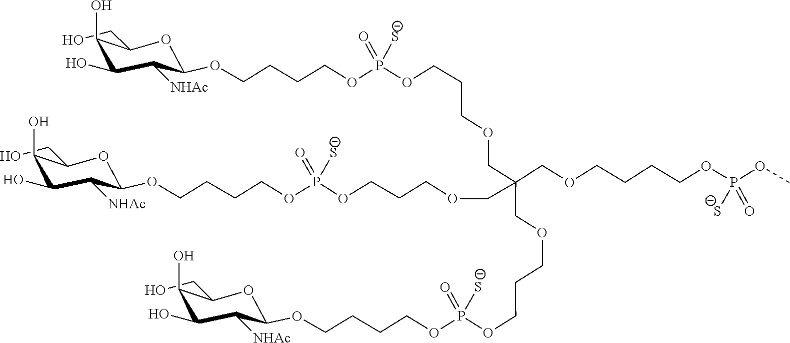 |
| [ST23]3 ST41 | 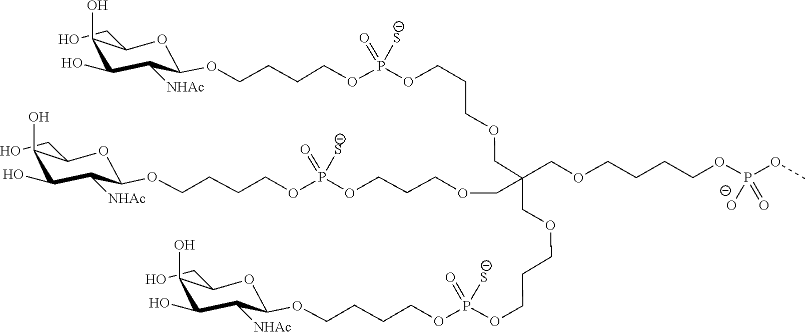 |

The abbreviations as shown in Table 4 may be used herein. The list of abbreviations may not be exhaustive and further abbreviations and their meaning may be found throughout this document.

### SUMMARY SEQUENCE TABLES

TABLE 5a
Unmodified duplexes

| Duplex | Strand Name | | |
|---|---|---|---|
| ID | (*) | Sequence (5′→3′) | SEQ ID No. |
| EM1001 | EM1001-A | GACUUGCCAAGGAAAAUCA | 1 |
| EM1001 | EM1001-B | UGAUUUUCCUUGGCAAGUC | 2 |
| EM1002 | EM1002-A | UGGGUAGAUCAUCAGGAGG | 3 |
| EM1002 | EM1002-B | CCUCCUGAUGAUCUACCCA | 4 |
| EM1003 | EM1003-A | UCCACAUCGAAGGACUCCA | 5 |
| EM1003 | EM1003-B | UGGAGUCCUUCGAUGUGGA | 6 |
| EM1004 | EM1004-A | UUGGCUUGCACAGGUGAAA | 7 |
| EM1004 | EM1004-B | UUUCACCUGUGCAAGCCAA | 8 |
| EM1005 | EM1005-A | AUUGUGUAGAAGGUCUCUU | 9 |
| EM1005 | EM1005-B | AAGAGACCUUCUACACAAU | 10 |
| EM1006 | EM1006-A | ACAGCUUUGUAGGUGGUCA | 11 |
| EM1006 | EM1006-B | UGACCACCUACAAAGCUGU | 12 |
| EM1007 | EM1007-A | AGUAGAGGCGCAGGCGGUA | 13 |
| EM1007 | EM1007-B | UACCGCCUGCGCCUCUACU | 14 |
| EM1008 | EM1008-A | UCUCCUUUGGAGCUCGUCC | 15 |
| EM1008 | EM1008-B | GGACGAGCUCCAAAGGAGA | 16 |
| EM1009 | EM1009-A | AGAAUCCAUCAGCCUCACA | 17 |
| EM1009 | EM1009-B | UGUGAGGCUGAUGGAUUCU | 18 |
| EM1010 | EM1010-A | GGUCCCAAGAUCCAUCUUU | 19 |
| EM1010 | EM1010-B | AAAGAUGGAUCUUGGGACC | 20 |
| EM1011 | EM1011-A | GCAUUGGCCGGUCCCAAGA | 21 |
| EM1011 | EM1011-B | UCUUGGGACCGGCCAAUGC | 22 |
| EM1012 | EM1012-A | CCACAAACAGGCUCACAGA | 23 |
| EM1012 | EM1012-B | UCUGUGAGCCUGUUUGUGG | 24 |
| EM1013 | EM1013-A | GUACUGAAUCACAGCUUUG | 25 |
| EM1013 | EM1013-B | CAAAGCUGUGAUUCAGUAC | 26 |
| EM1014 | EM1014-A | CUAAGCCAGCACAAAGCAU | 27 |
| EM1014 | EM1014-B | AUGCUUUGUGCUGGCUUAG | 28 |
| EM1015 | EM1015-A | UUCACAGCUGUACUGAAUC | 29 |
| EM1015 | EM1015-B | GAUUCAGUACAGCUGUGAA | 30 |
| EM1016 | EM1016-A | UAAGCCAGCACAAAGCAUG | 31 |
| EM1016 | EM1016-B | CAUGCUUUGUGCUGGCUUA | 32 |
| EM1017 | EM1017-A | AGUAGUCGGAGCGGAAGGU | 33 |
| EM1017 | EM1017-B | ACCUUCCGCUCCGACUACU | 34 |
| EM1018 | EM1018-A | UCUCGAUCCAGGGAAUAUA | 35 |
| EM1018 | EM1018-B | UAUAUUCCCUGGAUCGAGA | 36 |
| EM1019 | EM1019-A | UCGAAGUGGGUGAAGUAGA | 37 |
| EM1019 | EM1019-B | UCUACUUCACCCACUUCGA | 38 |
| EM1020 | EM1020-A | CUUGCCAAGGAAAAUCACC | 39 |
| EM1020 | EM1020-B | GGUGAUUUUCCUUGGCAAG | 40 |
| EM1021 | EM1021-A | GCAUGAGCAGCUGUUAGGA | 41 |
| EM1021 | EM1021-B | UCCUAACAGCUGCUCAUGC | 42 |
| EM1022 | EM1022-A | GGACACUAUUCCUCCCACA | 43 |
| EM1022 | EM1022-B | UGUGGGAGGAAUAGUGUCC | 44 |
| EM1023 | EM1023-A | UGUCUCCACAUCGAAGGAC | 45 |
| EM1023 | EM1023-B | GUCCUUCGAUGUGGAGACA | 46 |
| EM1024 | EM1024-A | GUGACAAAGGUGAUGGUCA | 47 |
| EM1024 | EM1024-B | UGACCAUCACCUUUGUCAC | 48 |
| EM1025 | EM1025-A | CUCUUCACAGCUGUACUGA | 49 |
| EM1025 | EM1025-B | UCAGUACAGCUGUGAAGAG | 50 |
| EM1026 | EM1026-A | AAGGUCUCUUCACAGCUGU | 51 |
| EM1026 | EM1026-B | ACAGCUGUGAAGAGACCUU | 52 |
| EM1027 | EM1027-A | GCAUAGAAGGCCUCGAACC | 53 |
| EM1027 | EM1027-B | GGUUCGAGGCCUUCUAUGC | 54 |
| EM1028 | EM1028-A | GUAGAUCAUCAGGAGGGCC | 55 |
| EM1028 | EM1028-B | GGCCCUCCUGAUGAUCUAC | 56 |
| EM1029 | EM1029-A | GAAUCACAGCUUUGUAGGU | 57 |
| EM1029 | EM1029-B | ACCUACAAAGCUGUGAUUC | 58 |
| EM1030 | EM1030-A | AAUCACAGCUUUGUAGGUG | 59 |
| EM1030 | EM1030-B | CACCUACAAAGCUGUGAUU | 60 |
| EM1031 | EM1031-A | UUAAUCAGUGCUAUGUCAU | 61 |
| EM1031 | EM1031-B | AUGACAUAGCACUGAUUAA | 62 |
| EM1032 | EM1032-A | UUAUGUUCUCGAUCCAGGG | 63 |
| EM1032 | EM1032-B | CCCUGGAUCGAGAACAUAA | 64 |
| EM1033 | EM1033-A | UUGCCAAGGAAAAUCACCA | 65 |
| EM1033 | EM1033-B | UGGUGAUUUUCCUUGGCAA | 66 |
| EM1034 | EM1034-A | CACAGGUUCAGGCCACUUC | 67 |
| EM1034 | EM1034-B | GAAGUGGCCUGAACCUGUG | 68 |
| EM1035 | EM1035-A | CAGCACAAAGCAUGUUAGC | 69 |
| EM1035 | EM1035-B | GCUAACAUGCUUUGUGCUG | 70 |
| EM1036 | EM1036-A | CACAUCGAAGGACUCCACA | 71 |
| EM1036 | EM1036-B | UGUGGAGUCCUUCGAUGUG | 72 |
| EM1037 | EM1037-A | UUGUGUAGACUCCAUACUG | 73 |
| EM1037 | EM1037-B | CAGUAUGGAGUCUACACAA | 74 |
| EM1038 | EM1038-A | CACAAUUCAUGGAACCCCA | 75 |
| EM1038 | EM1038-B | UGGGGUUCCAUGAAUUGUG | 76 |
| EM1039 | EM1039-A | UCCACAAACAGGCUCACAG | 77 |
| EM1039 | EM1039-B | CUGUGAGCCUGUUUGUGGA | 78 |
| EM1040 | EM1040-A | ACAGGUUCAGGCCACUUCG | 79 |
| EM1040 | EM1040-B | CGAAGUGGCCUGAACCUGU | 80 |
| EM1041 | EM1041-A | CUGUACUGAAUCACAGCUU | 81 |
| EM1041 | EM1041-B | AAGCUGUGAUUCAGUACAG | 82 |
| EM1042 | EM1042-A | GUCUCCACAUCGAAGGACU | 83 |
| EM1042 | EM1042-B | AGUCCUUCGAUGUGGAGAC | 84 |
| EM1043 | EM1043-A | UCGUCCAGAAUCCAUCAGC | 85 |
| EM1043 | EM1043-B | GCUGAUGGAUUCUGGACGA | 86 |
| EM1044 | EM1044-A | CCCAAGAUCCAUCUUUCUG | 87 |
| EM1044 | EM1044-B | CAGAAAGAUGGAUCUUGGG | 88 |
| EM1045 | EM1045-A | AAGUGGGUGAAGUAGAGGC | 89 |
| EM1045 | EM1045-B | GCCUCUACUUCACCCACUU | 90 |
| EM1046 | EM1046-A | CAUUAGAUUUCUAGCAAGA | 91 |
| EM1046 | EM1046-B | UCUUGCUAGAAAUCUAAUG | 92 |
| EM1047 | EM1047-A | CUCCACAUCGAAGGACUCC | 93 |
| EM1047 | EM1047-B | GGAGUCCUUCGAUGUGGAG | 94 |
| EM1048 | EM1048-A | CAGCUGUACUGAAUCACAG | 95 |
| EM1048 | EM1048-B | CUGUGAUUCAGUACAGCUG | 96 |
| EM1049 | EM1049-A | CAUGAGCAGCUGUUAGGAC | 97 |
| EM1049 | EM1049-B | GUCCUAACAGCUGCUCAUG | 98 |
| EM1050 | EM1050-A | GUCUGUUUGAAUCUUGAGA | 99 |
| EM1050 | EM1050-B | UCUCAAGAUUCAAACAGAC | 100 |
| EM1051 | EM1051-A | CCUUUGGAGCUCGUCCAGA | 101 |
| EM1051 | EM1051-B | UCUGGACGAGCUCCAAAGG | 102 |
| EM1052 | EM1052-A | GCUGUUAGGACCCAGUUGU | 103 |
| EM1052 | EM1052-B | ACAACUGGGUCCUAACAGC | 104 |
| EM1053 | EM1053-A | GGUAGAUCAUCAGGAGGGC | 105 |
| EM1053 | EM1053-B | GCCCUCCUGAUGAUCUACC | 106 |
| EM1054 | EM1054-A | GGGUAGAUCAUCAGGAGGG | 107 |
| EM1054 | EM1054-B | CCCUCCUGAUGAUCUACCC | 108 |
| EM1055 | EM1055-A | UGGCUUGCACAGGUGAAAC | 109 |
| EM1055 | EM1055-B | GUUUCACCUGUGCAAGCCA | 110 |
| EM1056 | EM1056-A | CUAUUCCUCCCACAAACCA | 111 |
| EM1056 | EM1056-B | UGGUUUGUGGGAGGAAUAG | 112 |
| EM1057 | EM1057-A | GAGUAGUCGGAGCGGAAGG | 113 |
| EM1057 | EM1057-B | CCUUCCGCUCCGACUACUC | 114 |
| EM1058 | EM1058-A | GAUCCAGGGAAUAUAGUUA | 115 |
| EM1058 | EM1058-B | UAACUAUAUUCCCUGGAUC | 116 |
| EM1059 | EM1059-A | AUUAGAUUUCUAGCAAGAA | 117 |
| EM1059 | EM1059-B | UUCUUGCUAGAAAUCUAAU | 118 |
| EM1060 | EM1060-A | GUUAGGACCCAGUUGUCAU | 119 |
| EM1060 | EM1060-B | AUGACAACUGGGUCCUAAC | 120 |
| EM1061 | EM1061-A | AUCCAGGGAAUAUAGUUAA | 121 |
| EM1061 | EM1061-B | UUAACUAUAUUCCCUGGAU | 122 |
| EM1062 | EM1062-A | AAGUCCAGAAUGACACUGA | 123 |
| EM1062 | EM1062-B | UCAGUGUCAUUCUGGACUU | 124 |
| EM1063 | EM1063-A | ACAUUUUUGAUGGUCAACA | 125 |
| EM1063 | EM1063-B | UGUUGACCAUCAAAAAUGU | 126 |
| EM1064 | EM1064-A | GAGCUCGUCCAGAAUCCAU | 127 |
| EM1064 | EM1064-B | AUGGAUUCUGGACGAGCUC | 128 |
| EM1065 | EM1065-A | GUGUAGAAGGUCUCUUCAC | 129 |
| EM1065 | EM1065-B | GUGAAGAGACCUUCUACAC | 130 |
| EM1066 | EM1066-A | CACUAUUCCUCCCACAAAC | 131 |
| EM1066 | EM1066-B | GUUUGUGGGAGGAAUAGUG | 132 |
| EM1067 | EM1067-A | ACACUAUUCCUCCCACAAA | 133 |
| EM1067 | EM1067-B | UUUGUGGGAGGAAUAGUGU | 134 |
| EM1068 | EM1068-A | UGUACUGAAUCACAGCUUU | 135 |
| EM1068 | EM1068-B | AAAGCUGUGAUUCAGUACA | 136 |
| EM1069 | EM1069-A | AACCCCAGGACACUAUUCC | 137 |
| EM1069 | EM1069-B | GGAAUAGUGUCCUGGGGUU | 138 |
| EM1070 | EM1070-A | CUUUGGAGCUCGUCCAGAA | 139 |
| EM1070 | EM1070-B | UUCUGGACGAGCUCCAAAG | 140 |
| EM1071 | EM1071-A | UAGUGGAUCUUCCAGCCUG | 141 |
| EM1071 | EM1071-B | CAGGCUGGAAGAUCCACUA | 142 |
| EM1072 | EM1072-A | GACACUAUUCCUCCCACAA | 143 |
| EM1072 | EM1072-B | UUGUGGGAGGAAUAGUGUC | 144 |
| EM1073 | EM1073-A | ACAUUAGAUUUCUAGCAAG | 145 |
| EM1073 | EM1073-B | CUUGCUAGAAAUCUAAUGU | 146 |
| EM1074 | EM1074-A | UCUCGUUGGAGUAGUCGGA | 147 |
| EM1074 | EM1074-B | UCCGACUACUCCAACGAGA | 148 |
| EM1075 | EM1075-A | CACAGCUGUACUGAAUCAC | 149 |
| EM1075 | EM1075-B | GUGAUUCAGUACAGCUGUG | 150 |
| EM1076 | EM1076-A | UGUCUGUUUGAAUCUUGAG | 151 |
| EM1076 | EM1076-B | CUCAAGAUUCAAACAGACA | 152 |
| EM1077 | EM1077-A | ACUGAAUCACAGCUUUGUA | 153 |
| EM1077 | EM1077-B | UACAAAGCUGUGAUUCAGU | 154 |
| EM1078 | EM1078-A | GUCCAGAAUCCAUCAGCCU | 155 |
| EM1078 | EM1078-B | AGGCUGAUGGAUUCUGGAC | 156 |
| EM1079 | EM1079-A | ACUUGCCAAGGAAAAUCAC | 157 |
| EM1079 | EM1079-B | GUGAUUUUCCUUGGCAAGU | 158 |
| EM1080 | EM1080-A | UCACAGCUGUACUGAAUCA | 159 |
| EM1080 | EM1080-B | UGAUUCAGUACAGCUGUGA | 160 |
| EM1081 | EM1081-A | AGCUCGUCCAGAAUCCAUC | 161 |
| EM1081 | EM1081-B | GAUGGAUUCUGGACGAGCU | 162 |
| EM1082 | EM1082-A | AUCUUCCAGCCUGUGUGGU | 163 |
| EM1082 | EM1082-B | ACCACACAGGCUGGAAGAU | 164 |
| EM1083 | EM1083-A | UGGAGUAGUCGGAGCGGAA | 165 |
| EM1083 | EM1083-B | UUCCGCUCCGACUACUCCA | 166 |
| EM1084 | EM1084-A | CUCGAUCCAGGGAAUAUAG | 167 |
| EM1084 | EM1084-B | CUAUAUUCCCUGGAUCGAG | 168 |
| EM1085 | EM1085-A | UACUGAAUCACAGCUUUGU | 169 |
| EM1085 | EM1085-B | ACAAAGCUGUGAUUCAGUA | 170 |
| EM1086 | EM1086-A | UCCAGAAUCCAUCAGCCUC | 171 |
| EM1086 | EM1086-B | GAGGCUGAUGGAUUCUGGA | 172 |
| EM1087 | EM1087-A | AAGGAUUCAGCUUCUUUUC | 173 |
| EM1087 | EM1087-B | GAAAAGAAGCUGAAUCCUU | 174 |
| EM1088 | EM1088-A | UGAAUCACAGCUUUGUAGG | 175 |
| EM1088 | EM1088-B | CCUACAAAGCUGUGAUUCA | 176 |
| EM1089 | EM1089-A | GUGGGUGAAGUAGAGGCGC | 177 |
| EM1089 | EM1089-B | GCGCCUCUACUUCACCCAC | 178 |
| EM1090 | EM1090-A | CCAGAAUCCAUCAGCCUCA | 179 |
| EM1090 | EM1090-B | UGAGGCUGAUGGAUUCUGG | 180 |
| EM1091 | EM1091-A | CAUUGUGUAGAAGGUCUCU | 181 |
| EM1091 | EM1091-B | AGAGACCUUCUACACAAUG | 182 |
| EM1092 | EM1092-A | UUUGGCUUGCACAGGUGAA | 183 |
| EM1092 | EM1092-B | UUCACCUGUGCAAGCCAAA | 184 |
| EM1093 | EM1093-A | CAAUUCAUGGAACCCCAGG | 185 |
| EM1093 | EM1093-B | CCUGGGGUUCCAUGAAUUG | 186 |
| EM1094 | EM1094-A | CUUCACAGCUGUACUGAAU | 187 |
| EM1094 | EM1094-B | AUUCAGUACAGCUGUGAAG | 188 |
| EM1095 | EM1095-A | GCUGCAUAGAAGGCCUCGA | 189 |
| EM1095 | EM1095-B | UCGAGGCCUUCUAUGCAGC | 190 |
| EM1096 | EM1096-A | GAAUCCAUCAGCCUCACAC | 191 |
| EM1096 | EM1096-B | GUGUGAGGCUGAUGGAUUC | 192 |
| EM1097 | EM1097-A | AAUCCAUCAGCCUCACACA | 193 |
| EM1097 | EM1097-B | UGUGUGAGGCUGAUGGAUU | 194 |
| EM1098 | EM1098-A | AGUGGGUGAAGUAGAGGCG | 195 |
| EM1098 | EM1098-B | CGCCUCUACUUCACCCACU | 196 |
| EM1099 | EM1099-A | CUCGUCCAGAAUCCAUCAG | 197 |
| EM1099 | EM1099-B | CUGAUGGAUUCUGGACGAG | 198 |
| EM1100 | EM1100-A | UUGGAGUAGUCGGAGCGGA | 199 |
| EM1100 | EM1100-B | UCCGCUCCGACUACUCCAA | 200 |
| EM1101 | EM1101-A | ACAAUUCAUGGAACCCCAG | 201 |
| EM1101 | EM1101-B | CUGGGGUUCCAUGAAUUGU | 202 |
| EM1102 | EM1102-A | GGUCUCUUCACAGCUGUAC | 203 |
| EM1102 | EM1102-B | GUACAGCUGUGAAGAGACC | 204 |
| EM1103 | EM1103-A | ACAGCUGUACUGAAUCACA | 205 |
| EM1103 | EM1103-B | UGUGAUUCAGUACAGCUGU | 206 |
| EM1104 | EM1104-A | CGAUCCAGGGAAUAUAGUU | 207 |
| EM1104 | EM1104-B | AACUAUAUUCCCUGGAUCG | 208 |
| EM1105 | EM1105-A | CUCCUUUGGAGCUCGUCCA | 209 |
| EM1105 | EM1105-B | UGGACGAGCUCCAAAGGAG | 210 |
| EM1106 | EM1106-A | UAGAUCAUCAGGAGGGCCA | 211 |
| EM1106 | EM1106-B | UGGCCCUCCUGAUGAUCUA | 212 |
| EM1107 | EM1107-A | CCACAAUUCAUGGAACCCC | 213 |
| EM1107 | EM1107-B | GGGGUUCCAUGAAUUGUGG | 214 |
| EM1108 | EM1108-A | CAUAGAAGGCCUCGAACCC | 215 |
| EM1108 | EM1108-B | GGGUUCGAGGCCUUCUAUG | 216 |
| EM1109 | EM1109-A | UUGGAGCUCGUCCAGAAUC | 217 |
| EM1109 | EM1109-B | GAUUCUGGACGAGCUCCAA | 218 |
| EM1110 | EM1110-A | UUCUCGUUGGAGUAGUCGG | 219 |
| EM1110 | EM1110-B | CCGACUACUCCAACGAGAA | 220 |
| EM1111 | EM1111-A | AUGUUCUCGAUCCAGGGAA | 221 |
| EM1111 | EM1111-B | UUCCCUGGAUCGAGAACAU | 222 |
| EM1112 | EM1112-A | AGGUGCGCUUGUUACGGUG | 223 |
| EM1112 | EM1112-B | CACCGUAACAAGCGCACCU | 224 |
| EM1113 | EM1113-A | CCUCCAUAUAUACGCCCUC | 225 |
| EM1113 | EM1113-B | GAGGGCGUAUAUAUGGAGG | 226 |
| EM1114 | EM1114-A | UGCUAUGUCAUUGUCAAAG | 227 |
| EM1114 | EM1114-B | CUUUGACAAUGACAUAGCA | 228 |
| EM1115 | EM1115-A | AAAAUCACCAGGUUUUGCC | 229 |
| EM1115 | EM1115-B | GGCAAAACCUGGUGAUUUU | 230 |
| EM1116 | EM1116-A | CUCAUAAAGGAUUCAGCUU | 231 |
| EM1116 | EM1116-B | AAGCUGAAUCCUUUAUGAG | 232 |
| EM1117 | EM1117-A | UUCAUAUGCAGCAGUACAU | 233 |
| EM1117 | EM1117-B | AUGUACUGCUGCAUAUGAA | 234 |
| EM1118 | EM1118-A | UUGACGAAGUCGUACUCGC | 235 |
| EM1118 | EM1118-B | GCGAGUACGACUUCGUCAA | 236 |
| EM1119 | EM1119-A | UAGUCGGAGCGGAAGGUAA | 237 |
| EM1119 | EM1119-B | UUACCUUCCGCUCCGACUA | 238 |
| EM1120 | EM1120-A | UGAUAGUCCACAAACAGGC | 239 |
| EM1120 | EM1120-B | GCCUGUUUGUGGACUAUCA | 240 |
| EM1121 | EM1121-A | GUCUUCACAGGCAUUUCUA | 241 |
| EM1121 | EM1121-B | UAGAAAUGCCUGUGAAGAC | 242 |
| EM1122 | EM1122-A | UGAAUCUUGAGAAAGUCGU | 243 |
| EM1122 | EM1122-B | ACGACUUUCUCAAGAUUCA | 244 |
| EM1123 | EM1123-A | UCGGAGCGGAAGGUAAUGU | 245 |
| EM1123 | EM1123-B | ACAUUACCUUCCGCUCCGA | 246 |
| EM1124 | EM1124-A | CACCUGGAGUCUGUUUUUU | 247 |
| EM1124 | EM1124-B | AAAAAACAGACUCCAGGUG | 248 |
| EM1125 | EM1125-A | UUAUUCAAUUUAAUCAGUG | 249 |
| EM1125 | EM1125-B | CACUGAUUAAAUUGAAUAA | 250 |
| EM1126 | EM1126-A | AAAGGAUUCAGCUUCUUUU | 251 |
| EM1126 | EM1126-B | AAAAGAAGCUGAAUCCUUU | 252 |
| EM1127 | EM1127-A | GUAGUCGGAGCGGAAGGUA | 253 |
| EM1127 | EM1127-B | UACCUUCCGCUCCGACUAC | 254 |
| EM1128 | EM1128-A | AGUCGGAGCGGAAGGUAAU | 255 |
| EM1128 | EM1128-B | AUUACCUUCCGCUCCGACU | 256 |
| EM1129 | EM1129-A | CAUAUAAAAGUGCACCUGC | 257 |
| EM1129 | EM1129-B | GCAGGUGCACUUUUAUAUG | 258 |
| EM1130 | EM1130-A | CAUAUAUACGCCCUCCUGU | 259 |
| EM1130 | EM1130-B | ACAGGAGGGCGUAUAUAUG | 260 |
| EM1131 | EM1131-A | UCCAUAUAUACGCCCUCCU | 261 |
| EM1131 | EM1131-B | AGGAGGGCGUAUAUAUGGA | 262 |
| EM1132 | EM1132-A | UAUUGAUUACAACUUUGUU | 263 |
| EM1132 | EM1132-B | AACAAAGUUGUAAUCAAUA | 264 |
| EM1133 | EM1133-A | UACAACUUUGUUAUUCAAU | 265 |
| EM1133 | EM1133-B | AUUGAAUAACAAAGUUGUA | 266 |
| EM1134 | EM1134-A | UUGCCUUUUGCCCUCCAUA | 267 |
| EM1134 | EM1134-B | UAUGGAGGGCAAAAGGCAA | 268 |
| EM1135 | EM1135-A | UAAUAUCAGGACUUGCCAA | 269 |
| EM1135 | EM1135-B | UUGGCAAGUCCUGAUAUUA | 270 |
| EM1136 | EM1136-A | UCGUAGGGACACAGGGUUU | 271 |
| EM1136 | EM1136-B | AAACCCUGUGUCCCUACGA | 272 |
| EM1137 | EM1137-A | UCGUCAAUGUCCUCGGCUG | 273 |
| EM1137 | EM1137-B | CAGCCGAGGACAUUGACGA | 274 |
| EM1138 | EM1138-A | CCCUCCAUAUAUACGCCCU | 275 |
| EM1138 | EM1138-B | AGGGCGUAUAUAUGGAGGG | 276 |
| EM1139 | EM1139-A | UCCAGAAUGACACUGAACC | 277 |
| EM1139 | EM1139-B | GGUUCAGUGUCAUUCUGGA | 278 |
| EM1140 | EM1140-A | AAAGCCAGCAUCAUGAGUA | 279 |
| EM1140 | EM1140-B | UACUCAUGAUGCUGGCUUU | 280 |
| EM1141 | EM1141-A | AUAUACGCCCUCCUGUUGU | 281 |
| EM1141 | EM1141-B | ACAACAGGAGGGCGUAUAU | 282 |
| EM1142 | EM1142-A | UAUGUCAUUGUCAAAGCCA | 283 |
| EM1142 | EM1142-B | UGGCUUUGACAAUGACAUA | 284 |
| EM1143 | EM1143-A | AUACUCCCCUGGAAAGCCG | 285 |
| EM1143 | EM1143-B | CGGCUUUCCAGGGGAGUAU | 286 |
| EM1144 | EM1144-A | AUCAUGAGUAUAACCUUCA | 287 |
| EM1144 | EM1144-B | UGAAGGUUAUACUCAUGAU | 288 |
| EM1145 | EM1145-A | UGGCAGACAAAUAGGCGUG | 289 |
| EM1145 | EM1145-B | CACGCCUAUUUGUCUGCCA | 290 |
| EM1146 | EM1146-A | UCUUUCAGGAUGUAUUUGG | 291 |
| EM1146 | EM1146-B | CCAAAUACAUCCUGAAAGA | 292 |
| EM1147 | EM1147-A | UUGAAUCUUGAGAAAGUCG | 293 |
| EM1147 | EM1147-B | CGACUUUCUCAAGAUUCAA | 294 |
| EM1148 | EM1148-A | CUUGCCCCCACUUUCUAAG | 295 |
| EM1148 | EM1148-B | CUUAGAAAGUGGGGGCAAG | 296 |
| EM1149 | EM1149-A | AGUUCCAAUGUCAUCUGUC | 297 |
| EM1149 | EM1149-B | GACAGAUGACAUUGGAACU | 298 |
| EM1150 | EM1150-A | CUUGAGAAAGUCGUAGGGA | 299 |
| EM1150 | EM1150-B | UCCCUACGACUUUCUCAAG | 300 |
| EM1151 | EM1151-A | UUUCAUUGUGUAGAAGGUC | 301 |
| EM1151 | EM1151-B | GACCUUCUACACAAUGAAA | 302 |
| EM1152 | EM1152-A | GCCAGUAUGAAAAGAGACU | 303 |
| EM1152 | EM1152-B | AGUCUCUUUUCAUACUGGC | 304 |
| EM1153 | EM1153-A | AGCUUGACGAAGUCGUACU | 305 |
| EM1153 | EM1153-B | AGUACGACUUCGUCAAGCU | 306 |
| EM1154 | EM1154-A | CAGGUGCGCUUGUUACGGU | 307 |
| EM1154 | EM1154-B | ACCGUAACAAGCGCACCUG | 308 |
| EM1155 | EM1155-A | GACGAAGUCGUACUCGCAG | 309 |
| EM1155 | EM1155-B | CUGCGAGUACGACUUCGUC | 310 |
| EM1156 | EM1156-A | AGGUAAUGUCCAGGCUGGA | 311 |
| EM1156 | EM1156-B | UCCAGCCUGGACAUUACCU | 312 |
| EM1157 | EM1157-A | UGACGAAGUCGUACUCGCA | 313 |
| EM1157 | EM1157-B | UGCGAGUACGACUUCGUCA | 314 |
| EM1158 | EM1158-A | CUGUGCUCGUGUAGUGGAU | 315 |
| EM1158 | EM1158-B | AUCCACUACACGAGCACAG | 316 |
| EM1159 | EM1159-A | UCAUAAAGGAUUCAGCUUC | 317 |
| EM1159 | EM1159-B | GAAGCUGAAUCCUUUAUGA | 318 |
| EM1160 | EM1160-A | ACAUACAUUAGAUUUCUAG | 319 |
| EM1160 | EM1160-B | CUAGAAAUCUAAUGUAUGU | 320 |
| EM1161 | EM1161-A | CAUACAUUAGAUUUCUAGC | 321 |
| EM1161 | EM1161-B | GCUAGAAAUCUAAUGUAUG | 322 |
| EM1162 | EM1162-A | AGCAUCAUGAGUAUAACCU | 323 |
| EM1162 | EM1162-B | AGGUUAUACUCAUGAUGCU | 324 |
| EM1163 | EM1163-A | CUUCAUGUAUAAAAACAGC | 325 |
| EM1163 | EM1163-B | GCUGUUUUUAUACAUGAAG | 326 |
| EM1164 | EM1164-A | AGACAAAUAGGCGUGAUGU | 327 |
| EM1164 | EM1164-B | ACAUCACGCCUAUUUGUCU | 328 |
| EM1165 | EM1165-A | ACAAAUAGGCGUGAUGUUG | 329 |
| EM1165 | EM1165-B | CAACAUCACGCCUAUUUGU | 330 |
| EM1166 | EM1166-A | AUUGAUUACAACUUUGUUA | 331 |
| EM1166 | EM1166-B | UAACAAAGUUGUAAUCAAU | 332 |
| EM1167 | EM1167-A | UGUCAUCUGUCCUCAUAAA | 333 |
| EM1167 | EM1167-B | UUUAUGAGGACAGAUGACA | 334 |
| EM1168 | EM1168-A | AUGAGUAUAACCUUCAUGU | 335 |
| EM1168 | EM1168-B | ACAUGAAGGUUAUACUCAU | 336 |
| EM1169 | EM1169-A | CUCGUCAAUGUCCUCGGCU | 337 |
| EM1169 | EM1169-B | AGCCGAGGACAUUGACGAG | 338 |
| EM1170 | EM1170-A | CUUGACGAAGUCGUACUCG | 339 |
| EM1170 | EM1170-B | CGAGUACGACUUCGUCAAG | 340 |
| EM1171 | EM1171-A | UUAAUCCCCAUCCAGAUGC | 341 |
| EM1171 | EM1171-B | GCAUCUGGAUGGGGAUUAA | 342 |
| EM1172 | EM1172-A | GUACUCGCAGAGGUGGGAG | 343 |
| EM1172 | EM1172-B | CUCCCACCUCUGCGAGUAC | 344 |
| EM1173 | EM1173-A | AUAGAAGGCCUCGAACCCC | 345 |
| EM1173 | EM1173-B | GGGGUUCGAGGCCUUCUAU | 346 |
| EM1174 | EM1174-A | AUUUAAUCAGUGCUAUGUC | 347 |
| EM1174 | EM1174-B | GACAUAGCACUGAUUAAAU | 348 |
| EM1175 | EM1175-A | AUGUCAUCUGUCCUCAUAA | 349 |
| EM1175 | EM1175-B | UUAUGAGGACAGAUGACAU | 350 |
| EM1176 | EM1176-A | AGUAUGAAAAGAGACUGGC | 351 |
| EM1176 | EM1176-B | GCCAGUCUCUUUUCAUACU | 352 |
| EM1177 | EM1177-A | GUUAGCAGUUACACUUCCC | 353 |
| EM1177 | EM1177-B | GGGAAGUGUAACUGCUAAC | 354 |
| EM1178 | EM1178-A | AGCCAGUAUGAAAAGAGAC | 355 |
| EM1178 | EM1178-B | GUCUCUUUUCAUACUGGCU | 356 |
| EM1179 | EM1179-A | GAUGUAUUUGGCUUGCACA | 357 |
| EM1179 | EM1179-B | UGUGCAAGCCAAAUACAUC | 358 |
| EM1180 | EM1180-A | AAUGCCAACAGCCAGUAUG | 359 |
| EM1180 | EM1180-B | CAUACUGGCUGUUGGCAUU | 360 |
| EM1181 | EM1181-A | AGCAUGUUAGCAGUUACAC | 361 |
| EM1181 | EM1181-B | GUGUAACUGCUAACAUGCU | 362 |
| EM1182 | EM1182-A | UGUCAUAUAAAAGUGCACC | 363 |
| EM1182 | EM1182-B | GGUGCACUUUUAUAUGACA | 364 |
| EM1183 | EM1183-A | GCCAACAGCCAGUAUGAAA | 365 |
| EM1183 | EM1183-B | UUUCAUACUGGCUGUUGGC | 366 |
| EM1184 | EM1184-A | ACAAAGUCCAGAAUGACAC | 367 |
| EM1184 | EM1184-B | GUGUCAUUCUGGACUUUGU | 368 |
| EM1185 | EM1185-A | AGGUUCAGGCCACUUCGGG | 369 |
| EM1185 | EM1185-B | CCCGAAGUGGCCUGAACCU | 370 |
| EM1186 | EM1186-A | CUAAUAUCAGGACUUGCCA | 371 |
| EM1186 | EM1186-B | UGGCAAGUCCUGAUAUUAG | 372 |
| EM1187 | EM1187-A | UCUGUGACAAAGGUGAUGG | 373 |
| EM1187 | EM1187-B | CCAUCACCUUUGUCACAGA | 374 |
| EM1188 | EM1188-A | UUUCAUAUGCAGCAGUACA | 375 |
| EM1188 | EM1188-B | UGUACUGCUGCAUAUGAAA | 376 |
| EM1189 | EM1189-A | AAGACCUGGCCGGAGCACA | 377 |
| EM1189 | EM1189-B | UGUGCUCCGGCCAGGUCUU | 378 |
| EM1190 | EM1190-A | UCAUGGUGUGCCCGUCCAG | 379 |
| EM1190 | EM1190-B | CUGGACGGGCACACCAUGA | 380 |
| EM1191 | EM1191-A | GAAGGUAAUGUCCAGGCUG | 381 |
| EM1191 | EM1191-B | CAGCCUGGACAUUACCUUC | 382 |
| EM1192 | EM1192-A | AAGUCGUACUCGCAGAGGU | 383 |
| EM1192 | EM1192-B | ACCUCUGCGAGUACGACUU | 384 |
| EM1214 | EM1214-A | UUGGGUGAAGUAGAGGCGC | 385 |
| EM1214 | EM1214-B | GCGCCUCUACUUCACCCAC | 178 |
| EM1215 | EM1215-A | UACGAAGUCGUACUCGCAG | 386 |
| EM1215 | EM1215-B | CUGCGAGUACGACUUCGUC | 310 |
| EM1216 | EM1216-A | UUAGAAGGCCUCGAACCCC | 387 |
| EM1216 | EM1216-B | GGGGUUCGAGGCCUUCUAU | 346 |
| EM1217 | EM1217-A | UAAGGUAAUGUCCAGGCUG | 388 |
| EM1217 | EM1217-B | CAGCCUGGACAUUACCUUC | 382 |
| EM1218 | EM1218-A | UAGUCGUACUCGCAGAGGU | 389 |
| EM1218 | EM1218-B | ACCUCUGCGAGUACGACUU | 384 |

The duplexes listed in Table 5b have various modifications as shown, with reference to Table 4 for an explanation of the abbreviations used. Where appropriate, the sequence of the equivalent unmodified strand from Table 5a is also indicated.

TABLE 5b
Modified duplexes

| | | | | Unmodified |
|---|---|---|---|---|
| | Strand | | | equivalent |
| Duplex ID | Name (*) | Sequence (5′→3′) | SEQ ID No. | SEQ ID No. |
| EM1001 | EM1001-A | mG (ps) fA (ps) mC fU mU fG mC fC mA fA mG fG mA fA mA fA mU (ps) fC (ps) mA | 390 | 1 |
| EM1001 | EM1001-B | mU (ps) mG (ps) mA mU mU mU fU fC fC mU mU mG mG mC mA mA mG (ps) mU (ps) mC | 391 | 2 |
| EM1002 | EM1002-A | mU (ps) fG (ps) mG fG mU fA mG fA mU fC mA fU mC fA mG fG mA (ps) fG (ps) mG | 392 | 3 |
| EM1002 | EM1002-B | mC (ps) mC (ps) mU mC mC mU fG fA fU mG mA mU mC mU mA mC mC (ps) mC (ps) mA | 393 | 4 |
| EM1003 | EM1003-A | mU (ps) fC (ps) mC fA mC fA mU fC mG fA mA fG mG fA mC fU mC (ps) fC (ps) mA | 394 | 5 |
| EM1003 | EM1003-B | mU (ps) mG (ps) mG mA mG mU fC fC fU mU mC mG mA mU mG mU mG (ps) mG (ps) mA | 395 | 6 |
| EM1004 | EM1004-A | mU (ps) fU (ps) mG fG mC fU mU fG mC fA mC fA mG fG mU fG mA (ps) fA (ps) mA | 396 | 7 |
| EM1004 | EM1004-B | mU (ps) mU (ps) mU mC mA mC fC fU fG mU mG mC mA mA mG mC mC (ps) mA (ps) mA | 397 | 8 |
| EM1005 | EM1005-A | mA (ps) fU (ps) mU fG mU fG mU fA mG fA mA fG mG fU mC fU mC (ps) fU (ps) mU | 398 | 9 |
| EM1005 | EM1005-B | mA (ps) mA (ps) mG mA mG mA fC fC fU mU mC mU mA mC mA mC mA (ps) mA (ps) mU | 399 | 10 |
| EM1006 | EM1006-A | mA (ps) fC (ps) mA fG mC fU mU fU mG fU mA fG mG fU mG fG mU (ps) fC (ps) mA | 400 | 11 |
| EM1006 | EM1006-B | mU (ps) mG (ps) mA mC mC mA fC fC fU mA mC mA mA mA mG mC mU (ps) mG (ps) mU | 401 | 12 |
| EM1007 | EM1007-A | mA (ps) fG (ps) mU fA mG fA mG fG mC fG mC fA mG fG mC fG mG (ps) fU (ps) mA | 402 | 13 |
| EM1007 | EM1007-B | mU (ps) mA (ps) mC mC mG mC fC fU fG mC mG mC mC mU mC mU mA (ps) mC (ps) mU | 403 | 14 |
| EM1008 | EM1008-A | mU (ps) fC (ps) mU fC mC fU mU fU mG fG mA fG mC fU mC fG mU (ps) fC (ps) mC | 404 | 15 |
| EM1008 | EM1008-B | mG (ps) mG (ps) mA mC mG mA fG fC fU mC mC mA mA mA mG mG mA (ps) mG (ps) mA | 405 | 16 |
| EM1009 | EM1009-A | mA (ps) fG (ps) mA fA mU fC mC fA mU fC mA fG mC fC mU fC mA (ps) fC (ps) mA | 406 | 17 |
| EM1009 | EM1009-B | mU (ps) mG (ps) mU mG mA mG fG FC fU mG mA mU mG mG mA mU mU (ps) mC (ps) mU | 407 | 18 |
| EM1010 | EM1010-A | mG (ps) fG (ps) mU fC mC fC mA fA mG fA mU fC mC fA mU fC mU (ps) fU (ps) mU | 408 | 19 |
| EM1010 | EM1010-B | mA (ps) mA (ps) mA mG mA mU fG fG fA mU mC mU mU mG mG mG mA (ps) mC (ps) mC | 409 | 20 |
| EM1011 | EM1011-A | mG (ps) fC (ps) mA fU mU fG mG fC mC fG mG fU mC fC mC fA mA (ps) fG (ps) mA | 410 | 21 |
| EM1011 | EM1011-B | mU (ps) mC (ps) mU mU mG mG fG fA fC mC mG mG mC mC mA mA mU (ps) mG (ps) mC | 411 | 22 |
| EM1012 | EM1012-A | mC (ps) fC (ps) mA fC mA fA mA fC mA fG mG fC mU fC mA fC mA (ps) fG (ps) mA | 412 | 23 |
| EM1012 | EM1012-B | mU (ps) mC (ps) mU mG mU mG fA fG fC mC mU mG mU mU mU mG mU (ps) mG (ps) mG | 413 | 24 |
| EM1013 | EM1013-A | mG (ps) fU (ps) mA fC mU fG mA fA mU fC mA fC mA fG mC fU mU (ps) fU (ps) mG | 414 | 25 |
| EM1013 | EM1013-B | mC (ps) mA (ps) mA mA mG mC fU fG fU mG mA mU mU mC mA mG mU (ps) mA (ps) mC | 415 | 26 |
| EM1014 | EM1014-A | mC (ps) fU (ps) mA fA mG fC mC fA mG fC mA fC mA fA mA fG mC (ps) fA (ps) mU | 416 | 27 |
| EM1014 | EM1014-B | mA (ps) mU (ps) mG mC mU mU fU fG fU mG mC mU mG mG mC mU mU (ps) mA (ps) mG | 417 | 28 |
| EM1015 | EM1015-A | mU (ps) fU (ps) mC fA mC fA mG fC mU fG mU fA mC fU mG fA mA (ps) fU (ps) mC | 418 | 29 |
| EM1015 | EM1015-B | mG (ps) mA (ps) mU mU mC mA fG fU fA mC mA mG mC mU mG mU mG (ps) mA (ps) mA | 419 | 30 |
| EM1016 | EM1016-A | mU (ps) fA (ps) mA fG mC fC mA fG mC fA mC fA mA fA mG fC mA (ps) fU (ps) mG | 420 | 31 |
| EM1016 | EM1016-B | mC (ps) mA (ps) mU mG mC mU fU fU fG mU mG mC mU mG mG mC mU (ps) mU (ps) mA | 42: | 32 |
| EM1017 | EM1017-A | mA (ps) fG (ps) mU fA mG fU mC fG mG fA mG fC mG fG mA fA mG (ps) fG (ps) mU | 422 | 33 |
| EM1017 | EM1017-B | mA (ps) mC (ps) mC mU mU mC fC fG fC mU mC mC mG mA mC mU mA (ps) mC (ps) mU | 423 | 34 |
| EM1018 | EM1018-A | mU (ps) fC (ps) mU fC mG fA mU fC mC fA mG fG mG fA mA fU mA (ps) fU (ps) mA | 424 | 35 |
| EM1018 | EM1018-B | mU (ps) mA (ps) mU mA mU mU fC fC fC mU mG mG mA mU mC mG mA (ps) mG (ps) mA | 425 | 36 |
| EM1019 | EM1019-A | mU (ps) fC (ps) mG fA mA fG mU fG mG fG mU fG mA fA mG fU mA (ps) fG (ps) mA | 426 | 37 |
| EM1019 | EM1019-B | mU (ps) mC (ps) mU mA mC mU fU fC fA mC mC mC mA mC mU mU mC (ps) mG (ps) mA | 427 | 38 |
| EM1020 | EM1020-A | mC (ps) fU (ps) mU fG mC fC mA fA mG fG mA fA mA fA mU fC mA (ps) fC (ps) mC | 428 | 39 |
| EM1020 | EM1020-B | mG (ps) mG (ps) mU mG mA mU fU fU fU mC mC mU mU mG mG mC mA (ps) mA (ps) mG | 429 | 40 |
| EM1021 | EM1021-A | mG (ps) fC (ps) mA fU mG fA mG fC mA fG mC fU mG fU mU fA mG (ps) fG (ps) mA | 430 | 41 |
| EM1021 | EM1021-B | mU (ps) mC (ps) mC mU mA mA fC fA fG mC mU mG mC mU mC mA mU (ps) mG (ps) mC | 431 | 42 |
| EM1022 | EM1022-A | mG (ps) fG (ps) mA fC mA fC mU fA mU fU mC fC mU fC mC fC mA (ps) fC (ps) mA | 432 | 43 |
| EM1022 | EM1022-B | mU (ps) mG (ps) mU mG mG mG fA fG fG mA mA mU mA mG mU mG mU (ps) mC (ps) mC | 433 | 44 |
| EM1023 | EM1023-A | mU (ps) fG (ps) mU fC mU fC mC fA mC fA mU fC mG fA mA fG mG (ps) fA (ps) mC | 434 | 45 |
| EM1023 | EM1023-B | mG (ps) mU (ps) mC mC mU mU fC fG fA mU mG mU mG mG mA mG mA (ps) mC (ps) mA | 435 | 46 |
| EM1024 | EM1024-A | mG (ps) fU (ps) mG fA mC fA mA fA mG fG mU fG mA fU mG fG mU (ps) fC (ps) mA | 436 | 47 |
| EM1024 | EM1024-B | mU (ps) mG (ps) mA mC mC mA fU fC fA mC mC mU mU mU mG mU mC (ps) mA (ps) mC | 437 | 48 |
| EM1025 | EM1025-A | mC (ps) fU (ps) mC fU mU fC mA fC mA fG mC fU mG fU mA fC mU (ps) fG (ps) mA | 438 | 49 |
| EM1025 | EM1025-B | mU (ps) mC (ps) mA mG mU mA fC fA fG mC mU mG mU mG mA mA mG (ps) mA (ps) mG | 439 | 50 |
| EM1026 | EM1026-A | mA (ps) fA (ps) mG fG mU fC mU fC mU fU mC fA mC fA mG fC mU (ps) fG (ps) mU | 440 | 51 |
| EM1026 | EM1026-B | mA (ps) mC (ps) mA mG mC mU fG fU fG mA mA mG mA mG mA mC mC (ps) mU (ps) mU | 441 | 52 |
| EM1027 | EM1027-A | mG (ps) fC (ps) mA fU mA fG mA fA mG fG mC fC mU fC mG fA mA (ps) fC (ps) mC | 442 | 53 |
| EM1027 | EM1027-B | mG (ps) mG (ps) mU mU mC mG fA fG fG mC mC mU mU mC mU mA mU (ps) mG (ps) mC | 443 | 54 |
| EM1028 | EM1028-A | mG (ps) fU (ps) mA fG mA fU mC fA mU fC mA fG mG fA mG fG mG (ps) fC (ps) mC | 444 | 55 |
| EM1028 | EM1028-B | mG (ps) mG (ps) mC mC mC mU fC fC fU mG mA mU mG mA mU mC mU (ps) mA (ps) mC | 445 | 56 |
| EM1029 | EM1029-A | mG (ps) fA (ps) mA fU mC fA mC fA mG fC mU fU mU fG mU fA mG (ps) fG (ps) mU | 446 | 57 |
| EM1029 | EM1029-B | mA (ps) mC (ps) mC mU mA mC fA fA fA mG mC mU mG mU mG mA mU (ps) mU (ps) mC | 447 | 58 |
| EM1030 | EM1030-A | mA (ps) fA (ps) mU fC mA fC mA fG mC fU mU fU mG fU mA fG mG (ps) fU (ps) mG | 448 | 59 |
| EM1030 | EM1030-B | mC (ps) mA (ps) mC mC mU mA fC fA fA mA mG mC mU mG mU mG mA (ps) mU (ps) mU | 449 | 60 |
| EM1031 | EM1031-A | mU (ps) fU (ps) mA fA mU fC mA fG mU fG mC fU mA fU mG fU mC (ps) fA (ps) mU | 450 | 61 |
| EM1031 | EM1031-B | mA (ps) mU (ps) mG mA mC mA fU fA fG mC mA mC mU mG mA mU mU (ps) mA (ps) mA | 451 | 62 |
| EM1032 | EM1032-A | mU (ps) fU (ps) mA fU mG fU mU fC mU fC mG fA mU fC mC fA mG (ps) fG (ps) mG | 452 | 63 |
| EM1032 | EM1032-B | mC (ps) mC (ps) mC mU mG mG fA fU fC mG mA mG mA mA mC mA mU (ps) mA (ps) mA | 453 | 64 |
| EM1033 | EM1033-A | mU (ps) fU (ps) mG fC mC fA mA fG mG fA mA fA mA fU mC fA mC (ps) fC (ps) mA | 454 | 65 |
| EM1033 | EM1033-B | mU (ps) mG (ps) mG mU mG mA fU fU fU mU mC mC mU mU mG mG mC (ps) mA (ps) mA | 45! | 66 |
| EM1034 | EM1034-A | mC (ps) fA (ps) mC fA mG fG mU fU mC fA mG fG mC fC mA fC mU (ps) fU (ps) mC | 456 | 67 |
| EM1034 | EM1034-B | mG (ps) mA (ps) mA mG mU mG fG FC fC mU mG mA mA mC mC mU mG (ps) mU (ps) mG | 457 | 68 |
| EM1035 | EM1035-A | mC (ps) fA (ps) mG fC mA fC mA fA mA fG mC fA mU fG mU fU mA (ps) fG (ps) mC | 458 | 69 |
| EM1035 | EM1035-B | mG (ps) mC (ps) mU mA mA mC fA fU fG mC mU mU mU mG mU mG mC (ps) mU (ps) mG | 459 | 70 |
| EM1036 | EM1036-A | mC (ps) fA (ps) mC fA mU fC mG fA mA fG mG fA mC fU mC fC mA (ps) fC (ps) mA | 460 | 71 |
| EM1036 | EM1036-B | mU (ps) mG (ps) mU mG mG mA fG fU fC mC mU mU mC mG mA mU mG (ps) mU (ps) mG | 461 | 72 |
| EM1037 | EM1037-A | mU (ps) fU (ps) mG fU mG fU mA fG mA fC mU fC mC fA mU fA mC (ps) fU (ps) mG | 462 | 73 |
| EM1037 | EM1037-B | mC (ps) mA (ps) mG mU mA mU fG fG fA mG mU mC mU mA mC mA mC (ps) mA (ps) mA | 463 | 74 |
| EM1038 | EM1038-A | mC (ps) fA (ps) mC fA mA fU mU fC mA fU mG fG mA fA mC fC mC (ps) fC (ps) mA | 464 | 75 |
| EM1038 | EM1038-B | mU (ps) mG (ps) mG mG mG mU fU fC fC mA mU mG mA mA mU mU mG (ps) mU (ps) mG | 465 | 76 |
| EM1039 | EM1039-A | mU (ps) fC (ps) mC fA mC fA mA fA mC fA mG fG mC fU mC fA mC (ps) fA (ps) mG | 466 | 77 |
| EM1039 | EM1039-B | mC (ps) mU (ps) mG mU mG mA fG fC fC mU mG mU mU mU mG mU mG (ps) mG (ps) mA | 467 | 78 |
| EM1040 | EM1040-A | mA (ps) fC (ps) mA fG mG fU mU fC mA fG mG fC mC fA mC fU mU (ps) fC (ps) mG | 468 | 79 |
| EM1040 | EM1040-B | mC (ps) mG (ps) mA mA mG mU fG fG fC mC mU mG mA mA mC mC mU (ps) mG (ps) mU | 469 | 80 |
| EM1041 | EM1041-A | mC (ps) fU (ps) mG fU mA fC mU fG mA fA mU fC mA fC mA fG mC (ps) fU (ps) mU | 470 | 81 |
| EM1041 | EM1041-B | mA (ps) mA (ps) mG mC mU mG fU fG fA mU mU mC mA mG mU mA mC (ps) mA (ps) mG | 471 | 82 |
| EM1042 | EM1042-A | mG (ps) fU (ps) mC fU mC fC mA fC mA fU mC fG mA fA mG fG mA (ps) fC (ps) mU | 472 | 83 |
| EM1042 | EM1042-B | mA (ps) mG (ps) mU mC mC mU fU fC fG mA mU mG mU mG mG mA mG (ps) mA (ps) mC | 473 | 84 |
| EM1043 | EM1043-A | mU (ps) fC (ps) mG fU mC fC mA fG mA fA mU fC mC fA mU fC mA (ps) fG (ps) mC | 474 | 85 |
| EM1043 | EM1043-B | mG (ps) mC (ps) mU mG mA mU fG fG fA mU mU mC mU mG mG mA mC (ps) mG (ps) mA | 475 | 86 |
| EM1044 | EM1044-A | mC (ps) fC (ps) mC fA mA fG mA fU mC fC mA fU mC fU mU fU mC (ps) fU (ps) mG | 476 | 87 |
| EM1044 | EM1044-B | mC (ps) mA (ps) mG mA mA mA fG fA fU mG mG mA mU mC mU mU mG (ps) mG (ps) mG | 477 | 88 |
| EM1045 | EM1045-A | mA (ps) fA (ps) mG fU mG fG mG fU mG fA mA fG mU fA mG fA mG (ps) fG (ps) mC | 478 | 89 |
| EM1045 | EM1045-B | mG (ps) mC (ps) mC mU mC mU fA fC fU mU mC mA mC mC mC mA mC (ps) mU (ps) mU | 479 | 90 |
| EM1046 | EM1046-A | mC (ps) fA (ps) mU fU mA fG mA fU mU fU mC fU mA fG mC fA mA (ps) fG (ps) mA | 480 | 91 |
| EM1046 | EM1046-B | mU (ps) mC (ps) mU mU mG mC fU fA fG mA mA mA mU mC mU mA mA (ps) mU (ps) mG | 481 | 92 |
| EM1047 | EM1047-A | mC (ps) fU (ps) mC fC mA fC mA fU mC fG mA fA mG fG mA fC mU (ps) fC (ps) mC | 482 | 93 |
| EM1047 | EM1047-B | mG (ps) mG (ps) mA mG mU mC fC fU fU mC mG mA mU mG mU mG mG (ps) mA (ps) mG | 483 | 94 |
| EM1048 | EM1048-A | mC (ps) fA (ps) mG fC mU fG mU fA mC fU mG fA mA fU mC fA mC (ps) fA (ps) mG | 484 | 95 |
| EM1048 | EM1048-B | mC (ps) mU (ps) mG mU mG mA fU fU fC mA mG mU mA mC mA mG mC (ps) mU (ps) mG | 485 | 96 |
| EM1049 | EM1049-A | mC (ps) fA (ps) mU fG mA fG mC fA mG fC mU fG mU fU mA fG mG (ps) fA (ps) mC | 486 | 97 |
| EM1049 | EM1049-B | mG (ps) mU (ps) mC mC mU mA fA fC fA mG mC mU mG mC mU mC mA (ps) mU (ps) mG | 487 | 98 |
| EM1050 | EM1050-A | mG (ps) fU (ps) mC fU mG fU mU fU mG fA mA fU mC fU mU fG mA (ps) fG (ps) mA | 488 | 99 |
| EM1050 | EM1050-B | mU (ps) mC (ps) mU mC mA mA fG fA fU mU mC mA mA mA mC mA mG (ps) mA (ps) mC | 489 | 100 |
| EM1051 | EM1051-A | mC (ps) fC (ps) mU fU mU fG mG fA mG fC mU fC mG fU mC fC mA (ps) fG (ps) mA | 490 | 101 |
| EM1051 | EM1051-B | mU (ps) mC (ps) mU mG mG mA fC fG fA mG mC mU mC mC mA mA mA (ps) mG (ps) mG | 491 | 102 |
| EM1052 | EM1052-A | mG (ps) fC (ps) mU fG mU fU mA fG mG fA mC fC mC fA mG fU mU (ps) fG (ps) mU | 492 | 103 |
| EM1052 | EM1052-B | mA (ps) mC (ps) mA mA mC mU fG fG fG mU mC mC mU mA mA mC mA (ps) mG (ps) mC | 493 | 104 |
| EM1053 | EM1053-A | mG (ps) fG (ps) mU fA mG fA mU fC mA fU mC fA mG fG mA fG mG (ps) fG (ps) mC | 494 | 105 |
| EM1053 | EM1053-B | mG (ps) mC (ps) mC mC mU mC fC fU fG mA mU mG mA mU mC mU mA (ps) mC (ps) mC | 495 | 106 |
| EM1054 | EM1054-A | mG (ps) fG (ps) mG fU mA fG mA fU mC fA mU fC mA fG mG fA mG (ps) fG (ps) mG | 496 | 107 |
| EM1054 | EM1054-B | mC (ps) mC (ps) mC mU mC mC fU fG fA mU mG mA mU mC mU mA mC (ps) mC (ps) mC | 497 | 108 |
| EM1055 | EM1055-A | mU (ps) fG (ps) mG fC mU fU mG fC mA fC mA fG mG fU mG fA mA (ps) fA (ps) mC | 498 | 109 |
| EM1055 | EM1055-B | mG (ps) mU (ps) mU mU mC mA fC fC fU mG mU mG mC mA mA mG mC (ps) mC (ps) mA | 499 | 110 |
| EM1056 | EM1056-A | mC (ps) fU (ps) mA fU mU fC mC fU mC fC mC fA mC fA mA fA mC (ps) fC (ps) mA | 500 | 111 |
| EM1056 | EM1056-B | mU (ps) mG (ps) mG mU mU mU fG fU fG mG mG mA mG mG mA mA mU (ps) mA (ps) mG | 501 | 112 |
| EM1057 | EM1057-A | mG (ps) fA (ps) mG fU mA fG mU fC mG fG mA fG mC fG mG fA mA (ps) fG (ps) mG | 502 | 113 |
| EM1057 | EM1057-B | mC (ps) mC (ps) mU mU mC mC fG fC fU mC mC mG mA mC mU mA mC (ps) mU (ps) mC | 503 | 114 |
| EM1058 | EM1058-A | mG (ps) fA (ps) mU fC mC fA mG fG mG fA mA fU mA fU mA fG mU (ps) fU (ps) mA | 504 | 115 |
| EM1058 | EM1058-B | mU (ps) mA (ps) mA mC mU mA fU fA fU mU mC mC mC mU mG mG mA (ps) mU (ps) mC | 505 | 116 |
| EM1059 | EM1059-A | mA (ps) fU (ps) mU fA mG fA mU fU mU fC mU fA mG fC mA fA mG (ps) fA (ps) mA | 506 | 117 |
| EM1059 | EM1059-B | mU (ps) mU (ps) mC mU mU mG fC fU fA mG mA mA mA mU mC mU mA (ps) mA (ps) mU | 507 | 118 |
| EM1060 | EM1060-A | mG (ps) fU (ps) mU fA mG fG mA fC mC fC mA fG mU fU mG fU mC (ps) fA (ps) mU | 508 | 119 |
| EM1060 | EM1060-B | mA (ps) mU (ps) mG mA mC mA fA fC fU mG mG mG mU mC mC mU mA (ps) mA (ps) mC | 509 | 120 |
| EM1061 | EM1061-A | mA (ps) fU (ps) mC fC mA fG mG fG mA fA mU fA mU fA mG fU mU (ps) fA (ps) mA | 510 | 121 |
| EM1061 | EM1061-B | mU (ps) mU (ps) mA mA mC mU fA fU fA mU mU mC mC mC mU mG mG (ps) mA (ps) mU | 511 | 122 |
| EM1062 | EM1062-A | mA (ps) fA (ps) mG fU mC fC mA fG mA fA mU fG mA fC mA fC mU (ps) fG (ps) mA | 512 | 123 |
| EM1062 | EM1062-B | mU (ps) mC (ps) mA mG mU mG fU fC fA mU mU mC mU mG mG mA mC (ps) mU (ps) mU | 513 | 124 |
| EM1063 | EM1063-A | mA (ps) fC (ps) mA fU mU fU mU fU mG fA mU fG mG fU mC fA mA (ps) fC (ps) mA | 514 | 125 |
| EM1063 | EM1063-B | mU (ps) mG (ps) mU mU mG mA fC fC fA mU mC mA mA mA mA mA mU (ps) mG (ps) mU | 515 | 126 |
| EM1064 | EM1064-A | mG (ps) fA (ps) mG fC mU fC mG fU mC fC mA fG mA fA mU fC mC (ps) fA (ps) mU | 516 | 127 |
| EM1064 | EM1064-B | mA (ps) mU (ps) mG mG mA mU fU fC fU mG mG mA mC mG mA mG mC (ps) mU (ps) mC | 517 | 128 |
| EM1065 | EM1065-A | mG (ps) fU (ps) mG fU mA fG mA fA mG fG mU fC mU fC mU fU mC (ps) fA (ps) mC | 518 | 129 |
| EM1065 | EM1065-B | mG (ps) mU (ps) mG mA mA mG fA fG fA mC mC mU mU mC mU mA mC (ps) mA (ps) mC | 519 | 130 |
| EM1066 | EM1066-A | mC (ps) fA (ps) mC fU mA fU mU fC mC fU mC fC mC fA mC fA mA (ps) fA (ps) mC | 520 | 131 |
| EM1066 | EM1066-B | mG (ps) mU (ps) mU mU mG mU fG fG fG mA mG mG mA mA mU mA mG (ps) mU (ps) mG | 521 | 132 |
| EM1067 | EM1067-A | mA (ps) fC (ps) mA fC mU fA mU fU mC fC mU fC mC fC mA fC mA (ps) fA (ps) mA | 522 | 133 |
| EM1067 | EM1067-B | mU (ps) mU (ps) mU mG mU mG fG fG fA mG mG mA mA mU mA mG mU (ps) mG (ps) mU | 523 | 134 |
| EM1068 | EM1068-A | mU (ps) fG (ps) mU fA mC fU mG fA mA fU mC fA mC fA mG fC mU (ps) fU (ps) mU | 524 | 135 |
| EM1068 | EM1068-B | mA (ps) mA (ps) mA mG mC mU fG fU fG mA mU mU mC mA mG mU mA (ps) mC (ps) mA | 525 | 136 |
| EM1069 | EM1069-A | mA (ps) fA (ps) mC fC mC fC mA fG mG fA mC fA mC fU mA fU mU (ps) fC (ps) mC | 526 | 137 |
| EM1069 | EM1069-B | mG (ps) mG (ps) mA mA mU mA fG fU fG mU mC mC mU mG mG mG mG (ps) mU (ps) mU | 527 | 138 |
| EM1070 | EM1070-A | mC (ps) fU (ps) mU fU mG fG mA fG mC fU mC fG mU fC mC fA mG (ps) fA (ps) mA | 528 | 139 |
| EM1070 | EM1070-B | mU (ps) mU (ps) mC mU mG mG fA fC fG mA mG mC mU mC mC mA mA (ps) mA (ps) mG | 529 | 140 |
| EM1071 | EM1071-A | mU (ps) fA (ps) mG fU mG fG mA fU mC fU mU fC mC fA mG fC mC (ps) fU (ps) mG | 530 | 141 |
| EM1071 | EM1071-B | mC (ps) mA (ps) mG mG mC mU fG fG fA mA mG mA mU mC mC mA mC (ps) mU (ps) mA | 531 | 142 |
| EM1072 | EM1072-A | mG (ps) fA (ps) mC fA mC fU mA fU mU fC mC fU mC fC mC fA mC (ps) fA (ps) mA | 532 | 143 |
| EM1072 | EM1072-B | mU (ps) mU (ps) mG mU mG mG fG fA fG mG mA mA mU mA mG mU mG (ps) mU (ps) mC | 533 | 144 |
| EM1073 | EM1073-A | mA (ps) fC (ps) mA fU mU fA mG fA mU fU mU fC mU fA mG fC mA (ps) fA (ps) mG | 534 | 145 |
| EM1073 | EM1073-B | mC (ps) mU (ps) mU mG mC mU fA fG fA mA mA mU mC mU mA mA mU (ps) mG (ps) mU | 535 | 146 |
| EM1074 | EM1074-A | mU (ps) fC (ps) mU fC mG fU mU fG mG fA mG fU mA fG mU fC mG (ps) fG (ps) mA | 536 | 147 |
| EM1074 | EM1074-B | mU (ps) mC (ps) mC mG mA mC fU fA fC mU mC mC mA mA mC mG mA (ps) mG (ps) mA | 537 | 148 |
| EM1075 | EM1075-A | mC (ps) fA (ps) mC fA mG fC mU fG mU fA mC fU mG fA mA fU mC (ps) fA (ps) mC | 538 | 149 |
| EM1075 | EM1075-B | mG (ps) mU (ps) mG mA mU mU fC fA fG mU mA mC mA mG mC mU mG (ps) mU (ps) mG | 539 | 150 |
| EM1076 | EM1076-A | mU (ps) fG (ps) mU fC mU fG mU fU mU fG mA fA mU fC mU fU mG (ps) fA (ps) mG | 540 | 151 |
| EM1076 | EM1076-B | mC (ps) mU (ps) mC mA mA mG fA fU fU mC mA mA mA mC mA mG mA (ps) mC (ps) mA | 541 | 152 |
| EM1077 | EM1077-A | mA (ps) fC (ps) mU fG mA fA mU fC mA fC mA fG mC fU mU fU mG (ps) fU (ps) mA | 542 | 153 |
| EM1077 | EM1077-B | mU (ps) mA (ps) mC mA mA mA fG fC fU mG mU mG mA mU mU mC mA (ps) mG (ps) mU | 543 | 154 |
| EM1078 | EM1078-A | mG (ps) fU (ps) mC fC mA fG mA fA mU fC mC fA mU fC mA fG mC (ps) fC (ps) mU | 544 | 155 |
| EM1078 | EM1078-B | mA (ps) mG (ps) mG mC mU mG fA fU fG mG mA mU mU mC mU mG mG (ps) mA (ps) mC | 545 | 156 |
| EM1079 | EM1079-A | mA (ps) fC (ps) mU fU mG fC mC fA mA fG mG fA mA fA mA fU mC (ps) fA (ps) mC | 546 | 157 |
| EM1079 | EM1079-B | mG (ps) mU (ps) mG mA mU mU fU fU fC mC mU mU mG mG mC mA mA (ps) mG (ps) mU | 547 | 158 |
| EM1080 | EM1080-A | mU (ps) fC (ps) mA fC mA fG mC fU mG fU mA fC mU fG mA fA mU (ps) fC (ps) mA | 548 | 159 |
| EM1080 | EM1080-B | mU (ps) mG (ps) mA mU mU mC fA fG fU mA mC mA mG mC mU mG mU (ps) mG (ps) mA | 549 | 160 |
| EM1081 | EM1081-A | mA (ps) fG (ps) mC fU mC fG mU fC mC fA mG fA mA fU mC fC mA (ps) fU (ps) mC | 550 | 161 |
| EM1081 | EM1081-B | mG (ps) mA (ps) mU mG mG mA fU fU fC mU mG mG mA mC mG mA mG (ps) mC (ps) mU | 551 | 162 |
| EM1082 | EM1082-A | mA (ps) fU (ps) mC fU mU fC mC fA mG fC mC fU mG fU mG fU mG (ps) fG (ps) mU | 552 | 163 |
| EM1082 | EM1082-B | mA (ps) mC (ps) mC mA mC mA fC fA fG mG mC mU mG mG mA mA mG (ps) mA (ps) mU | 553 | 164 |
| EM1083 | EM1083-A | mU (ps) fG (ps) mG fA mG fU mA fG mU fC mG fG mA fG mC fG mG (ps) fA (ps) mA | 554 | 165 |
| EM1083 | EM1083-B | mU (ps) mU (ps) mC mC mG mC fU fC fC mG mA mC mU mA mC mU mC (ps) mC (ps) mA | 555 | 166 |
| EM1084 | EM1084-A | mC (ps) fU (ps) mC fG mA fU mC fC mA fG mG fG mA fA mU fA mU (ps) fA (ps) mG | 556 | 167 |
| EM1084 | EM1084-B | mC (ps) mU (ps) mA mU mA mU fU fC fC mC mU mG mG mA mU mC mG (ps) mA (ps) mG | 557 | 168 |
| EM1085 | EM1085-A | mU (ps) fA (ps) mC fU mG fA mA fU mC fA mC fA mG fC mU fU mU (ps) fG (ps) mU | 558 | 169 |
| EM1085 | EM1085-B | mA (ps) mC (ps) mA mA mA mG fC fU fG mU mG mA mU mU mC mA mG (ps) mU (ps) mA | 559 | 170 |
| EM1086 | EM1086-A | mU (ps) fC (ps) mC fA mG fA mA fU mC fC mA fU mC fA mG fC mC (ps) fU (ps) mC | 560 | 171 |
| EM1086 | EM1086-B | mG (ps) mA (ps) mG mG mC mU fG fA fU mG mG mA mU mU mC mU mG (ps) mG (ps) mA | 561 | 172 |
| EM1087 | EM1087-A | mA (ps) fA (ps) mG fG mA fU mU fC mA fG mC fU mU fC mU fU mU (ps) fU (ps) mC | 562 | 173 |
| EM1087 | EM1087-B | mG (ps) mA (ps) mA mA mA mG fA fA fG mC mU mG mA mA mU mC mC (ps) mU (ps) mU | 563 | 174 |
| EM1088 | EM1088-A | mU (ps) fG (ps) mA fA mU fC mA fC mA fG mC fU mU fU mG fU mA (ps) fG (ps) mG | 564 | 175 |
| EM1088 | EM1088-B | mC (ps) mC (ps) mU mA mC mA fA fA fG mC mU mG mU mG mA mU mU (ps) mC (ps) mA | 565 | 176 |
| EM1089 | EM1089-A | mG (ps) fU (ps) mG fG mG fU mG fA mA fG mU fA mG fA mG fG mC (ps) fG (ps) mC | 566 | 177 |
| EM1089 | EM1089-B | mG (ps) mC (ps) mG mC mC mU fC fU fA mC mU mU mC mA mC mC mC (ps) mA (ps) mC | 567 | 178 |
| EM1090 | EM1090-A | mC (ps) fC (ps) mA fG mA fA mU fC mC fA mU fC mA fG mC fC mU (ps) fC (ps) mA | 568 | 179 |
| EM1090 | EM1090-B | mU (ps) mG (ps) mA mG mG mC fU fG fA mU mG mG mA mU mU mC mU (ps) mG (ps) mG | 569 | 180 |
| EM1091 | EM1091-A | mC (ps) fA (ps) mU fU mG fU mG fU mA fG mA fA mG fG mU fC mU (ps) fC (ps) mU | 570 | 181 |
| EM1091 | EM1091-B | mA (ps) mG (ps) mA mG mA mC fC fU fU mC mU mA mC mA mC mA mA (ps) mU (ps) mG | 571 | 182 |
| EM1092 | EM1092-A | mU (ps) fU (ps) mU fG mG fC mU fU mG fC mA fC mA fG mG fU mG (ps) fA (ps) mA | 572 | 183 |
| EM1092 | EM1092-B | mU (ps) mU (ps) mC mA mC mC fU fG fU mG mC mA mA mG mC mC mA (ps) mA (ps) mA | 573 | 184 |
| EM1093 | EM1093-A | mC (ps) fA (ps) mA fU mU fC mA fU mG fG mA fA mC fC mC fC mA (ps) fG (ps) mG | 574 | 185 |
| EM1093 | EM1093-B | mC (ps) mC (ps) mU mG mG mG fG fU fU mC mC mA mU mG mA mA mU (ps) mU (ps) mG | 575 | 186 |
| EM1094 | EM1094-A | mC (ps) fU (ps) mU fC mA fC mA fG mC fU mG fU mA fC mU fG mA (ps) fA (ps) mU | 576 | 187 |
| EM1094 | EM1094-B | mA (ps) mU (ps) mU mC mA mG fU fA fC mA mG mC mU mG mU mG mA (ps) mA (ps) mG | 577 | 188 |
| EM1095 | EM1095-A | mG (ps) fC (ps) mU fG mC fA mU fA mG fA mA fG mG fC mC fU mC (ps) fG (ps) mA | 578 | 189 |
| EM1095 | EM1095-B | mU (ps) mC (ps) mG mA mG mG fC fC fU mU mC mU mA mU mG mC mA (ps) mG (ps) mC | 579 | 190 |
| EM1096 | EM1096-A | mG (ps) fA (ps) mA fU mC fC mA fU mC fA mG fC mC fU mC fA mC (ps) fA (ps) mC | 580 | 191 |
| EM1096 | EM1096-B | mG (ps) mU (ps) mG mU mG mA fG fG fC mU mG mA mU mG mG mA mU (ps) mU (ps) mC | 581 | 192 |
| EM1097 | EM1097-A | mA (ps) fA (ps) mU fC mC fA mU fC mA fG mC fC mU fC mA fC mA (ps) fC (ps) mA | 582 | 193 |
| EM1097 | EM1097-B | mU (ps) mG (ps) mU mG mU mG fA fG fG mC mU mG mA mU mG mG mA (ps) mU (ps) mU | 583 | 194 |
| EM1098 | EM1098-A | mA (ps) fG (ps) mU fG mG fG mU fG mA fA mG fU mA fG mA fG mG (ps) fC (ps) mG | 584 | 195 |
| EM1098 | EM1098-B | mC (ps) mG (ps) mC mC mU mC fU fA fC mU mU mC mA mC mC mC mA (ps) mC (ps) mU | 585 | 196 |
| EM1099 | EM1099-A | mC (ps) fU (ps) mC fG mU fC mC fA mG fA mA fU mC fC mA fU mC (ps) fA (ps) mG | 586 | 197 |
| EM1099 | EM1099-B | mC (ps) mU (ps) mG mA mU mG fG fA fU mU mC mU mG mG mA mC mG (ps) mA (ps) mG | 587 | 198 |
| EM1100 | EM1100-A | mU (ps) fU (ps) mG fG mA fG mU fA mG fU mC fG mG fA mG fC mG (ps) fG (ps) mA | 588 | 199 |
| EM1100 | EM1100-B | mU (ps) mC (ps) mC mG mC mU fC fC fG mA mC mU mA mC mU mC mC (ps) mA (ps) mA | 589 | 200 |
| EM1101 | EM1101-A | mA (ps) fC (ps) mA fA mU fU mC fA mU fG mG fA mA fC mC fC mC (ps) fA (ps) mG | 590 | 201 |
| EM1101 | EM1101-B | mC (ps) mU (ps) mG mG mG mG fU fU fC mC mA mU mG mA mA mU mU (ps) mG (ps) mU | 591 | 202 |
| EM1102 | EM1102-A | mG (ps) fG (ps) mU fC mU fC mU fU mC fA mC fA mG fC mU fG mU (ps) fA (ps) mC | 592 | 203 |
| EM1102 | EM1102-B | mG (ps) mU (ps) mA mC mA mG fC fU fG mU mG mA mA mG mA mG mA (ps) mC (ps) mC | 593 | 204 |
| EM1103 | EM1103-A | mA (ps) fC (ps) mA fG mC fU mG fU mA fC mU fG mA fA mU fC mA (ps) fC (ps) mA | 594 | 205 |
| EM1103 | EM1103-B | mU (ps) mG (ps) mU mG mA mU fU fC fA mG mU mA mC mA mG mC mU (ps) mG (ps) mU | 595 | 206 |
| EM1104 | EM1104-A | mC (ps) fG (ps) mA fU mC fC mA fG mG fG mA fA mU fA mU fA mG (ps) fU (ps) mU | 596 | 207 |
| EM1104 | EM1104-B | mA (ps) mA (ps) mC mU mA mU fA fU fU mC mC mC mU mG mG mA mU (ps) mC (ps) mG | 597 | 208 |
| EM1105 | EM1105-A | mC (ps) fU (ps) mC fC mU fU mU fG mG fA mG fC mU fC mG fU mC (ps) fC (ps) mA | 598 | 209 |
| EM1105 | EM1105-B | mU (ps) mG (ps) mG mA mC mG fA fG fC mU mC mC mA mA mA mG mG (ps) mA (ps) mG | 599 | 210 |
| EM1106 | EM1106-A | mU (ps) fA (ps) mG fA mU fC mA fU mC fA mG fG mA fG mG fG mC (ps) fC (ps) mA | 600 | 211 |
| EM1106 | EM1106-B | mU (ps) mG (ps) mG mC mC mC fU fC fC mU mG mA mU mG mA mU mC (ps) mU (ps) mA | 601 | 212 |
| EM1107 | EM1107-A | mC (ps) fC (ps) mA fC mA fA mU fU mC fA mU fG mG fA mA fC mC (ps) fC (ps) mC | 602 | 213 |
| EM1107 | EM1107-B | mG (ps) mG (ps) mG mG mU mU fC fC fA mU mG mA mA mU mU mG mU (ps) mG (ps) mG | 603 | 214 |
| EM1108 | EM1108-A | mC (ps) fA (ps) mU fA mG fA mA fG mG fC mC fU mC fG mA fA mC (ps) fC (ps) mC | 604 | 215 |
| EM1108 | EM1108-B | mG (ps) mG (ps) mG mU mU mC fG fA fG mG mC mC mU mU mC mU mA (ps) mU (ps) mG | 605 | 216 |
| EM1109 | EM1109-A | mU (ps) fU (ps) mG fG mA fG mC fU mC fG mU fC mC fA mG fA mA (ps) fU (ps) mC | 606 | 217 |
| EM1109 | EM1109-B | mG (ps) mA (ps) mU mU mC mU fG fG fA mC mG mA mG mC mU mC mC (ps) mA (ps) mA | 607 | 218 |
| EM1110 | EM1110-A | mU (ps) fU (ps) mC fU mC fG mU fU mG fG mA fG mU fA mG fU mC (ps) fG (ps) mG | 608 | 219 |
| EM1110 | EM1110-B | mC (ps) mC (ps) mG mA mC mU fA fC fU mC mC mA mA mC mG mA mG (ps) mA (ps) mA | 609 | 220 |
| EM1111 | EM1111-A | mA (ps) fU (ps) mG fU mU fC mU fC mG fA mU fC mC fA mG fG mG (ps) fA (ps) mA | 610 | 221 |
| EM1111 | EM1111-B | mU (ps) mU (ps) mC mC mC mU fG fG fA mU mC mG mA mG mA mA mC (ps) mA (ps) mU | 611 | 222 |
| EM1112 | EM1112-A | mA (ps) fG (ps) mG fU mG fC mG fC mU fU mG fU mU fA mC fG mG (ps) fU (ps) mG | 612 | 223 |
| EM1112 | EM1112-B | mC (ps) mA (ps) mC mC mG mU fA fA fC mA mA mG mC mG mC mA mC (ps) mC (ps) mU | 613 | 224 |
| EM1113 | EM1113-A | mC (ps) fC (ps) mU fC mC fA mU fA mU fA mU fA mC fG mC fC mC (ps) fU (ps) mC | 614 | 225 |
| EM1113 | EM1113-B | mG (ps) mA (ps) mG mG mG mC fG fU fA mU mA mU mA mU mG mG mA (ps) mG (ps) mG | 615 | 226 |
| EM1114 | EM1114-A | mU (ps) fG (ps) mC fU mA fU mG fU mC fA mU fU mG fU mC fA mA (ps) fA (ps) mG | 616 | 227 |
| EM1114 | EM1114-B | mC (ps) mU (ps) mU mU mG mA fC fA fA mU mG mA mC mA mU mA mG (ps) mC (ps) mA | 617 | 228 |
| EM1115 | EM1115-A | mA (ps) fA (ps) mA fA mU fC mA fC mC fA mG fG mU fU mU fU mG (ps) fC (ps) mC | 618 | 229 |
| EM1115 | EM1115-B | mG (ps) mG (ps) mC mA mA mA fA fC fC mU mG mG mU mG mA mU mU (ps) mU (ps) mU | 619 | 230 |
| EM1116 | EM1116-A | mC (ps) fU (ps) mC fA mU fA mA fA mG fG mA fU mU fC mA fG mC (ps) fU (ps) mU | 620 | 231 |
| EM1116 | EM1116-B | mA (ps) mA (ps) mG mC mU mG fA fA fU mC mC mU mU mU mA mU mG (ps) mA (ps) mG | 621 | 232 |
| EM1117 | EM1117-A | mU (ps) fU (ps) mC fA mU fA mU fG mC fA mG fC mA fG mU fA mC (ps) fA (ps) mU | 622 | 233 |
| EM1117 | EM1117-B | mA (ps) mU (ps) mG mU mA mC fU fG fC mU mG mC mA mU mA mU mG (ps) mA (ps) mA | 623 | 234 |
| EM1118 | EM1118-A | mU (ps) fU (ps) mG fA mC fG mA fA mG fU mC fG mU fA mC fU mC (ps) fG (ps) mC | 624 | 235 |
| EM1118 | EM1118-B | mG (ps) mC (ps) mG mA mG mU fA fC fG mA mC mU mU mC mG mU mC (ps) mA (ps) mA | 625 | 236 |
| EM1119 | EM1119-A | mU (ps) fA (ps) mG fU mC fG mG fA mG fC mG fG mA fA mG fG mU (ps) fA (ps) mA | 626 | 237 |
| EM1119 | EM1119-B | mU (ps) mU (ps) mA mC mC mU fU fC fC mG mC mU mC mC mG mA mC (ps) mU (ps) mA | 627 | 238 |
| EM1120 | EM1120-A | mU (ps) fG (ps) mA fU mA fG mU fC mC fA mC fA mA fA mC fA mG (ps) fG (ps) mC | 628 | 239 |
| EM1120 | EM1120-B | mG (ps) mC (ps) mC mU mG mU fU fU fG mU mG mG mA mC mU mA mU (ps) mC (ps) mA | 629 | 240 |
| EM1121 | EM1121-A | mG (ps) fU (ps) mC fU mU fC mA fC mA fG mG fC mA fU mU fU mC (ps) fU (ps) mA | 630 | 241 |
| EM1121 | EM1121-B | mU (ps) mA (ps) mG mA mA mA fU fG fC mC mU mG mU mG mA mA mG (ps) mA (ps) mC | 631 | 242 |
| EM1122 | EM1122-A | mU (ps) fG (ps) mA fA mU fC mU fU mG fA mG fA mA fA mG fU mC (ps) fG (ps) mU | 632 | 243 |
| EM1122 | EM1122-B | mA (ps) mC (ps) mG mA mC mU fU fU fC mU mC mA mA mG mA mU mU (ps) mC (ps) mA | 633 | 244 |
| EM1123 | EM1123-A | mU (ps) fC (ps) mG fG mA fG mC fG mG fA mA fG mG fU mA fA mU (ps) fG (ps) mU | 634 | 245 |
| EM1123 | EM1123-B | mA (ps) mC (ps) mA mU mU mA fC fC fU mU mC mC mG mC mU mC mC (ps) mG (ps) mA | 635 | 246 |
| EM1124 | EM1124-A | mC (ps) fA (ps) mC fC mU fG mG fA mG fU mC fU mG fU mU fU mU (ps) fU (ps) mU | 636 | 247 |
| EM1124 | EM1124-B | mA (ps) mA (ps) mA mA mA mA fC fA fG mA mC mU mC mC mA mG mG (ps) mU (ps) mG | 637 | 248 |
| EM1125 | EM1125-A | mU (ps) fU (ps) mA fU mU fC mA fA mU fU mU fA mA fU mC fA mG (ps) fU (ps) mG | 638 | 249 |
| EM1125 | EM1125-B | mC (ps) mA (ps) mC mU mG mA fU fU fA mA mA mU mU mG mA mA mU (ps) mA (ps) mA | 639 | 250 |
| EM1126 | EM1126-A | mA (ps) fA (ps) mA fG mG fA mU fU mC fA mG fC mU fU mC fU mU (ps) fU (ps) mU | 640 | 251 |
| EM1126 | EM1126-B | mA (ps) mA (ps) mA mA mG mA fA fG fC mU mG mA mA mU mC mC mU (ps) mU (ps) mU | 641 | 252 |
| EM1127 | EM1127-A | mG (ps) fU (ps) mA fG mU fC mG fG mA fG mC fG mG fA mA fG mG (ps) fU (ps) mA | 642 | 253 |
| EM1127 | EM1127-B | mU (ps) mA (ps) mC mC mU mU fC fC fG mC mU mC mC mG mA mC mU (ps) mA (ps) mC | 643 | 254 |
| EM1128 | EM1128-A | mA (ps) fG (ps) mU fC mG fG mA fG mC fG mG fA mA fG mG fU mA (ps) fA (ps) mU | 644 | 255 |
| EM1128 | EM1128-B | mA (ps) mU (ps) mU mA mC mC fU fU fC mC mG mC mU mC mC mG mA (ps) mC (ps) mU | 645 | 256 |
| EM1129 | EM1129-A | mC (ps) fA (ps) mU fA mU fA mA fA mA fG mU fG mC fA mC fC mU (ps) fG (ps) mC | 646 | 257 |
| EM1129 | EM1129-B | mG (ps) mC (ps) mA mG mG mU fG fC fA mC mU mU mU mU mA mU mA (ps) mU (ps) mG | 647 | 258 |
| EM1130 | EM1130-A | mC (ps) fA (ps) mU fA mU fA mU fA mC fG mC fC mC fU mC fC mU (ps) fG (ps) mU | 648 | 259 |
| EM1130 | EM1130-B | mA (ps) mC (ps) mA mG mG mA fG fG fG mC mG mU mA mU mA mU mA (ps) mU (ps) mG | 649 | 260 |
| EM1131 | EM1131-A | mU (ps) fC (ps) mC fA mU fA mU fA mU fA mC fG mC fC mC fU mC (ps) fC (ps) mU | 650 | 261 |
| EM1131 | EM1131-B | mA (ps) mG (ps) mG mA mG mG fG fC fG mU mA mU mA mU mA mU mG (ps) mG (ps) mA | 651 | 262 |
| EM1132 | EM1132-A | mU (ps) fA (ps) mU fU mG fA mU fU mA fC mA fA mC fU mU fU mG (ps) fU (ps) mu | 652 | 263 |
| EM1132 | EM1132-B | mA (ps) mA (ps) mC mA mA mA fG fU fU mG mU mA mA mU mC mA mA (ps) mU (ps) mA | 653 | 264 |
| EM1133 | EM1133-A | mU (ps) fA (ps) mC fA mA fC mU fU mU fG mU fU mA fU mU fC mA (ps) fA (ps) mU | 654 | 265 |
| EM1133 | EM1133-B | mA (ps) mU (ps) mU mG mA mA fU fA fA mC mA mA mA mG mU mU mG (ps) mU (ps) mA | 655 | 266 |
| EM1134 | EM1134-A | mU (ps) fU (ps) mG fC mC fU mU fU mU fG mC fC mC fU mC fC mA (ps) fU (ps) mA | 656 | 267 |
| EM1134 | EM1134-B | mU (ps) mA (ps) mU mG mG mA fG fG fG mC mA mA mA mA mG mG mC (ps) mA (ps) mA | 657 | 268 |
| EM1135 | EM1135-A | mU (ps) fA (ps) mA fU mA fU mC fA mG fG mA fC mU fU mG fC mC (ps) fA (ps) mA | 658 | 269 |
| EM1135 | EM1135-B | mU (ps) mU (ps) mG mG mC mA fA fG fU mC mC mU mG mA mU mA mU (ps) mU (ps) mA | 659 | 270 |
| EM1136 | EM1136-A | mU (ps) fC (ps) mG fU mA fG mG fG mA fC mA fC mA fG mG fG mU (ps) fU (ps) mU | 660 | 271 |
| EM1136 | EM1136-B | mA (ps) mA (ps) mA mC mC mC fU fG fU mG mU mC mC mC mU mA mC (ps) mG (ps) mA | 661 | 272 |
| EM1137 | EM1137-A | mU (ps) fC (ps) mG fU mC fA mA fU mG fU mC fC mU fC mG fG mC (ps) fU (ps) mG | 662 | 273 |
| EM1137 | EM1137-B | mC (ps) mA (ps) mG mC mC mG fA fG fG mA mC mA mU mU mG mA mC (ps) mG (ps) mA | 663 | 274 |
| EM1138 | EM1138-A | mC (ps) fC (ps) mC fU mC fC mA fU mA fU mA fU mA fC mG fC mC (ps) fC (ps) mU | 664 | 275 |
| EM1138 | EM1138-B | mA (ps) mG (ps) mG mG mC mG fU fA fU mA mU mA mU mG mG mA mG (ps) mG (ps) mG | 665 | 276 |
| EM1139 | EM1139-A | mU (ps) fC (ps) mC fA mG fA mA fU mG fA mC fA mC fU mG fA mA (ps) fC (ps) mC | 666 | 277 |
| EM1139 | EM1139-B | mG (ps) mG (ps) mU mU mC mA fG fU fG mU mC mA mU mU mC mU mG (ps) mG (ps) mA | 667 | 278 |
| EM1140 | EM1140-A | mA (ps) fA (ps) mA fG mC fC mA fG mC fA mU fC mA fU mG fA mG (ps) fU (ps) mA | 668 | 279 |
| EM1140 | EM1140-B | mU (ps) mA (ps) mC mU mC mA fU fG fA mU mG mC mU mG mG mC mU (ps) mU (ps) mU | 669 | 280 |
| EM1141 | EM1141-A | mA (ps) fU (ps) mA fU mA fC mG fC mC fC mU fC mC fU mG fU mU (ps) fG (ps) mU | 670 | 281 |
| EM1141 | EM1141-B | mA (ps) mC (ps) mA mA mC mA fG fG fA mG mG mG mC mG mU mA mU (ps) mA (ps) mU | 671 | 282 |
| EM1142 | EM1142-A | mU (ps) fA (ps) mU fG mU fC mA fU mU fG mU fC mA fA mA fG mC (ps) fC (ps) mA | 672 | 283 |
| EM1142 | EM1142-B | mU (ps) mG (ps) mG mC mU mU fU fG fA mC mA mA mU mG mA mC mA (ps) mU (ps) mA | 673 | 284 |
| EM1143 | EM1143-A | mA (ps) fU (ps) mA fC mU fC mC fC mC fU mG fG mA fA mA fG mC (ps) fC (ps) mG | 674 | 285 |
| EM1143 | EM1143-B | mC (ps) mG (ps) mG mC mU mU fU fC fC mA mG mG mG mG mA mG mU (ps) mA (ps) mU | 675 | 286 |
| EM1144 | EM1144-A | mA (ps) fU (ps) mC fA mU fG mA fG mU fA mU fA mA fC mC fU mU (ps) fC (ps) mA | 676 | 287 |
| EM1144 | EM1144-B | mU (ps) mG (ps) mA mA mG mG fU fU fA mU mA mC mU mC mA mU mG (ps) mA (ps) mU | 677 | 288 |
| EM1145 | EM1145-A | mU (ps) fG (ps) mG fC mA fG mA fC mA fA mA fU mA fG mG fC mG (ps) fU (ps) mG | 678 | 289 |
| EM1145 | EM1145-B | mC (ps) mA (ps) mC mG mC mC fU fA fU mU mU mG mU mC mU mG mC (ps) mC (ps) mA | 679 | 290 |
| EM1146 | EM1146-A | mU (ps) fC (ps) mU fU mU fC mA fG mG fA mU fG mU fA mU fU mU (ps) fG (ps) mG | 680 | 291 |
| EM1146 | EM1146-B | mC (ps) mC (ps) mA mA mA mU fA fC fA mU mC mC mU mG mA mA mA (ps) mG (ps) mA | 681 | 292 |
| EM1147 | EM1147-A | mU (ps) fU (ps) mG fA mA fU mC fU mU fG mA fG mA fA mA fG mU (ps) fC (ps) mG | 682 | 293 |
| EM1147 | EM1147-B | mC (ps) mG (ps) mA mC mU mU fU fC fU mC mA mA mG mA mU mU mC (ps) mA (ps) mA | 683 | 294 |
| EM1148 | EM1148-A | mC (ps) fU (ps) mU fG mC fC mC fC mC fA mC fU mU fU mC fU mA (ps) fA (ps) mG | 684 | 295 |
| EM1148 | EM1148-B | mC (ps) mU (ps) mU mA mG mA fA fA fG mU mG mG mG mG mG mC mA (ps) mA (ps) mG | 685 | 296 |
| EM1149 | EM1149-A | mA (ps) fG (ps) mU fU mC fC mA fA mU fG mU fC mA fU mC fU mG (ps) fU (ps) mC | 686 | 297 |
| EM1149 | EM1149-B | mG (ps) mA (ps) mC mA mG mA fU fG fA mC mA mU mU mG mG mA mA (ps) mC (ps) mU | 687 | 298 |
| EM1150 | EM1150-A | mC (ps) fU (ps) mU fG mA fG mA fA mA fG mU fC mG fU mA fG mG (ps) fG (ps) mA | 688 | 299 |
| EM1150 | EM1150-B | mU (ps) mC (ps) mC mC mU mA fC fG fA mC mU mU mU mC mU mC mA (ps) mA (ps) mG | 689 | 300 |
| EM1151 | EM1151-A | mU (ps) fU (ps) mU fC mA fU mU fG mU fG mU fA mG fA mA fG mG (ps) fU (ps) mC | 690 | 301 |
| EM1151 | EM1151-B | mG (ps) mA (ps) mC mC mU mu fc fU fA mC mA mC mA mA mU mG mA (ps) mA (ps) mA | 691 | 302 |
| EM1152 | EM1152-A | mG (ps) fC (ps) mC fA mG fU mA fU mG fA mA fA mA fG mA fG mA (ps) fC (ps) mU | 692 | 303 |
| EM1152 | EM1152-B | mA (ps) mG (ps) mU mC mU mC fU fU fU mU mC mA mU mA mC mU mG (ps) mG (ps) mC | 693 | 304 |
| EM1153 | EM1153-A | mA (ps) fG (ps) mC fU mU fG mA fC mG fA mA fG mU fC mG fU mA (ps) fC (ps) mU | 694 | 305 |
| EM1153 | EM1153-B | mA (ps) mG (ps) mU mA mC mG fA fC fU mU mC mG mU mC mA mA mG (ps) mC (ps) mU | 695 | 306 |
| EM1154 | EM1154-A | mC (ps) fA (ps) mG fG mU fG mC fG mC fU mU fG mU fU mA fC mG (ps) fG (ps) mU | 696 | 307 |
| EM1154 | EM1154-B | mA (ps) mC (ps) mC mG mU mA fA fC fA mA mG mC mG mC mA mC mC (ps) mU (ps) mG | 697 | 308 |
| EM1155 | EM1155-A | mG (ps) fA (ps) mC fG mA fA mG fU mC fG mU fA mC fU mC fG mC (ps) fA (ps) mG | 698 | 309 |
| EM1155 | EM1155-B | mC (ps) mU (ps) mG mC mG mA fG fU fA mC mG mA mC mU mU mC mG (ps) mU (ps) mC | 699 | 310 |
| EM1156 | EM1156-A | mA (ps) fG (ps) mG fU mA fA mU fG mU fC mC fA mG fG mC fU mG (ps) fG (ps) mA | 700 | 311 |
| EM1156 | EM1156-B | mU (ps) mC (ps) mC mA mG mC fC fU fG mG mA mC mA mU mU mA mC (ps) mC (ps) mU | 701 | 312 |
| EM1157 | EM1157-A | mU (ps) fG (ps) mA fC mG fA mA fG mU fC mG fU mA fC mU fC mG (ps) fC (ps) mA | 702 | 313 |
| EM1157 | EM1157-B | mU (ps) mG (ps) mC mG mA mG fU fA fC mG mA mC mU mU mC mG mU (ps) mC (ps) mA | 703 | 314 |
| EM1158 | EM1158-A | mC (ps) fU (ps) mG fU mG fC mU fC mG fU mG fU mA fG mU fG mG (ps) fA (ps) mU | 704 | 315 |
| EM1158 | EM1158-B | mA (ps) mU (ps) mC mC mA mC fU fA fC mA mC mG mA mG mC mA mC (ps) mA (ps) mG | 705 | 316 |
| EM1159 | EM1159-A | mU (ps) fC (ps) mA fU mA fA mA fG mG fA mU fU mC fA mG fC mU (ps) fU (ps) mC | 706 | 317 |
| EM1159 | EM1159-B | mG (ps) mA (ps) mA mG mC mU fG fA fA mU mC mC mU mU mU mA mU (ps) mG (ps) mA | 707 | 318 |
| EM1160 | EM1160-A | mA (ps) fC (ps) mA fU mA fC mA fU mU fA mG fA mU fU mU fC mU (ps) fA (ps) mG | 708 | 319 |
| EM1160 | EM1160-B | mC (ps) mU (ps) mA mG mA mA fA fU fC mU mA mA mU mG mU mA mU (ps) mG (ps) mU | 709 | 320 |
| EM1161 | EM1161-A | mC (ps) fA (ps) mU fA mC fA mU fU mA fG mA fU mU fU mC fU mA (ps) fG (ps) mC | 710 | 321 |
| EM1161 | EM1161-B | mG (ps) mC (ps) mU mA mG mA fA fA fU mC mU mA mA mU mG mU mA (ps) mU (ps) mG | 711 | 322 |
| EM1162 | EM1162-A | mA (ps) fG (ps) mC fA mU fC mA fU mG fA mG fU mA fU mA fA mC (ps) fC (ps) mU | 712 | 323 |
| EM1162 | EM1162-B | mA (ps) mG (ps) mG mU mU mA fU fA fC mU mC mA mU mG mA mU mG (ps) mC (ps) mU | 713 | 324 |
| EM1163 | EM1163-A | mC (ps) fU (ps) mU fC mA fU mG fU mA fU mA fA mA fA mA fC mA (ps) fG (ps) mC | 714 | 325 |
| EM1163 | EM1163-B | mG (ps) mC (ps) mU mG mU mU fU fU fU mA mU mA mC mA mU mG mA (ps) mA (ps) mG | 715 | 326 |
| EM1164 | EM1164-A | mA (ps) fG (ps) mA fC mA fA mA fU mA fG mG fC mG fU mG fA mU (ps) fG (ps) mU | 716 | 327 |
| EM1164 | EM1164-B | mA (ps) mC (ps) mA mU mC mA fC fG fC mC mU mA mU mU mU mG mU (ps) mC (ps) mU | 717 | 328 |
| EM1165 | EM1165-A | mA (ps) fC (ps) mA fA mA fU mA fG mG fC mG fU mG fA mU fG mU (ps) fU (ps) mG | 718 | 329 |
| EM1165 | EM1165-B | mC (ps) mA (ps) mA mC mA mU fC fA fC mG mC mC mU mA mU mU mU (ps) mG (ps) mU | 719 | 330 |
| EM1166 | EM1166-A | mA (ps) fU (ps) mU fG mA fU mU fA mC fA mA fC mU fU mU fG mU (ps) fU (ps) mA | 720 | 331 |
| EM1166 | EM1166-B | mU (ps) mA (ps) mA mC mA mA fA fG fU mU mG mU mA mA mU mC mA (ps) mA (ps) mU | 721 | 332 |
| EM1167 | EM1167-A | mU (ps) fG (ps) mU fC mA fU mC fU mG fU mC fC mU fC mA fU mA (ps) fA (ps) mA | 722 | 333 |
| EM1167 | EM1167-B | mU (ps) mU (ps) mU mA mU mG fA fG fG mA mC mA mG mA mU mG mA (ps) mC (ps) mA | 723 | 334 |
| EM1168 | EM1168-A | mA (ps) fU (ps) mG fA mG fU mA fU mA fA mC fC mU fU mC fA mU (ps) fG (ps) mU | 724 | 335 |
| EM1168 | EM1168-B | mA (ps) mC (ps) mA mU mG mA fA fG fG mU mU mA mU mA mC mU mC (ps) mA (ps) mU | 725 | 336 |
| EM1169 | EM1169-A | mC (ps) fU (ps) mC fG mU fC mA fA mU fG mU fC mC fU mC fG mG (ps) fC (ps) mU | 726 | 337 |
| EM1169 | EM1169-B | mA (ps) mG (ps) mC mC mG mA fG fG fA mC mA mU mU mG mA mC mG (ps) mA (ps) mG | 727 | 338 |
| EM1170 | EM1170-A | mC (ps) fU (ps) mU fG mA fC mG fA mA fG mU fC mG fU mA fC mU (ps) fC (ps) mG | 728 | 339 |
| EM1170 | EM1170-B | mC (ps) mG (ps) mA mG mU mA fC fG fA mC mU mU mC mG mU mC mA (ps) mA (ps) mG | 729 | 340 |
| EM1171 | EM1171-A | mU (ps) fU (ps) mA fA mU fC mC fC mC fA mU fC mC fA mG fA mU (ps) fG (ps) mC | 730 | 341 |
| EM1171 | EM1171-B | mG (ps) mC (ps) mA mU mC mU fG fG fA mU mG mG mG mG mA mU mU (ps) mA (ps) mA | 731 | 342 |
| EM1172 | EM1172-A | mG (ps) fU (ps) mA fC mU fC mG fC mA fG mA fG mG fU mG fG mG (ps) fA (ps) mG | 732 | 343 |
| EM1172 | EM1172-B | mC (ps) mU (ps) mC mC mC mA fC fC fU mC mU mG mC mG mA mG mU (ps) mA (ps) mC | 733 | 344 |
| EM1173 | EM1173-A | mA (ps) fU (ps) mA fG mA fA mG fG mC fC mU fC mG fA mA fC mC (ps) fC (ps) mC | 734 | 345 |
| EM1173 | EM1173-B | mG (ps) mG (ps) mG mG mU mU fC fG fA mG mG mC mC mU mU mC mU (ps) mA (ps) mU | 735 | 346 |
| EM1174 | EM1174-A | mA (ps) fU (ps) mU fU mA fA mU fC mA fG mU fG mC fU mA fU mG (ps) fU (ps) mC | 736 | 347 |
| EM1174 | EM1174-B | mG (ps) mA (ps) mC mA mU mA fG fC fA mC mU mG mA mU mU mA mA (ps) mA (ps) mU | 737 | 348 |
| EM1175 | EM1175-A | mA (ps) fU (ps) mG fU mC fA mU fC mU fG mU fC mC fU mC fA mU (ps) fA (ps) mA | 738 | 349 |
| EM1175 | EM1175-B | mU (ps) mU (ps) mA mU mG mA fG fG fA mC mA mG mA mU mG mA mC (ps) mA (ps) mU | 739 | 350 |
| EM1176 | EM1176-A | mA (ps) fG (ps) mU fA mU fG mA fA mA fA mG fA mG fA mC fU mG (ps) fG (ps) mC | 740 | 351 |
| EM1176 | EM1176-B | mG (ps) mC (ps) mC mA mG mU fC fU fC mU mU mU mU mC mA mU mA (ps) mC (ps) mU | 741 | 352 |
| EM1177 | EM1177-A | mG (ps) fU (ps) mU fA mG fC mA fG mU fU mA fC mA fC mU fU mC (ps) fC (ps) mC | 742 | 353 |
| EM1177 | EM1177-B | mG (ps) mG (ps) mG mA mA mG fU fG fU mA mA mC mU mG mC mU mA (ps) mA (ps) mC | 743 | 354 |
| EM1178 | EM1178-A | mA (ps) fG (ps) mC fC mA fG mU fA mU fG mA fA mA fA mG fA mG (ps) fA (ps) mC | 744 | 355 |
| EM1178 | EM1178-B | mG (ps) mU (ps) mC mU mC mU fU fU fU mC mA mU mA mC mU mG mG (ps) mC (ps) mU | 745 | 356 |
| EM1179 | EM1179-A | mG (ps) fA (ps) mU fG mU fA mU fU mU fG mG fC mU fU mG fC mA (ps) fC (ps) mA | 746 | 357 |
| EM1179 | EM1179-B | mU (ps) mG (ps) mU mG mC mA fA fG fC mC mA mA mA mU mA mC mA (ps) mU (ps) mC | 747 | 358 |
| EM1180 | EM1180-A | mA (ps) fA (ps) mU fG mC fC mA fA mC fA mG fC mC fA mG fU mA (ps) fU (ps) mG | 748 | 359 |
| EM1180 | EM1180-B | mC (ps) mA (ps) mU mA mC mU fG fG fC mU mG mU mU mG mG mC mA (ps) mU (ps) mU | 749 | 360 |
| EM1181 | EM1181-A | mA (ps) fG (ps) mC fA mU fG mU fU mA fG mC fA mG fU mU fA mC (ps) fA (ps) mC | 750 | 361 |
| EM1181 | EM1181-B | mG (ps) mU (ps) mG mU mA mA fC fU fG mC mU mA mA mC mA mU mG (ps) mC (ps) mU | 751 | 362 |
| EM1182 | EM1182-A | mU (ps) fG (ps) mU fC mA fU mA fU mA fA mA fA mG fU mG fC mA (ps) fC (ps) mC | 752 | 363 |
| EM1182 | EM1182-B | mG (ps) mG (ps) mU mG mC mA fC fU fU mU mU mA mU mA mU mG mA (ps) mC (ps) mA | 753 | 364 |
| EM1183 | EM1183-A | mG (ps) fC (ps) mC fA mA fC mA fG mC fC mA fG mU fA mU fG mA (ps) fA (ps) mA | 754 | 365 |
| EM1183 | EM1183-B | mU (ps) mU (ps) mU mC mA mU fA fC fU mG mG mC mU mG mU mU mG (ps) mG (ps) mC | 755 | 366 |
| EM1184 | EM1184-A | mA (ps) fC (ps) mA fA mA fG mU fC mC fA mG fA mA fU mG fA mC (ps) fA (ps) mC | 756 | 367 |
| EM1184 | EM1184-B | mG (ps) mU (ps) mG mU mC mA fU fU fC mU mG mG mA mC mU mU mU (ps) mG (ps) mU | 757 | 368 |
| EM1185 | EM1185-A | mA (ps) fG (ps) mG fU mU fC mA fG mG fC mC fA mC fU mU fC mG (ps) fG (ps) mG | 758 | 369 |
| EM1185 | EM1185-B | mC (ps) mC (ps) mC mG mA mA fG fU fG mG mC mC mU mG mA mA mC (ps) mC (ps) mU | 759 | 370 |
| EM1186 | EM1186-A | mC (ps) fU (ps) mA fA mU fA mU fC mA fG mG fA mC fU mU fG mC (ps) fC (ps) mA | 760 | 371 |
| EM1186 | EM1186-B | mU (ps) mG (ps) mG mC mA mA fG fU fC mC mU mG mA mU mA mU mU (ps) mA (ps) mG | 761 | 372 |
| EM1187 | EM1187-A | mU (ps) fC (ps) mU fG mU fG mA fC mA fA mA fG mG fU mG fA mU (ps) fG (ps) mG | 762 | 373 |
| EM1187 | EM1187-B | mC (ps) mC (ps) mA mU mC mA fC fC fU mU mU mG mU mC mA mC mA (ps) mG (ps) mA | 763 | 374 |
| EM1188 | EM1188-A | mU (ps) fU (ps) mU fC mA fU mA fU mG fC mA fG mC fA mG fU mA (ps) fC (ps) mA | 764 | 375 |
| EM1188 | EM1188-B | mU (ps) mG (ps) mU mA mC mU fG fC fU mG mC mA mU mA mU mG mA (ps) mA (ps) mA | 765 | 376 |
| EM1189 | EM1189-A | mA (ps) fA (ps) mG fA mC fC mU fG mG fC mC fG mG fA mG fC mA (ps) fC (ps) mA | 766 | 377 |
| EM1189 | EM1189-B | mU (ps) mG (ps) mU mG mC mU fC fC fG mG mC mC mA mG mG mU mC (ps) mU (ps) mU | 767 | 378 |
| EM1190 | EM1190-A | mU (ps) fC (ps) mA fU mG fG mU fG mU fG mC fC mC fG mU fC mC (ps) fA (ps) mG | 768 | 379 |
| EM1190 | EM1190-B | mC (ps) mU (ps) mG mG mA mC fG fG fG mC mA mC mA mC mC mA mU (ps) mG (ps) mA | 769 | 380 |
| EM1191 | EM1191-A | mG (ps) fA (ps) mA fG mG fU mA fA mU fG mU fC mC fA mG fG mC (ps) fU (ps) mG | 770 | 381 |
| EM1191 | EM1191-B | mC (ps) mA (ps) mG mC mC mU fG fG fA mC mA mU mU mA mC mC mU (ps) mU (ps) mC | 771 | 382 |
| EM1192 | EM1192-A | mA (ps) fA (ps) mG fU mC fG mU fA mC fU mC fG mC fA mG fA mG (ps) fG (ps) mU | 772 | 383 |
| EM1192 | EM1192-B | mA (ps) mC (ps) mC mU mC mU fG fC fG mA mG mU mA mC mG mA mC (ps) mU (ps) mU | 773 | 384 |
| EM1214 | EM1214-A | (vp)-mU fU mG fG mG fU mG fA mA fG mU fA mG fA mG fG mC (ps) fG (ps) mC | 774 | 385 |
| EM1214 | EM1214-B | mG mC mG mC mC mU fC fU fA mC mU mU mC mA mC mC mC (ps) mA (ps) mC | 775 | 178 |
| EM1215 | EM1215-A | (vp)-mU fA mC fG mA fA mG fU mC fG mU fA mC fU mC fG mC (ps) fA (ps) mG | 776 | 386 |
| EM1215 | EM1215-B | mC mU mG mC mG mA fG fU fA mC mG mA mC mU mU mC mG (ps) mU (ps) mC | 777 | 310 |
| EM1216 | EM1216-A | (vp)-mU fU mA fG mA fA mG fG mC fC mU fC mG fA mA fC mC (ps) fC (ps) mC | 778 | 387 |
| EM1216 | EM1216-B | mG mG mG mG mU mU fC fG fA mG mG mC mC mU mU mC mU (ps) mA (ps) mU | 779 | 346 |
| EM1217 | EM1217-A | (vp)-mU fA mA fG mG fU mA fA mU fG mU fC mC fA mG fG mC (ps) fU (ps) mG | 780 | 388 |
| EM1217 | EM1217-B | mC mA mG mC mC mU fG fG fA mC mA mU mU mA mC mC mU (ps) mU (ps) mC | 781 | 382 |
| EM1218 | EM1218-A | (vp)-mU fA mG fU mC fG mU fA mC fU mC fG mC fA mG fA mG (ps) fG (ps) mU | 782 | 389 |
| EM1218 | EM1218-B | mA mC mC mU mC mU fG fC fG mA mG mU mA mC mG mA mC (ps) mU (ps) mU | 783 | 384 |
| EM1219 | EM1219-A | (vp)-mU fC mU fC mG fU mU fG mG fA mG fU mA fG mU fC mG (ps) fG (ps) mA | 794 | 147 |
| EM1219 | EM1219-B | mU mC mC mG mA mC fU fA fC mU mC mC mA mA mC mG mA (ps) mG (ps) mA | 816 | 148 |
| EM1220 | EM1220-A | (vp)-mU fU mG fG mA fG mU fA mG fU mC fG mG fA mG fC mG (ps) fG (ps) mA | 798 | 199 |
| EM1220 | EM1220-B | mU mC mC mG mC mU fC fC fG mA mC mU mA mC mU mC mC (ps) mA (ps) mA | 817 | 200 |
| EM1221 | EM1221-A | (vp)-mU fU mG fA mC fG mA fA mG fU mC fG mU fA mC fU mC (ps) fG (ps) mC | 800 | 235 |
| EM1221 | EM1221-B | mG mC mG mA mG mU fA fC fG mA mC mU mU mC mG mU mC (ps) mA (ps) mA | 818 | 236 |
| EM1222 | EM1222-A | (vp)-mU fA mG fU mC fG mG fA mG fC mG fG mA fA mG fG mU (ps) fA (ps) mA | 802 | 237 |
| EM1222 | EM1222-B | mU mU mA mC mC mU fU fC fC mG mC mU mC mC mG mA mC (ps) mU (ps) mA | 819 | 238 |
| EM1223 | EM1223-A | (vp)-mU fC mG fG mA fG mC fG mG fA mA fG mG fU mA fA mU (ps) fG (ps) mU | 804 | 245 |
| EM1223 | EM1223-B | mA mC mA mU mU mA fC fC fU mU mC mC mG mC mU mC mC (ps) mG (ps) mA | 820 | 246 |

TABLE 5c
Modified GalNAc-conjugated duplexes

| | | | | Unmodified |
|---|---|---|---|---|
| Duplex | Strand | | | equivalent |
| ID | Name (*) | Sequence (5′→3′) | SEQ ID No. | SEQ ID No. |
| EM1193 | EM1074-A | mU (ps) fC (ps) mU fC mG fU mU fG mG fA mG fU mA fG mU fC mG (ps) fG (ps) mA | 536 | 147 |
| EM1193 | EM1193-B | [ST23(ps)]3 ST41 (ps) mU mC mC mG mA mC fU fA fC mU mC mC mA mA mC mG | 784 | 148 |
| | | mA (ps) mG (ps) mA | | |
| EM1194 | EM1089-A | mG (ps) fU (ps) mG fG mG fU mG fA mA fG mU fA mG fA mG fG mC (ps) fG (ps) mC | 566 | 177 |
| EM1194 | EM1194-B | [ST23(ps)]3 ST41 (ps) mG mC mG mC mC mU fC fU fA mC mU mU mC mA mC mC | 785 | 178 |
| | | mC (ps) mA (ps) mC | | |
| EM1195 | EM1100-A | mU (ps) fU (ps) mG fG mA fG mU fA mG fU mC fG mG fA mG fC mG (ps) fG (ps) mA | 588 | 199 |
| EM1195 | EM1195-B | [ST23(ps)]3 ST41 (ps) mU mC mC mG mC mU fC fC fG mA mC mU mA mC mU mC | 786 | 200 |
| | | mC (ps) mA (ps) mA | | |
| EM1196 | EM1118-A | mU (ps) fU (ps) mG fA mC fG mA fA mG fU mC fG mU fA mC fU mC (ps) fG (ps) mC | 624 | 235 |
| EM1196 | EM1196-B | [ST23(ps)]3 ST41 (ps) mG mC mG mA mG mU fA fC fG mA mC mU mU mC mG mU | 787 | 236 |
| | | mC (ps) mA (ps) mA | | |
| EM1197 | EM1119-A | mU (ps) fA (ps) mG fU mC fG mG fA mG fC mG fG mA fA mG fG mU (ps) fA (ps) mA | 626 | 237 |
| EM1197 | EM1197-B | [ST23(ps)]3 ST41 (ps) mU mU mA mC mC mU fU fC fC mG mC mU mC mC mG mA | 788 | 238 |
| | | mC (ps) mU (ps) mA | | |
| EM1198 | EM1123-A | mU (ps) fC (ps) mG fG mA fG mC fG mG fA mA fG mG fU mA fA mU (ps) fG (ps) mU | 634 | 245 |
| EM1198 | EM1198-B | [ST23(ps)]3 ST41 (ps) mA mC mA mU mU mA fC fC fU mU mC mC mG mC mU mC | 789 | 246 |
| | | mC (ps) mG (ps) mA | | |
| EM1199 | EM1155-A | mG (ps) fA (ps) mC fG mA fA mG fU mC fG mU fA mC fU mC fG mC (ps) fA (ps) mG | 698 | 309 |
| EM1199 | EM1199-B | [ST23(ps)]3 ST41 (ps) mC mU mG mC mG mA fG fU fA mC mG mA mC mU mU mC | 790 | 310 |
| | | mG (ps) mU (ps) mC | | |
| EM1200 | EM1173-A | mA (ps) fU (ps) mA fG mA fA mG fG mC fC mU fC mG fA mA fC mC (ps) fC (ps) mC | 734 | 345 |
| EM1200 | EM1200-B | [ST23(ps)]3 ST41 (ps) mG mG mG mG mU mU fC fG fA mG mG mC mC mU mU mC | 791 | 346 |
| | | mU (ps) mA (ps) mU | | |
| EM1201 | EM1191-A | mG (ps) fA (ps) mA fG mG fU mA fA mU fG mU fC mC fA mG fG mC (ps) fU (ps) mG | 770 | 381 |
| EM1201 | EM1201-B | [ST23(ps)]3 ST41 (ps) mC mA mG mC mC mU fG fG fA mC mA mU mU mA mC mC | 792 | 382 |
| | | mU (ps) mU (ps) mC | | |
| EM1202 | EM1192-A | mA (ps) fA (ps) mG fU mC fG mU fA mC fU mC fG mC fA mG fA mG (ps) fG (ps) mU | 772 | 383 |
| EM1202 | EM1202-B | [ST23(ps)]3 ST41 (ps) mA mC mC mU mC mU fG fC fG mA mG mU mA mC mG mA | 793 | 384 |
| | | mC (ps) mU (ps) mU | | |
| EM1203 | EM1203-A | (vp)-mU fC mU fC mG fU mU fG mG fA mG fU mA fG mU fC mG (ps) fG (ps) mA | 794 | 147 |
| EM1203 | EM1193-B | [ST23(ps)]3 ST41 (ps) mU mC mC mG mA mC fU fA fC mU mC mC mA mA mC mG | 795 | 148 |
| | | mA (ps) mG (ps) mA | | |
| EM1204 | EM1204-A | (vp)-mU fU mG fG mG fU mG fA mA fG mU fA mG fA mG fG mC (ps) fG (ps) mC | 796 | 385 |
| EM1204 | EM1194-B | [ST23(ps)]3 ST41 (ps) mG mC mG mC mC mU fC fU fA mC mU mU mC mA mC mC | 797 | 178 |
| | | mC (ps) mA (ps) mC | | |
| EM1205 | EM1205-A | (vp)-mU fU mG fG mA fG mU fA mG fU mC fG mG fA mG fC mG (ps) fG (ps) mA | 798 | 199 |
| EM1205 | EM1195-B | [ST23(ps)]3 ST41 (ps) mU mC mC mG mC mU fC fC fG mA mC mU mA mC mU mC | 799 | 200 |
| | | mC (ps) mA (ps) mA | | |
| EM1206 | EM1206-A | (vp)-mU fU mG fA mC fG mA fA mG fU mC fG mU fA mC fU mC (ps) fG (ps) mC | 800 | 235 |
| EM1206 | EM1196-B | [ST23(ps)]3 ST41 (ps) mG mC mG mA mG mU fA fC fG mA mC mU mU mC mG mU | 801 | 236 |
| | | mC (ps) mA (ps) mA | | |
| EM1207 | EM1207-A | (vp)-mU fA mG fU mC fG mG fA mG fC mG fG mA fA mG fG mU (ps) fA (ps) mA | 802 | 237 |
| EM1207 | EM1197-B | [ST23(ps)]3 ST41 (ps) mU mU mA mC mC mU fU fC fC mG mC mU mC mC mG mA | 803 | 238 |
| | | mC (ps) mU (ps) mA | | |
| EM1208 | EM1208-A | (vp)-mU fC mG fG mA fG mC fG mG fA mA fG mG fU mA fA mU (ps) fG (ps) mU | 804 | 245 |
| EM1208 | EM1198-B | [ST23(ps)]3 ST41 (ps) mA mC mA mU mU mA fC fC fU mU mC mC mG mC mU mC | 805 | 246 |
| | | mC (ps) mG (ps) mA | | |
| EM1209 | EM1209-A | (vp)-mU fA mC fG mA fA mG fU mC fG mU fA mC fU mC fG mC (ps) fA (ps) mG | 806 | 386 |
| EM1209 | EM1199-B | [ST23(ps)]3 ST41 (ps) mC mU mG mC mG mA fG fU fA mC mG mA mC mU mU mC | 807 | 310 |
| | | mG (ps) mU (ps) mC | | |
| EM1210 | EM1210-A | (vp)-mU fU mA fG mA fA mG fG mC fC mU fC mG fA mA fC mC (ps) fC (ps) mC | 808 | 387 |
| EM1210 | EM1200-B | [ST23(ps)]3 ST41 (ps) mG mG mG mG mU mU fC fG fA mG mG mC mC mU mU mC | 809 | 346 |
| | | mU (ps) mA (ps) mU | | |
| EM1211 | EM1211-A | (vp)-mU fA mA fG mG fU mA fA mU fG mU fC mC fA mG fG mC (ps) fU (ps) mG | 810 | 388 |
| EM1211 | EM1201-B | [ST23(ps)]3 ST41 (ps) mC mA mG mC mC mU fG fG fA mC mA mU mU mA mC mC | 811 | 382 |
| | | mU (ps) mU (ps) mC | | |
| EM1212 | EM1212-A | (vp)-mU fA mG fU mC fG mU fA mC fU mC fG mC fA mG fA mG (ps) fG (ps) mU | 812 | 389 |
| EM1212 | EM1202-B | [ST23(ps)]3 ST41 (ps) mA mC mC mU mC mU fG fC fG mA mG mU mA mC mG mA | 813 | 384 |
| | | mC (ps) mU (ps) mU | | |
| EM1213 | EM1213-A | (vp)-mU (ps) fG (ps) mG fC mA fG mA fC mA fA mA fU mA fG mG fC mG (ps) fU (ps) | 814 | 289 |
| | | mG | | |
| EM1213 | EM1204-B | [ST23(ps)]3 ST41 (ps) mC mA mC mG mC mC fU fA fU mU mU mG mU mC mU mG | 815 | 290 |
| | | mC (ps) mC (ps) mA | | |

>NM_006610.4 Homo sapiens MBL associated serine protease 2 (MASP-2),
transcript variant 1, mRNA
SEQ ID No. 821
AGACCAGGCCAGGCCAGCTGGACGGGCACACCATGAGGCTGCTGACCCTCCTGGGCCTTCTGTGTGGCTC
GGTGGCCACCCCCTTGGGCCCGAAGTGGCCTGAACCTGTGTTCGGGCGCCTGGCATCCCCCGGCTTTCCA
GGGGAGTATGCCAATGACCAGGAGCGGCGCTGGACCCTGACTGCACCCCCCGGCTACCGCCTGCGCCTCT
ACTTCACCCACTTCGACCTGGAGCTCTCCCACCTCTGCGAGTACGACTTCGTCAAGCTGAGCTCGGGGGC
CAAGGTGCTGGCCACGCTGTGCGGGCAGGAGAGCACAGACACGGAGCGGGCCCCTGGCAAGGACACTTTC
TACTCGCTGGGCTCCAGCCTGGACATTACCTTCCGCTCCGACTACTCCAACGAGAAGCCGTTCACGGGGT
TCGAGGCCTTCTATGCAGCCGAGGACATTGACGAGTGCCAGGTGGCCCCGGGAGAGGCGCCCACCTGCGA
CCACCACTGCCACAACCACCTGGGCGGTTTCTACTGCTCCTGCCGCGCAGGCTACGTCCTGCACCGTAAC
AAGCGCACCTGCTCAGCCCTGTGCTCCGGCCAGGTCTTCACCCAGAGGTCTGGGGAGCTCAGCAGCCCTG
AATACCCACGGCCGTATCCCAAACTCTCCAGTTGCACTTACAGCATCAGCCTGGAGGAGGGGTTCAGTGT
CATTCTGGACTTTGTGGAGTCCTTCGATGTGGAGACACACCCTGAAACCCTGTGTCCCTACGACTTTCTC
AAGATTCAAACAGACAGAGAAGAACATGGCCCATTCTGTGGGAAGACATTGCCCCACAGGATTGAAACAA
AAAGCAACACGGTGACCATCACCTTTGTCACAGATGAATCAGGAGACCACACAGGCTGGAAGATCCACTA
CACGAGCACAGCGCAGCCTTGCCCTTATCCGATGGCGCCACCTAATGGCCACGTTTCACCTGTGCAAGCC
AAATACATCCTGAAAGACAGCTTCTCCATCTTTTGCGAGACTGGCTATGAGCTTCTGCAAGGTCACTTGC
CCCTGAAATCCTTTACTGCAGTTTGTCAGAAAGATGGATCTTGGGACCGGCCAATGCCCGCGTGCAGCAT
TGTTGACTGTGGCCCTCCTGATGATCTACCCAGTGGCCGAGTGGAGTACATCACAGGTCCTGGAGTGACC
ACCTACAAAGCTGTGATTCAGTACAGCTGTGAAGAGACCTTCTACACAATGAAAGTGAATGATGGTAAAT
ATGTGTGTGAGGCTGATGGATTCTGGACGAGCTCCAAAGGAGAAAAATCACTCCCAGTCTGTGAGCCTGT
TTGTGGACTATCAGCCCGCACAACAGGAGGGCGTATATATGGAGGGCAAAAGGCAAAACCTGGTGATTTT
CCTTGGCAAGTCCTGATATTAGGTGGAACCACAGCAGCAGGTGCACTTTTATATGACAACTGGGTCCTAA
CAGCTGCTCATGCCGTCTATGAGCAAAAACATGATGCATCCGCCCTGGACATTCGAATGGGCACCCTGAA
AAGACTATCACCTCATTATACACAAGCCTGGTCTGAAGCTGTTTTTATACATGAAGGTTATACTCATGAT
GCTGGCTTTGACAATGACATAGCACTGATTAAATTGAATAACAAAGTTGTAATCAATAGCAACATCACGC
CTATTTGTCTGCCAAGAAAAGAAGCTGAATCCTTTATGAGGACAGATGACATTGGAACTGCATCTGGATG
GGGATTAACCCAAAGGGGTTTTCTTGCTAGAAATCTAATGTATGTCGACATACCGATTGTTGACCATCAA
AAATGTACTGCTGCATATGAAAAGCCACCCTATCCAAGGGGAAGTGTAACTGCTAACATGCTTTGTGCTG
GCTTAGAAAGTGGGGGCAAGGACAGCTGCAGAGGTGACAGCGGAGGGGCACTGGTGTTTCTAGATAGTGA
AACAGAGAGGTGGTTTGTGGGAGGAATAGTGTCCTGGGGTTCCATGAATTGTGGGGAAGCAGGTCAGTAT
GGAGTCTACACAAAAGTTATTAACTATATTCCCTGGATCGAGAACATAATTAGTGATTTTTAACTTGCGT
GTCTGCAGTCAAGGATTCTTCATTTTTAGAAATGCCTGTGAAGACCTTGGCAGCGACGTGGCTCGAGAAG
CATTCATCATTACTGTGGACATGGCAGTTGTTGCTCCACCCAAAAAAACAGACTCCAGGTGAGGCTGCTG
TCATTTCTCCACTTGCCAGTTTAATTCCAGCCTTACCCATTGACTCAAGGGGACATAAACCACGAGAGTG
ACAGTCATCTTTGCCCACCCAGTGTAATGTCACTGCTCAAATTACATTTCATTACCTTAAAAAGCCAGTC
TCTTTTCATACTGGCTGTTGGCATTTCTGTAAACTGCCTGTCCATGCTCTTTGTTTTTAAACTTGTTCTT
ATTGA

### Statements

The following statements represent aspects of the invention.
1. A double-stranded nucleic acid for inhibiting expression of MASP-2, wherein the nucleic acid comprises a first strand and a second strand, wherein the unmodified equivalent of the first strand sequence comprises a sequence of at least 15 nucleotides differing by no more than 3 nucleotides from any one of the first strand sequences shown in Table 5a, or of Table 1.
2. The nucleic acid of statement 1, wherein the first strand and the second strand are separate strands and are each 18-25 nucleotides in length.
3. The nucleic acid of statement 1 or statement 2, wherein the first strand and the second strand form a duplex region of from 17-25 nucleotides in length.
4. The nucleic acid of any one of the preceding statements, wherein the duplex region consists of 17-25 consecutive nucleotide base pairs.
5. The nucleic acid of any one of the preceding statements, wherein said nucleic acid:
   a) is blunt ended at both ends;
   b) has an overhang at one end and a blunt end at the other end; or
   c) has an overhang at both ends.
6. The nucleic acid of any one of the preceding statements, wherein the nucleic acid is a siRNA.
7. The nucleic acid of any one of the preceding statements, wherein the nucleic acid mediates RNA interference.
8. The nucleic acid of any one of the preceding statements, wherein:
(a) the unmodified equivalent of the first strand sequence comprises a sequence differing by no more than 3 nucleotides from any one of the first strand sequences of Table 5a, and optionally wherein the unmodified equivalent of the second strand sequence comprises a sequence differing by no more than 3 nucleotides from the corresponding second strand sequence of Table 5a;
(b) the unmodified equivalent of the first strand sequence comprises a sequence differing by no more than 2 nucleotides from any one of the first strand sequences of Table 5a, and optionally wherein the unmodified equivalent of the second strand sequence comprises a sequence differing by no more than 2 nucleotides from the corresponding second strand sequence of Table 5a;
(c) the unmodified equivalent of the first strand sequence comprises a sequence differing by no more than 1 nucleotide from any one of the first strand sequences of Table 5a, and optionally wherein the unmodified equivalent of the second strand sequence comprises a sequence differing by no more than 1 nucleotide from the corresponding second strand sequence of Table 5a;
(d) the unmodified equivalent of the first strand sequence comprises a sequence corresponding to nucleotides 2 to 17 from the 5′ end of any one of the first strand sequences with a given SEQ ID No. shown in Table 5a, and optionally wherein the unmodified equivalent of the second strand sequence comprises a sequence corresponding to nucleotides 2 to 17 from the 5′ end of the corresponding second strand sequence with a given SEQ ID No. shown in Table 5a;
(e) the unmodified equivalent of the first strand sequence comprises a sequence corresponding to nucleotides 2 to 18 from the 5′ end of any one of the first strand sequences with a given SEQ ID No. shown in Table 5a, and optionally wherein the unmodified equivalent of the second strand sequence comprises a sequence corresponding to nucleotides 2 to 18 from the 5′ end of the corresponding second strand sequence with a given SEQ ID No. shown in Table 5a;
(f) the unmodified equivalent of the first strand sequence comprises a sequence corresponding to nucleotides 2 to 19 from the 5′ end of any one of the first strand sequences with a given SEQ ID No. shown in Table 5a, and optionally wherein the unmodified equivalent of the second strand sequence comprises a sequence corresponding to nucleotides 2 to 19 from the 5′ end of the corresponding second strand sequence with a given SEQ ID No. shown in Table 5a;
(g) the unmodified equivalent of the first strand sequence comprises a sequence corresponding to nucleotides 2 to 19 from the 5′ end of any one of the first strand sequences with a given SEQ ID No. shown in Table 5a, and optionally wherein the unmodified equivalent of the second strand sequence comprises a sequence corresponding to nucleotides 1 to 18 from the 5′ end of the corresponding second strand sequence with a given SEQ ID No. shown in Table 5a;
(h) the unmodified equivalent of the first strand sequence comprises a sequence of any one of the first strand sequences with a given SEQ ID No. shown in Table 5a, and optionally wherein the unmodified equivalent of the second strand sequence comprises a sequence of the corresponding second strand sequence with a given SEQ ID No. shown in Table 5a; or
(i) the unmodified equivalent of the first strand sequence essentially consists of any one of the first strand sequences with a given SEQ ID No. shown in Table 5a, and optionally wherein the unmodified equivalent of the second strand sequence essentially consists of the sequence of the corresponding second strand sequence with a given SEQ ID No. shown in Table 5a.
(j) the unmodified equivalent of the first strand sequence consists of any one of the first strand sequences with a given SEQ ID No. shown in Table 5a, and optionally wherein the unmodified equivalent of the second strand sequence consists of the sequence of the corresponding second strand sequence with a given SEQ ID No. shown in Table 5a.
9. The nucleic acid of any one of the preceding statements, wherein:
(a) the unmodified equivalent of the first strand sequence comprises a sequence differing by no more than 3 nucleotides from any one of the first strand sequences of Table 1, and optionally wherein the unmodified equivalent of the second strand sequence comprises a sequence differing by no more than 3 nucleotides from the corresponding second strand sequence of Table 1;
(b) the unmodified equivalent of the first strand sequence comprises a sequence differing by no more than 2 nucleotides from any one of the first strand sequences of Table 1, and optionally wherein the unmodified equivalent of the second strand sequence comprises a sequence differing by no more than 2 nucleotides from the corresponding second strand sequence of Table 1;
(c) the unmodified equivalent of the first strand sequence comprises a sequence differing by no more than 1 nucleotide from any one of the first strand sequences of Table 1, and optionally wherein the unmodified equivalent of the second strand sequence comprises a sequence differing by no more than 1 nucleotide from the corresponding second strand sequence of Table 1;
(d) the unmodified equivalent of the first strand sequence comprises a sequence corresponding to nucleotides 2 to 17 from the 5′ end of any one of the first strand sequences of Table 1, and optionally wherein the unmodified equivalent of the second strand sequence comprises a sequence corresponding to nucleotides 2 to 17 from the 5′ end of the corresponding second strand sequence of Table 1;
(e) the unmodified equivalent of the first strand sequence comprises a sequence corresponding to nucleotides 2 to 18 from the 5′ end of any one of the first strand sequences of Table 1, and optionally wherein the unmodified equivalent of the second strand sequence comprises a sequence corresponding to nucleotides 2 to 18 from the 5′ end of the corresponding second strand sequence of Table 1;
(f) the unmodified equivalent of the first strand sequence comprises a sequence corresponding to nucleotides 2 to 19 from the 5′ end of any one of the first strand sequences of Table 1, and optionally wherein the unmodified equivalent of the second strand sequence comprises a sequence corresponding to nucleotides 2 to 19 from the 5′ end of the corresponding second strand sequence of Table 1;
(g) the unmodified equivalent of the first strand sequence comprises a sequence corresponding to nucleotides 2 to 19 from the 5′ end of any one of the first strand sequences of Table 1, and optionally wherein the unmodified equivalent of the second strand sequence comprises a sequence corresponding to nucleotides 1 to 18 from the 5′ end of the corresponding second strand sequence of Table 1;
(h) the unmodified equivalent of the first strand sequence comprises a sequence of any one of the first strand sequences of Table 1, and optionally wherein the unmodified equivalent of the second strand sequence comprises a sequence of the corresponding second strand sequence of Table 1; or
(i) the unmodified equivalent of the first strand sequence essentially consists of any one of the first strand sequences of Table 1, and optionally wherein the unmodified equivalent of the second strand sequence essentially consists of the sequence of the corresponding second strand sequence of Table 1.
(j) the unmodified equivalent of the first strand sequence consists of any one of the first strand sequences of Table 1, and optionally wherein the unmodified equivalent of the second strand sequence consists of the sequence of the corresponding second strand sequence of Table 1.
(k) the unmodified equivalent of the first strand sequence consists of a sequence corresponding to nucleotides 1 to 19 from the 5′ end of any one of the first strand sequences with a given SEQ ID No. shown in Table 1, wherein said unmodified equivalent of the first strand sequence further consists of 1 (nucleotide 20 counted from the 5′end), 2 (nucleotides 20 and 21), 3 (nucleotides 20, 21 and 22), 4 (nucleotides 20, 21, 22 and 23), 5 (nucleotides 20, 21, 22, 23 and 24) or 6 (nucleotides 20, 21, 22, 23, 24 and 25) additional nucleotide(s) at the 3′end of any one of the first strand sequences with a given SEQ ID No. shown in Table 1, and optionally wherein the unmodified equivalent of the second strand sequence comprises or consists essentially of or consists of a sequence of the corresponding second strand sequence with a given SEQ ID No. shown Table 1;
(l) the unmodified equivalent of the first strand sequence consists of a sequence corresponding to nucleotides 1 to 19 from the 5′ end of any one of the first strand sequences with a given SEQ ID No. shown in Table 1, wherein said unmodified equivalent of the first strand sequence further consists of 1 (nucleotide 20 counted from the 5′end), 2 (nucleotides 20 and 21), 3 (nucleotides 20, 21 and 22), 4 (nucleotides 20, 21, 22 and 23), 5 (nucleotides 20, 21, 22, 23 and 24) or 6 (nucleotides 20, 21, 22, 23, 24 and 25) additional nucleotide(s) at the 3′end of any one of the first strand sequences with a given SEQ ID No. shown in Table 1, and wherein said unmodified equivalent of the first strand sequence consists of a contiguous region of from 17-25 nucleotides in length, preferably of from 18-24 nucleotides in length, complementary to the MASP-2 transcript of SEQ ID NO. 821; and optionally wherein the unmodified equivalent of the second strand sequence comprises or consists essentially of or consists of a sequence of the corresponding second strand sequence with a given SEQ ID No. shown in Table 1;
(m) unmodified equivalent of the first strand and the unmodified equivalent of the second strand of any one of the nucleic acid molecules of subsections (a) to (l) above are present on a single strand wherein the unmodified equivalent of the first strand and the unmodified equivalent of the second strand are able to hybridise to each other and to thereby form a double-stranded nucleic acid with a duplex region of 17, 18, 19, 20, 21, 22, 23, 24 or 25 nucleotides in length; or
(n) the unmodified equivalent of the first strand and the unmodified equivalent of the second strand of any one of the nucleic acid molecules of subsections (a) to (l) above are on two separate strands that are able to hybridise to each other and to thereby form a double-stranded nucleic acid with a duplex region of 17, 18, 19, 20, 21, 22, 23, 24 or 25 nucleotides in length.
10. The nucleic acid of any one of the preceding statements, wherein at least one nucleotide of the first and/or second strand is a modified nucleotide.
11. The nucleic acid of statement 10, wherein at least nucleotides 2 and 14 of the first strand are modified by a first modification, the nucleotides being numbered consecutively starting with nucleotide number 1 at the 5′ end of the first strand with a given SEQ ID No.
12 The nucleic acid of statement 10 or statement 11, wherein each of the even-numbered nucleotides of the first strand are modified by a first modification, the nucleotides being numbered consecutively starting with nucleotide number 1 at the 5′ end of the first strand with a given SEQ ID No.
13. The nucleic acid of statement 11 or statement 12, wherein the odd-numbered nucleotides of the first strand with a given SEQ ID No. are modified by a second modification, wherein the second modification is different from the first modification.
14. The nucleic acid of any one of statements 11 to 13, wherein the nucleotides of the second strand with a given SEQ ID No. in a position corresponding to an even-numbered nucleotide of the first strand with a given SEQ ID No. are modified by a third modification, wherein the third modification is different from the first modification.
15. The nucleic acid of any one of statements 11 to 14, wherein the nucleotides of the second strand with a given SEQ ID No. in a position corresponding to an odd-numbered nucleotide of the first strand with a given SEQ ID No. are modified by a fourth modification, wherein the fourth modification is different from the second modification and different from the third modification when a second and/or a third modification are present.
16. The nucleic acid of any one of statements 11 to 13, wherein the nucleotide/nucleotides of the second strand with a given SEQ ID No. in a position corresponding to nucleotide 11 or nucleotide 13 or nucleotides 11 and 13 or nucleotides 11-13 of the first strand with a given SEQ ID No. is/are modified by a fourth modification and preferably wherein the nucleotides of the second strand that are not modified by a fourth modification are modified by a third modification.
17. The nucleic acid of any one of statements 11 to 16, wherein the first modification is the same as the fourth modification if both modifications are present in the nucleic acid and preferably wherein the second modification is the same as the third modification if both modifications are present in the nucleic acid.
18. The nucleic acid of any one of statements 11 to 17, wherein the first modification is a 2′-F modification; the second modification, if present in the nucleic acid, is preferably a 2′-OMe modification; the third modification, if present in the nucleic acid, is preferably a 2′-OMe modification; and the fourth modification, if present in the nucleic acid, is preferably a 2′-F modification.
19. The nucleic acid of any one of statements 10 to 18, wherein each of the nucleotides of the first strand and of the second strand is a modified nucleotide.
20. The nucleic acid of any one of statements 10 to 19, wherein the first strand with a given SEQ ID No. has a terminal 5′ (E)-vinylphosphonate nucleotide at its 5′ end and wherein the terminal 5′ (E)-vinylphosphonate nucleotide is preferably linked to the second nucleotide in the first strand by a phosphodiester linkage.
21. The nucleic acid of any one of the preceding statements, wherein the nucleic acid comprises a phosphorothioate linkage between the terminal two or three 3′ nucleotides and/or 5′ nucleotides of the first strand with a given SEQ ID No. and/or the second strand with a given SEQ ID No. and preferably wherein the linkages between the remaining nucleotides are phosphodiester linkages.
22. The nucleic acid of any one of statements 1 to 20, comprising a phosphorodithioate linkage between each of the two, three or four terminal nucleotides at the 3′ end of the first strand with a given SEQ ID No. and/or comprising a phosphorodithioate linkage between each of the two, three or four terminal nucleotides at the 3′ end of the second strand with a given SEQ ID No. and/or a phosphorodithioate linkage between each of the two, three or four terminal nucleotides at the 5′ end of the second strand with a given SEQ ID No. and comprising a linkage other than a phosphorodithioate linkage between the two, three or four terminal nucleotides at the 5′ end of the first strand.
23. The nucleic acid of any one of statements 1 to 20, wherein the nucleic acid comprises a phosphorothioate linkage between each of the two, three of four terminal nucleotides at the 3′ end of the first strand with a given SEQ ID No. and/or between each of the two, three or four terminal nucleotides at the 5′end of the first strand with a given SEQ ID No., and/or between each of the two, three or four terminal nucleotides at the 3′end of the second strand with a given SEQ ID No. and/or between each of the two, three or four terminal nucleotides at the 5′end of the second strand with a given SEQ ID No.
24. The nucleic acid of any one of statements 1 to 20, wherein all the linkages between the nucleotides of both strands other than the linkage between the two terminal nucleotides at the 3′ end of the first strand and the linkages between the two terminal nucleotides at the 3′ end and at the 5′ end of the second strand are phosphodiester linkages.
25. The nucleic acid of any one of statements 10 to 24, wherein:
(a) the unmodified equivalent of the first strand sequence comprises a sequence differing by no more than 3 nucleotides from any one of the first strand sequences of Table 5b, and optionally wherein the unmodified equivalent of the second strand sequence comprises a sequence differing by no more than 3 nucleotides from the corresponding second strand sequence of Table 5b;
(b) the unmodified equivalent of the first strand sequence comprises a sequence differing by no more than 2 nucleotides from any one of the first strand sequences of Table 5b, and optionally wherein the unmodified equivalent of the second strand sequence comprises a sequence differing by no more than 2 nucleotides from the corresponding second strand sequence of Table 5b;
(c) the unmodified equivalent of the first strand sequence comprises a sequence differing by no more than 1 nucleotide from any one of the first strand sequences of Table 5b, and optionally wherein the unmodified equivalent of the second strand sequence comprises a sequence differing by no more than 1 nucleotide from the corresponding second strand sequence of Table 5b;
(d) the unmodified equivalent of the first strand sequence comprises a sequence corresponding to nucleotides 2 to 17 from the 5′ end of any one of the first strand sequences with a given SEQ ID No. shown in Table 5b, and optionally wherein the unmodified equivalent of the second strand sequence comprises a sequence corresponding to nucleotides 2 to 17 from the 5′ end of the corresponding second strand sequence with a given SEQ ID No. shown in Table 5b;
(e) the unmodified equivalent of the first strand sequence comprises a sequence corresponding to nucleotides 2 to 18 from the 5′ end of any one of the first strand sequences with a given SEQ ID No. shown in Table 5b, and optionally wherein the unmodified equivalent of the second strand sequence comprises a sequence corresponding to nucleotides 2 to 18 from the 5′ end of the corresponding second strand sequence with a given SEQ ID No. shown in Table 5b;
(f) the unmodified equivalent of the first strand sequence comprises a sequence corresponding to nucleotides 2 to 19 from the 5′ end of any one of the first strand sequences with a given SEQ ID No. shown in Table 5b, and optionally wherein the unmodified equivalent of the second strand sequence comprises a sequence corresponding to nucleotides 2 to 19 from the 5′ end of the corresponding second strand sequence with a given SEQ ID No. shown in Table 5b;
(g) the unmodified equivalent of the first strand sequence comprises a sequence corresponding to nucleotides 2 to 19 from the 5′ end of any one of the first strand sequences with a given SEQ ID No. shown in Table 5b, and optionally wherein the unmodified equivalent of the second strand sequence comprises a sequence corresponding to nucleotides 1 to 18 from the 5′ end of the corresponding second strand sequence with a given SEQ ID No. shown in Table 5b;
(h) the unmodified equivalent of the first strand sequence comprises a sequence of any one of the first strand sequences with a given SEQ ID No. shown in Table 5b, and optionally wherein the unmodified equivalent of the second strand sequence comprises a sequence of the corresponding second strand sequence with a given SEQ ID No. shown in Table 5b; or
(i) the unmodified equivalent of the first strand sequence essentially consists of any one of the first strand sequences with a given SEQ ID No. shown in Table 5b, and optionally wherein the unmodified equivalent of the second strand sequence essentially consists of the sequence of the corresponding second strand sequence with a given SEQ ID No. shown in Table 5.
(j) the unmodified equivalent of the first strand sequence consists of any one of the first strand sequences with a given SEQ ID No. shown in Table 5b, and optionally wherein the unmodified equivalent of the second strand sequence consists of the sequence of the corresponding second strand sequence with a given SEQ ID No. shown in Table 5.
26. The nucleic acid of any one of statements 10 to 25, wherein:
(a) the unmodified equivalent of the first strand sequence comprises a sequence differing by no more than 3 nucleotides from any one of the first strand sequences of Table 2, and optionally wherein the unmodified equivalent of the second strand sequence comprises a sequence differing by no more than 3 nucleotides from the corresponding second strand sequence of Table 2;
(b) the unmodified equivalent of the first strand sequence comprises a sequence differing by no more than 2 nucleotides from any one of the first strand sequences of Table 2, and optionally wherein the unmodified equivalent of the second strand sequence comprises a sequence differing by no more than 2 nucleotides from the corresponding second strand sequence of Table 2;
(c) the unmodified equivalent of the first strand sequence comprises a sequence differing by no more than 1 nucleotide from any one of the first strand sequences of Table 2, and optionally wherein the unmodified equivalent of the second strand sequence comprises a sequence differing by no more than 1 nucleotide from the corresponding second strand sequence of Table 2;
(d) the unmodified equivalent of the first strand sequence comprises a sequence corresponding to nucleotides 2 to 17 from the 5′ end of any one of the first strand sequences with a given SEQ ID No. shown in Table 2, and optionally wherein the unmodified equivalent of the second strand sequence comprises a sequence corresponding to nucleotides 2 to 17 from the 5′ end of the corresponding second strand sequence with a given SEQ ID No. shown in Table 2;
(e) the unmodified equivalent of the first strand sequence comprises a sequence corresponding to nucleotides 2 to 18 from the 5′ end of any one of the first strand sequences with a given SEQ ID No. shown in Table 2, and optionally wherein the unmodified equivalent of the second strand sequence comprises a sequence corresponding to nucleotides 2 to 18 from the 5′ end of the corresponding second strand sequence with a given SEQ ID No. shown in Table 2;
(f) the unmodified equivalent of the first strand sequence comprises a sequence corresponding to nucleotides 2 to 19 from the 5′ end of any one of the first strand sequences with a given SEQ ID No. shown in Table 2, and optionally wherein the unmodified equivalent of the second strand sequence comprises a sequence corresponding to nucleotides 2 to 19 from the 5′ end of the corresponding second strand sequence with a given SEQ ID No. shown in Table 2;
(g) the unmodified equivalent of the first strand sequence comprises a sequence corresponding to nucleotides 2 to 19 from the 5′ end of any one of the first strand sequences with a given SEQ ID No. shown in Table 2, and optionally wherein the unmodified equivalent of the second strand sequence comprises a sequence corresponding to nucleotides 1 to 18 from the 5′ end of the corresponding second strand sequence with a given SEQ ID No. shown in Table 2;
(h) the unmodified equivalent of the first strand sequence comprises a sequence of any one of the first strand sequences with a given SEQ ID No. shown in Table 2, and optionally wherein the unmodified equivalent of the second strand sequence comprises a sequence of the corresponding second strand sequence with a given SEQ ID No. shown in Table 2;
(i) the unmodified equivalent of the first strand sequence essentially consists of any one of the first strand sequences with a given SEQ ID No. shown in Table 2, and optionally wherein the unmodified equivalent of the second strand sequence essentially consists of the sequence of the corresponding second strand sequence with a given SEQ ID No. shown in Table 2;
(j) the unmodified equivalent of the first strand sequence consists of any one of the first strand sequences with a given SEQ ID No. shown in Table 2, and optionally wherein the unmodified equivalent of the second strand sequence consists of the sequence of the corresponding second strand sequence with a given SEQ ID No. shown in Table 2;
(k) the first strand sequence consists of a sequence corresponding to nucleotides 1 to 19 from the 5′ end of any one of the first strand sequences with a given SEQ ID No. shown in Table 2, wherein said first strand sequence further consists of 1 (nucleotide 20 counted from the 5′end), 2 (nucleotides 20 and 21), 3 (nucleotides 20, 21 and 22), 4 (nucleotides 20, 21, 22 and 23), 5 (nucleotides 20, 21, 22, 23 and 24) or 6 (nucleotides 20, 21, 22, 23, 24 and 25) additional nucleotide(s) at the 3′end of any one of the first strand sequences with a given SEQ ID No. shown in Table 2, and optionally wherein the second strand sequence comprises or consists essentially of or consists of a sequence of the corresponding second strand sequence with a given SEQ ID No. shown in Table 2;
(l) the first strand sequence consists of a sequence corresponding to nucleotides 1 to 19 from the 5′ end of any one of the first strand sequences with a given SEQ ID No. shown in Table 2, wherein said first strand sequence further consists of 1 (nucleotide 20 counted from the 5′end), 2 (nucleotides 20 and 21), 3 (nucleotides 20, 21 and 22), 4 (nucleotides 20, 21, 22 and 23), 5 (nucleotides 20, 21, 22, 23 and 24) or 6 (nucleotides 20, 21, 22, 23, 24 and 25) additional nucleotide(s) at the 3′end of any one of the first strand sequences with a given SEQ ID No. shown in Table 2, and wherein said first strand sequence consists of a contiguous region of from 17-25 nucleotides in length, preferably of from 18-24 nucleotides in length, complementary to the MASP-2 transcript of SEQ ID NO. 821, and
   optionally wherein the second strand sequence comprises or consists essentially of or consists of a sequence of the corresponding second strand sequence with a given SEQ ID No. shown in Table 2;
(m) the first strand and the second strand of any one of the nucleic acid molecules of subsections (a) to (l) above are present on a single strand wherein the first strand and the second strand are able to hybridise to each other and to thereby form a double-stranded nucleic acid with a duplex region of 17, 18, 19, 20, 21, 22, 23, 24 or 25 nucleotides in length; or
(n) the first strand and the second strand of any one of the nucleic acid molecules of subsections (a) to (l) above are on two separate strands that are able to hybridise to each other and to thereby form a double-stranded nucleic acid with a duplex region of 17, 18, 19, 20, 21, 22, 23, 24 or 25 nucleotides in length.
27. The nucleic acid of any one of the preceding statements, wherein the nucleic acid is conjugated to a heterologous moiety.
28. The nucleic acid of statement 27, wherein the heterologous moiety comprises (i) one or more N-acetyl galactosamine (GalNAc) moieties or derivatives thereof, and (ii) a linker, wherein the linker conjugates the at least one GalNAc moiety or derivative thereof to the nucleic acid.
29. The nucleic acid of statement 27 or statement 28, wherein the nucleic acid is conjugated to a heterologous moiety comprising a compound of formula (II):

[S—X¹—P—X²]3-A-X³— (II)
wherein:
   S represents a functional component, e.g., a ligand, such as a saccharide, preferably wherein the saccharide is N-acetyl galactosamine;
   X¹ represents C₃-C₆ alkylene or (—CH₂—CH₂—O)ₘ(—CH₂)₂— wherein m is 1, 2, or 3;
   P is a phosphate or modified phosphate, preferably a thiophosphate;
   X² is alkylene or an alkylene ether of the formula (—CH₂)ₙ—O—CH₂— where n=1-6;
   A is a branching unit;
   X₃ represents a bridging unit;
   wherein a nucleic acid as defined in any of statements 1 to 27 is conjugated to X³ via a phosphate or modified phosphate, preferably a thiophosphate.
30. The nucleic acid of any one of statements 27 to 29, wherein the first strand of the nucleic acid is a compound of formula (V):

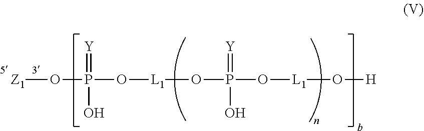

wherein b is 0 or 1; and
wherein the second strand is a compound of formula (VI):

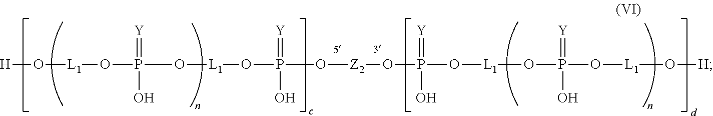

wherein:
   c and d are independently 0 or 1;
   Z₁ and Z₂ are respectively the first and second strand of the nucleic acid;
   Y is independently O or S;
   n is independently 0, 1, 2 or 3; and
   L₁ is a linker to which a ligand is attached, wherein L₁ is the same or different in formulae (V) and (VI), and is the same or different within formulae (V) and (VI) when L₁ is present more than once within the same formula;
and wherein b+c+d is 2 or 3.
31. The nucleic acid of any one of statements 27 to 30 which is one of the duplexes with a given Duplex ID No. shown in Table 5c.
32. A composition comprising a nucleic acid of any of the previous statements and a solvent and/or a delivery vehicle and/or a physiologically acceptable excipient and/or a carrier and/or a salt and/or a diluent and/or a buffer and/or a preservative.
33. A composition comprising a nucleic acid of any one of statements 1 to 31 and a further therapeutic agent selected from the group comprising an oligonucleotide, a small molecule, a monoclonal antibody, a polyclonal antibody and a peptide.
34. A nucleic acid of any one of statements 1 to 31 or a composition of statement 32 or 33 for use as a therapeutic agent.
35. A nucleic acid of any one of statements 1 to 31 or a composition of statement 32 or 33 for use in the prophylaxis or treatment of a disease, disorder or syndrome.
36. The nucleic acid or composition for use according to statement 35, wherein the disease, disorder or syndrome is a complement-mediated disease, disorder or syndrome.
37. The nucleic acid or composition for use according to statement 35 or 36, wherein the disease, disorder or syndrome is associated with aberrant activation or over-activation of the complement pathway and/or with over-expression or ectopic expression or localisation or accumulation of MASP-2.
38. The nucleic acid or composition for use according to any one of statements 35 to 37, wherein the disease, disorder or syndrome is:
   a) selected from the group comprising C3 glomerulopathy (C3G), paroxysmal nocturnal hemoglobinuria (PNH), atypical hemolytic uremic syndrome (aHUS), lupus nephritis, IgA nephropathy (IgA N), cold agglutinin disease (CAD), myasthenia gravis (MG), primary membranous nephropathy, immune complex-mediated glomerulonephritis (IC-mediated GN), post-infectious glomerulonephritis (PIGN), systemic lupus erythematosus (SLE), ischemia/reperfusion injury, age-related macular degeneration (AMD), rheumatoid arthritis (RA), antineutrophil cytoplasmic autoantibodies-associated vasculitis (ANCA-AV), dysbiotic periodontal disease, malarial anaemia, neuromyelitis optica, post-HCT/solid organ transplant (TMAs), Guillain-Barré syndrome, membranous glomerulonephritis, thrombotic thrombocytopenic purpura and sepsis;
   b) selected from the group comprising C3 glomerulopathy (C3G), paroxysmal nocturnal hemoglobinuria (PNH), atypical hemolytic uremic syndrome (aHUS), lupus nephritis, IgA nephropathy (IgA N) and primary membranous nephropathy;
   c) selected from the group comprising C3 glomerulopathy (C3G), antineutrophil cytoplasmic autoantibodies-associated vasculitis (ANCA-AV), atypical hemolytic uremic syndrome (aHUS), cold agglutinin disease (CAD), myasthenia gravis (MG), IgA nephropathy (IgA N), paroxysmal nocturnal hemoglobinuria (PNH);
   d) selected from the group comprising C3 glomerulopathy (C3G), cold agglutinin disease (CAD), myasthenia gravis (MG), neuromyelitis optica, atypical hemolytic uremic syndrome (aHUS), antineutrophil cytoplasmic autoantibodies-associated vasculitis (ANCA-AV), IgA nephropathy (IgA N), post-HCT/Solid Organ Transplant (TMAs), Guillain-Barré syndrome, paroxysmal nocturnal hemoglobinuria (PNH), membranous glomerulonephritis, lupus nephritis and thrombotic thrombocytopenic purpura
   e) selected from the group comprising C3 glomerulopathy (C3G), cold agglutinin disease (CAD) and IgA nephropathy (IgA N); or
   f) C3 glomerulopathy (C3G).
39. Use of a nucleic acid of any one of statements 1 to 31 or a composition of statement 32 or 33 in the preparation of a medicament for prophylaxis or treatment of a disease, disorder or syndrome.
40. A method of prophylaxis or treatment of a disease, disorder or syndrome comprising administering a pharmaceutically effective dose of a nucleic acid of any one of statements 1 to 31 or a composition of statement 32 or 33 to an individual in need of treatment, preferably wherein the nucleic acid or composition is administered to the subject subcutaneously, intravenously or by oral, rectal or intraperitoneal administration.

1. A double-stranded nucleic acid for inhibiting expression of MASP-2, wherein the nucleic acid comprises a first strand and a second strand, wherein the unmodified equivalent of the first strand sequence consists of a sequence from any one of the first strand sequences of SEQ ID No. 245, 147, 385, 199, 235, 237, 309, 381, 386, 387, 388, 389, 289.

2. The nucleic acid of claim 1, wherein the unmodified equivalent of the second strand sequence comprises a sequence from any one of the corresponding second strand sequences of SEQ ID No. 246, 148, 178, 200, 236, 238, 310, 346, 382, 384, 290.

3. The nucleic acid of claim 1, wherein the first strand and the second strand form a duplex region of from 17-25 nucleotides in length.

4. The nucleic acid of claim 1, wherein the nucleic acid mediates RNA interference.

5. The nucleic acid of claim 1, wherein the first strand sequence consists of SEQ ID No. 245 and wherein the second strand consists of SEQ ID No. 246.

6. The nucleic acid of claim 5, wherein at least one nucleotide of the first and/or second strand is a modified nucleotide.

7. The nucleic acid of claim 6, wherein the first strand has a terminal 5′ (E)-vinylphosphonate nucleotide at its 5′ end.

8. The nucleic acid of claim 6, wherein the nucleic acid comprises a phosphorothioate linkage between each of the terminal three 3′ nucleotides of the first strand.

9. The nucleic acid of claim 6, wherein the nucleic acid comprises a phosphorothioate linkage between each of the terminal three 3′ nucleotides of the second strand.

10. The nucleic acid of claim 8, wherein the linkages between the remaining nucleotides of the first strand and/or of the second strand are phosphodiester linkages.

11. The nucleic acid of claim 1, wherein the first strand sequence comprises (vp)-mU fC mG fG mA fG mC fG mG fA mA fG mG fU mA fA mU (ps) fG (ps) mU (SEQ ID No. 804) and optionally wherein the second strand sequence comprises mA mC mA mU mU mA fC fC fU mU mC mC mG mC mU mC mC (ps) mG (ps) mA (SEQ ID No. 820).

12. The nucleic acid of claim 1, wherein the nucleic acid is conjugated to a heterologous moiety.

13. The nucleic acid of claim 12, wherein the heterologous moiety comprises (i) one or more N-acetyl galactosamine (GalNAc) moieties or derivatives thereof, and (ii) a linker, wherein the linker conjugates the at least one GalNAc moiety or derivative thereof to the nucleic acid.

14. The nucleic acid of claim 13, wherein the first strand sequence comprises (vp)-mU fC mG fG mA fG mC fG mG fA mA fG mG fU mA fA mU (ps) fG (ps) mU (SEQ ID No. 804) and optionally wherein the second strand sequence comprises [ST23 (ps)]₃ ST41 (ps) mA mC mA mU mU mA fC fC fU mU mC mC mG mC mU mC mC (ps) mG (ps) mA (SEQ ID No. 805).

15. A composition comprising a nucleic acid of claim 1 and a solvent and/or a delivery vehicle and/or a physiologically acceptable excipient and/or a carrier and/or a salt and/or a diluent and/or a buffer and/or a preservative and/or a further therapeutic agent selected from the group comprising an oligonucleotide, a small molecule, a monoclonal antibody, a polyclonal antibody and a peptide.

16. A method of prophylaxis or treatment of a disease, disorder or syndrome comprising administering a pharmaceutically effective dose of a nucleic acid of claim 1 to an individual in need of treatment, wherein the disease, disorder or syndrome is
(a) selected from the group consisting of C3 glomerulopathy (C3G), paroxysmal nocturnal hemoglobinuria (PNH), atypical hemolytic uremic syndrome (aHUS), lupus nephritis, IgA nephropathy (IgA N), cold agglutinin disease (CAD), myasthenia gravis (MG), primary membranous nephropathy, immune complex-mediated glomerulonephritis (IC-mediated GN), post-infectious glomerulonephritis (PIGN), systemic lupus erythematosus (SLE), ischemia/reperfusion injury, age-related macular degeneration (AMD), rheumatoid arthritis (RA), antineutrophil cytoplasmic autoantibodies-associated vasculitis (ANCA-AV), dysbiotic periodontal disease, malarial anaemia, neuromyelitis optica, post-HCT/solid organ transplant (TMAs), Guillain-Barré syndrome, membranous glomerulonephritis, thrombotic thrombocytopenic purpura and sepsis;
(b) selected from the group consisting of C3 glomerulopathy (C3G), paroxysmal nocturnal hemoglobinuria (PNH), atypical hemolytic uremic syndrome (aHUS), lupus nephritis, IgA nephropathy (IgA N) and primary membranous nephropathy;
(c) selected from the group consisting of C3 glomerulopathy (C3G), antineutrophil cytoplasmic autoantibodies-associated vasculitis (ANCA-AV), atypical hemolytic uremic syndrome (aHUS), cold agglutinin disease (CAD), myasthenia gravis (MG), IgA nephropathy (IgA N), paroxysmal nocturnal hemoglobinuria (PNH);
(d) selected from the group consisting of C3 glomerulopathy (C3G), cold agglutinin disease (CAD), myasthenia gravis (MG), neuromyelitis optica, atypical hemolytic uremic syndrome (aHUS), antineutrophil cytoplasmic autoantibodies-associated vasculitis (ANCA-AV), IgA nephropathy (IgA N), post-HCT/Solid Organ Transplant (TMAs), Guillain-Barré syndrome, paroxysmal nocturnal hemoglobinuria (PNH), membranous glomerulonephritis, lupus nephritis and thrombotic thrombocytopenic purpura;
(e) selected from the group consisting of C3 glomerulopathy (C3G), cold agglutinin disease (CAD) and IgA nephropathy (IgA N); or
(f) C3 glomerulopathy (C3G).
